(19) United States
(12) Patent Application Publication
(10) Pub. No.: US 2025/0134907 A1
(43) Pub. Date: May 1, 2025

(54) FUSED DIAZEPINES AS AGONISTS OF THE INSULIN-LIKE 3 (INSL3) PEPTIDE RECEPTOR RXFP2 AND METHODS OF USE THEREOF

(71) Applicants: The United States of America,as represented by the Secretary,Department of Health and Human Services, Bethesda, MD (US); The Florida International University Board of Trustees, Miami, FL (US)

(72) Inventors: Juan J. Marugan, Gaithersburg, MD (US); Noel T. Southall, Potomac, MD (US); Marc Ferrer, Potomac, MD (US); Mark J. Henderson, Damascus, MD (US); Kenneth J. Wilson, Germantown, MD (US); Alexander I. Agoulnik, Doral, FL (US); Courtney B. Myhr, Coral Springs, FL (US); Maria Esteban-Lopez, Miami, FL (US); Elena Barnaeva, Poolesville, MD (US); Xin Hu, Frederick, MD (US); Wenjuan Ye, Gaithersburg, MD (US); Irina Agoulnik, Doral, FL (US)

(73) Assignees: The United States of America,as represented by the Secretary,Department of Health and Human Services, Bethesda, MD (US); The Florida International University Board of Trustees, Miami, FL (US)

(21) Appl. No.: 18/837,100

(22) PCT Filed: Jan. 31, 2023
(86) PCT No.: PCT/US2023/012013

§ 371 Date: Aug. 8, 2024

Related U.S. Application Data
(60) Provisional application No. 63/308,768, filed on Feb. 10, 2022.

(51) Int. Cl. A61K 31/5513; C07D 487/04

(52) U.S. Cl. CPC A61K 31/5513; C07D 487/04

(57) ABSTRACT

Disclosed is a compound of formula (I), in which R¹, R², R³, R⁴, R⁵, X¹, X², X³, X⁴, and X⁵ are described herein. The small molecule compounds of formula (I) activate the functional activity of relaxin family peptide receptor 2 (RXFP2), thereby providing therapeutic treatments for a variety of disorders, such as a bone disorder, hypogonadism, cryptorchidism, polycystic ovary syndrome, cancer, infertility, or an ocular wound.

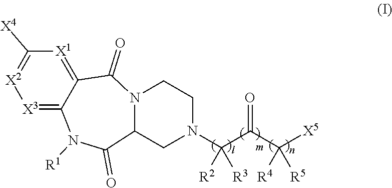

### CROSS-REFERENCE TO A RELATED APPLICATION

This application claims the benefit of U.S. Provisional Patent Application No. 63/308,768, filed Feb. 10, 2022, the disclosure of which is incorporated herein in its entirety for all purposes.

### STATEMENT REGARDING FEDERALLY SPONSORED RESEARCH OR DEVELOPMENT

This invention was made with Government support under project number 1ZIATR000086-06 by the National Institutes of Health, National Center for Advancing Translational Sciences (NCATS) and under project number 1R01AR070093 by the National Institutes of Health, National Institute of Arthritis and Musculoskeletal and Skin Diseases (NIAMS). The Government has certain rights in the invention.

### BACKGROUND OF THE INVENTION

Osteoporosis is a skeletal disorder characterized by low bone density and disrupted bone tissue architecture, which leads to the increased risk of fracture. Existing pharmacological treatments inhibit osteoclast bone-resorption activity or stimulate differentiation and increase the functional activity of osteoblasts. For example, bisphosphonates (e.g., alendronate) increase structural bone strength by promoting increased bone volume and density (Appelman-Dijkstra et al., Clinical Endocrinology & Metabolism, 28, 843-857 (2014)). However, with the approval of the receptor activator of NF-kappaB ligand (RANKL) inhibitor denosumab, the most potent antiresorptive agent, new approaches in this class of therapeutics have been mainly exhausted. In contrast, the use of parathyroid hormone peptides is the first and only available anabolic approach to stimulate osteoblast differentiation through activation of several cellular transduction pathways, including canonical WNT signaling. However, peptide treatment requires daily injections and is expensive. Other agents such as sclerostin, GSK-30, Dickkopf-1 (Dkk1) inhibitors, activins, bone morphogenetic protein (BMP) stimulators, and nitroglycerin have been tested in clinical trials with mixed success. All these drugs have some drawbacks. For example, bisphosphonate treatments are only beneficial for 3-5 years, while alendronate and reloxifene decrease fractures in the spine but not in other sites.

Insulin-like3 hormone is a peptide in the relaxin/insulin-like family. It is produced continuously after birth, primarily in testicular Leydig cells and follicular ovarian cells (Sozubir et al., J Urol, 183, 2373-2379 (2010); and Nef et al., Nat Genet, 22, 295-299 (1999)). It was discovered that human patients with mutations of the INSL3 G protein-coupled receptor (RXFP2) manifested osteoporosis or osteopenia (Ferlin et al., Journal of Bone andMineral Research, 23, 683-693 (2008)). The abnormalities detected in patients with non-functional alleles suggested the role of INSL3/RXFP2 signaling in bone metabolism. The level of INSL3 declines in men with the loss of normal testicular function during aging (Anand-Ivell et al., Int J Androl, 29, 618-626 (2006)) whereas the risk of osteoporosis increases. The XXY patients with Klinefelter Syndrome (KS), a disease associated with small testes, have a high risk of developing osteoporosis and osteopenia and a consequent increased risk of fractures.

Consistent with the human phenotype, bone histomorphometric, and micro computed tomography (pCT) analyses of Rxfp2−/−knockout mice generated showed decreased bone mass, mineralizing surface, bone formation, and osteoclast surface compared with wild-type littermates. These data suggested that the low bone mass phenotype in the Rxfp2−/− mice is linked to functional osteoblast impairment causing little bone formation, little mineralizing surface, and ultimately, a negative balance between bone formation and bone resorption.

Alternatively, relaxin peptide acting through its receptor RXFP1, expressed on human peripheral blood monocytes, regulate their differentiation into mature osteoclasts, osteoclast survival and activation (Ferlin et al., Bone, 46, 504-513 (2010)). Thus, two members of the same peptide family play opposing roles in bone metabolism: INSL3 stimulates bone growth, relaxin has the opposite effect. Studies in mice with the RXFP2 deletion and INSL3 overexpression show that INSL3 is the only cognate ligand for RXFP2 in vivo (Bogatcheva et al., Molecular Endocrinology, 17, 2639-2646 (2003)). Moreover, an analysis of ligand-receptor interaction suggests that INSL3 and relaxin utilize different binding modes and distinct sites within the receptor (Scott et al., Mol Endocrinol, 26(11), 1896-1906 (2012)).

Besides the use of RXFP2 agonists as therapeutic agents in the induction of bone differentiation, there are numerous other potential research and clinical applications for such activators. A major role for INSL3/RXFP2 signaling has been demonstrated in testicular descent and cryptorchidism, which typically is treated by orchiopexy surgery (Agoulnik et al., Methods in Molecular Biology, 825, 127-147 (2012); Bogatcheva et al., Reproductive Biomedicine online, 10, 49-54 (2005); and Ferguson et al., Frontiers in Endocrinology, 4, 32 (2013)). However, there are notable risks associated with orchiopexy surgery, such as infection, bleeding, anomalous healing, damage to the blood vessels and other structures in the spermatic cord resulting in the loss of testis, failure of the testis to remain in the scrotum, urination problems, pathology, and slow recovery. No reliable pharmacological intervention exists for treating cryptorchidism apart from a GNRH/hCG gonadotropin hormone regiment with an overall 15-20% success rate.

In addition, a role for INSL3/RXFP2 has been described in uterine structural integrity (Li et al., Endocrinology, 152, 2474-2482 (2011)), as a female anti-apoptotic germ cell agent (Nef et al., Nat Genet, 22, 295-299 (1999); amd Spanel-Borowski et al., Mol Reprod Dev, 58, 281-286 (2001)), and as a therapeutic agent in wound healing at the ocular surface (Hampel et al., Endocrinology, 154, 2034-2045 (2013)). Importantly, the INSL3 overexpression transgenic model showed that the excess of INSL3 had no effect on survival, development (other than gonadal position), viability, or fertility of mutant mice (Adham et al., Mol Endocrinol, 16, 244-252 (2002)). The INSL3/RXFP2 system also has been observed in certain cancers, including prostate carcinoma and human thyroid carcinoma tissues (Esteban-Lopez et al., Journal of Endocrinology, 247 (1), R1-R12 (2020)).

To date, no small molecule agonists of RXFP2 have been reported in the literature. Thus, there remains an unmet need to provide small molecule compounds that activate the functional activity of RXFP2 to be used in various therapeutic methods, including treating osteoporosis, hypogonadism, and cancer.

### BRIEF SUMMARY OF THE INVENTION

The invention provides a compound of formula (I)

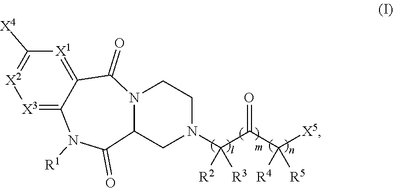

in which R¹, R², R³, R⁴, R⁵, X¹, X², X³, X⁴, and X⁵ are as described herein.

The small molecule compounds have been discovered to activate the functional activity of relaxin family peptide receptor 2 (RXFP2), thereby providing therapeutic treatments for a variety of disorders, such as a bone disorder, hypogonadism, cryptorchidism, polycystic ovary syndrome, cancer, infertility, or an ocular wound.

### BRIEF DESCRIPTION OF THE SEVERAL VIEWS OF THE DRAWINGS

FIG. 1 is a chemical scheme of the synthesis of 8-iodo-1,3,4,12a-tetrahydrobenzo[e]pyrazino[1,2-a][1,4]diazepine6,12 (2H, 11H)-dione core.

FIG. 2 is a chemical scheme of the synthesis of 8-bromo-1,3,4,12a-tetrahydrobenzo[e]pyrazino[1,2-a]-[1,4]diazepine-6,12 (2H, 11H)-dione 2,2,2-trifluoroacetate core.

FIG. 3 is a chemical scheme of the synthesis of racemic Eastern hemisphere substituted derivatives 1.

FIG. 4 is a chemical scheme of the synthesis of substituted 2-phenoxyacetic acids.

FIG. 5 is a chemical scheme of the synthesis of N-alkylated Eastern hemisphere compounds.

FIG. 6 is a chemical scheme of the synthesis of racemic Eastern hemisphere substituted derivatives 2.

FIG. 7 is a chemical scheme of the synthesis of N-carbonylated piperazines.

FIG. 8 is a chemical scheme of the synthesis of racemic Western hemisphere substituted derivatives.

FIG. 9 is a chemical scheme of the synthesis of 2-(2-oxo-2-phenylethyl)-8-(3-(trifluoromethyl)phenyl)-1,3,4,12a-tetrahydrobenzo[e]pyrazino[1,2-a][1,4]diazepine-6,12 (2H, 11H)-diones.

FIG. 10 is a chemical scheme of the synthesis of (R)-2-(2-(2-chloro-4-(trifluoromethoxy)phenoxy)acetyl)-8-(3-methyl-5-(trifluoromethyl)phenyl)-1,3,4,11,12,12a-hexahydrobenzo[e]pyrazino[1,2-a][1,4]diazepin-6 (2H)-one-2,2,2-trifluoroacetate.

FIG. 11 is a chemical scheme of the synthesis of 4-((tert-butoxycarbonyl)amino)-[1,1′-biphenyl]-3-carboxylic acids and 2-amino-5-substituted phenylnicotinic acids, 5-amino-2-substituted phenylisonicotinic acids, and 3-amino-6-substituted phenylpicolinic acids.

FIG. 12 is a chemical scheme of the synthesis of Northern hemisphere des-carbonyl compound (S)-8-(3,5-bis(trifluoromethyl)phenyl)-2-(2-(2-iodo-4-(trifluoromethoxy)phenoxy)acetyl)-1,3,4,6,11,12a-hexahydrobenzo[e]pyrazino[1,2-a][1,4]diazepin-12 (2H)-one.

FIG. 13 is a graph of the RXFP2 agonist efficacy in HEK-CRE-Luc-RXFP2 cells for compounds 1715 (●), 4340 (▴), 4337 (▪), and INSL3 (*).

FIGS. 14A and 14B are the graphs of the RXFP2 agonist specificity in HEK-RXFP1 cells.

FIGS. 15A and 15 B are tables of the RXFP2 agonist PRESTO-Tango GPCRome screening in HTLA cells for the compound of Example 187 (FIG. 15A) and the compound of Example 158 (FIG. 15B).

FIGS. 16A and 16B are graphs of the RXFP2 agonist cytotoxicity in HEK293T cells for the compounds of Examples 123 (●), 153 (▴), and 158 (▪) (FIG. 16A) and in HCO cells for the compounds of Examples 187 (●), 153 (▴), and 158 (▪) (FIG. 16B).

FIGS. 17A-C are graphs of the RXFP2 agonist efficacy in HEK293T cells transiently transfected with mouse RXFP2 for the compounds of Examples 123 (FIG. 17A), 153 (FIG. 17B), and 158 (FIG. 17C).

FIGS. 18A and 18B are graphs of the effects of INSL3 antagonist on INSL3 and RXFP2 agonist induced cAMP responses in HEK-RXFP2 cells for INSL3 B dimer (FIG. 18A) and the compound of Example 187 (FIG. 18B).

FIG. 19 is a graph of the surface and total expression of the chimeric receptors normalized to the expression of the WT receptor RXFP2-1 (white bars) and RXFP1-2 (black bars) (means±SEM of 3 independent experiments). The expression of the RXFP2-1 chimera was normalized to the expression of WT RXFP2 and the RXFP1-2 normalized to WT RXFP1.

FIG. 20 is a graph of RXFP2 agonist mineralization activity in HCO cells.

FIG. 21 is a schematic representation of the assay principle and histological results.

FIG. 22 is a series of graphs demonstrating bone formation activity in vivo of the compound of Example 187. The measured microCT parameters were bone volume per tissue volume (BV/TV, %) (FIG. 22A), trabecular number (Tb.N, mm-1) (FIG. 22B), trabecular thickness (Tb.Th, mm) (FIG. 22C), and trabecular separation (Tb.Sp, mm) (FIG. 22D). The results represent the mean±SEM of 12-15 mice per group. *p<0.05 vs vehicle, two tailed unpaired Student's t-test.

FIG. 23 depicts the gene expression levels of osteoblast markers in tibias from WT and INSL3 female mice resulting from treatment with vehicle or compound of Example 187, measured by quantitative RT-PCR. Results represent the mean±SEM of 7 mice per group. *p<0.05, **p<0.01 vs. WT using Student's t-test.

FIG. 24 depicts the results of a pharmacokinetic study on the compound of Example 187, after one 3 mg/kg IV administration, one 10 mg/kg PO administration, and three 10 mg/kg PO administrations (QD*3) in female mice. FIG. 24A depicts the plasma profile. FIG. 24B depicts the liver profile and FIG. 24C depicts the bone profiles. The actual concentration (ng/g) is the detected value (ng/mL) multiplied by 4. Drug vehicle is 25% aq. 40% HP-b-CD-75% PEG300. Three mice were used per time point. Results are expressed as the mean SEM.

### DETAILED DESCRIPTION OF THE INVENTION

In an aspect, the invention provides a compound of formula (I)

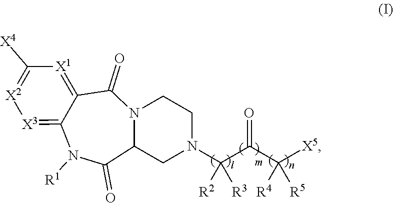

wherein

X¹, X², and X³ are the same or different and each is CH or N;

X⁴ is an optionally substituted aryl or heteroaryl;

X⁵ is selected from the group consisting of

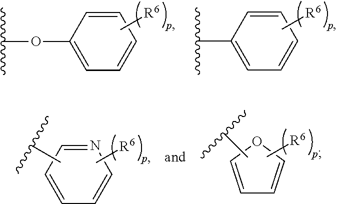

R¹ is hydrogen or alkyl;

R², R³, R⁴, and R^{S} are the same or different and each is hydrogen, alkyl, or halo;

each instance of R⁶ is the same or different and is selected from the group consisting of alkyl, cycloalkyl, heterocycloalkyl, halo, haloalkyl, hydroxy, cyano, nitro, amino, alkylamino, dialkylamino, alkoxy, haloalkoxy, alkylthio, alkylsulfonyl, haloalkylsulfonyl, alkylsulfonamido, carboxylato, —C(═NH)OH, aryl, heteroaryl, and 2-oxoazetidinyl; or

two instances of R⁶ along with the cyclic moiety to which they are bound form phenyl, cycloalkyl, or —O—C(R⁷R⁸)—O—;

R⁷ and R⁸ are the same or different and each is hydrogen, alkyl, or halo;

l is 0 or an integer of 1 to 6;

m is 0 or 1;

n is 0 or an integer of 1 to 6; and

p is 0 or an integer of 1 to 4;

or a pharmaceutically acceptable salt and/or an enantiomer thereof.

It has been surprisingly discovered that the small molecule compound of formula (I) acts as an RXFP2 agonist. Small molecule agonists of RXFP2 are preferred over peptide ligands due to improved stability, potential oral bioavailability, and/or reduced production cost. In some aspects, the compound of formula (I) does not have relaxin receptor agonism, thereby minimizing and possibly preventing potential side effects in cardiovascular, renal, reproductive and other systems, where relaxin signaling is important.

In an aspect of the compound of formula (I), X¹, X², and X³ are each CH to form a compound of formula (Ia) or a pharmaceutically acceptable salt and/or an enantiomer thereof

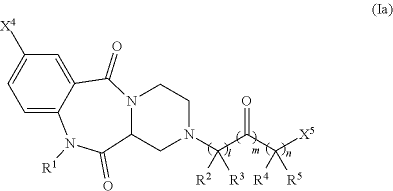

In an aspect of the compound of formula (I), one of X¹, X², and X³ is N and the remaining two moieties are each CH to form a compound of formula (Ib), (Ic), or (Id):

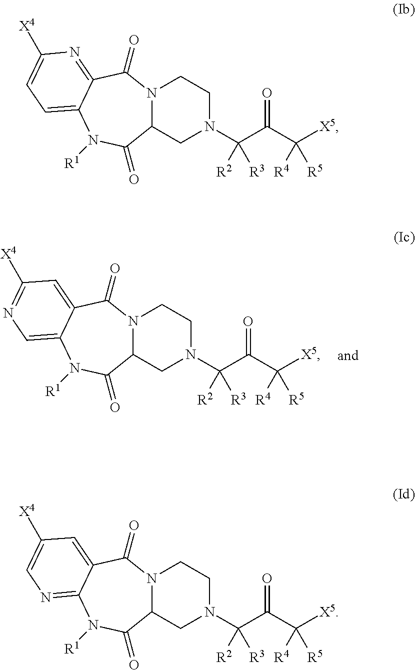

In any of the foregoing aspects of the compound of formula (I), R¹ preferably is hydrogen.

In any of the foregoing aspects of the compound of formula (I), R², R³, R⁴, and R^{S} preferably are each hydrogen.

In any of the foregoing aspects of the compound of formula (I), X⁴ is selected from the group consisting of

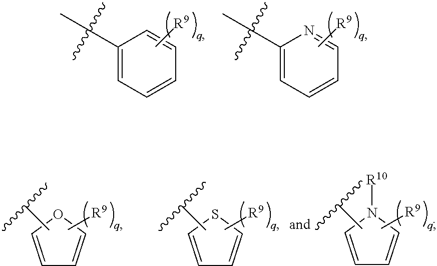

wherein

each instance of R⁹ is the same or different and is selected from the group consisting of alkyl, cycloalkyl, halo, haloalkyl, hydroxy, cyano, nitro, amino, alkylamino, dialkylamino, alkoxy, haloalkoxy, alkylthio, alkylsulfonyl, carboxylato, and —C(═NH)OH; or

two instances of R⁹ along with the cyclic moiety to which they are bound form phenyl (e.g., to form naphthyl, quinolinyl, etc.), cycloalkyl, or —O—C(R⁷R⁸)—O—;

R⁷ and R⁸ are the same or different and each is hydrogen, alkyl, or halo;

R¹⁰ is hydrogen or alkyl; and

q is 0 or an integer of 1 to 4.

In a preferred aspect, X⁴ is

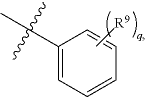

each instance of R⁹ is the same or different and is selected from the group consisting of alkyl, cycloalkyl, halo, haloalkyl, hydroxy, cyano, nitro, haloalkoxy, alkylthio, alkylsulfonyl, and —C(═NH)OH; or two instances of R⁹ along with the cyclic moiety to which they are bound form —O—C(R⁷R⁸)—O—; R⁷ and R⁸ are the same or different and each is hydrogen, alkyl, or halo; and q is 0 or an integer of 1 to 3. Even more preferably, each instance of R⁹ is the same or different and is selected from the group consisting of alkyl, cyclopropyl, fluoro, chloro, trifluoromethyl, and hydroxy; and q is 1 or 2.

In a preferred aspect, X⁴ is

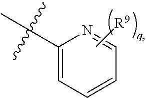

in which each instance of R⁹ is the same or different and is selected from the group consisting of alkyl, cycloalkyl, halo, haloalkyl, nitro, amino, alkylamino, dialkylamino, and haloalkoxy; and q is 0 or an integer of 1 to 2.

In a preferred aspect, X⁴ is

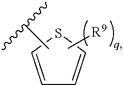

in which R⁹ is selected from the group consisting of alkyl, halo, and haloalkyl; and q is 0 or 1.

In any of these aspects of X⁴, at least one R⁹ subsituent is —CF₃.

In any of the foregoing aspects of the compound of formula (I), X⁵ is

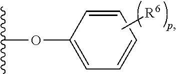

in which each instance of R⁶ is the same or different and is selected from the group consisting of alkyl, pyrrolidinyl, halo, haloalkyl, cyano, alkoxy, haloalkoxy, alkylthio, alkylsulfonyl, haloalkylsulfonyl, alkylsulfonamido, and 2-oxoazetidinyl; or two instances of R⁶ along with the cyclic moiety to which they are bound form phenyl or —O—C(R⁷R⁸)—O—; R⁷ and R⁸ are the same or different and each is hydrogen or halo; 1 is 0; m is 1; n is an integer of 1 to 6; and p is 0 or an integer of 1 to 4.

In an aspect of this aspect of X⁵, the compound of formula (I) preferably is of formula (Ie):

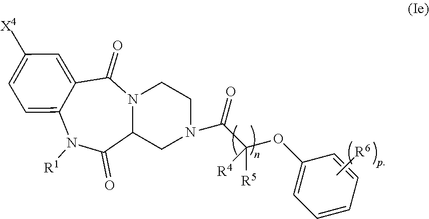

In a preferred aspect of formula (Ie), R¹, R⁴, and R^{S} are each hydrogen, n is 1, and X⁴, R⁶, and p are as described herein, including a racemic mixture, S enantiomer, or R enantiomer thereof. In another preferred aspect of formula (Ie), X⁴ is 3-trifluoromethylbenzyl, R¹, R⁴, and R⁴ are each hydrogen, n is 1, and R⁶ and p are as described herein, including a racemic mixture, S enantiomer, or R enantiomer thereof.

In an aspect of this aspect of X⁵, the compound of formula (I) preferably is of formula (If), (Ig), or (Ih):

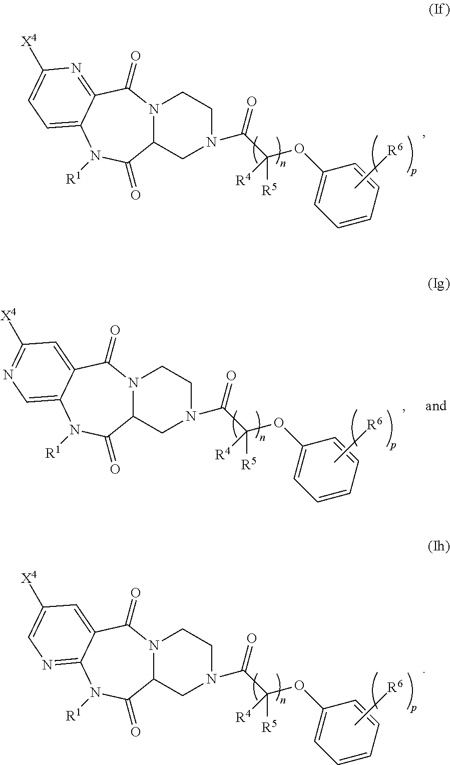

In a preferred aspect of formula (If), (Ig), and (Ih), R¹, R⁴, and R⁵ are each hydrogen, n is 1, and X⁴, R⁶, and p are as described herein, including a racemic mixture, S enantiomer, or R enantiomer thereof.

In any of the foregoing aspects of the compound of formula (I):
(i) p is 1, and R⁶ is fluoro, trifluoromethyl, cyano, trifluoromethoxy, difluoromethoxy, methylthio, methylsulfonyl, trifluoromethylsulfonyl, methylsulfon(methyl)amido, and 2-oxoazetidinyl; or
(ii) p is 2, and each instance of R⁶ is the same or different and is selected from the group consisting of methyl, fluoro, chloro, bromo, iodo, trifluoromethyl, cyano, methoxy, trifluoromethoxy, and difluoromethoxy; or
two instances of R⁶ along with the cyclic moiety to which they are bound form phenyl or —O—CF₂—O—; or
(iii) p is 3, and each instance of R⁶ is the same or different and is selected from the group consisting of methyl, fluoro, trifluoromethyl, cyano, and trifluoromethoxy; or
(iv) p is 4, and each instance of R⁶ is the same or different and is selected from the group consisting of methyl and cyano.

In any of the foregoing aspects of the compound of formula (I), X⁵ is

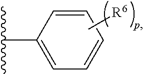

in which each instance of R⁶ is the same or different and is selected from the group consisting of alkyl, cycloalkyl, heterocycloalkyl, halo, haloalkyl, hydroxy, cyano, nitro, amino, alkylamino, dialkylamino, alkoxy, haloalkoxy, alkylthio, alkylsulfonyl, haloalkylsulfonyl, alkylsulfonamido, carboxylato, —C(═NH)OH, aryl, heteroaryl, and 2-oxoazetidinyl; or two instances of R⁶ along with the cyclic moiety to which they are bound form phenyl or —O—C(R⁷R⁸)—O—; R⁷ and R⁸ are the same or different and each is hydrogen, alkyl, or halo. In such aspect of X⁵, preferably either
(i) l is 0; m is 0; and n is an integer of 1 to 6;
(ii) l is 0; m is 1; and n is 0; or
(iii) l is an integer of 1 to 6; m is 1; and n is 0;
and
p is 0 or an integer of 1 to 3.

In a preferred aspect of this aspect of X⁵, each instance of R⁶ is the same or different and is selected from the group consisting of hydroxy and alkoxy; or two instances of R⁶ along with the cyclic moiety to which they are bound form —O—CH₂—O—.

In any of the foregoing aspects of the compound of formula (I), X⁵ is

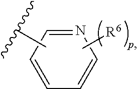

in which each instance of R⁶ is the same or different and is selected from the group consisting of alkyl, cycloalkyl, heterocycloalkyl, halo, haloalkyl, hydroxy, cyano, nitro, amino, alkylamino, dialkylamino, alkoxy, haloalkoxy, alkylthio, alkylsulfonyl, haloalkylsulfonyl, alkylsulfonamido, carboxylato, —C(═NH)OH, aryl, heteroaryl, and 2-oxoazetidinyl; or two instances of R⁶ along with the cyclic moiety to which they are bound form phenyl; l is 0; m is 0; and n is an integer of 1 to 6; and p is 0 or an integer of 1 to 3.

In a preferred aspect of this aspect of X⁵, each instance of R⁶ is the same or different and is selected from the group consisting of halo and haloalkyl; or two instances of R⁶ along with the cyclic moiety to which they are bound form phenyl; and p is 0 or an integer of 1 or 2.

In any of the foregoing aspects of the compound of formula (I), X⁵ is

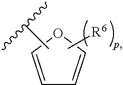

in which each instance of R⁶ is the same or different and is selected from the group consisting of alkyl, cycloalkyl, heterocycloalkyl, halo, haloalkyl, hydroxy, cyano, nitro, amino, alkylamino, dialkylamino, alkoxy, haloalkoxy, alkylthio, alkylsulfonyl, haloalkylsulfonyl, alkylsulfonamido, carboxylato, —C(═NH)OH, aryl, heteroaryl, and 2-oxoazetidinyl; or two instances of R⁶ along with the cyclic moiety to which they are bound form phenyl; 1 is 0; m is 1; and n is 0; and p is 0 or an integer of 1 to 3.

In a preferred aspect of this aspect of X⁵, each instance of R⁶ is haloalkyl, or two instances of R⁶ along with the cyclic moiety to which they are bound form phenyl; and p is 1 or 2.

The compound of formula (I) can have any suitable stereochemistry and can be in the form of a single stereoisomer, a mixture of two or more stereoisomers (e.g., an epimer, a mixture of diastereomers and/or enantiomers, a racemic mixture). In an aspect, the compound of formula (I) has the following stereochemistry:

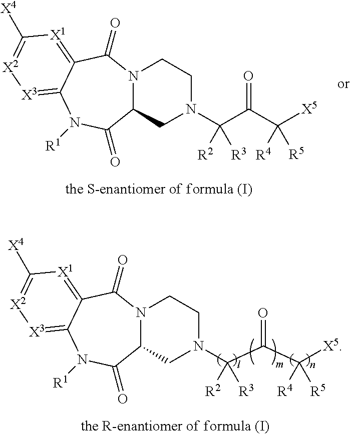

In an aspect, the compound of formula (I) is an S-enantiomer. In other aspects, the compound of formula (I) is an R-enantiomer. In some aspects, the compound of formula (I) is a racemic mixture.

Exemplary compounds of formula (I) are set forth in the examples and Tables 1-6 and includes racemic mixtures and enantiomers thereof

TABLE 1
Western hemisphere substitutions

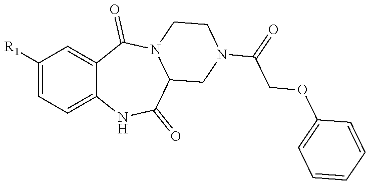

| CMPD | R₁ |
|---|---|
| KJW006-068 EXAMPLE 6 | 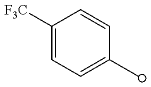 |
| KJW006-070 EXAMPLE 7 | 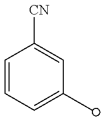 |
| KJW006-071 EXAMPLE 8 | 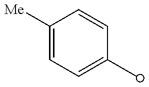 |
| KJW006-072 EXAMPLE 9 | 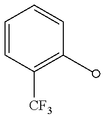 |
| KJW006-073 EXAMPLE 10 | 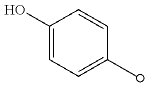 |
| KJW006-075 EXAMPLE 11 | 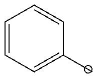 |
| KJW007-020 EXAMPLE 12 | 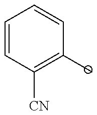 |
| KJW013-014 EXAMPLE 13 | 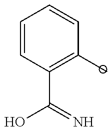 |
| KJW013-014 EXAMPLE 14 | 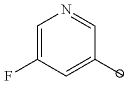 |
| KJW007-022 EXAMPLE 15 | 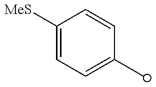 |
| KJW007-023 EXAMPLE 16 | 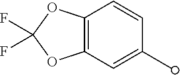 |
| KJW007-025 EXAMPLE 17 | 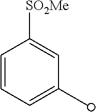 |
| KJW007-027 EXAMPLE 18 | 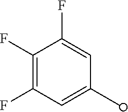 |
| KJW007-028 EXAMPLE 19 | 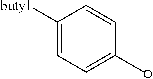 |
| KJW007-029 EXAMPLE 20 | 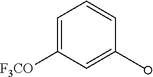 |
| KJW007-030 EXAMPLE 21 | 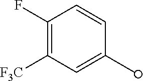 |
| KJW007-031 EXAMPLE 22 | 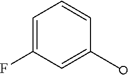 |
| KJW007-026 EXAMPLE 23 | 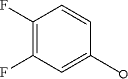 |
| KJW013-006 EXAMPLE 27 | 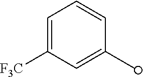 |

TABLE 2
Eastern hemisphere substitutions

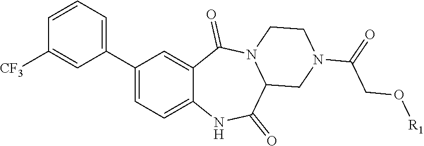

| CMPD | R₁ |
|---|---|
| KJW008-052 EXAMPLE 31 | 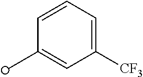 |
| KJW008-053 EXAMPLE 33 | 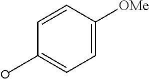 |
| KJW008-079 EXAMPLE 38 | 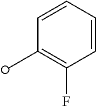 |
| KJW008-099 EXAMPLE 43 | 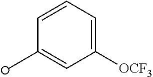 |
| KJW008-100 EXAMPLE 45 | 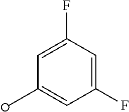 |
| KJW009-002 EXAMPLE 47 | 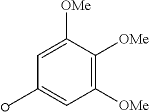 |
| KJW009-003 EXAMPLE 49 | 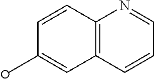 |
| KJW009-012 EXAMPLE 51 | 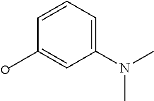 |
| KJW009-005 EXAMPLE 53 | 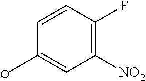 |
| KJW009-036 EXAMPLE 55 | 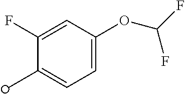 |
| KJW009-093 EXAMPLE 57 | 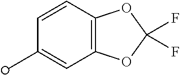 |
| KJW009-094 EXAMPLE 59 | 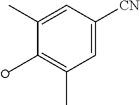 |
| KJW009-099 EXAMPLE 61 | 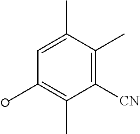 |
| KJW010-049 EXAMPLE 64 | 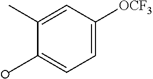 |
| KJW010-004 EXAMPLE 66 | 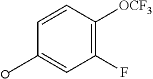 |
| KJW010-005 EXAMPLE 68 | 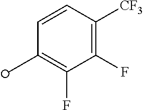 |
| KJW007-031 EXAMPLE 70 | 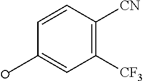 |
| KJW010-012 EXAMPLE 72 | 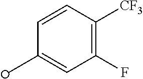 |
| KJW010-008 EXAMPLE 74 | 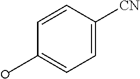 |
| KJW010-020 EXAMPLE 78 | 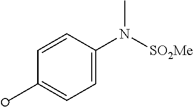 |
| KJW010-021 EXAMPLE 82 | 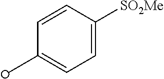 |
| KJW010-017 EXAMPLE 86 | 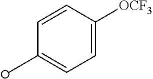 |
| KJW010-015 EXAMPLE 90 | 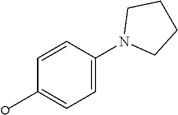 |
| KJW010-033 EXAMPLE 96 | 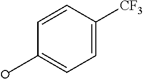 |
| KJW010-068 EXAMPLE 101 | 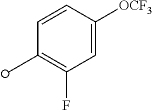 |
| KJW010-018 EXAMPLE 76 | 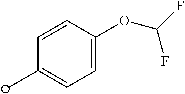 |
| KJW010-022 EXAMPLE 80 | 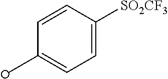 |
| KJW010-016 EXAMPLE 84 | 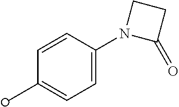 |
| KJW010-019 EXAMPLE 88 | 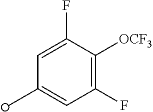 |
| KJW010-011 EXAMPLE 94 | 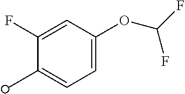 |
| KJW010-054 EXAMPLE 98 | 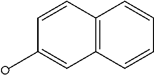 |

TABLE 3
Eastern and Western hemisphere substitutions

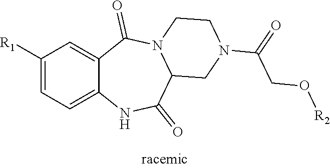

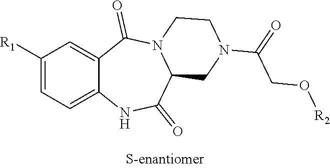

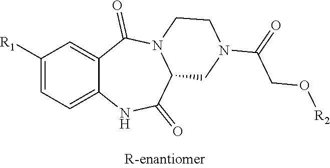

| CMPD | R₁ | R₂ | configuration |
|---|---|---|---|
| KJW010-100 EXAMPLE 104 | 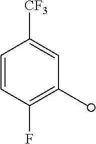 | 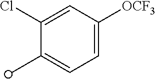 | RACEMIC |
| KJW011-002 EXAMPLE 106 | 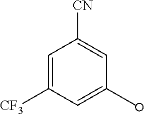 | 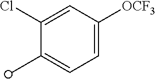 | RACEMIC |
| KJW011-004 EXAMPLE 107 | 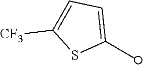 | 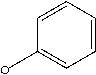 | RACEMIC |
| KJW010-045 EXAMPLE 108 | 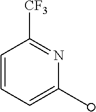 | 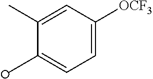 | RACEMIC |
| KJW010-083 EXAMPLE 112 | 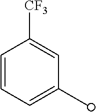 | 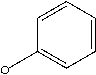 | (S) |
| KJW010-082 EXAMPLE 115 | 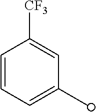 | 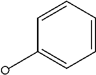 | (R) |
| KJW010-095 EXAMPLE 117 | 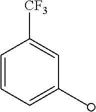 | 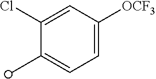 | (S) |
| KJW010-096 EXAMPLE 118 | 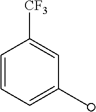 | 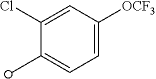 | (R) |
| KJW011-005 EXAMPLE 120 | 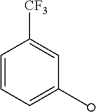 | 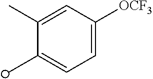 | (S) |
| KJW011-006 EXAMPLE 121 | 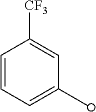 | 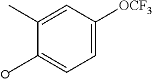 | (R) |
| KJW011-042 EXAMPLE 123 | 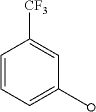 | 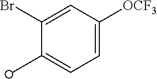 | (S) |
| KJW011-046 EXAMPLE 127 | 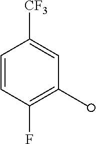 | 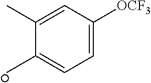 | (S) |
| KJW011-063 EXAMPLE 130 | 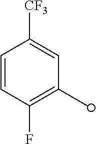 | 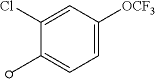 | (R) |
| KJW011-047 EXAMPLE 132 | 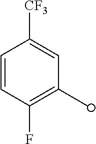 | 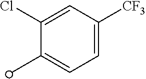 | (S) |
| KJW011-053 EXAMPLE 136 | 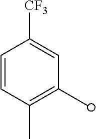 | 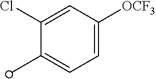 | (S) |
| KJW011-060 EXAMPLE 137 | 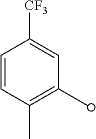 | 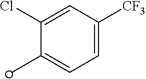 | (R) |
| KJW011-055 EXAMPLE 141 | 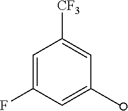 | 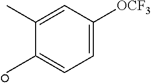 | (S) |
| KJW011-059 EXAMPLE 142 | 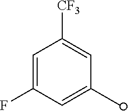 | 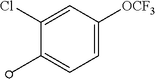 | (S) |
| KJW011-066 EXAMPLE 146 | 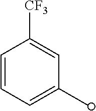 | 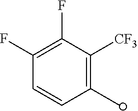 | (S) |
| KJW011-067 EXAMPLE 150 | 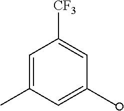 | 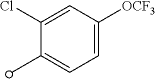 | (S) |
| KJW011-078 EXAMPLE 153 | 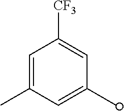 | 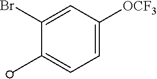 | (S) |
| KJW011-079 EXAMPLE 157 | 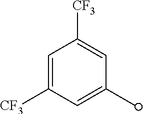 | 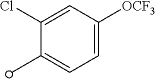 | (S) |
| KJW011-080 EXAMPLE 158 | 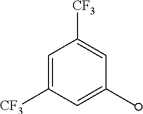 | 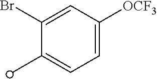 | (S) |
| KJW011-092 EXAMPLE 162 | 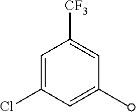 | 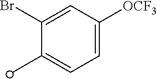 | (S) |
| KJW011-093 EXAMPLE 163 | 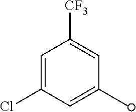 | 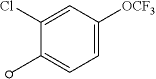 | (S) |
| KJW011-059 EXAMPLE 142 | 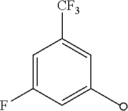 | 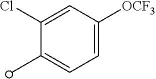 | (S) |
| KJW011-066 EXAMPLE 146 | 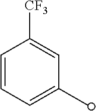 | 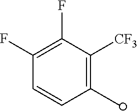 | (S) |
| KJW011-067 EXAMPLE 150 | 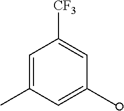 | 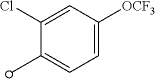 | (S) |
| KJW011-078 EXAMPLE 153 | 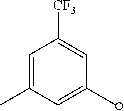 | 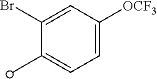 | (S) |
| KJW011-079 EXAMPLE 157 | 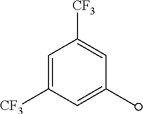 | 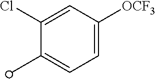 | (S) |
| KJW011-080 EXAMPLE 158 | 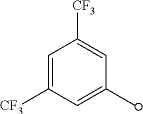 | 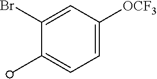 | (S) |
| KJW011-092 EXAMPLE 162 | 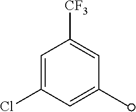 | 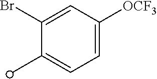 | (S) |
| KJW011-093 EXAMPLE 163 | 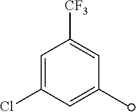 | 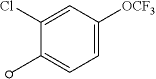 | (S) |
| KJW011-094 EXAMPLE 164 | 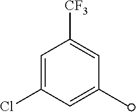 | 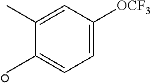 | (S) |
| KJW011-099 EXAMPLE 166 | 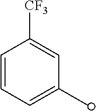 | 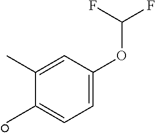 | (S) |
| KJW011-100 EXAMPLE 167 | 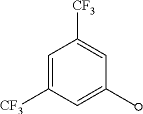 | 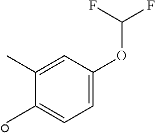 | (S) |
| KJW012-001 EXAMPLE 168 | 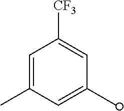 | 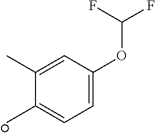 | (S) |
| KJW012-004 EXAMPLE 172 | 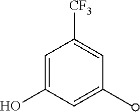 | 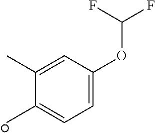 | (S) |
| KJW012-005 EXAMPLE 173 | 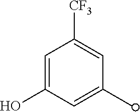 | 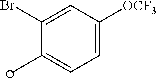 | (S) |
| KJW012-015 EXAMPLE 177 | 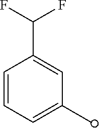 | 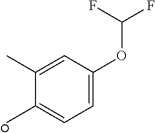 | (S) |
| KJW012-016 EXAMPLE 178 | 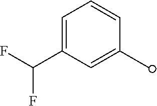 | 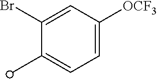 | (S) |
| KJW012-017 EXAMPLE 179 | 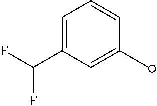 | 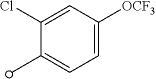 | (S) |
| KJW012-018 EXAMPLE 180 | 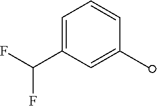 | 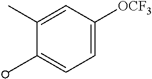 | (S) |
| KJW011-013 EXAMPLE 183 | 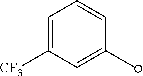 | 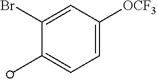 | (R) |
| KJW012-050 EXAMPLE 185 | 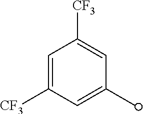 | 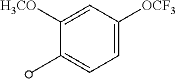 | (S) |
| KJW012-055 EXAMPLE 187 | 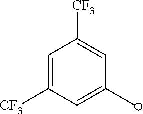 | 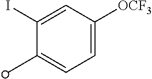 | (S) |
| KJW012-056 EXAMPLE 190 | 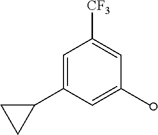 | 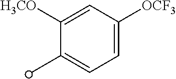 | (S) |
| KJW012-015 EXAMPLE 191 | 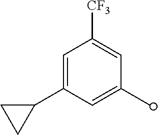 | 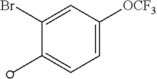 | (S) |
| KJW012-058 EXAMPLE 192 | 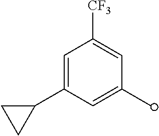 | 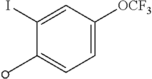 | (S) |

TABLE 4
Pyrazine Eastern and Western hemisphere substitutions

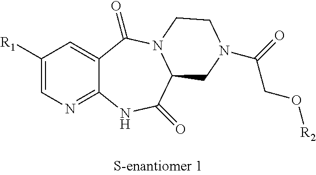

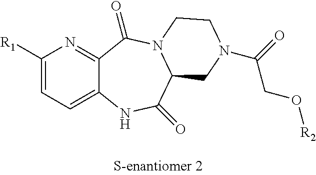

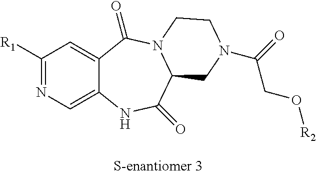

| CMPD | R₁ | R₂ | derivative |
|---|---|---|---|
| KJW012-058 EXAMPLE 196 | 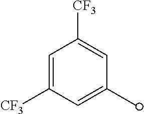 | 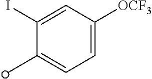 | S-enantiomer 3 |
| KJW012-081 EXAMPLE 197 | 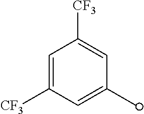 | 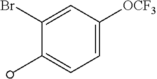 | S-enantiomer 3 |
| KJW012-081 EXAMPLE 197 | 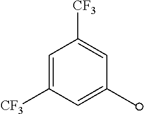 | 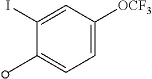 | S-enantiomer 3 |
| KJW012-083 EXAMPLE 202 | 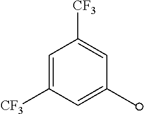 | 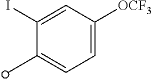 | S-enantiomer 1 |
| KJW012-100 EXAMPLE 206 | 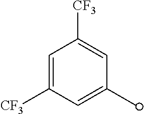 | 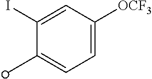 | S-enantiomer 2 |

TABLE 5

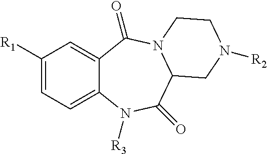

| CMPD | R₁ | R₂ | R₃ |
|---|---|---|---|
| KJW009-023 EXAMPLE 209 | 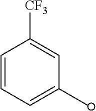 | 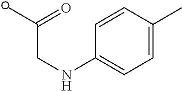 | H |
| KJW009-025 EXAMPLE 210 | 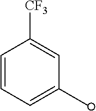 | 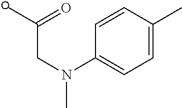 | CH₃ |
| KJW009-049 EXAMPLE 211 | 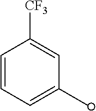 | 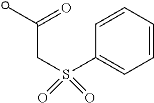 | H |
| KJW008-060 EXAMPLE 213 | 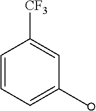 | 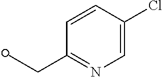 | H |
| KJW008-062 EXAMPLE 215 | 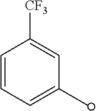 | 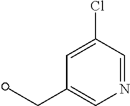 | H |
| KJW008-081 EXAMPLE 217 | 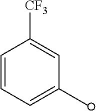 | 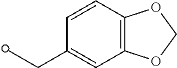 | H |
| KJW008-082 EXAMPLE 219 | 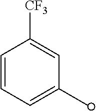 | 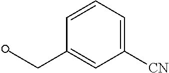 | H |
| KJW008-090 EXAMPLE 221 | 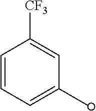 | 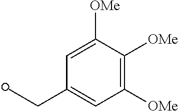 | H |
| KJW008-061 EXAMPLE 223 | 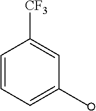 | 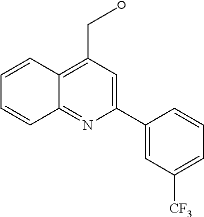 | H |
| KJW009-025 EXAMPLE 225 | 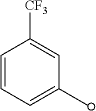 | 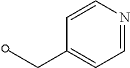 | H |
| KJW008-064 EXAMPLE 227 | 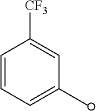 | 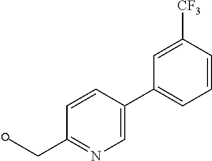 | H |
| KJW008-075 EXAMPLE 229 | 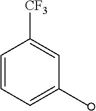 | 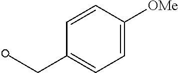 | H |
| KJW008-081 EXAMPLE 217 | 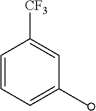 | 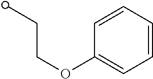 | H |
| KJW013-013 EXAMPLE 233 | 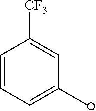 | 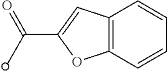 | H |
| KJW010-051-1 EXAMPLE 235 | 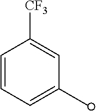 | 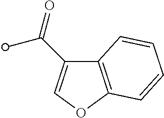 | H |
| KJW008-057 EXAMPLE 36 | 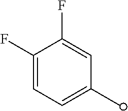 | 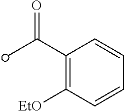 | H |
| KJW008-056 EXAMPLE 35 | 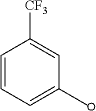 | 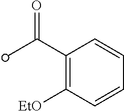 | H |
| KJW008-071 EXAMPLE 238 | 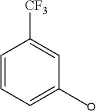 | 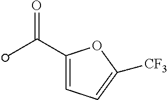 | H |
| KJW008-072 EXAMPLE 240 | 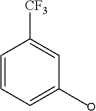 | 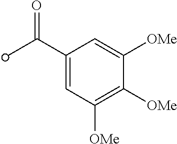 | H |
| KJW008-093 EXAMPLE 242 | 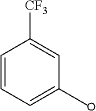 | 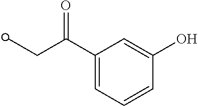 | H |
| KJW008-095 EXAMPLE 244 | 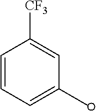 | 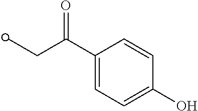 | H |
| KJW009-037 EXAMPLE 246 | 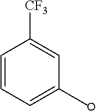 | 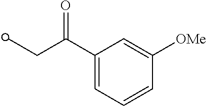 | H |
| KJW009-038 EXAMPLE 248 | 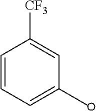 | 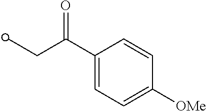 | H |

TABLE 6
Des-carbonyl diazapineone compounds

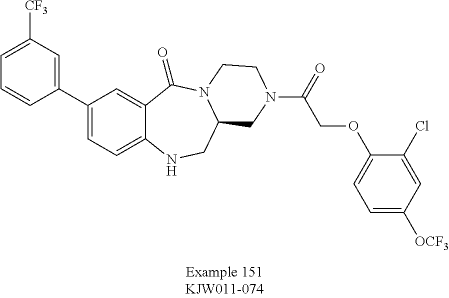 [SMILES: O=C(COc1ccc(OC(F)(F)F)cc1Cl)N1CCN2C(=O)c3cc(-c4cccc(C(F)(F)F)c4)ccc3NC[C@@H]2C1]

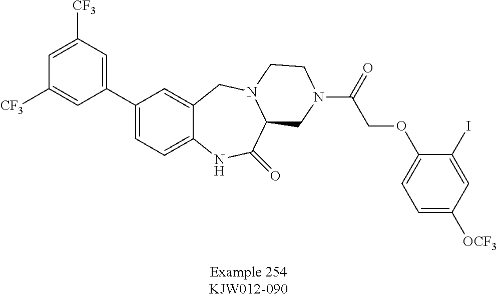 [SMILES: O=C1Nc2ccc(-c3cc(C(F)(F)F)cc(C(F)(F)F)c3)cc2CN2CCN(C(=O)COc3ccc(OC(F)(F)F)cc3I)C[C@@H]12]

In a preferred aspect, the compound of formula (I) is

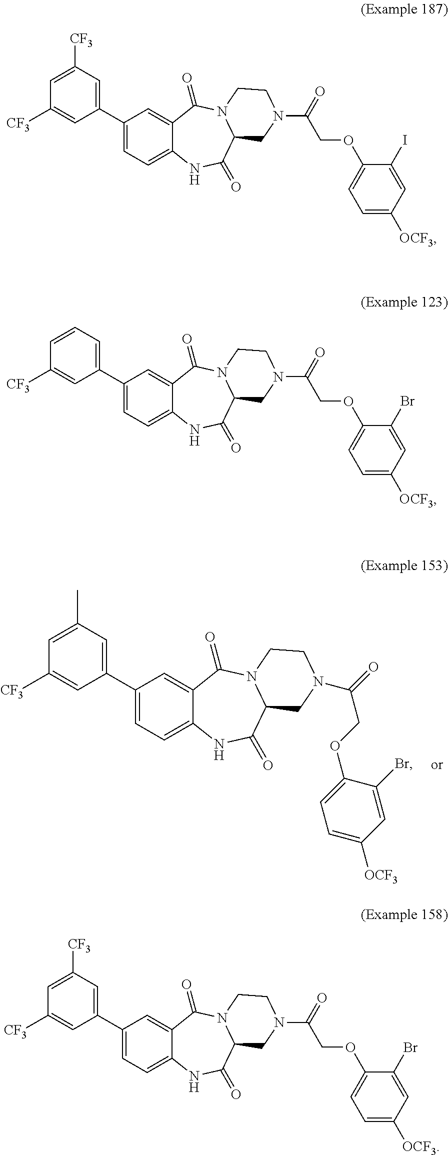 [SMILES: O=C1Nc2ccc(-c3cc(C(F)(F)F)cc(C(F)(F)F)c3)cc2C(=O)N2CCN(C(=O)COc3ccc(OC(F)(F)F)cc3I)C[C@@H]12 ; O=C1Nc2ccc(-c3cccc(C(F)(F)F)c3)cc2C(=O)N2CCN(C(=O)COc3ccc(OC(F)(F)F)cc3Br)C[C@@H]12 ; Cc1cc(-c2ccc3c(c2)C(=O)N2CCN(C(=O)COc4ccc(OC(F)(F)F)cc4Br)C[C@H]2C(=O)N3)cc(C(F)(F)F)c1 ; O=C1Nc2ccc(-c3cc(C(F)(F)F)cc(C(F)(F)F)c3)cc2C(=O)N2CCN(C(=O)COc3ccc(OC(F)(F)F)cc3Br)C[C@@H]12]

A compound of formula (I) can be provided using any suitable synthetic method, including the methods described herein and depicted in the figures.

In any of the aspects above, the term “alkyl” implies a straight-chain or branched alkyl substituent containing from, for example, from about 1 to about 8 carbon atoms, e.g., from about 1 to about 6 carbon atoms, from about 1 to about 4 carbon atoms. Examples of alkyl group include methyl, ethyl, n-propyl, isopropyl, n-butyl, sec-butyl, isobutyl, tert-butyl, n-pentyl, isopentyl, n-hexyl, heptyl, octyl, and the like. This definition also applies wherever “alkyl” occurs as part of a group, such as, e.g., in C₃-C₆ cycloalkylalkyl, hydroxyalkyl, haloalkyl (e.g., monohaloalkyl, dihaloalkyl, and trihaloalkyl), cyanoalkyl, aminoalkyl, alkylamino, dialkylamino, alkylaminoalkyl, dialkylaminoalkyl, arylcarbonylalkyl (-(alkyl)C(O)aryl), arylalkyl, etc. The alkyl can be substituted or unsubstituted, as described herein. Even in instances in which the alkyl is an alkylene chain (e.g., —(CH₂)ₙ—, in which n is 1 to 10, 1 to 8, 1 to 6, 1 to 4, 1 to 3,1 to 2, or 2), the alkyl group can be substituted or unsubstituted as described herein.

In any of the aspects above, the term “cycloalkyl,” as used herein, means a cyclic alkyl moiety containing from, for example, 3 to 6 carbon atoms or from 5 to 6 carbon atoms. Examples of such moieties include cyclopropyl, cyclobutyl, cyclopentyl, cyclohexyl, and the like. The cycloalkyl can be substituted or unsubstituted, as described herein.

In any of the aspects above, the term “aryl” refers to a mono, bi, or tricyclic carbocyclic ring system having one, two, or three aromatic rings, for example, phenyl, naphthyl, anthracenyl, or biphenyl. The term “aryl” refers to an unsubstituted or substituted aromatic carbocyclic moiety, as commonly understood in the art, and includes monocyclic and polycyclic aromatics such as, for example, phenyl, biphenyl, naphthyl, anthracenyl, pyrenyl, and the like. An aryl moiety generally contains from, for example, 6 to 30 carbon atoms, from 6 to 18 carbon atoms, from 6 to 14 carbon atoms, or from 6 to 10 carbon atoms. It is understood that the term aryl includes carbocyclic moieties that are planar and comprise 4n+2 xT electrons, according to Hickel's Rule, wherein n=1, 2, or 3. This definition also applies wherever “aryl” occurs as part of a group, such as, e.g., in haloaryl (e.g., monohaloaryl, dihaloaryl, and trihaloaryl), arylalkyl, etc. The aryl can be substituted or unsubstituted, as described herein.

In any of the aspects above, the term “heteroaryl” refers to aromatic 5 or 6 membered monocyclic groups, 9 or 10 membered bicyclic groups, and 11 to 14 membered tricyclic groups which have at least one heteroatom (O, S, or N) in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized, and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. Illustrative examples of heteroaryl groups are pyridinyl, pyridazinyl, pyrimidyl, pyrazinyl, benzimidazolyl, triazinyl, imidazolyl, (1,2,3)- and (1,2,4)-triazolyl, pyrazinyl, tetrazolyl, furyl, pyrrolyl, thienyl, isothiazolyl, thiazolyl, isoxazolyl, and oxadiazolyl. The heteroaryl can be substituted or unsubstituted, as described herein.

The term “heterocycloalkyl” means a stable, saturated, or partially unsaturated monocyclic, bicyclic, and spiro ring system containing 3 to 7 ring members of carbon atoms and other atoms selected from nitrogen, sulfur, and/or oxygen. In an aspect, a heterocycloalkyl is a 5, 6, or 7-membered monocyclic ring and contains one, two, or three heteroatoms selected from nitrogen, oxygen, and sulfur. The heterocycloalkyl may be attached to the parent structure through a carbon atom or through any heteroatom of the heterocycloalkyl that results in a stable structure. Examples of such heterocycloalkyl rings are isoxazolyl, thiazolinyl, imidazolidinyl, piperazinyl, homopiperazinyl, pyrrolyl, pyrrolinyl, pyrazolyl, pyranyl, piperidyl, oxazolyl, and morpholinyl. The heterocycloalkyl can be substituted or unsubstituted, as described herein.

In any of the aspects above, the term “hydroxy” refers to the group —OH.

In any of the aspects above, the term “cyano” refers to the group —CN.

In any of the aspects above, the terms “alkoxy” and “haloalkoxy” embrace linear or branched alkyl and haloalkyl groups, respectively, that are attached to a divalent oxygen. The alkyl and haloalkyl groups are the same as described herein.

In any of the aspects above, the term “halo” refers to a halogen selected from fluorine, chlorine, bromine, and iodine.

In any of the aspects above, the term “carboxylato” refers to the group —C(O)OH.

In any of the aspects above, the term “amino” refers to the group —NH₂. The term “alkylamino” refers to —NHR, whereas the term “dialkylamino” refers to —NRR′. R and R′ are the same or different and each is a substituted or unsubstituted alkyl group, as described herein.

In any of the aspects above, the term “amido” refers to the group —C(O)NRR′, which R and R′ are the same or different and each is hydrogen (“amido”) or a substituted or unsubstituted alkyl group (“alkylamido”), as described herein.

In any of the aspects above, the term “alkylthio” refers to the group —SR, in which R is a substituted or unsubstituted alkyl group, as described herein.

In any of the aspects above, the term “alkylsulfonyl” refers to the group —SO₂R, in which R is a substituted or unsubstituted alkyl group, as described herein. A “haloalkylsulfonyl” refers to an alkylsulfonyl that includes at least one halo substituent on the alkyl moiety.

In any of the aspects above, the term “alkylsulfonamido” refers to the group —NR′SO₂R, in which R is a substituted or unsubstituted alkyl group and R′ is hydrogen or a substituted or unsubstituted, as described herein.

In any of the aspects above, the term “2-oxoazetidinyl” refers to the group

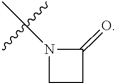 [SMILES: *N1CCC1=O]

In other aspects, any substituent that is not hydrogen (e.g., alkyl, cycloalkyl, cycloalkylalkyl, aryl, arylalkyl, heteroaryl, heteroarylalkyl, heterocycloalkyl, or heterocycloalkylalkyl) can be an optionally substituted moiety. The substituted moiety typically comprises at least one substituent (e.g., 1, 2, 3, 4, 5, 6, etc.) in any suitable position (e.g., 1-, 2-, 3-, 4-, 5-, or 6-position, etc.). When an aryl group is substituted with a substituent, e.g., halo, amino, alkyl, OH, alkoxy, and others, the aromatic ring hydrogen is replaced with the substituent and this can take place in any of the available hydrogens, e.g., 2, 3, 4, 5, and/or 6-position wherein the 1-position is the point of attachment of the aryl group in the compound of the present invention. Suitable substituents include, e.g., halo, alkyl, alkenyl, hydroxy, nitro, cyano, amino, alkylamino, alkoxy, aryloxy, aralkoxy, carboxyl, carboxyalkyl, carboxyalkyloxy, amido, alkylamido, haloalkylamido, aryl, heteroaryl, and heterocycloalkyl, each of which is described herein. In some instances, the substituent is at least one alkyl, halo, and/or haloalkyl (e.g., 1 or 2).

In any of the aspects above, whenever a range of the number of atoms in a structure is indicated (e.g., a C₁₋₁₂, C₁₋₈, C₁₋₆, C₁₋₄, etc.), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, cycloalkyl, etc.) referenced herein encompasses and specifically describes 1, 2, 3, 4, 5, 6, 7, and/or 8 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 2-3 carbon atoms, 2-4 carbon atoms, 2-5 carbon atoms, 2-6 carbon atoms, 2-7 carbon atoms, 2-8 carbon atoms, 3-4 carbon atoms, 3-5 carbon atoms, 3-6 carbon atoms, 3-7 carbon atoms, 3-8 carbon atoms, 4-5 carbon atoms, 4-6 carbon atoms, 4-7 carbon atoms, 4-8 carbon atoms, etc., as appropriate).

The subscripts “1” and “n” represent the number of substituted or unsubstituted methylene repeat units. The subscripts 1 and n are either 0 or an integer from 1-6 (i.e., 1, 2, 3, 4, 5, or 6). When 1 and/or n is 0, then the compound does not contain the corresponding methylene repeat unit. In some aspects of formula (I), 1 is 0 and n is 1-6; both 1 and n are 0; or 1 is 1-6 and n is 0.

The subscript “m” represents the presence or absence of a carbonyl (—C(═O)—) moiety. The subscript m can be 0 (no carbonyl is present) or an integer of 1 (a carbonyl is present). In some aspects of formula (I), m preferably is 1.

The subscript “p” represents the number of R⁶ substituents. The subscript p can be 0 (no substituents are present) or an integer of 1-4 (i.e., 1, 2, or 3, or 4). In some aspects of formula (I), p preferably is 1.

The subscript “q” represents the number of R⁹ substituents. The subscript q can be 0 (no substituents are present) or an integer of 1-4 (i.e., 1, 2, or 3, or 4). In some aspects of formula (I), q preferably is 0, 1, or 2, more preferably 1.

In any of the aspects herein, the phrase “salt” or “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. For example, an inorganic acid (e.g., hydrochloric acid, sulfuric acid, phosphoric acid, or hydrobromic acid), an organic acid (e.g., oxalic acid, malonic acid, citric acid, fumaric acid, lactic acid, malic acid, succinic acid, tartaric acid, acetic acid, trifluoroacetic acid, gluconic acid, ascorbic acid, methylsulfonic acid, or benzylsulfonic acid), an inorganic base (e.g., sodium hydroxide, potassium hydroxide, calcium hydroxide, magnesium hydroxide, or ammonium hydroxide), an organic base (e.g., methylamine, diethylamine, triethylamine, triethanolamine, ethylenediamine, tris(hydroxymethyl)methylamine, guanidine, choline, or cinchonine), or an amino acid (e.g., lysine, arginine, or alanine) can be used. Generally, nonaqueous media such as ether, ethyl acetate, ethanol, isopropanol, or acetonitrile are typical. Lists of suitable salts are found in Remington's Pharmaceutical Sciences, 18th ed., Mack Publishing Company, Easton, PA, 1990, p. 1445, and Journal of Pharmaceutical Science, 66, 2-19 (1977). For example, they can be a salt of an alkali metal (e.g., sodium or potassium), alkaline earth metal (e.g., calcium), or ammonium of salt.

The methods described herein comprise administering, to a subject in need thereof, a compound of formula (I) or a pharmaceutically acceptable salt and/or enantiomer thereof in the form of a pharmaceutical composition. In particular, a pharmaceutical composition will comprise at least one compound of formula (I), including a compound of formula (Ia), (Ib), (Ic), (Id), (Ie), (If), (Ig), and (Ih), or a pharmaceutically acceptable salt and/or enantiomer thereof and a pharmaceutically acceptable carrier. The pharmaceutically acceptable excipients described herein, for example, carriers, vehicles, adjuvants, and diluents, are well-known to those who are skilled in the art and are readily available to the public. Typically, the pharmaceutically acceptable carrier is one that is chemically inert to the active compound(s) and one that has no detrimental side effects or toxicity under the conditions of use.

The pharmaceutical compositions can be administered as oral, sublingual, transdermal, subcutaneous, topical, absorption through epithelial or mucocutaneous linings, intravenous, intranasal, intraarterial, intraperitoneal, intramuscular, intratumoral, peritumoral, intraperitoneal, intrathecal, rectal, vaginal, or aerosol formulations. In some aspects, the pharmaceutical composition is administered orally or intravenously.

In accordance with any of the aspects, the compound of formula (I) or a pharmaceutically acceptable salt and/or enantiomer thereof can be administered orally to a subject in need thereof. Formulations suitable for oral administration can consist of (a) liquid solutions, such as an effective amount of the compound dissolved in diluents, such as water, saline, or orange juice and include an additive, such as cyclodextrin (e.g., α-, β-, or γ-cyclodextrin, hydroxypropyl cyclodextrin, 2-hydroxypropyl-β-cyclodextrin) or polyethylene glycol (e.g., PEG 300; PEG 400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, com starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound of formula (I) or a pharmaceutically acceptable salt and/or enantiomer thereof can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as PEG 300 or PEG 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, com, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (e) mixtures thereof.

The parenteral formulations will typically contain from about 0.5 to about 25% by weight of the compound of formula (I) in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compound of formula (I) can be made into an injectable formulation. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topically applied compositions are generally in the form of liquids (e.g., mouthwash), creams, pastes, lotions and gels. Topical administration includes application to the oral mucosa, which includes the oral cavity, oral epithelium, palate, gingival, and the nasal mucosa. In some aspects, the composition contains at least one active component and a suitable vehicle or carrier. It may also contain other components, such as an anti-irritant. The carrier can be a liquid, solid or semi-solid. In aspects, the composition is an aqueous solution, such as a mouthwash. Alternatively, the composition can be a dispersion, emulsion, gel, lotion or cream vehicle for the various components. In one aspect, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In aspects of the invention, the vehicle for topical application to the skin can include water, buffered solutions, various alcohols, glycols such as glycerin, lipid materials such as fatty acids, mineral oils, phosphoglycerides, collagen, gelatin and silicone based materials.

The compound of formula (I) or a pharmaceutically acceptable salt thereof, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

The dose administered to the subject, particularly human and other mammals, in accordance with the present invention should be sufficient to affect the desired response. One skilled in the art will recognize that dosage will depend upon a variety of factors, including the age, condition or disease state, predisposition to disease, genetic defect or defects, and body weight of the subject. The size of the dose will also be determined by the route, timing and frequency of administration as well as the existence, nature, and extent of any adverse side-effects that might accompany the administration of a particular compound of formula (I) and the desired effect. It will be appreciated by one of skill in the art that various conditions or disease states may require prolonged treatment involving multiple administrations.

The inventive methods comprise administering an effective amount of a compound of formula (I) or a pharmaceutically acceptable salt thereof. An “effective amount” means an amount sufficient to show a meaningful benefit in an individual, e.g., promoting bone growth, reducing bone loss, maintaining bone density, increasing vertebrae trabecular number and/or thickness, inhibiting of glomerular cell proliferation, maintaining muscle density, increasing wound healing, increasing corneal healing, promoting testis descent, treating polycystic ovary syndrome (PCOS), treating cryptorchidism, promoting germ cell survival, promoting Leydig cell maturation, promoting at least one aspect of tumor cell cytotoxicity (e.g., inhibition of growth, inhibiting survival of a cancer cell, reducing proliferation, reducing size and/or mass of a tumor (e.g., solid tumor)), or treatment, healing, prevention, delay of onset, halting, or amelioration of other relevant medical condition(s) associated with a particular disorder. The meaningful benefit observed in the subject can be to any suitable degree (e.g., 10% or more, 20% or more, 30% or more, 40% or more, 50% or more, 60% or more, 70% or more, 80% or more, or 90% or more). In some aspects, one or more symptoms of the disorder (e.g., osteoporosis, hypogonadism, cancer) are prevented, reduced, halted, or eliminated subsequent to administration of a compound of formula (I) or a pharmaceutically acceptable salt and/or enantiomer thereof, thereby effectively treating the disorder to at least some degree.

Effective amounts may vary depending upon the biological effect desired in the individual, condition to be treated, and/or the specific characteristics of the compound of formula (I) or a pharmaceutically acceptable salt and/or enantiomer thereof, and the individual. In this respect, any suitable dose of the compound of formula (I) or a pharmaceutically acceptable salt and/or enantiomer thereof can be administered to the subject (e.g., human), according to the type of disorder to be treated. Various general considerations taken into account in determining the “effective amount” are known to those of skill in the art and are described, e.g., in Gilman et al., eds., Goodman and Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the compound of formula (I) or a pharmaceutically acceptable salt and/or enantiomer thereof desirably comprises about 0.01 mg per kilogram (kg) of the body weight of the subject (mg/kg) or more (e.g., about 0.05 mg/kg or more, 0.1 mg/kg or more, 0.5 mg/kg or more, 1 mg/kg or more, 2 mg/kg or more, 3 mg/kg or more, 5 mg/kg or more, 10 mg/kg or more, 15 mg/kg or more, 20 mg/kg or more, 30 mg/kg or more, 40 mg/kg or more, 50 mg/kg or more, 75 mg/kg or more, 100 mg/kg or more, 125 mg/kg or more, 150 mg/kg or more, 175 mg/kg or more, 200 mg/kg or more, 225 mg/kg or more, 250 mg/kg or more, 275 mg/kg or more, 300 mg/kg or more, 325 mg/kg or more, 350 mg/kg or more, 375 mg/kg or more, 400 mg/kg or more, 425 mg/kg or more, 450 mg/kg or more, or 475 mg/kg or more) per day. Typically, the dose will be about 500 mg/kg or less (e.g., about 475 mg/kg or less, about 450 mg/kg or less, about 425 mg/kg or less, about 400 mg/kg or less, about 375 mg/kg or less, about 350 mg/kg or less, about 325 mg/kg or less, about 300 mg/kg or less, about 275 mg/kg or less, about 250 mg/kg or less, about 225 mg/kg or less, about 200 mg/kg or less, about 175 mg/kg or less, about 150 mg/kg or less, about 125 mg/kg or less, about 100 mg/kg or less, about 75 mg/kg or less, about 50 mg/kg or less, about 40 mg/kg or less, about 30 mg/kg or less, about 20 mg/kg or less, about 15 mg/kg or less, about 10 mg/kg or less, about 5 mg/kg or less, about 2 mg/kg or less, about 1 mg/kg or less, about 0.5 mg/kg or less, or about 0.1 mg/kg or less). Any two of the foregoing endpoints can be used to define a close-ended range, or a single endpoint can be used to define an open-ended range.

For purposes of the present invention, the term “subject” preferably is directed to a mammal. Mammals include, but are not limited to, the order Rodentia, such as mice, and the order Lagomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perissodactyla, including Equines (horses). It is most preferred that the mammals are of the order Primates, Cebids, or Simioids (monkeys) or of the order Anthropoids (humans and apes). An especially preferred mammal is a human.

In an aspect, a compound of formula (I) or a pharmaceutically acceptable salt and/or enantiomer thereof activates the functional activity of (i.e., agonizes) relaxin family peptide receptor 2 (RXFP2). The RXFP2 activity can be measured by any suitable assay, including those described herein. Accordingly, the invention provides a method of activating the functional activity of RXFP2 in a cell comprising contacting the cell with the compound of formula (I) or a pharmaceutically acceptable salt and/or enantiomer thereof. The cell can be in vivo or ex vivo and can be from any suitable tissue whose cells express RXFP2. Suitable tissue includes, for example, bone, eye, muscle, ovary, uterus, testis, prostate, thyroid, muscle, brain, and combinations thereof.

Agonism of the INSL3/RXFP2 signaling pathway is considered a viable treatment of disorders associated with RXFP2, including mutations of RXFP2. Accordingly, the invention provides a method of treating a disorder mediated by RXFP2 in a subject comprising administering the compound of formula (I) or a pharmaceutically acceptable salt and/or enantiomer thereof to the subject. The disorder mediated by RXFP2 can be, for example, a bone disorder, hypogonadism, cryptorchidism, polycystic ovary syndrome (PCOS), cancer, infertility, or an ocular (eye) wound. The bone disorder can be, for example, osteoporosis, osteopenia, or osteogenesis imperfecta. The cancer can be, for example, testicular cancer, prostate cancer, or thyroid cancer.

In another aspect, the invention provides a method of growing (e.g., increasing the amount of) bone or muscle in a subject comprising contacting the subject with the compound of formula (I) or a pharmaceutically acceptable salt and/or enantiomer thereof. The term “growing” refers to the amount of bone or muscle that is increased relative to a control sample in which no compound of formula (I) is administered in the same time frame.

The invention is further illustrated by the following aspects.

1. A compound of formula (I)

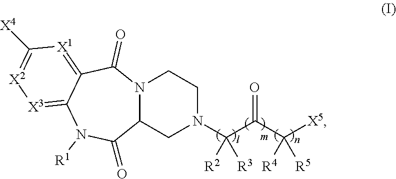

wherein

X¹, X², and X³ are the same or different and each is CH or N;

X⁴ is an optionally substituted aryl or heteroaryl;

X⁵ is selected from the group consisting of

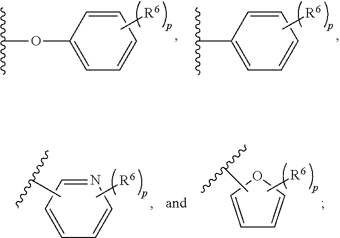

R¹ is hydrogen or alkyl;

R², R³, R⁴, and R⁵ are the same or different and each is hydrogen, alkyl, or halo;

each instance of R⁶ is the same or different and is selected from the group consisting of alkyl, cycloalkyl, heterocycloalkyl, halo, haloalkyl, hydroxy, cyano, nitro, amino, alkylamino, dialkylamino, alkoxy, haloalkoxy, alkylthio, alkylsulfonyl, haloalkylsulfonyl, alkylsulfonamido, carboxylato, —C(═NH)OH, aryl, heteroaryl, and 2-oxoazetidinyl; or

two instances of R⁶ along with the cyclic moiety to which they are bound form phenyl, cycloalkyl, or —O—C(R⁷R⁸)—O—;

R⁷ and R⁸ are the same or different and each is hydrogen, alkyl, or halo;

l is 0 or an integer of 1 to 6;

m is 0 or 1;

n is 0 or an integer of 1 to 6; and

p is 0 or an integer of 1 to 4;

or a pharmaceutically acceptable salt and/or an enantiomer thereof.

2. The compound of aspect 1 or a pharmaceutically acceptable salt and/or enantiomer thereof, wherein X¹, X², and X³ are each CH.

3. The compound of aspect 1 or a pharmaceutically acceptable salt and/or enantiomer thereof, wherein one of X¹, X², and X³ is N and the remaining two are each CH.

4. The compound of any one of aspects 1-3 or a pharmaceutically acceptable salt and/or enantiomer thereof, wherein R¹ is hydrogen.

5. The compound of any one of aspects 1-4 or a pharmaceutically acceptable salt and/or enantiomer thereof, wherein R², R³, R⁴, and R⁵ are each hydrogen.

6. The compound of any one of aspects 1-5 or a pharmaceutically acceptable salt and/or enantiomer thereof, wherein X⁴ is selected from the group consisting of

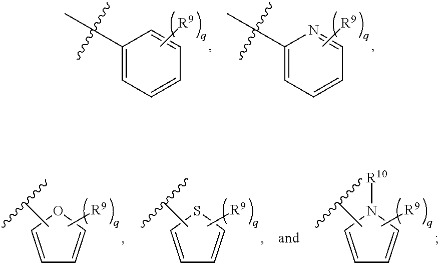

wherein

each instance of R⁹ is the same or different and is selected from the group consisting of alkyl, cycloalkyl, halo, haloalkyl, hydroxy, cyano, nitro, amino, alkylamino, dialkylamino, alkoxy, haloalkoxy, alkylthio, alkylsulfonyl, carboxylato, and —C(═NH)OH; or

two instances of R⁹ along with the cyclic moiety to which they are bound form phenyl, cycloalkyl, or —O—C(R⁷R⁸)—O—;

R¹⁰ is hydrogen or alkyl; and

q is 0 or an integer of 1 to 4.

7. The compound of aspect 6 or a pharmaceutically acceptable salt and/or enantiomer thereof, wherein X⁴ is

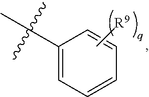

each instance of R⁹ is the same or different and is selected from the group consisting of alkyl, cycloalkyl, halo, haloalkyl, hydroxy, cyano, nitro, haloalkoxy, alkylthio, alkylsulfonyl, and —C(═NH)OH; or

two instances of R⁹ along with the cyclic moiety to which they are bound form —O—C(R⁷R⁸)—O—; and

q is 0 or an integer of 1 to 3.

8. The compound of aspect 7 or a pharmaceutically acceptable salt and/or enantiomer thereof, wherein each instance of R⁹ is the same or different and is selected from the group consisting of alkyl, cyclopropyl, fluoro, chloro, trifluoromethyl, and hydroxy; and q is 1 or 2.

9. The compound of aspect 6 or a pharmaceutically acceptable salt and/or enantiomer thereof, wherein

X⁴ is

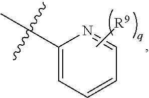

each instance of R⁹ is the same or different and is selected from the group consisting of alkyl, cycloalkyl, halo, haloalkyl, nitro, amino, alkylamino, dialkylamino, and haloalkoxy; and

q is 0 or an integer of 1 to 2.

10. The compound of aspect 6 or a pharmaceutically acceptable salt and/or enantiomer thereof, wherein X⁴ is

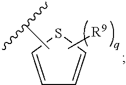

R⁹ is selected from the group consisting of alkyl, halo, and haloalkyl; and

q is 0 or 1.

11. The compound of any one of aspects 1-10 or a pharmaceutically acceptable salt and/or enantiomer thereof, wherein

X⁵ is

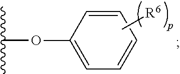

each instance of R⁶ is the same or different and is selected from the group consisting of alkyl, pyrrolidinyl, halo, haloalkyl, cyano, alkoxy, haloalkoxy, alkylthio, alkylsulfonyl, haloalkylsulfonyl, alkylsulfonamido, and 2-oxoazetidinyl; or

two instances of R⁶ along with the cyclic moiety to which they are bound form phenyl or —O—C(R⁷R⁸)—O—;

R⁷ and R⁸ are the same or different and each is hydrogen or halo;

l is 0; m is 1; n is an integer of 1 to 6; and

p is 0 or an integer of 1 to 4.

12. The compound of aspect 11 or a pharmaceutically acceptable salt and/or enantiomer thereof, wherein
(i) p is 1, and R⁶ is fluoro, trifluoromethyl, cyano, trifluoromethoxy, difluoromethoxy, methylthio, methylsulfonyl, trifluoromethylsulfonyl, methylsulfon(methyl)amido, and 2-oxoazetidinyl; or
(ii) p is 2, and each instance of R⁶ is the same or different and is selected from the group consisting of methyl, fluoro, chloro, bromo, iodo, trifluoromethyl, cyano, methoxy, trifluoromethoxy, and difluoromethoxy; or
two instances of R⁶ along with the cyclic moiety to which they are bound form phenyl or —O—CF₂—O—; or
(iii) p is 3, and each instance of R⁶ is the same or different and is selected from the group consisting of methyl, fluoro, trifluoromethyl, cyano, and trifluoromethoxy; or
(iv) p is 4, and each instance of R⁶ is the same or different and is selected from the group consisting of methyl and cyano.

13. The compound of any one of aspects 1-10 or a pharmaceutically acceptable salt and/or enantiomer thereof, wherein

X⁵ is

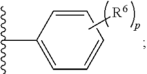

each instance of R⁶ is the same or different and is selected from the group consisting of alkyl, cycloalkyl, heterocycloalkyl, halo, haloalkyl, hydroxy, cyano, nitro, amino, alkylamino, dialkylamino, alkoxy, haloalkoxy, alkylthio, alkylsulfonyl, haloalkylsulfonyl, alkylsulfonamido, carboxylato, —C(═NH)OH, aryl, heteroaryl, and 2-oxoazetidinyl; or

two instances of R⁶ along with the cyclic moiety to which they are bound form phenyl or —O—C(R⁷R⁸)—O—;

R⁷ and R⁸ are the same or different and each is hydrogen, alkyl, or halo; and either

l is 0; m is 0; and n is an integer of 1 to 6;

l is 0; m is 1; and n is 0; or

l is an integer of 1 to 6; m is 1; and n is 0;

and

p is 0 or an integer of 1 to 3.

14. The compound of aspect 13 or a pharmaceutically acceptable salt and/or enantiomer thereof, wherein

each instance of R⁶ is the same or different and is selected from the group consisting of hydroxy and alkoxy; or

two instances of R⁶ along with the cyclic moiety to which they are bound form —O—CH₂—O—.

15. The compound of any one of aspects 1-10 or a pharmaceutically acceptable salt and/or enantiomer thereof, wherein

X⁵ is

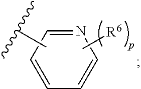

each instance of R⁶ is the same or different and is selected from the group consisting of alkyl, cycloalkyl, heterocycloalkyl, halo, haloalkyl, hydroxy, cyano, nitro, amino, alkylamino, dialkylamino, alkoxy, haloalkoxy, alkylthio, alkylsulfonyl, haloalkylsulfonyl, alkylsulfonamido, carboxylato, —C(═NH)OH, aryl, heteroaryl, and 2-oxoazetidinyl; or

two instances of R⁶ along with the cyclic moiety to which they are bound form phenyl;

l is 0; m is 0; and n is an integer of 1 to 6; and

p is 0 or an integer of 1 to 3.

16. The compound of aspect 15 or a pharmaceutically acceptable salt and/or enantiomer thereof, wherein

each instance of R⁶ is the same or different and is selected from the group consisting of halo and haloalkyl; or

two instances of R⁶ along with the cyclic moiety to which they are bound form phenyl; and

p is 0 or an integer of 1 or 2.

17. The compound of any one of aspects 1-10 or a pharmaceutically acceptable salt and/or enantiomer thereof, wherein

X⁵ is

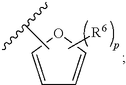

each instance of R⁶ is the same or different and is selected from the group consisting of alkyl, cycloalkyl, heterocycloalkyl, halo, haloalkyl, hydroxy, cyano, nitro, amino, alkylamino, dialkylamino, alkoxy, haloalkoxy, alkylthio, alkylsulfonyl, haloalkylsulfonyl, alkylsulfonamido, carboxylato, —C(═NH)OH, aryl, heteroaryl, and 2-oxoazetidinyl; or

two instances of R⁶ along with the cyclic moiety to which they are bound form phenyl;

l is 0; m is 1; and n is 0; and

p is 0 or an integer of 1 to 3.

18. The compound of aspect 17 or a pharmaceutically acceptable salt and/or enantiomer thereof, wherein

each instance of R⁶ is haloalkyl, or

two instances of R⁶ along with the cyclic moiety to which they are bound form phenyl; and

p is 1 or 2.

19. The compound of any one of aspects 1-18 or a pharmaceutically acceptable salt and/or enantiomer thereof, wherein the compound of formula (I) is an S-enantiomer.

20. The compound of any one of aspects 1-18 or a pharmaceutically acceptable salt and/or enantiomer thereof, wherein the compound of formula (I) is an R-enantiomer.

21. The compound of aspect 1 or a pharmaceutically acceptable salt thereof, that is selected from the group consisting of Tables 1, 2, 3, 4,5, and 6 or a racemic mixture or enantiomer thereof.

22. The compound of aspect 1 or a pharmaceutically acceptable salt and/or enantiomer thereof, that is

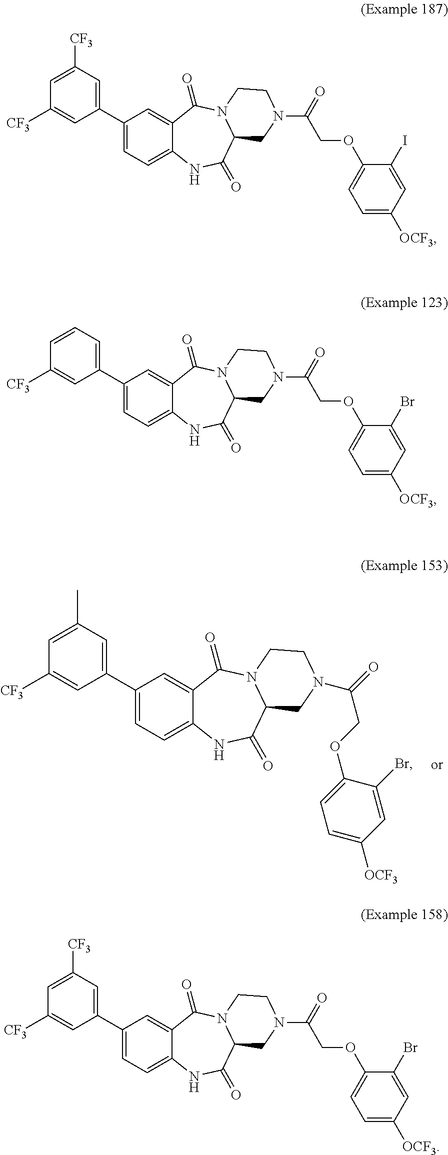 [SMILES: O=C1Nc2ccc(-c3cc(C(F)(F)F)cc(C(F)(F)F)c3)cc2C(=O)N2CCN(C(=O)COc3ccc(OC(F)(F)F)cc3I)C[C@@H]12 ; O=C1Nc2ccc(-c3cccc(C(F)(F)F)c3)cc2C(=O)N2CCN(C(=O)COc3ccc(OC(F)(F)F)cc3Br)C[C@@H]12 ; Cc1cc(-c2ccc3c(c2)C(=O)N2CCN(C(=O)COc4ccc(OC(F)(F)F)cc4Br)C[C@H]2C(=O)N3)cc(C(F)(F)F)c1 ; O=C1Nc2ccc(-c3cc(C(F)(F)F)cc(C(F)(F)F)c3)cc2C(=O)N2CCN(C(=O)COc3ccc(OC(F)(F)F)cc3Br)C[C@@H]12]

23. A pharmaceutical composition comprising the compound of any one of aspects 1-22 or a pharmaceutically acceptable salt and/or enantiomer thereof and at least one carrier.

24. A method of treating a disorder mediated by relaxin family peptide receptor 2 (RXFP2) in a subject comprising administering the compound of any one of aspects 1-22 or a pharmaceutically acceptable salt and/or enantiomer thereof to the subject.

25. The method of aspect 24, wherein the disorder mediated by RXFP2 is a bone disorder, hypogonadism, cryptorchidism, polycystic ovary syndrome, cancer, infertility, or an ocular wound.

26. The method of aspect 25, wherein the bone disorder is osteoporosis, osteopenia, or osteogenesis imperfecta.

27. The method of aspect 25, wherein the cancer is testicular cancer, prostate cancer, or thyroid cancer.

28. A method of activating the functional activity of relaxin family peptide receptor 2 (RXFP2) in a cell comprising contacting the cell with the compound of any one of aspects 1-22 or a pharmaceutically acceptable salt and/or enantiomer thereof.

29. A method of growing bone or muscle in a subject comprising contacting the subject with the compound of any one of aspects 1-22 or a pharmaceutically acceptable salt and/or enantiomer thereof.

The following examples further illustrate the invention but, of course, should not be construed as in any way limiting its scope.

### EXAMPLES

General Methods. All air or moisture sensitive reactions were performed under positive pressure of nitrogen or argon with oven-dried glassware. Anhydrous solvents such as dichloromethane (DCM), N,N-dimethylformamide (DMF), tetrahydrofuran (THF), acetonitrile (ACN), methanol (MeOH), and triethylamine (TEA) were purchased from Sigma-Aldrich (St. Louis, MO). Preparative purification was performed on a Waters semi-preparative HPLC system (Waters Corp., Milford, MA). The column used was a Phenomenex Luna C18 (5 micron, 30×75 mm; Phenomenex, Inc., Torrance, CA) at a flow rate of 45.0 mL/min. The mobile phase consisted of acetonitrile and water (each containing 0.1% trifluoroacetic acid). A gradient of 10% to 50% acetonitrile over 8 minutes was used during the purification. Fraction collection was triggered by ultraviolet (UV) detection at 220 nM. Analytical analysis was performed on an Agilent liquid chromatography/mass spectrometry (LC/MS) (Agilent Technologies, Santa Clara, CA). Method 1 (t₁): A 7-minute gradient of 4% to 100% acetonitrile (containing 0.025% trifluoroacetic acid) in water (containing 0.05% trifluoroacetic acid) was used with an 8-minute run time at a flow rate of 1.0 mL/min. Method 2 (t₂): A 3-minute gradient of 4% to 100% acetonitrile (containing 0.025% trifluoroacetic acid) in water (containing 0.05% trifluoroacetic acid) was used with a 4.5-minute run time at a flow rate of 1.0 mL/min. A Phenomenex Luna C18 column (3 micron, 3×75 mm) was used at a temperature of 50° C. Method 3 (t₃): A 3-minute gradient 4% to 100% acetonitrile (containing 0.1% NH₄OH in water) was used with a 4.5-minute run time at a flow rate of 1.0 mL/min. Method 4 (t₄):−d4 A 3-minute gradient 4% to 100% acetonitrile (containing 0.1% NH₄OH in water) was used with a 4.5-minute run time at a flow rate of 1.0 mL/min. Purity determination was performed using an Agilent diode array detector for Method 1, Method 2, Method 3, and Method 4. Mass determination was performed using an Agilent 6130 mass spectrometer with electrospray ionization in the positive mode (Method 1, Method 2, and Method 3) and in the negative mode for Method 4. ¹H nuclear magnetic resonance (NMR) and ¹⁹F NMR spectra were recorded on Varian 400 MHz or 600 spectrometers (Agilent Technologies, Santa Clara, CA). Chemical shifts are reported in ppm with deuterated solvent (DMSO-d₆) at 2.50 ppm, Acetic Acid-d₄ at 2.03 ppm, CDCl₃ at 7.26 and ACD-d₄ at 1.96 ppm as internal standard reference chemical shifts for NMR sample solutions. All of the analogs tested in the biological assays had a purity of greater than 95% based on both analytical methods. High resolution mass spectrometry was recorded on Agilent 6210 Time-of-Flight (TOF) LC/MS system. Confirmation of molecular formula was accomplished using electrospray ionization in the positive mode with the Agilent Masshunter software (Version B.02). Chiral and racemic 1-(tert-butyl)-3-methyl piperazine-1,3-dicarboxylates (R([α]_{D}²⁰=−27 (c=1, CHCl₃)), S [α]_{D}²⁰=+25 (c=1, CHCl₃)), and the racemic compound were purchased from Combiblocks (Combi-Blocks, Inc. San Diego, CA). Chimeric receptor constructs were obtained from Dr Ross Bathgate (Florey Institute, Australia). The design of the plasmids was in accordance with the methods of Sudo et at. (The Journal of Biological Chemistry, 278(10), 7855-7862 (2002)), the contents of which are fully incorporated by reference.

### General Procedure A for the coupling of either racemic, (R) or (S) 1-(tert-butyl) 3-methyl piperazine-1,3-dicarboxylate with 5-bromo-2-((tert-butoxycarbonyl)amino)benzoic acid (for racemic compounds) or phenyl substituted 2-((tert-butoxycarbonyl)amino)benzoic acid (for chiral compounds)

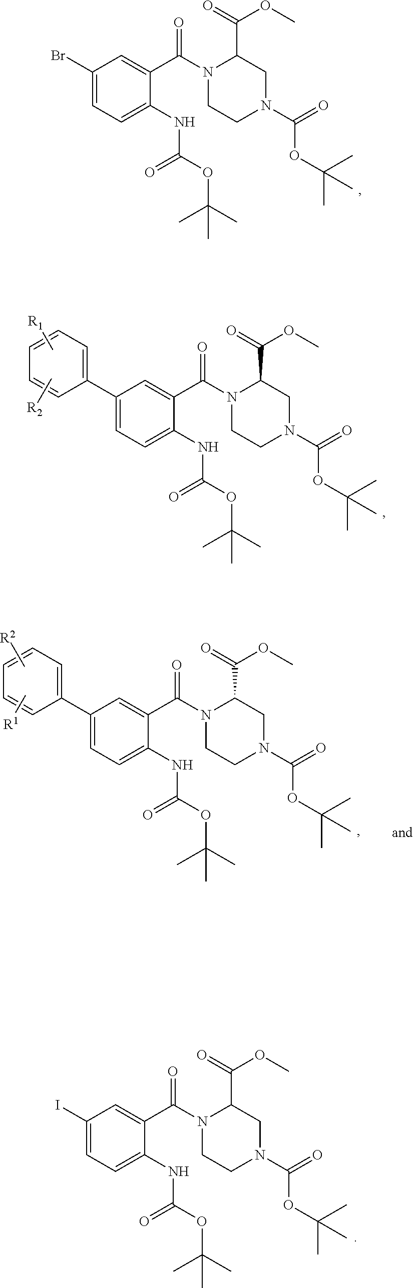

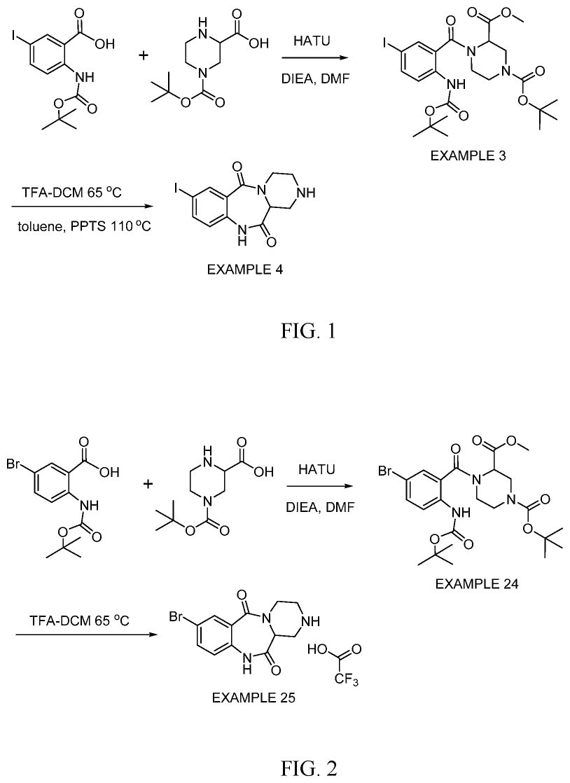

To a solution of either R, S, or racemic 1-(tert-butyl)-3-methyl piperazine-1,3-dicarboxylate (1.2 eq.) and 4-((tert-butoxycarbonyl)amino)-[1,1′-biphenyl] or 4-((tert-butoxycarbonyl)amino)-3′-(trifluoromethyl)-[1,1′-substituted biphenyl]-3-carboxylic acid (1 eq.) or 5-bromo-2-((tert-butoxycarbonyl)amino)benzoic acid or 5-iodo-2-((tert-butoxycarbonyl)amino)benzoic acid (1.0 eq.) in dimethylformamide (DMF) was added (1-[bis(dimethylamino)methylene]-1H-1,2,3-triazolo[4,5-b]pyridinium 3-oxide hexafluorophosphate (HATU) (1.3 eq.) followed by N-methylmorpholine (3 eq.). The resultant reaction mixture was allowed to stir 18 hours at room temperature. The reaction mixture was poured into ethyl acetate (EtOAc) and washed twice with water. The organic layer was dried with Na₂SO₄ and concentrated in vacuo. The crude mixture was purified by flash column chromatography (silica gel, 0-50% EtOAc-hexanes) to afford the corresponding 1-(tert-butyl)-3-methyl 4-(5-bromo-2-((tert-iodo-2-((tert-butoxycarbonyl)amino)benzoyl)piperazine-1,3-dicarboxylates or 1-(tert-butyl)-3-methyl 4-(4-((tert-butoxycarbonyl)amino)-[1,1′-substituted-biphenyl]-3-carbonyl)piperazine-1,3-dicarboxylates. See FIGS. 1 and 2.

### General Procedure B for the synthesis of 8-bromo or 8-iodo-1,3,4,12a-tetrahydrobenzo[e]pyrazino[1,2-a][1,4]diazepine-6,12 (2H, 11H)-diones or 8-iodo-1,3,4,12a-tetrahydrobenzo[e]pyrazino[1,2-a][1,4]diazepine-6,12 (2H, 11H)-diones (for racemic compounds) and 8-phenyl-1,3,4,12a-tetrahydrobenzo[e]pyrazino[1,2-a][1,4]diazepine-6,12 (2H, 11H)-diones (for chiral compounds) as trifluoroacetate (TFA salts)

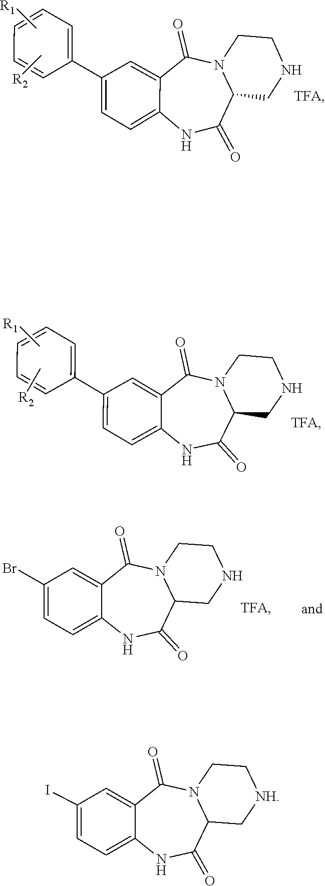

To 1-(tert-butyl) 3-methyl 4-(5-bromo-2-((tert-butoxycarbonyl)amino)benzoyl)piperazine-1,3-dicarboxylates or 1-(tert-butyl)-3-methyl 4-(4-((tert-butoxycarbonyl)amino)-[1,1′<substituted>-biphenyl]-3-carbonyl)piperazine-1,3-dicarboxylates (General Procedure A) in a round bottom flask equipped with a reflux condenser was added 1:1 dichloromethane (DCM)-trifluoroacetic acid (TFA) to give a 0.02 M solution. The resultant solution was heated to 65° C. for 18-24 hours. The reaction was allowed to cool, the solvent was removed in vacuo, and the result was azeotroped with methanol (MeOH) three times. The residue was dried under high vacuum and then diluted with hexane-ether (2:1) and sonicated for an hour or until a powder is visible in the flask. The solvent was decanted, and the resultant diazapinediones were dried under vacuum and used without further purification. See FIGS. 1 and 2.

### General Procedure C for the synthesis of 2-(2-phenoxyacetyl)-8-phenyl-1,3,4,12a-tetrahydrobenzo[e]pyrazino[1,2-a][1,4]diazepine-6,12 (2H, 11H)-diones and 8-bromo-2-(2-phenoxyacetyl)-1,3,4,12a-tetrahydrobenzo[e]pyrazino[1,2-a][1,4]diazepine-6,12 (2H, 11H)-diones

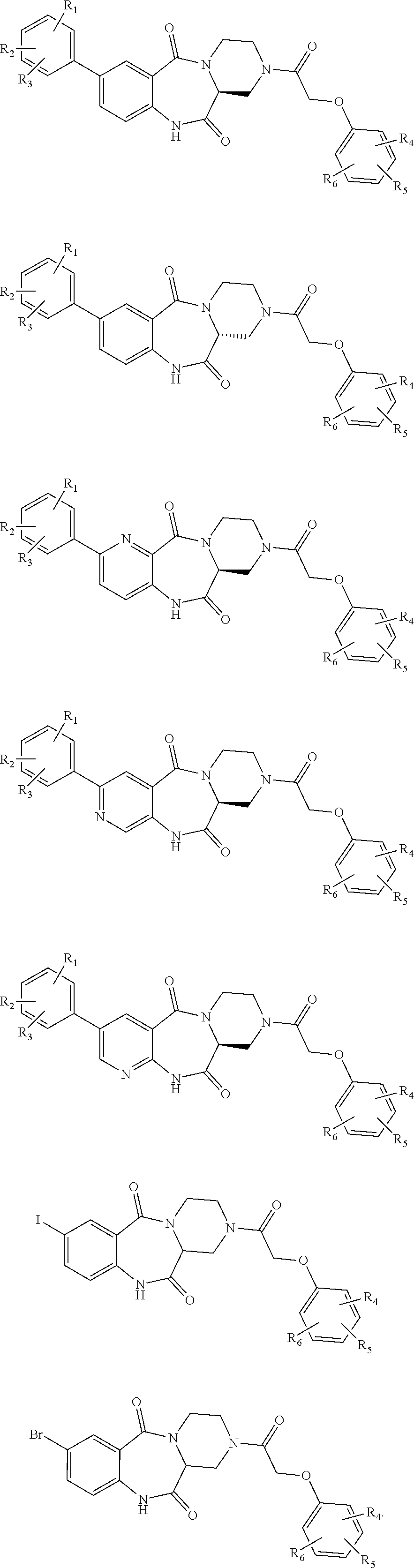

To a stirring solution of 8-bromo-1,3,4,12a-tetrahydrobenzo[e]pyrazino[1,2-a][1,4]diazepine-6,12 (2H, 11H)-dione trifluoroacetate or 8-iodo-1,3,4,12a-tetrahydrobenzo[e]pyrazino[1,2-a][1,4]diazepine-6,12 (2H, 11H)-dione or chiral 8-phenyl-1,3,4,12a-tetrahydrobenzo[e]pyrazino[1,2-a][1,4]diazepine-6,12 (2H, 11H)-dione (1 eq.) (General Procedure B) in dimethylformamide (DMF) was added 2-phenoxyacetic acid (1.2 eq.) or a substituted 2-phenoxyacetic acid (1.2 eq., General Procedure E), (1-[bis(dimethylamino)methylene]-1-1,2,3-triazolo[4,5-b]pyridinium 3-oxide hexafluorophosphate (HATU) (1.3 eq.) followed by N-methylmorpholine (3 eq.). The resultant reaction mixture was allowed to stir 6-18 hours at room temperature. The reaction mixture was poured into 10% MeOH-DCM and washed twice with water. The organic layer was dried with Na₂SO₄ and concentrated in vacuo. The crude mixture was purified by flash column chromatography (silica gel, 0-10% MeOH-DCM) to afford the corresponding 1-(tert-butyl) 3-methyl 4-(5-bromo-2-((tert-butoxycarbonyl)amino)benzoyl)piperazine-1,3-dicarboxylates or 1-(tert-butyl) 3-methyl 4-(4-((tert-butoxycarbonyl)amino)-[1,1′-<substituted>biphenyl]-3-carbonyl)piperazine-1,3-dicarboxylates.

### General Procedure D for the synthesis of 8-bromo-2-(2-phenoxyacetyl)-1,3,4,12a-tetrahydrobenzo[e]pyrazino[1,2-a][1,4]diazepine-6,12 (2H, 11H)-diones

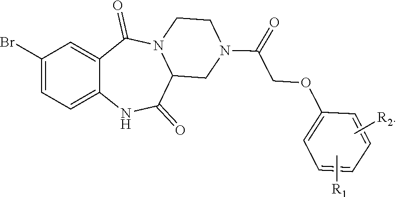

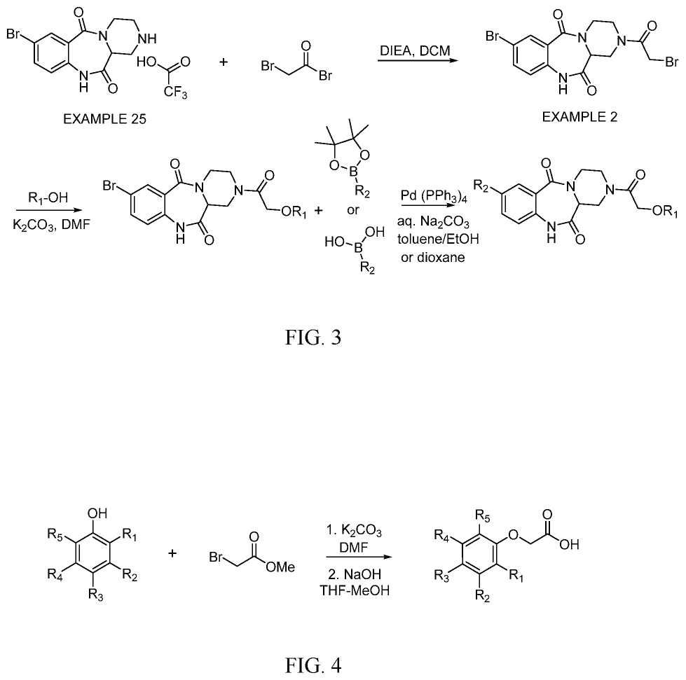

To a stirring DMF solution of 8-bromo-2-(2-bromoacetyl)-1,3,4,12a-tetrahydrobenzo[e]pyrazino[1,2-a][1,4]diazepine-6,12 (2H, 11H)-dione (KJW009-015) (1 eq.) was added a substituted phenol (1.4 eq.) followed by K₂CO₃ (3 eq.). The resultant slurry was heated to 65° C. for 2 hours at which time it was poured into EtOAc and washed twice with saturated NaHCO₃. The solvent was dried (Na₂SO₄) and concentrated under vacuum. The residue was purified by flash column chromatography (silica gel, 0-10% MeOH-DCM) to afford the product. See FIG. 3.

### General Procedure E for the synthesis of substituted 2-phenoxyacetic acids and esters

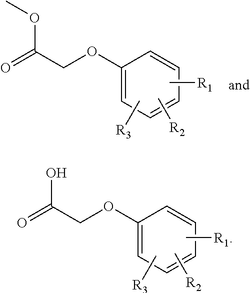

To a DMF solution of the appropriate substituted phenol (1 eq.) was added methyl 2-bromoacetate or methyl 2-chloroacetate (1.2 eq.) followed by potassium carbonate (K₂CO₃) (3 eq.). The resultant mixture was heated to 65° C. for 18 hours at which time it was allowed to cool and poured into EtOAc. The EtOAc was washed two times with saturated sodium bicarbonate (Na₂HCO₃), dried (Na₂SO₄), and concentrated in vacuo. The product methyl 2-phenoxyacetates (1 eq.) were dissolved in 1:1 MeOH-THF and 4N sodium hydroxide (NaOH) (1.4 eq.) was added. The reaction mixture was heated to 65° C. for 6-18 hours at which time it was allowed to cool, and 2N HCl (1.6 eq.) was added. The solvent was removed, the crude product was dissolved in 10% MeOH-DCM and washed twice with saturated NaHCO₃, the solvent was dried (Na₂SO₄) and concentrated in vacuo to yield the substituted phenoxyacetic acids with purities >95%. See FIG. 4.

### General Procedure F for the synthesis of racemic N-alkylated 8-bromo-1,3,4,12a-tetrahydrobenzo[e]pyrazino[1,2-a][1,4]diazepine-6,12 (2H, 11H)-dione compounds

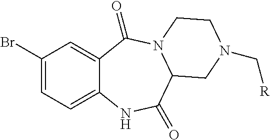

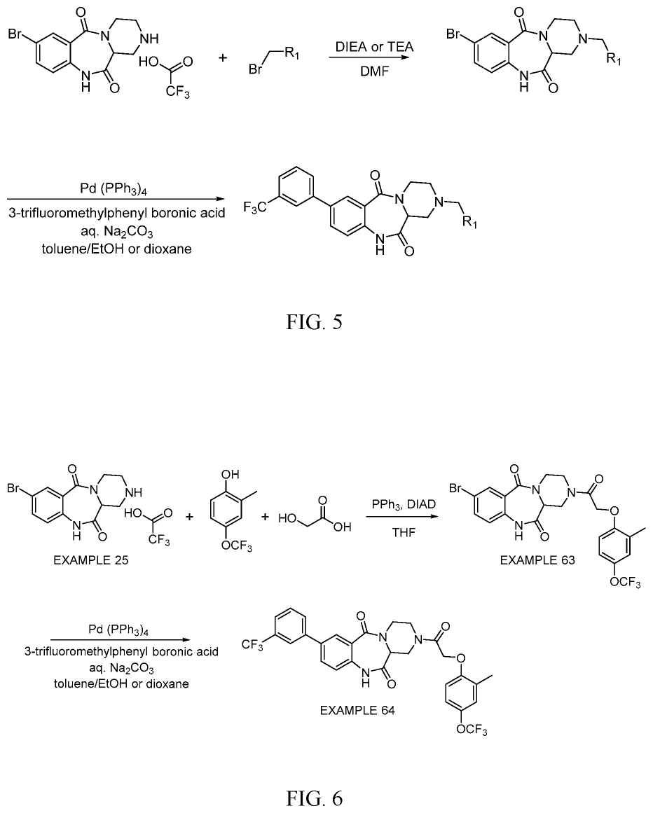

To 8-bromo-1,3,4,12a-tetrahydrobenzo[e]pyrazino[1,2-a][1,4]diazepine-6,12 (2H, 11H)-dione 2,2,2-trifluoroacetate (1 eq.) (General Procedure B) in DMF was added a benzylic or alkyl halide (1.3 eq.) followed by triethylamine (TEA) (3.5 eq.). The reaction mixture was heated to 70° C. for 6-18 hours. The DMF was removed under vacuum, and the crude products were purified by flash column chromatography (silica gel, 0-5% MeOH-DCM) to afford the N-alkylated 8-bromo-1,3,4,12a-tetrahydrobenzo[e]pyrazino[1,2-a][1,4]diazepine-6,12 (2H, 11H)-diones. See FIG. 5.

### General Procedure G for the synthesis of racemic N-substituted-8-(3-(trifluoromethyl)phenyl)-1,3,4,12a-tetrahydrobenzo[e]pyrazino[1,2-a][1,4]diazepine-6,12 (2H, 11H)-diones)

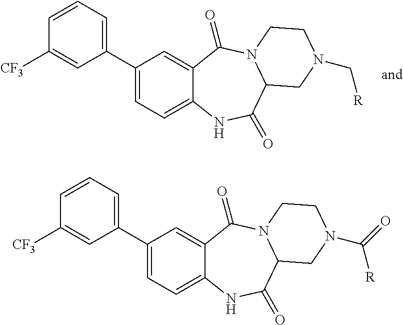

The N-substituted-8-bromo-1,3,4,12a-tetrahydrobenzo[e]pyrazino[1,2-a][1,4]diazepine-6,12 (2H, 11H)-dione (1 eq.) was slurried in 2:1 toluene-EtOH. Pd(PPh₃)₄ (0.12 eq.) was added, followed by an aryl or heterocyclic boronic acid (1.4 eq.) and 2M Na₂CO₃ (5 eq.). The resultant mixture was heated to 90° C. under N₂ for 18 hours. The reaction mixture was poured into DCM, washed twice with water, dried (Na₂SO₄), and concentrated in vacuo. The residue was taken up in DCM, filtered, and the filtrate was concentrated. The residue was purified via standard reverse phase high performance liquid chromatography (HPLC) conditions using a gradient of 10-100% ACN in H₂O with 0.1% TFA to afford the product as a TFA salt (for amines) or by flash column chromatography (silica gel, 0-10% MeOH-DCM) for carbonyl-substituted piperizines to afford the product. See FIGS. 3, 5, 6, and 7.

### General Procedure H for the synthesis of 2-benzoyl-8-bromo-1,3,4,12a-tetrahydrobenzo[e]pyrazino[1,2-a][1,4]diazepine-6,12 (2H, 11H)-diones

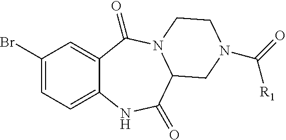

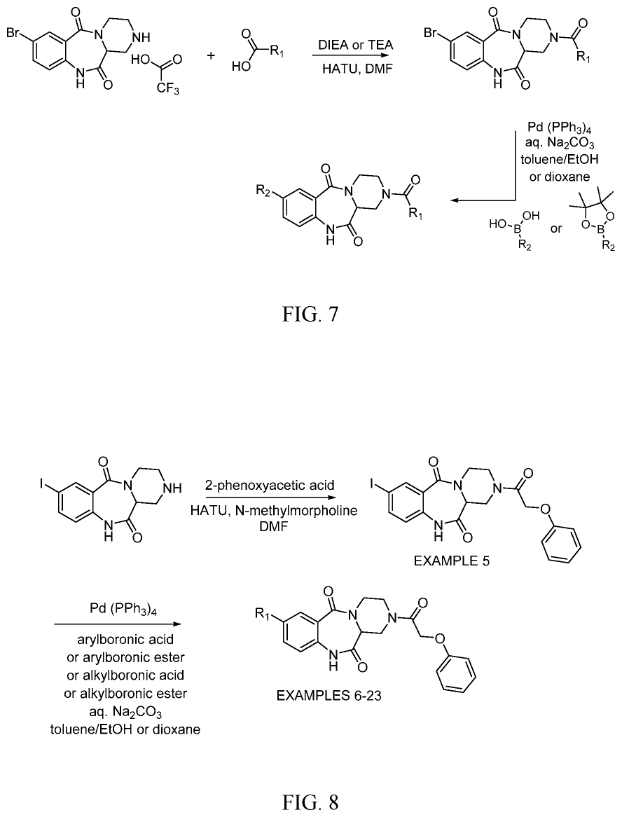

To 8-bromo-1,3,4,12a-tetrahydrobenzo[e]pyrazino[1,2-a][1,4]diazepine-6,12 (2H, 11H)-dione 2,2,2-trifluoroacetate (KJW008-042) (1 eq.) in DMF was added a substituted benzoic acid, furan-carboxylic acid, thiophene carboxylic acid, or benzofuran-carboxylic acid (1.3 eq.), HATU (1.3 eq.), followed by TEA (3.5 eq.). The reaction mixture was allowed to stir for 6-18 hours. The DMF was removed under vacuum, and the crude products were purified by flash column chromatography (silica gel, 0-5% MeOH-DCM). See FIG. 7.

### General Procedure I for the synthesis of 2-benzoyl-8-phenyl-1,3,4,12a-tetrahydrobenzo[e]pyrazino[1,2-a][1,4]diazepine-6,12 (2H, 11H)-diones

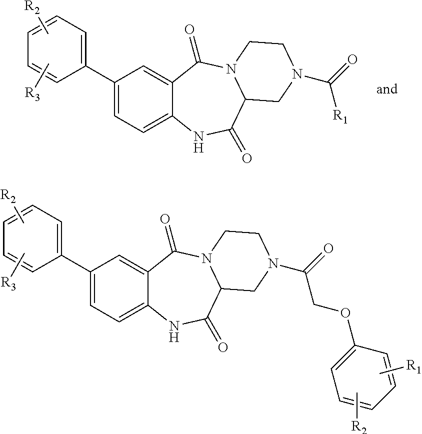

The N-substituted 8-bromo-1,3,4,12a-tetrahydrobenzo[e]pyrazino[1,2-a][1,4]diazepine-6,12 (2H, 11H)-dione or 8-iodo-2-(2-phenoxyacetyl)-1,3,4,12a-tetrahydrobenzo[e]pyrazino[1,2-a][1,4]diazepine-6,12 (2H, 11H)-dione (1 eq.) (General Procedure H) was slurried in 2:1 toluene-EtOH. Pd(PPh₃)₄ (0.12 eq.) was added, followed by a substituted boronic acid (1.4 eq.) and 2M Na₂CO₃ (5 eq.). The resultant mixture was heated to 90° C. under N₂ for 18 hours. The reaction mixture was poured into DCM, washed twice with water, dried (Na₂SO₄), and concentrated in vacuo. The residue was purified by flash column chromatography (silica gel, 0-10% MeOH-DCM) to afford the product. See. FIG. 8.

### General Procedure J for the synthesis of 2-(2-oxo-2-phenylethyl)-8-(3-(trifluoromethyl)phenyl)-1,3,4,12a-tetrahydrobenzo[e]pyrazino[1,2-a][1,4]diazepine-6,12 (2H, 11H)-diones

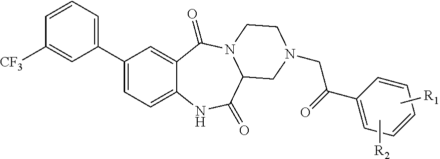

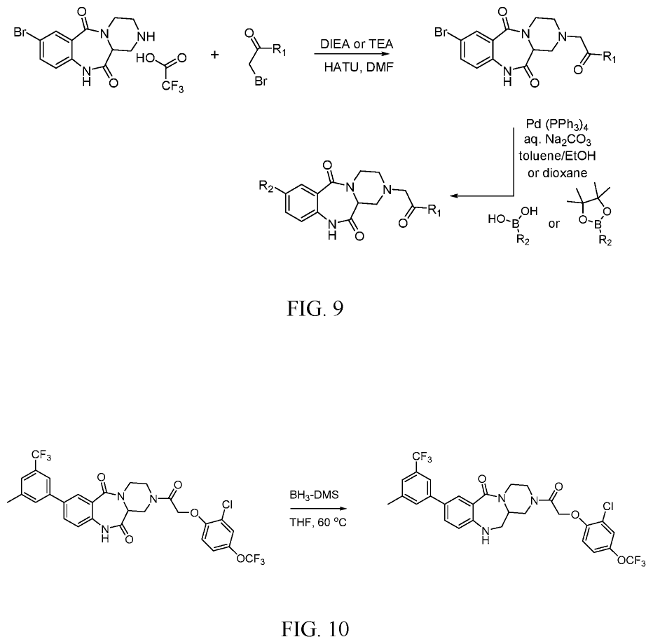

To a stirring solution of 8-bromo-1,3,4,12a-tetrahydrobenzo[e]pyrazino[1,2-a][1,4]diazepine-6,12 (2H, 11H)-dione 2,2,2-trifluoroacetate (1 eq.) (General Procedure B) and triethylamine (TEA) (2.5) in DMF at room temperature (RT) was added a substituted 2-bromo-1-phenylethan-1-one. The reaction was stirred for 2 hours, poured into EtOAc, washed twice with saturated NaHCO₃, dried (Na₂SO₄), and concentrated in vacuo. The resultant 8-bromo-2-(2-oxo-2-phenylethyl)-1,3,4,12a-tetrahydrobenzo[e]pyrazino[1,2-a][1,4]diazepine-6,12 (2H, 11H)-dione products were used without further purification. The product 8-bromo-2-(2-oxo-2-phenylethyl)-1,3,4,12a-tetrahydrobenzo[e]pyrazino[1,2-a][1,4]diazepine-6,12 (2H, 11H)-dione (1 eq.) was slurried in 2:1 toluene-EtOH. Pd(PPh₃)₄ (0.12 eq.) was added followed by 3-trifluoromethylboronic acid (1.4 eq.) and 2M Na₂CO₃ (5 eq.). The resultant mixture was heated to 90° C. under N₂ for 18 hours. The reaction mixture was poured into DCM, washed twice with water, dried (Na₂SO₄) and concentrated in vacuo. The residue was taken up in DCM, filtered, and the filtrate concentrated. The residue was purified by flash column chromatography (silica gel, 0-10% MeOH-DCM) to afford the product. See FIG. 9.

### General Procedure K for the synthesis of 4-((tert-butoxycarbonyl)amino)-[1,1′-biphenyl]-3-carboxylic acids

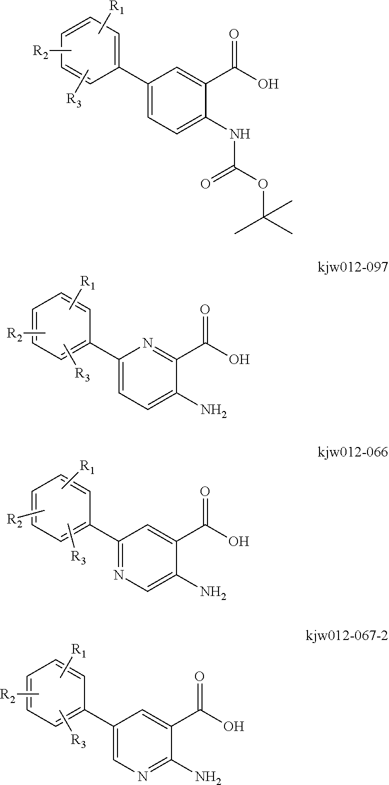

To a round bottom flask equipped with a reflux condenser was added 5-bromo-2-((tert-butoxycarbonyl)amino)benzoic acid (1 eq.) or a bromo-pyridyl amino carboxylic acid or ester (1 eq.), phenyl, substituted phenylboronic acid (1.4 eq.), and tetrakis(triphenylphosphine)palladium(0) (Pd(PPh₃)₄ (0.12 eq.) or Pd(dppf)Cl₂-DCM ([1,1′-bis(diphenylphosphino)ferrocene]dichloropalladium(II), complex with dichloromethane) (0.15 eq.) was added dioxane followed by 2M Na₂CO₃ (5 eq.). The result was heated to 90° C. under N₂ for 3-18 hours and then allowed to cool. The reaction was filtered through CELITE™ (Sigma Aldrich, St. Louis, MO) and concentrated under vacuum. The residue was purified by flash column chromatography (silica gel, 0-5% MeOH-DCM or 0-100% EtOAc-hexanes) to afford the product.

### Example 1

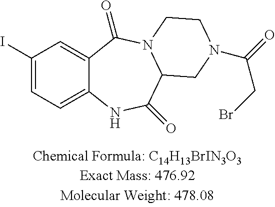 [SMILES: O=C1Nc2ccc(I)cc2C(=O)N2CCN(C(=O)CBr)CC12]

This example is directed to the synthesis of 2-(2-bromoacetyl)-8-iodo-1,3,4,12a-tetrahydrobenzo[e]pyrazino[1,2-a][1,4]diazepine-6,12 (2H, 11H)-dione (KJW006-054) in an aspect of the invention.

To 8-iodo-1,3,4,12a-tetrahydrobenzo[e]pyrazino[1,2-a][1,4]diazepine-6,12 (2H, 11H)-dione (1 eq.) (KJW006-059) in DCM at 0° C. was added DIEA (3 eq.) followed by 2-bromoacetyl bromide (1.15 eq.) via syringe. The reaction mixture was allowed to warm to room temperature and stirred for 1 hour. The reaction was quenched with saturated aq. Na₂HCO₃, diluted with DCM, and the layers were separated. The organic layer was dried (Na₂SO₄) and concentrated in vacuo to afford the title compound which was used without further purification. LCMS RT (Method 2)=2.70 min, m/z 478.00 [M⁺).

### Example 2

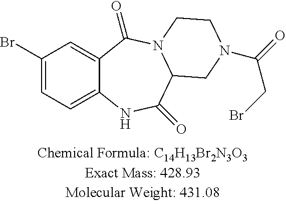 [SMILES: O=C1Nc2ccc(Br)cc2C(=O)N2CCN(C(=O)CBr)CC12]

This example is directed to the synthesis of 8-bromo-2-(2-bromoacetyl)-1,3,4,12a-tetrahydrobenzo[e]pyrazino[1,2-a][1,4]diazepine-6,12 (2H, 11H)-dione (KJW009-015) in an aspect of the invention. See FIG. 3.

To a slurry of 8-bromo-1,3,4,12a-tetrahydrobenzo[e]pyrazino[1,2-a][1,4]diazepine-6,12 (2H, 11H)-dione 2,2,2-trifluoroacetate (1 eq.) (KJW008-042) in DCM at 0° C. was added DIEA (3 eq.) followed by 2-bromoacetyl bromide (1.15 eq.) via syringe. The reaction mixture was allowed to stir at 0° C. for 1 hour. The reaction was then quenched with saturated aq. Na₂HCO₃, diluted with DCM and the layers were separated. The organic layer was dried (Na₂SO₄) and concentrated in vacuo to afford the title compound which was used without further purification. LCMS RT (Method 2)=2.65 min, m/z 431.9 [M+H⁺).

### Example 3

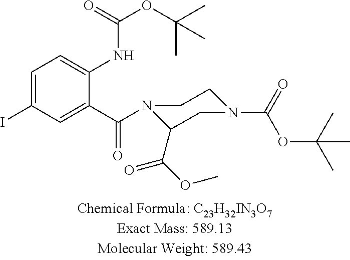 [SMILES: COC(=O)C1CN(C(=O)OC(C)(C)C)CCN1C(=O)c1cc(I)ccc1NC(=O)OC(C)(C)C]

This example is directed to the synthesis of 1-(tert-butyl)-3-methyl-4-(2-((tert-butoxycarbonyl)amino)-5-iodobenzoyl)piperazine-1,3-dicarboxylate (KJW005-098) in an aspect of the invention. See FIG. 1.

The compound was prepared following General Procedure A using racemic 1-(tert-butyl)-3-methyl piperazine-1,3-dicarboxylate and 5-bromo-2-((tert-butoxycarbonyl)amino)benzoic acid. ¹H NMR (400 MHz, DMSO-d₆) δ 8.74 (s, 1H), 8.01 (s, 1H), 7.57-7.53 (m, 1H), 7.02 (d, J=8.0 Hz, 1H).), 5.11 (s, 1H), 4.33 (m, 2H), 3.69 (d, J=23.0 Hz, 3H), 3.47-3.20 (m, 4H), 1.44 (s, 9H), 1.38 (s, 9H). LCMS RT (Method 4)=3.616 min, m/z 588.0 [M−H]⁻.

### Example 4

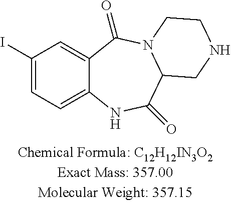 [SMILES: O=C1Nc2ccc(I)cc2C(=O)N2CCNCC12]

This example is directed to the synthesis of 8-iodo-1,3,4,12a-tetrahydrobenzo[e]pyrazino[1,2-a][1,4]diazepine-6,12 (2H, 11H)-dione (KJW006-059) in an aspect of the invention. See FIG. 1.

1-(tert-Butyl) 3-methyl 4-(2-((tert-butoxycarbonyl)amino)-5-iodobenzoyl)piperazine-1,3-dicarboxylate (KJW005-098) was dissolved in a 1:1 mixture of DCM-TFA and heated at 65° C. overnight. The resultant solution was concentrated under vacuum, azeotroped with MeOH and dried. Toluene was added followed by pyridinium p-toluenesulfonate (1 eq.) and the reaction was refluxed for 4 hours, concentrated under vacuum, diluted with 10% MeOH-DCM and washed twice with saturated NaHCO₃. The organic layer was dried (Na₂SO₄) and concentrated under vacuum to afford the title compound. ¹H NMR (400 MHz, DMSO-d₆) δ 10.43 (s, 1H), 7.57 (dd, J=8.1, 1.7 Hz, 1H), 7.48-7.42 (m, 2H), 4.12 (ddd, J=13.4, 4.0, 1.7 Hz, 1H), 3.93 (dd, J=5.1, 1.8 Hz, 1H), 3.33-3.22 (m, 2H), 3.00 (dd, J=13.2, 4.0 Hz, 1H), 2.84-2.56 (m, 3H), 1.23 (s, 1H); LCMS RT (Method 2)=2.20 min, m/z 358.0 [M+H⁺).

### Example 5

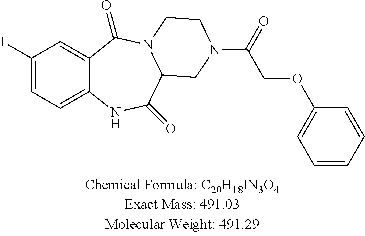 [SMILES: O=C1Nc2ccc(I)cc2C(=O)N2CCN(C(=O)COc3ccccc3)CC12]

This example is directed to the synthesis of 8-Iodo-2-(2-phenoxyacetyl)-1,3,4,12a-tetrahydrobenzo[e]pyrazino[1,2-a][1,4]diazepine-6,12 (2H, 11H)-dione (KJW006-066) in an aspect of the invention. See FIG. 8.

The compound was prepared following General Procedure C using 8-iodo-1,3,4,12a-tetrahydrobenzo[e]pyrazino[1,2-a][1,4]diazepine-6,12 (2H, 11H)-dione ((KJW006-059) and 2-phenoxyacetic acid. ¹H NMR (400 MHz, DMSO-d₆) δ 10.64 (s, 0.68×1H), 10.55 (s, 0.32×1H), 7.61 (ddd, J=8.3, 4.4, 1.6 Hz, 1H), 7.54 (s, 1H), 7.53-7.49 (m, 1H), 7.30-7.22 (m, 2H), 6.99-6.89 (m, 3H), 5.06-4.90 (m, 0.67×2H), 4.88-4.68 (m, 0.33×2H), 4.36-4.27 (m, 1H), 4.21-3.78 (m, 2H), 3.77-3.49 (m, 2H), 3.43-3.34 (m, 1H). LCMS RT (Method 1)=4.935 min. m/z 492.1 [M+H⁺].

### Example 6

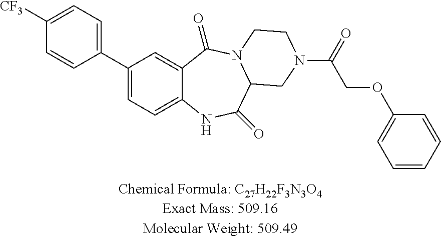 [SMILES: O=C1Nc2ccc(-c3ccc(C(F)(F)F)cc3)cc2C(=O)N2CCN(C(=O)COc3ccccc3)CC12]

This example is directed to the synthesis of 2-(2-Phenoxyacetyl)-8-(4-(trifluoromethyl)phenyl)-1,3,4,12a-tetrahydrobenzo[e]pyrazino[1,2-a][1,4]diazepine-6,12 (2H, 11H)-dione (KJW006-068) in an aspect of the invention. See FIG. 8.

The compound was prepared following General Procedure I using 8-iodo-2-(2-phenoxyacetyl)-1,3,4,12a-tetrahydrobenzo[e]pyrazino[1,2-a][1,4]diazepine-6,12 (2H, 11H)-dione (KJW006-066) and 4-trifluoromethylphenylboronic acid. ¹H NMR (400 MHz, Acetic Acid-d₄) δ 8.13 (d, J=8.2 Hz, 1H), 7.91-7.78 (m, 4H), 7.68 (dd, J=8.3, 1.8 Hz, 1H), 7.46 (t, J=2.4 Hz, 1H), 7.37-7.22 (m, 2H), 6.96 (dd, J=13.4, 7.5 Hz, 3H), 5.09-4.99 (m, 0.56×2H), 4.92-4.80 (m, 0.44×2H), 4.60-4.37 (m, 2H), 4.30-4.08 (m, 1H), 4.07-3.68 (m, 4H). ¹⁹F NMR (376 MHz, DMSO-d₆) δ−61.03, −61.04. LCMS RT (Method 1)=7.045 min, m/z 510.2 (M+H⁺].

### Example 7

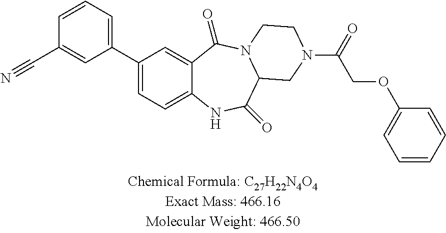 [SMILES: N#Cc1cccc(-c2ccc3c(c2)C(=O)N2CCN(C(=O)COc4ccccc4)CC2C(=O)N3)c1]

This example is directed to the synthesis of 3-(6,12-Dioxo-2-(2-phenoxyacetyl)-1,2,3,4,6,11,12,12a-octahydrobenzo[e]pyrazino[1,2-a][1,4]diazepin-8-yl)benzonitrile (KJW006-070) in an aspect of the invention. See FIG. 8.

The compound was prepared following General Procedure I using 8-iodo-2-(2-phenoxyacetyl)-1,3,4,12a-tetrahydrobenzo[e]pyrazino[1,2-a][1,4]diazepine-6,12 (2H, 11H)-dione (KJW006-066) and 3-cyanophenylboronic acid. ¹H NMR (400 MHz, Acetic Acid-d₄) δ 8.13 (dd, J=8.2, 2.7 Hz, 1H), 8.07 (s, 1H), 8.00 (d, J=8.2 Hz, 1H), 7.83-7.64 (m, 3H), 7.45 (d, J=2.5 Hz, 1H), 7.28 (dt, J=10.9, 7.7 Hz, 2H), 6.97 (d, J=8.0 Hz, 3H), 5.11-4.96 (m, 0.58×2H), 4.94-4.79 (m, 0.42×2H), 4.60-4.38 (m, 2H), 4.29-3.87 (m, 4H), 3.77-3.65 (m, 1H). LCMS RT (Method 1)=4.65 min, m/z 467.2 [M+H⁺].

### Example 8

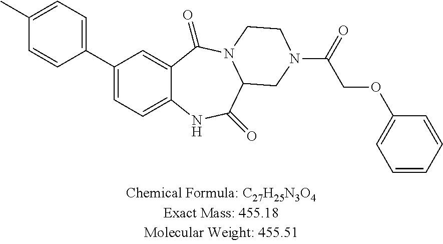 [SMILES: Cc1ccc(-c2ccc3c(c2)C(=O)N2CCN(C(=O)COc4ccccc4)CC2C(=O)N3)cc1]

This example is directed to the synthesis of 2-(2-Phenoxyacetyl)-8-(p-tolyl)-1,3,4,12a-tetrahydrobenzo[e]pyrazino[1,2-a][1,4]diazepine-6,12 (2H, 11H)-dione (KJW006-071) in an aspect of the invention. See FIG. 8.

The compound was prepared following General Procedure I using 8-iodo-2-(2-phenoxyacetyl)-1,3,4,12a-tetrahydrobenzo[e]pyrazino[1,2-a]1[1,4]diazepine-6,12 (2H, 11H)-dione (KJW006-066) and 4-tolyllboronic acid. ¹H NMR (400 MHz, DMSO-d₆) δ 10.68 (s, 0.70×1H), 10.59 (s, 0.30×1H), 7.86 (d, J=8.2 Hz, 1H), 7.63-7.50 (m, 3H), 7.42-7.23 (m, 5H), 7.03-6.86 (m, 3H), 5.08-4.91 (m, 0.68×2H), 4.89-4.68 (m, 0.32×2H), 4.37 (dt, J=8.9, 4.5 Hz, 1H), 4.27-3.34 (m, 6H), 2.36 (s, 3H). LCMS RT (Method 1)=5.16 min, m/z 456.2 [M+H⁺].

### Example 9

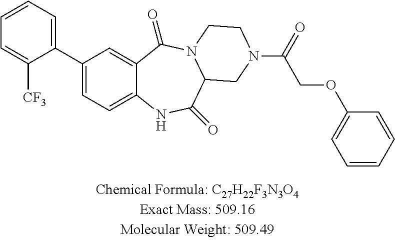 [SMILES: O=C1Nc2ccc(-c3ccccc3C(F)(F)F)cc2C(=O)N2CCN(C(=O)COc3ccccc3)CC12]

This example is directed to the synthesis of 2-(2-Phenoxyacetyl)-8-(2-(trifluoromethyl)phenyl)-1,3,4,12a-tetrahydrobenzo[e]pyrazino[1,2-a][1,4]diazepine-6,12 (2H, 11H)-dione (KJW006-072, KJW007-024) in an aspect of the invention. See FIG. 8.

The compound was prepared following General Procedure I using 8-iodo-2-(2-phenoxyacetyl)-1,3,4,12a-tetrahydrobenzo[e]pyrazino[1,2-a][1,4]diazepine-6,12 (2H, 11H)-dione (KJW006-066) and 2-trifluoromethylboronic acid. ¹H NMR (400 MHz, Acetic Acid-d₄) δ 8.06 (d, J=8.1 Hz, 1H), 7.84 (d, J=7.9 Hz, 1H), 7.69 (t, J=7.6 Hz, 1H), 7.61 (t, J=7.7 Hz, 1H), 7.42 (d, J=7.6 Hz, 1H), 7.34 (d, J=8.2 Hz, 1H), 7.28 (q, J=8.1 Hz, 2H), 7.16 (s, 1H), 6.97 (dd, J=7.7, 5.1 Hz, 3H), 5.04 (q, J=14.6 Hz, 0.57×2H), 4.94-4.79 (m, 0.43×2H), 4.58-4.35 (m, 2H), 4.30-4.09 (m, 1H), 4.04-3.86 (m, 3H), 3.71 (ddd, J=12.7, 8.1, 4.3 Hz, 1H). ¹⁹F NMR (376 MHz, Acetic Acid-d₄) 6-57.30. LCMS RT (Method 1)=5.21 min, m/z 510.1 [M+H⁺].

### Example 10

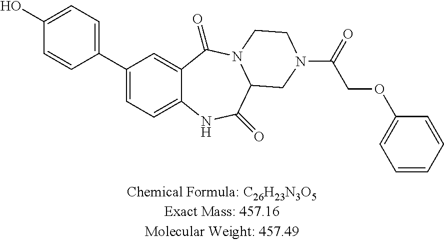 [SMILES: O=C1Nc2ccc(-c3ccc(O)cc3)cc2C(=O)N2CCN(C(=O)COc3ccccc3)CC12]

This example is directed to the synthesis of 8-(4-Hydroxyphenyl)-2-(2-phenoxyacetyl)-1,3,4,12a-tetrahydrobenzo[e]pyrazino[1,2-a][1,4]diazepine-6,12 (2H, 11H)-dione (KJW006-073) in an aspect of the invention. See FIG. 8.

The compound was prepared following General Procedure I using 8-iodo-2-(2-phenoxyacetyl)-1,3,4,12a-tetrahydrobenzo[e]pyrazino[1,2-a][1,4]diazepine-6,12 (2H, 11H)-dione (KJW006-066) and 4-hydroxyboronic acid. ¹H NMR (400 MHz, Acetic Acid-d₄) δ 8.03 (d, J=8.3 Hz, 1H), 7.58 (dt, J=9.3, 2.2 Hz, 3H), 7.34 (dd, J=3.8, 1.6 Hz, 1H), 7.27 (dt, J=11.1, 7.8 Hz, 2H)), 7.00-6.92 (m, 5H), 5.11-4.96 (m, 0.55×2H), 4.93-4.78 (m, 0.45×2H), 4.60-4.36 (m, 2H), 4.27-3.66 (m, 5H). LCMS RT (Method 1)=4.192 min, m/z 458.2 [M+H⁺].

### Example 11

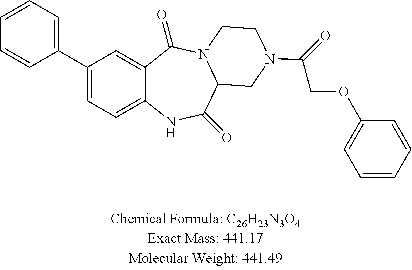 [SMILES: O=C1Nc2ccc(-c3ccccc3)cc2C(=O)N2CCN(C(=O)COc3ccccc3)CC12]

This example is directed to the synthesis of 2-(2-Phenoxyacetyl)-8-phenyl-1,3,4,12a-tetrahydrobenzo[e]pyrazino[1,2-a][1,4]diazepine-6,12 (2H, 11H)-dione (KJW006-075) in an aspect of the invention. See FIG. 8.

The compound was prepared following General Procedure I using 8-iodo-2-(2-phenoxyacetyl)-1,3,4,12a-tetrahydrobenzo[e]pyrazino[1,2-a][1,4]diazepine-6,12 (2H, 11H)-dione (KJW006-066) and 4-hydroxyphenylboronic acid. ¹H NMR (400 MHz, DMSO-d₆) δ 10.70 (s, 0.69×1H), 10.61 (s, 0.31×H), 7.88 (d, J=8.2 Hz, 1H), 7.69-7.49 (m, 6H), 7.48-7.36 (m, 2H), 7.32-7.22 (m, 2H), 7.02-6.89 (m, 2H), 5.08-4.91 (m, 0.69×2H), 4.89-4.70 (m, 0.31×2H), 4.37 (dd, J=8.3, 4.6 Hz, 1H), 4.24-3.35 (m, 5H). LCMS RT (Method 1)=4.864 min, m/z 442.2 [M+H⁺].

### Example 12

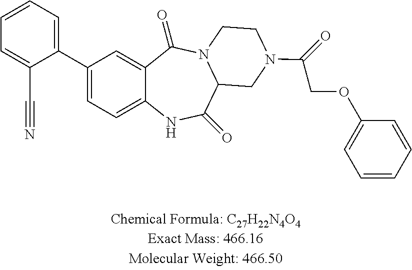 [SMILES: N#Cc1ccccc1-c1ccc2c(c1)C(=O)N1CCN(C(=O)COc3ccccc3)CC1C(=O)N2]

This example is directed to the synthesis of 2-(6,12-Dioxo-2-(2-phenoxyacetyl)-1,2,3,4,6,11,12,12a-octahydrobenzo[e]pyrazino[1,2-a][1,4]diazepin-8-yl)benzonitrile (KJW007-020) in an aspect of the invention. See FIG. 8.

The compound was prepared following General Procedure I using 8-iodo-2-(2-phenoxyacetyl)-1,3,4,12a-tetrahydrobenzo[e]pyrazino[1,2-a][1,4]diazepine-6,12 (2H, 11H)-dione (KJW006-066) and 2-cyanophenylboronic acid. ¹H NMR (400 MHz, Acetic Acid-d₄) δ 8.19 (t, J=2.1 Hz, 1H), 7.92-7.83 (m, 2H), 7.77 (t, J=7.7 Hz, 1H), 7.67 (d, J=7.9 Hz, 1H), 7.56 (t, J=7.6 Hz, 1H), 7.34 (d, J=8.4 Hz, 1H), 7.28 (q, J=8.1 Hz, 2H), 7.02-6.91 (m, 3H), 5.05 (q, J=14.6 Hz, 0.60×2H), 4.94-4.76 (m, 0.40×2H), 4.58 (dd, J=14.3, 4.6 Hz, 0.40×1H), 4.54-4.47 (m, 1H), 4.41 (dd, J=14.6, 3.9 Hz, 0.60×1H), 4.25-3.65 (m, 5H). LCMS RT (Method 1)=4.64 min, m/z 467.2 [M+H⁺].

### Example 13

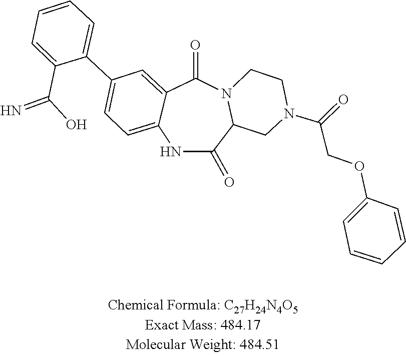 [SMILES: N=C(O)c1ccccc1-c1ccc2c(c1)C(=O)N1CCN(C(=O)COc3ccccc3)CC1C(=O)N2]

This example is directed to the synthesis of 2-(6,12-Dioxo-2-(2-phenoxyacetyl)-1,2,3,4,6,11,12,12a-octahydrobenzo[e]pyrazino[1,2-a][1,4]diazepin-8-yl)benzimidic acid (KJW013-014) in an aspect of the invention. See FIG. 8.

The compound was prepared following General Procedure I using 8-iodo-2-(2-phenoxyacetyl)-1,3,4,12a-tetrahydrobenzo[e]pyrazino[1,2-a][1,4]diazepine-6,12 (2H, 11H)-dione (KJW006-066) and 2-cyanophenylboronic acid. ¹H NMR (400 MHz, Acetic Acid-d₄) δ 8.07 (d, J=2.2 Hz, 1H), 7.69 (dt, J=7.5, 2.6 Hz, 2H), 7.59 (t, J=7.6 Hz, 1H), 7.52-7.40 (m, 3H), 7.32-7.22 (m, 3H), 7.02-6.93 (m, 4H), 5.16-4.97 (m, 0.60×2H), 4.94-4.77 (m, 0.40×2H), 4.58 (dd, J=14.5, 4.6 Hz, 0.42×1H), 4.53-4.45 (m, 1H), 4.42 (dd, J=14.5, 3.9 Hz, 0.58×1H), 4.26-3.62 (m, 5H). LCMS RT (Method 2)=2.989 min. m/z 485.2 [M+H⁺].

### Example 14

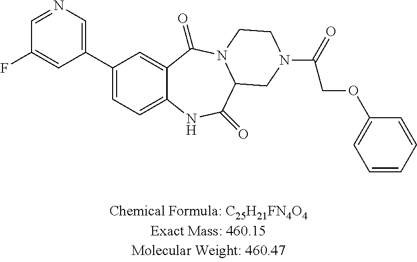 [SMILES: O=C1Nc2ccc(-c3cncc(F)c3)cc2C(=O)N2CCN(C(=O)COc3ccccc3)CC12]

This example is directed to the synthesis of 8-(5-fluoropyridin-3-yl)-2-(2-phenoxyacetyl)-1,3,4,12a-tetrahydrobenzo[e]pyrazino[1,2-a][1,4]diazepine-6,12 (2H, 11H)-dione (KJW007-021) in an aspect of the invention. See FIG. 8.

The compound was prepared following General Procedure I using 8-iodo-2-(2-phenoxyacetyl)-1,3,4,12a-tetrahydrobenzo[e]pyrazino[1,2-a]1[1,4]diazepine-6,12 (2H, 11H)-dione (KJW006-066) and (5-fluoropyridin-3-yl)-boronic acid. ¹H NMR (400 MHz, DMSO-d₆) δ 10.64 (s, 0.67×1H), 10.55 (s, 0.33×1H), 7.61 (ddd, J=8.3, 4.4, 1.6 Hz, 2H), 7.54 (s, 1H), 7.51 (dd, J=2.7, 1.4 Hz, 2H), 7.26 (tt, J=7.3, 1.3 Hz, 3H), 6.97-6.89 (m, 3H), 5.06-4.90 (m, 0.67×2H), 4.88-4.69 (m, 0.33×2H), 4.31 (dt, J=7.4, 4.6 Hz, 1H), 4.21-3.31 (m, 6H). ¹⁹F NMR (376 MHz, DMSO-d₆) δ−74.45.

### Example 15

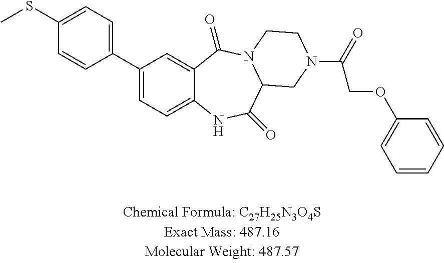 [SMILES: CSc1ccc(-c2ccc3c(c2)C(=O)N2CCN(C(=O)COc4ccccc4)CC2C(=O)N3)cc1]

This example is directed to the synthesis of 8-(4-(methylthio)phenyl)-2-(2-phenoxyacetyl)-1,3,4,12a-tetrahydrobenzo[e]pyrazino[1,2-a]1[1,4]diazepine-6,12 (2H, 11H)-dione (KJW007-022) in an aspect of the invention. See FIG. 8.

The compound was prepared following General Procedure I using 8-iodo-2-(2-phenoxyacetyl)-1,3,4,12a-tetrahydrobenzo[e]pyrazino[1,2-a][1,4]diazepine-6,12 (2H, 11H)-dione (KJW006-066) and 4-methylthiophenylboronic acid. ¹H NMR (400 MHz, DMSO-d₆) δ 10.68 (s, 0.68×1H), 10.59 (s, 0.32×1H), 7.86 (d, J=8.3 Hz, 1H), 7.65-7.50 (m, 3H), 7.44-7.35 (m, 3H), 7.26 (td, J=8.6, 7.2, 2.8 Hz, 2H), 7.00-6.88 (m, 3H), 5.08-4.91 (m, 0.68×2H), 4.89-4.70 (m, 0.32×2H), 4.37 (q, J=5.0 Hz, 1H), 4.24-3.35 (m, 6H), 2.53 (s, 3H). LCMS RT (Method 1)=5.234 min, m/z 488.2 [M+H⁺].

### Example 16

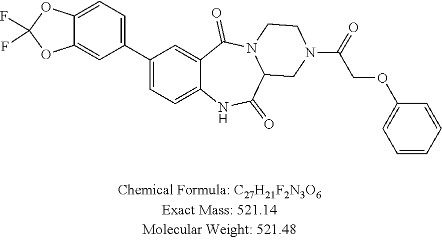 [SMILES: O=C1Nc2ccc(-c3ccc4c(c3)OC(F)(F)O4)cc2C(=O)N2CCN(C(=O)COc3ccccc3)CC12]

This example is directed to the synthesis of 8-(2,2-difluorobenzo[d][1,3]dioxol-5-yl)-2-(2-phenoxyacetyl)-1,3,4,12a-tetrahydrobenzo[e]pyrazino[1,2-a][1,4]diazepine-6,12 (2H, 11H)-dione (KJW007-023) in an aspect of the invention. See FIG. 8.

The compound was prepared following (General Procedure I using 8-iodo-2-(2-phenoxyacetyl)-1,3,4,12a-tetrahydrobenzo[e]pyrazino[1,2-a][1,4]diazepine-6,12 (2H, 11H)-dione (KJW006-066) and (2,2-difluorobenzo[d][1,3]dioxol-5-yl)boronic acid. LCMS RT (Method 1)=5.443 min, m/z 522.1 [M+H⁺]. ¹H NMR (400 MHz, Acetic Acid-d₄) δ 8.09 (dd, J=8.4, 2.0 Hz, 1H), 7.60 (dd, J=8.4, 1.8 Hz, 1H), 7.54 (t, J=2.1 Hz, 1H), 7.50 (dt, J=8.4, 1.6 Hz, 1H), 7.38 (dd, J=3.4, 1.8 Hz, 1H), 7.34-7.23 (m, 3H), 7.00-6.96 (m, 3H), 5.09-4.98 (m, 0.55×2H), 4.91-4.80 (m, 0.45×1H), 4.59-4.37 (m, 2H), 4.27-3.67 (m, 5H). ¹⁹F NMR (376 MHz, Acetic Acid-d₄) δ−51.21. LCMS RT (Method 1)=5.443 min, m/z 522.1 [M+H⁺].

### Example 17

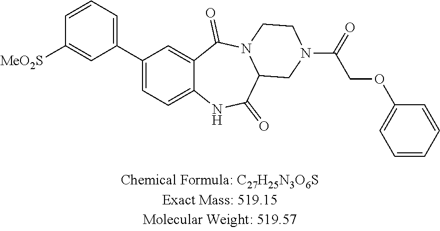 [SMILES: CS(=O)(=O)c1cccc(-c2ccc3c(c2)C(=O)N2CCN(C(=O)COc4ccccc4)CC2C(=O)N3)c1]

This example is directed to the synthesis of 8-(3-(methylsulfonyl)phenyl)-2-(2-phenoxyacetyl)-1,3,4,12a-tetrahydrobenzo[e]pyrazino[1,2-a][1,4]diazepine-6,12 (2H, 11H)-dione (KJW007-025) in an aspect of the invention. See FIG. 8.

The compound was prepared following General Procedure I using 8-iodo-2-(2-phenoxyacetyl)-1,3,4,12a-tetrahydrobenzo[e]pyrazino[1,2-a]1[1,4]diazepine-6,12 (2H, 11H)-dione (KJW006-066) and 3-methylsulfonylphenylboronic acid. ¹H NMR (400 MHz, Acetic Acid-d₄) δ 8.29 (q, J=2.1 Hz, 1H), 8.13 (d, J=8.3 Hz, 1H), 8.05 (t, J=7.2 Hz, 2H), 7.77 (t, J=7.8 Hz, 1H), 7.70 (d, J=8.3 Hz, 1H), 7.50 (t, J=2.1 Hz, 1H), 7.32-7.23 (m, 2H), 6.97 (dd, J=8.4, 5.4 Hz, 3H), 5.10-4.96 (m, 0.58×2H), 4.92-4.80 (m, 0.42×2H), 4.60-4.36 (m, 2H), 4.28-4.09 (m, 1H), 4.07-3.67 (m, 4H), 3.20 (s, 3H). LCMS RT (Method 1)=4.324 min, m/z 520.2 [M+H⁺].

### Example 18

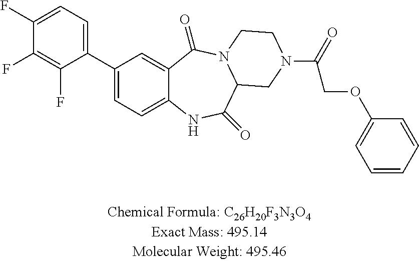 [SMILES: O=C1Nc2ccc(-c3ccc(F)c(F)c3F)cc2C(=O)N2CCN(C(=O)COc3ccccc3)CC12]

This example is directed to the synthesis of 2-(2-phenoxyacetyl)-8-(2,3,4-trifluorophenyl)-1,3,4,12a-tetrahydrobenzo[e]pyrazino[1,2-a]1[1,4]diazepine-6,12 (2H, 11H)-dione (KJW007-027) in an aspect of the invention. See FIG. 8.

The compound was prepared following General Procedure I using 8-iodo-2-(2-phenoxyacetyl)-1,3,4,12a-tetrahydrobenzo[e]pyrazino[1,2-a]1[1,4]diazepine-6,12 (2H, 11H)-dione (KJW006-066) and 2,3,4-trfluorophenylboronic acid. ¹H NMR (400 MHz, Acetic Acid-d₄) δ 8.11 (d, J=8.2 Hz, 1H), 7.54 (dd, J=8.4, 2.1 Hz, 1H), 7.36 (s, 2H), 7.26 (qd, J=10.9, 10.3, 7.7 Hz, 3H), 6.95 (dd, J=13.2, 7.6 Hz, 3H), 5.10-4.97 (m, 0.43×2H), 4.92-4.79 (m, 0.57×2H), 4.58-4.36 (m, 2H), 4.28-3.65 (m, 5H). ¹⁹F NMR (376 MHz, Acetic Acid-d₄) δ−135.11 (dqd, J=19.8, 10.3, 9.8, 6.0 Hz, 1F), −139.43 (ddd, J=36.7, 18.5, 9.0 Hz, 1F), −161.07-−161.25 (m, 1F). LCMS RT (Method 1)=5.183 min, m/z 496.1 [M+H⁺].

### Example 19

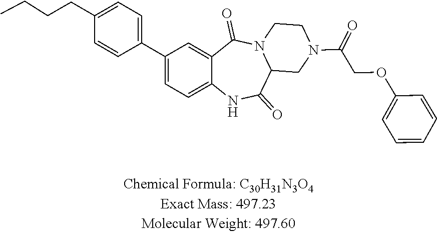 [SMILES: CCCCc1ccc(-c2ccc3c(c2)C(=O)N2CCN(C(=O)COc4ccccc4)CC2C(=O)N3)cc1]

This example is directed to the synthesis of 8-(4-butylphenyl)-2-(2-phenoxyacetyl)-1,3,4,12a-tetrahydrobenzo[e]pyrazino[1,2-a][1,4]diazepine-6,12 (2H, 11H)-dione (KJW007-028) in an aspect of the invention. See FIG. 8.

The compound was prepared following General Procedure I using 8-iodo-2-(2-phenoxyacetyl)-1,3,4,12a-tetrahydrobenzo[e]pyrazino[1,2-a][1,4]diazepine-6,12 (2H, 11H)-dione (KJW006-066) and 4-butylphenylboronic acid. 1H NMR (400 MHz, Acetic Acid-d₄) δ 8.06 (d, J=8.3 Hz, 1H), 7.62 (ddd, J=8.2, 3.7, 1.7 Hz, 3H), 7.39 (dd, J=3.5, 1.7 Hz, 1H), 7.36-7.23 (m, 4H), 7.01-6.90 (m, 3H), 5.10-4.98 (m, 0.57×2H), 4.92-4.80 (m, 0.43×2H), 4.59-4.36 (m, 2H), 4.27-4.10 (m, 1H), 4.06-3.66 (m, 4H), 2.68 (t, J=7.7 Hz, 2H), 1.65 (tt, J=7.9, 6.4 Hz, 2H), 1.39 (h, J=7.4 Hz, 2H), 0.96 (t, J=7.4 Hz, 3H). LCMS RT (Method 1)=6.140 min, m/z 498.3 [M+H⁺].

### Example 20

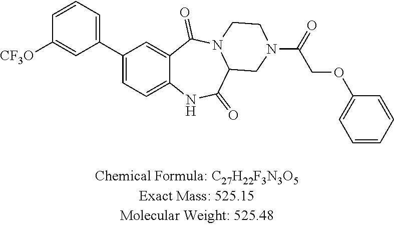

This example is directed to the synthesis of 2-(2-phenoxyacetyl)-8-(3-(trifluoromethoxy)phenyl)-1,3,4,12a-tetrahydrobenzo[e]pyrazino[1,2-a][1,4]diazepine-6,12 (2H, 11H)-dione (KJW007-029) in an aspect of the invention. See FIG. 8.

The compound was prepared following General Procedure I using 8-iodo-2-(2-phenoxyacetyl)-1,3,4,12a-tetrahydrobenzo[e]pyrazino[1,2-a][1,4]diazepine-6,12 (2H, 11H)-dione (KJW006-066) and 3-trifluoromethoxyphenylboronic acid. ¹H NMR (400 MHz, Acetic Acid-d₄) δ 8.11 (d, J=8.3 Hz, 1H), 7.71 (d, J=7.8 Hz, 1H), 7.66-7.56 (m, 3H), 7.43 (t, J=2.3 Hz, 1H), 7.38 (d, J=8.2 Hz, 1H), 7.32-7.22 (m, 2H), 6.96 (dd, J=13.6, 7.6 Hz, 3H), 5.09-4.97 (m, 0.55×2H), 4.92-4.80 (m, 0.45×2H), 4.59-4.09 (m, 4H), 4.07-3.66 (m, 3H). ¹⁹F NMR (376 MHz, Acetic Acid-d₄) δ−58.63. LCMS RT (Method 1)=5.531 min, m/z 526.2 [M+H⁺].

### Example 21

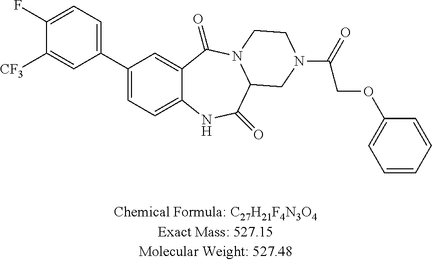 [SMILES: O=C1Nc2ccc(-c3ccc(F)c(C(F)(F)F)c3)cc2C(=O)N2CCN(C(=O)COc3ccccc3)CC12]

This example is directed to the synthesis of 8-(4-fluoro-3-(trifluoromethyl)phenyl)-2-(2-phenoxyacetyl)-1,3,4,12a-tetrahydrobenzo[e]pyrazino[1,2-a][1,4]diazepine-6,12 (2H, 11H)-dione (KJW007-030) in an aspect of the invention. See FIG. 8.

General Procedure I using 8-iodo-2-(2-phenoxyacetyl)-1,3,4,12a-tetrahydrobenzo[e]pyrazino[1,2-a][1,4]diazepine-6,12 (2H, 11H)-dione (KJW006-066) and 3-trifluoromethyl-4-fluorophenylboronic acid. ¹H NMR (400 MHz, Acetic Acid-d₄) δ 8.12 (d, J=8.2 Hz, 1H), 7.98 (dt, J=6.2, 2.7 Hz, 2H), 7.64 (dd, J=8.3, 1.8 Hz, 1H), 7.50-7.41 (m, 2H), 7.32-7.23 (m, 2H), 6.97 (dq, J=8.8, 2.6 Hz, 3H), 5.10-4.97 (m, 0.57×2H), 4.92-4.79 (m, 0.43×2H), 4.60-4.37 (m, 2H), 4.30-3.83 (m, 4H), 3.70 (ddd, J=12.8, 8.2, 4.4 Hz, 1H). ¹⁹F NMR (376 MHz, Acetic Acid-d₄) δ−62.16 (dd, J=12.6, 3.0 Hz, 3F), −114.33-−117.92 (m, 1F). LCMS RT (Method 1)=5.466 min. m/z 528.2 [M+H⁺].

### Example 22

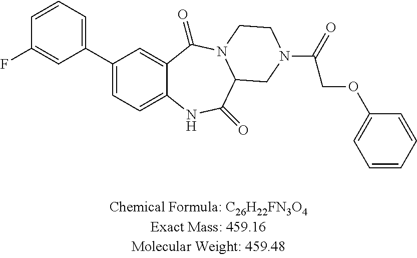 [SMILES: O=C1Nc2ccc(-c3cccc(F)c3)cc2C(=O)N2CCN(C(=O)COc3ccccc3)CC12]

This example is directed to the synthesis of 8-(3-fluorophenyl)-2-(2-phenoxyacetyl)-1,3,4,12a-tetrahydrobenzo[e]pyrazino[1,2-a][1,4]diazepine-6,12 (2H, 11H)-dione (KJW007-031) in an aspect of the invention. See FIG. 8.

The compound was prepared following General Procedure I using 8-iodo-2-(2-phenoxyacetyl)-1,3,4,12a-tetrahydrobenzo[e]pyrazino[1,2-a]1[1,4]diazepine-6,12 (2H, 11H)-dione (KJW006-066) and 3-fluorophenylboronic acid. ¹H NMR (400 MHz, Acetic Acid-d₄) δ 8.09 (d, J=8.3 Hz, 1H), 7.63 (dd, J=8.4, 1.8 Hz, 1H), 7.56-7.39 (m, 4H), 7.33-7.15 (m, 3H), 6.97 (td, J=6.3, 5.8, 2.8 Hz, 3H), 5.10-4.97 (m, 0.57×2H), 4.93-4.79 (m, 0.43×2H), 4.61-4.36 (m, 2H), 4.27-3.64 (m, 5H). ¹⁹F NMR (376 MHz, Acetic Acid-d₄) δ, −113.38-−113.48 (m, 1F). LCMS RT (Method 1)=4.975 min, m/z 460.1 [M+H⁺].

### Example 23

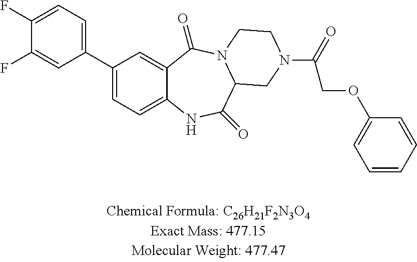 [SMILES: O=C1Nc2ccc(-c3ccc(F)c(F)c3)cc2C(=O)N2CCN(C(=O)COc3ccccc3)CC12]

This example is directed to the synthesis of 8-(3,4-difluorophenyl)-2-(2-phenoxyacetyl)-1,3,4,12a-tetrahydrobenzo[e]pyrazino[1,2-a]1[1,4]diazepine-6,12 (2H, 11H)-dione (KJW007-026) in an aspect of the invention. See FIG. 8.

General Procedure I using 8-iodo-2-(2-phenoxyacetyl)-1,3,4,12a-tetrahydrobenzo[e]pyrazino[1,2-a][1,4]diazepine-6,12 (2H, 11H)-dione (KJW006-066) and 3,4-difluorophenylboronic acid. ¹H NMR (400 MHz, Acetic Acid-d₄) δ 8.09 (dd, J=8.4, 2.1 Hz, 1H), 7.60 (dd, J=8.4, 1.8 Hz, 1H), 7.54 (t, J=2.1 Hz, 1H), 7.51-7.48 (m, 1H), 7.38 (dd, J=3.4, 1.8 Hz, 1H), 7.32-7.18 (m, 3H), 7.03-6.92 (m, 3H), 5.10-4.96 (m, 0.56×2H), 4.92-4.79 (m, 0.44×2H), 4.59-4.46 (m, 1H), 4.45-4.37 (m, 1H), 4.29-3.85 (m, 4H), 3.71 (ddd, J=13.0, 8.1, 4.5 Hz, 1H). ¹⁹F NMR (376 MHz, Acetic Acid-d₄) δ−51.21. LCMS RT (Method 1)=5.082 min, m/z 478.1 [M+H⁺].

### Example 24

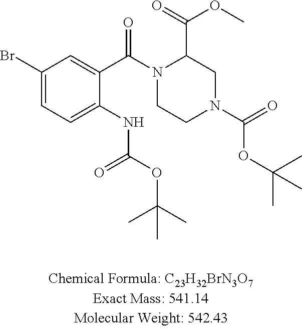 [SMILES: COC(=O)C1CN(C(=O)OC(C)(C)C)CCN1C(=O)c1cc(Br)ccc1NC(=O)OC(C)(C)C]

This example is directed to the synthesis of 1-(tert-butyl) 3-methyl 4-(5-bromo-2-((tert-butoxycarbonyl)amino)benzoyl)piperazine-1,3-dicarboxylate (KJW010-051) in an aspect of the invention. See FIG. 2.

The compound was prepared following General Procedure A using 1-(tert-butyl) 3-methyl piperazine-1,3-dicarboxylate. ¹H NMR (400 MHz, DMSO-d₆) δ 7.59 (td, J=9.1, 2.4 Hz, 2H), 7.52 (s, 1H), 7.36 (s, 1H), 7.26 (s, 1H), 5.10 (s, 1H), 4.46-4.23 (m, 2H), 3.73 (s, 0.77×3H), 3.68 (s, 0.33×3H), 3.45-3.19 (m, 4H), 1.44 (s, 9H), 1.38 (s, 9H). LCMS RT (Method 3)=3.609 min, m/z 564.2 [M+Na⁺].

### Example 25

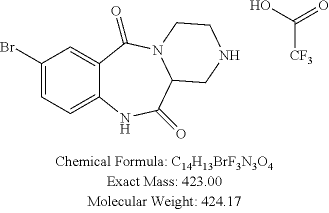 [SMILES: O=C1Nc2ccc(Br)cc2C(=O)N2CCNCC12 ; O=C(O)C(F)(F)F]

This example is directed to the synthesis of 8-bromo-1,3,4,12a-tetrahydrobenzo[e]pyrazino[1,2-a][1,4]diazepine-6,12 (2H, 11H)-dione 2,2,2-trifluoroacetate (KJW008-042) in an aspect of the invention. See FIG. 2.

The compound was prepared following General Procedure B using 1-(tert-butyl) 3-methyl 4-(5-bromo-2-((tert-butoxycarbonyl)amino)benzoyl)piperazine-1,3-dicarboxylate (KJW010-051). ¹H NMR (400 MHz, DMSO-d₆) δ 10.93 (s, 1H), 9.60 (bs, 1H), 8.74 (bs, 1H), 7.84 (d, J=2.4 Hz, 1H), 7.76 (dd, J=8.6, 2.4 Hz, 1H), 7.11 (d, J=8.6 Hz, 1H), 4.51 (dd, J=5.2, 3.0 Hz, 1H), 4.17 (dt, J=14.4, 4.4 Hz, 1H), 3.65 (dd, J=13.7, 3.1 Hz, 1H), 3.55-3.13 (m, 4H). ¹⁹F NMR (376 MHz, DMSO-d₆) δ 73.75. LCMS RT (Method 2)=2.208 min, m/z 311.9 [M+2H⁺].

### Example 26

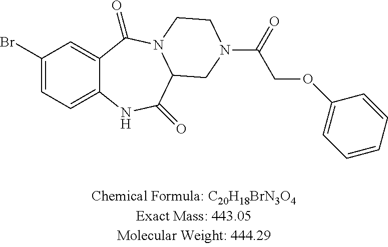 [SMILES: O=C1Nc2ccc(Br)cc2C(=O)N2CCN(C(=O)COc3ccccc3)CC12]

This example is directed to the synthesis of 8-bromo-2-(2-phenoxyacetyl)-1,3,4,12a-tetrahydrobenzo[e]pyrazino[1,2-a][1,4]diazepine-6,12 (2H, 11H)-dione in an aspect of the invention.

To a slurry of 8-bromo-1,3,4,12a-tetrahydrobenzo[e]pyrazino[1,2-a][1,4]diazepine-6,12 (2H, 11H)-dione 2,2,2-trifluoroacetate (Example 25) in DCM was added disopropylethylamine (DIEA) (3 eq.) or triethylamine (TEA) (3.0 eq.) followed by 2-phenoxyacetyl chloride (1.2 eq.) via syringe. The resultant solution was stirred for 2 hours and then diluted with DCM. The result was washed twice with saturated NaHCO₃, dried (Na₂SO₄) and concentrated under vacuum. The residue was purified by flash column chromatography (silica gel, 0-10% MeOH-DCM) to afford the title compound. ¹H NMR (400 MHz, Acetic Acid-d₄) δ 8.10 (dd, J=4.0, 2.3 Hz, 1H), 7.70 (dt, J=8.7, 1.9 Hz, 1H), 7.33-7.22 (m, 2H), 7.09 (d, J=8.6 Hz, 1H), 7.00-6.91 (m, 3H), 5.09-4.97 (m, 0.58×2H), 4.91-4.78 (m, 0.42×2H), 4.56-4.34 (m, 2H), 4.24-4.13 (m, 1H), 4.13-3.63 (m, 4H). LCMS RT (Method 2)=2.85 min, m/z 445.1 [M+H⁺).

### Example 27

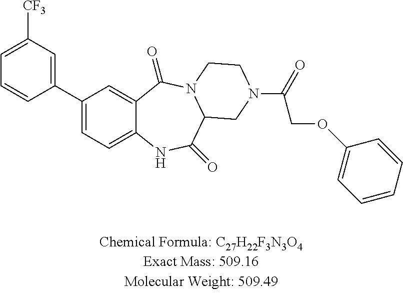 [SMILES: O=C1Nc2ccc(-c3cccc(C(F)(F)F)c3)cc2C(=O)N2CCN(C(=O)COc3ccccc3)CC12]

This example is directed to the synthesis of 2-(2-phenoxyacetyl)-8-(3-(trifluoromethyl)phenyl)-1,3,4,12a-tetrahydrobenzo[e]pyrazino[1,2-a][1,4]diazepine-6,12 (2H, 11H)-dione (KJW013-006) in an aspect of the invention.

The compound was prepared following General Procedure G. ¹H NMR (400 MHz, Acetic Acid-d₄) δ 8.29 (d, J=2.4 Hz, 1H), 7.99 (d, J=4.0 Hz, 1H), 7.97-7.89 (m, 2H), 7.75-7.64 (m, 2H), 7.28 (dt, J=19.7, 8.2 Hz, 3H), 7.01-6.90 (m, 3H), 5.10-4.98 (m, 0.56×2H), 4.94-4.79 (m, 0.44×2H), 4.60-4.37 (m, 2H), 4.29-3.65 (m, 5H). ¹⁹F NMR (376 MHz, DMSO-d₆) δ−61.04, −61.06. LCMS RT (Method 1)=5.235 min, m/z 510.1 [M+H⁺].

### Example 28

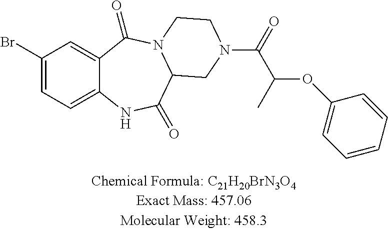 [SMILES: CC(Oc1ccccc1)C(=O)N1CCN2C(=O)c3cc(Br)ccc3NC(=O)C2C1]

This example is directed to the synthesis of 8-bromo-2-(2-phenoxypropanoyl)-1,3,4,12a-tetrahydrobenzo[e]pyrazino[1,2-a][1,4]diazepine-6,12 (2H, 11H)-dione (KJW008-049) in an aspect of the invention.

To a stirring solution of 8-bromo-1,3,4,12a-tetrahydrobenzo[e]pyrazino[1,2-a][1,4]diazepine-6,12 (2H, 11H)-dione 2,2,2-trifluoroacetate (1 eq.) (KJW008-042) and N-methylmorpholine (3 eq.) in THF at 0° C. was added 2-phenoxypropanoyl chloride (1.05 eq.) via syringe. After 1 hour the reaction was diluted with EtOAc and saturated NaHCO₃. The layers were separated, the organic layer was dried (Na₂SO₄) and concentrated under vacuum. The residue was purified by flash column chromatography (silica gel, 0-5% MeOH-DCM) to afford the title compound. ¹H NMR (400 MHz, DMSO-d₆) δ 10.84 (s, 0.40×1H), 10.70 (s, 0.45×1H), 10.61 (s, 0.15×1H), 7.86 (td, J=3.9, 2.5 Hz, 1H), 7.73 (ddt, J=8.4, 5.7, 2.6 Hz, 1H), 7.28-7.19 (m, 2H), 7.11-6.99 (m, 2H), 6.96-6.82 (m, 2H), 5.30 (q, J=6.5 Hz, 0.66×2H), 5.18 (q, J=6.5 Hz, 0.15×2H), 5.05 (q, J=6.4 Hz, 0.20×2H), 4.36-4.19 (m, 2H), 4.10-3.43 (m, 4H), 1.49-1.41 (m, 3H). LCMS RT (Method 2)=2.91 min, m/z 458.1 [M+].

### Example 29

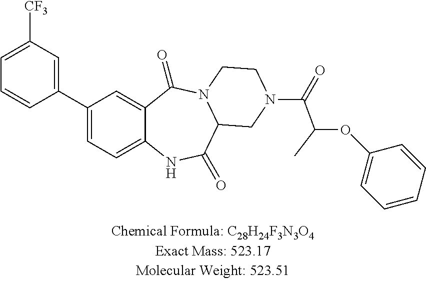 [SMILES: CC(Oc1ccccc1)C(=O)N1CCN2C(=O)c3cc(-c4cccc(C(F)(F)F)c4)ccc3NC(=O)C2C1]

This example is directed to the synthesis of 2-(2-phenoxypropanoyl)-8-(3-(trifluoromethyl)phenyl)-1,3,4,12a-tetrahydrobenzo[e]pyrazino[1,2-a][1,4]diazepine-6,12 (2H, 11H)-dione (KJW008-048) in an aspect of the invention.

The compound was prepared following General Procedure G using 8-bromo-2-(2-phenoxypropanoyl)-1,3,4,12a-tetrahydrobenzo[e]pyrazino[1,2-a][1,4]diazepine-6,12 (2H, 11H)-dione (KJW008-049). ¹H NMR (400 MHz, Acetic Acid-d₄) δ 8.28 (ddd, J=14.3, 9.1, 2.2 Hz, 1H), 8.00-7.87 (m, 3H), 7.69 (dq, J=14.3, 7.4 Hz, 2H), 7.36-7.16 (m, 3H), 7.01-6.75 (m, 3H), 5.35 (q, J=6.5 Hz, 0.21×2H), 5.26 (q, J=6.8 Hz, 0.38×2H), 5.17 (q, J=6.7 Hz, 0.19×2H), 5.10 (q, J=6.6 Hz, 0.21×2H), 4.67-4.40 (m, 2H), 4.38-3.67 (m, 5H), 1.69 (d, J=6.8 Hz, 0.34×3H), 1.65-1.59 (m, 0.65×3H). ¹⁹F NMR (376 MHz, Acetic Acid-d₄) δ−63.40. LCMS RT (Method 1)=5.506 min. m/z 524.2 [M+H⁺].

### Example 30

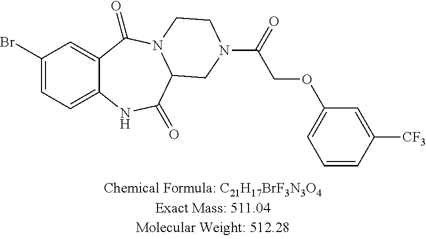 [SMILES: O=C1Nc2ccc(Br)cc2C(=O)N2CCN(C(=O)COc3cccc(C(F)(F)F)c3)CC12]

This example is directed to the synthesis of 8-bromo-2-(2-(3-(trifluoromethyl)phenoxy)acetyl)-1,3,4,12a-tetrahydrobenzo[e]pyrazino[1,2-a][1,4]diazepine-6,12 (2H, 11H)-dione (KJW008-047-1) in an aspect of the invention.

To a stirring solution of 8-bromo-1,3,4,12a-tetrahydrobenzo[e]pyrazino[1,2-a][1,4]diazepine-6,12 (2H, 11H)-dione 2,2,2-trifluoroacetate (1 eq.) (KJW008-042) and N-methylmorpholine (3 eq.) in THF at 0° C. was added 2-(3-(trifluoromethyl)-phenoxyacetyl chloride (1.05 eq.) via syringe. After 1 hour the reaction was diluted with EtOAc and saturated NaHCO₃. The layers were separated, the organic layer was dried (Na₂SO₄) and concentrated under vacuum. The residue was purified by flash column chromatography (silica gel, 0-5% MeOH-DCM) to afford the title compound. ¹H NMR (400 MHz, Chloroform-d) δ 9.11 (s, 0.65×1H), 8.98 (s, 0.35×1H), 8.04 (dd, J=8.3, 2.4 Hz, 1H), 7.51 (ddd, J=8.6, 2.4, 1.8 Hz, 1H), 7.40-7.32 (m, 1H), 7.22 (dddt, J=7.7, 7.1, 1.6, 0.8 Hz, 1H), 7.17-7.12 (m, 1H), 7.11-7.06 (m, 1H), 6.89 (dd, J=12.6, 8.6 Hz, 1H), 4.99 (d, J=1.2 Hz, 0.66×2H), 4.73 (s, 0.34×2H), 4.31 (ddd, J=14.4, 6.8, 4.6 Hz, 1H), 4.23-4.13 (m, 1H), 4.13-3.97 (m, 2H), 3.87-3.65 (m, 2H), 3.40 (ddd, J=13.0, 8.5, 4.0 Hz, 1H). ¹⁹F NMR (376 MHz, Chloroform-d) 6-62.61. LCMS RT (Method 2)=3.224 min, m/z 511.8 [M+H⁺].

### Example 31

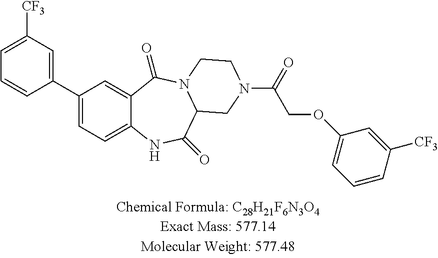 [SMILES: O=C1Nc2ccc(-c3cccc(C(F)(F)F)c3)cc2C(=O)N2CCN(C(=O)COc3cccc(C(F)(F)F)c3)CC12]

This example is directed to the synthesis of 2-(2-(3-(trifluoromethyl)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW008-052) in an aspect of the invention.

The compound was prepared following General Procedure G using 8-bromo-2-(2-(3-(trifluoromethyl)phenoxy)acetyl)-1,3,4,12a-tetrahydrobenzo[e]pyrazino[1,2-a][1,4]diazepine-6,12 (2H, 11H)-dione (KJW008-047-1). ¹H NMR (400 MHz, Acetic Acid-d₄) δ 8.30 (t, J=2.6 Hz, 1H), 7.99 (d, J=4.6 Hz, 1H), 7.93 (ddd, J=8.4, 6.1, 4.2 Hz, 2H), 7.69 (dt, J=15.3, 7.8 Hz, 2H), 7.47 (td, J=8.0, 5.1 Hz, 1H), 7.35-7.19 (m, 4H), 5.05 (m, 0.55×2H), 4.96 (q, J=14.9 Hz, 0.45×2H), 4.55 (dd, J=14.3, 4.9 Hz, 0.42×1H), 4.49 (q, J=4.9 Hz, 1H), 4.39 (dd, J=14.6, 4.0 Hz, 0.58×1H), 4.31-4.10 (m, 1H), 4.06-3.66 (m, 4H). ¹⁹F NMR (376 MHz, Acetic Acid-d₄) δ−63.41, −63.41. LCMS RT (Method 1)=5.943 min, m/z 578.2 [M+H⁺].

### Example 32

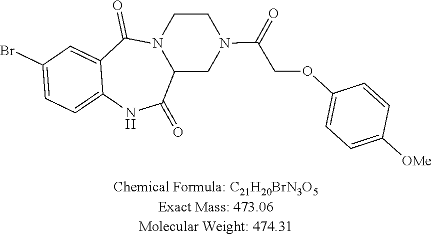 [SMILES: COc1ccc(OCC(=O)N2CCN3C(=O)c4cc(Br)ccc4NC(=O)C3C2)cc1]

This example is directed to the synthesis of 8-bromo-2-(2-(4-methoxyphenoxy)acetyl)-1,3,4,12a-tetrahydrobenzo[e]pyrazino[1,2-a][1,4]diazepine-6,12 (2H, 11H)-dione (KJW008-047-2) in an aspect of the invention.

To a stirring solution of 8-bromo-1,3,4,12a-tetrahydrobenzo[e]pyrazino[1,2-a][1,4]diazepine-6,12 (2H, 11H)-dione 2,2,2-trifluoroacetate (1 eq.) (KJW008-042) and N-methylmorpholine (3 eq.) in THF at 0° C. was added 2-(4-methoxyphenoxy)-acetyl chloride (1.05 eq.) via syringe. After 1 hour the reaction was diluted with EtOAc and saturated NaHCO₃. The layers were separated, the organic layer was dried (Na₂SO₄) and concentrated under vacuum. The residue was purified by flash column chromatography (silica gel, 0-5% MeOH-DCM) to afford the title compound. ¹H NMR (400 MHz, Chloroform-d) δ 9.30 (s, 0.64×1H), 9.17 (s, 0.36×1H), 8.03 (dd, J=10.7, 2.4 Hz, 1H), 7.51 (dd, J=8.6, 2.4 Hz, 1H), 6.95-6.69 (m, 5H), 4.95-4.73 (m, 0.65×2H), 4.63 (s, 0.35×2H), 4.35-4.12 (m, 2H), 4.09-3.90 (m, 2H), 3.82-3.75 (m, 1H), 3.73 (s, 0.38, 3H), 3.72 (s, 0.68×3H), (3.71-3.64 (m, 1H), 3.44 (ddd, J=13.1, 8.6, 4.2 Hz, 1H). LCMS RT (Method 2)=2.998 mn, m/z 473.9 [M⁺].

### Example 33

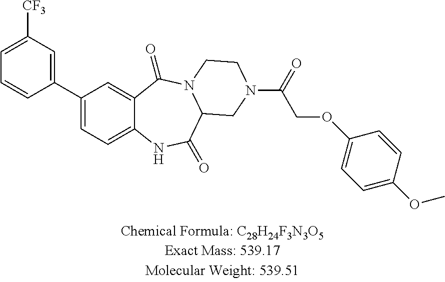 [SMILES: COc1ccc(OCC(=O)N2CCN3C(=O)c4cc(-c5cccc(C(F)(F)F)c5)ccc4NC(=O)C3C2)cc1]

This example is directed to the synthesis of 2-(2-(4-methoxyphenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW008-053) in an aspect of the invention.

The compound was prepared following General Procedure G using 8-bromo-2-(2-(4-methoxyphenoxy)acetyl)-1,3,4,12a-tetrahydrobenzo[e]pyrazino[1,2-a][1,4]diazepine-6,12 (2H, 11H)-dione (KJW008-047-2). ¹H NMR (400 MHz, DMSO-d₆) δ 10.78 (s, 0.68×1H), 10.70 (s, 0.32×1H), 8.09 (d, J=2.5 Hz, 1H), 8.06-7.92 (m, 3H), 7.73 (d, J=8.1 Hz, 2H), 7.26 (d, J=8.4 Hz, 1H), 6.89 (dd, J=9.5, 3.3 Hz, 2H), 6.85-6.79 (m, 2H), 4.98-4.83 (m, 0.70×2H), 4.81-4.62 (m, 0.30×2H), 4.38-4.35 (m, 1H), 4.23-4.09 (m, 2H), 4.03-3.72 (m, 2H), 3.69 (s, 0.33×3H), 3.68 (s, 0.67×3H), 3.65-3.53 (m, 1H), 3.39 (ddd, J=12.9, 8.5, 4.4 Hz, 1H). ¹⁹F NMR (376 MHz, DMSO-d₆) δ−61.04. LCMS RT (Method 1)=5.436 min, m/z 540.2 [M+H⁺].

### Example 34

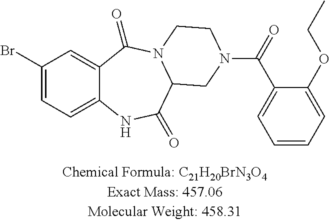 [SMILES: CCOc1ccccc1C(=O)N1CCN2C(=O)c3cc(Br)ccc3NC(=O)C2C1]

This example is directed to the synthesis of 8-bromo-2-(2-ethoxybenzoyl)-1,3,4,12a-tetrahydrobenzo[e]pyrazino[1,2-a][1,4]diazepine-6,12 (2H, 11H)-dione (KJW008-054) in an aspect of the invention.

To a stirring solution of 8-bromo-1,3,4,12a-tetrahydrobenzo[e]pyrazino[1,2-a][1,4]diazepine-6,12 (2H, 11H)-dione 2,2,2-trifluoroacetate (1 eq.) (KJW008-042) and N-methylmorpholine (3 eq.) in THF at 0° C. was added 2-(2-ethoxyphenoxy)-acetyl chloride (1.05 eq.) via syringe. After 1 hour the reaction was diluted with EtOAc and saturated NaHCO₃. The layers were separated, the organic layer was dried (Na₂SO₄) and concentrated under vacuum. The residue was purified by flash column chromatography (silica gel, 0-5% MeOH-DCM) to afford the title compound. ¹H NMR (400 MHz, Chloroform-d) δ 8.49 (bs, 1H), 8.19-8.03 (m, 1H), 7.65-7.54 (m, 1H), 7.46-7.28 (m, 2H), 7.02-6.79 (m, 3H), 4.46-3.41 (m, 9H), 1.38 (m, 0.59×3H), 1.11 (t, J=7.0 Hz, 0.41×3H). LCMS RT (Method 2)=3.037 min, m/z 457.9 [M⁺].

### Example 35

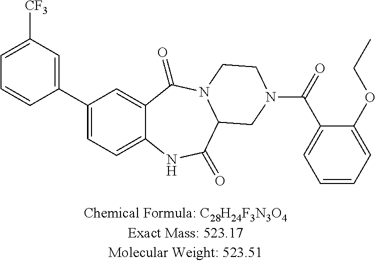 [SMILES: CCOc1ccccc1C(=O)N1CCN2C(=O)c3cc(-c4cccc(C(F)(F)F)c4)ccc3NC(=O)C2C1]

This example is directed to the synthesis of 2-(2-(2-ethoxyphenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW008-056) in an aspect of the invention.

The compound was prepared following General Procedure G using 8-bromo-2-(2-ethoxybenzoyl)-1,3,4,12a-tetrahydrobenzo[e]pyrazino[1,2-a][1,4]diazepine-6,12 (2H, 11H)-dione (KJW008-054). ¹H NMR (400 MHz, Acetic Acid-d₄) δ 8.77 (dd, J=11.2, 2.2 Hz, 1H), 8.47-8.32 (m, 3H), 8.21-8.08 (m, 2H), 8.01-7.66 (m, 3H), 7.56-7.39 (m, 2H), 5.22-4.94 (m, 2H), 4.83 (t, J=5.1 Hz, 2H), 4.79-4.54 (m, 3H), 4.54-4.43 (m, 3H), 4.44-3.98 (m, 4H), 1.49-1.28 (m, 0.69×3H), 1.23 (t, J=7.0 Hz, 0.31×3H). ¹⁹F NMR (376 MHz, Acetic Acid-d₄) δ−63.40, −63.41. LCMS RT (Method 1)=5.396 min, m/z 524.2 [M+H]⁺.

### Example 36

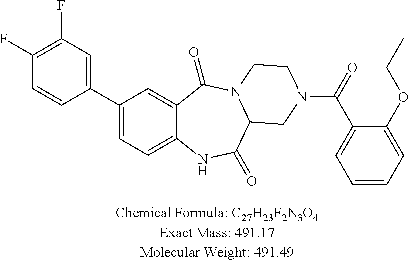 [SMILES: CCOc1ccccc1C(=O)N1CCN2C(=O)c3cc(-c4ccc(F)c(F)c4)ccc3NC(=O)C2C1]

This example is directed to the synthesis of 8-(3,4-difluorophenyl)-2-(2-(2-ethoxyphenoxy)acetyl)-1,3,4,12a-tetrahydrobenzo[e]pyrazino[1,2-a][1,4]diazepine-6,12 (2H, 11H)-dione (KJW008-057) in an aspect of the invention.

The compound was prepared following General Procedure I using 8-bromo-2-(2-ethoxybenzoyl)-1,3,4,12a-tetrahydrobenzo[e]pyrazino[1,2-a][1,4]diazepine-6,12 (2H, 11H)-dione (KJW008-054) and 3,4 difluorophenylboronic acid. ¹H NMR (400 MHz, Acetic Acid-d₄) δ 8.24 (dd, J=10.9, 2.3 Hz, 1H), 7.83 (ddd, J=16.0, 8.5, 2.3 Hz, 1H), 7.58 (dddd, J=12.1, 7.6, 5.2, 2.3 Hz, 1H), 7.49 (td, J=7.2, 3.2 Hz, 2H), 7.45-7.21 (m, 4H), 7.10-6.95 (m, 2H), 4.67 (dd, J=14.6, 4.1 Hz, 0.50×2H), 4.55 (s, 0.50×2H), 4.36 (t, J=5.0 Hz, 0.56×1H), 4.25 (m, 0.44×1H), 4.19-3.96 (m, 6H), 3.96-3.45 (m, 2H), 1.41 (m, 0.57×3H), 1.22 (t, J=7.0 Hz, 0.43×3H). ¹⁹F NMR (376 MHz, Acetic Acid-d₄) δ−138.42 (dt, J=20.3, 10.2 Hz, 1F), −140.65 (m, 1F). LCMS RT (Method 1)=5.104 min. m/z 492.2 [M+H]⁺.

### Example 37

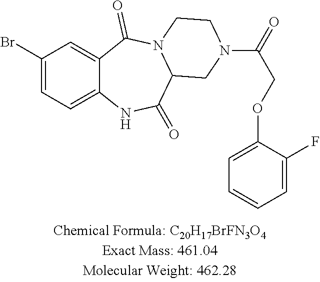 [SMILES: O=C1Nc2ccc(Br)cc2C(=O)N2CCN(C(=O)COc3ccccc3F)CC12]

This example is directed to the synthesis of 8-bromo-2-(2-(2-fluorophenoxy)acetyl)-1,3,4,12a-tetrahydrobenzo[e]pyrazino[1,2-a][1,4]diazepine-6,12 (2H, 11H)-dione (KJW008-073-1) in an aspect of the invention.

To a stirring solution of 8-bromo-1,3,4,12a-tetrahydrobenzo[e]pyrazino[1,2-a][1,4]diazepine-6,12 (2H, 11H)-dione 2,2,2-trifluoroacetate (1 eq.) (KJW008-042) and N-methylmorpholine (3 eq.) in THF at 0° C. was added 2-fluorophenoxyacetyl chloride (1.05 eq.) via syringe. After 1 hour the reaction was diluted with EtOAc and saturated NaHCO₃. The layers were separated, the organic layer was dried (Na₂SO₄) and concentrated under vacuum. The residue was purified by flash column chromatography (silica gel, 0-5% MeOH-DCM) to afford the title compound. ¹H NMR (400 MHz, DMSO-d₆) δ 10.76 (s, 0.68×1H), 10.66 (s, 0.32×1H), 7.87 (dd, J=2.5, 1.4 Hz, 1H), 7.73 (ddd, J=8.7, 3.2, 2.5 Hz, 1H), 7.23-7.12 (m, 1H), 7.11-7.03 (m, 3H), 6.92 (dddd, J=8.1, 7.5, 4.7, 1.6 Hz, 1H), 5.18-5.02 (m, 0.69×2H), 4.99-4.81 (m, 0.31×2H), 4.35 (t, J=4.3 Hz, 0.70×1H), 4.33-4.30 (m, 0.30×1H), 4.20-3.78 (m, 2H), 3.79-3.60 (m, 2H), 3.59-3.49 (m, 1H), 3.43-3.34 (m, 1H). ¹⁹F NMR (376 MHz, DMSO-d₆) δ−135.03-−135.16 (m, 1F). LCMS RT (Method 2)=3.015 min, m/z 463.8 [M+H⁺].

### Example 38

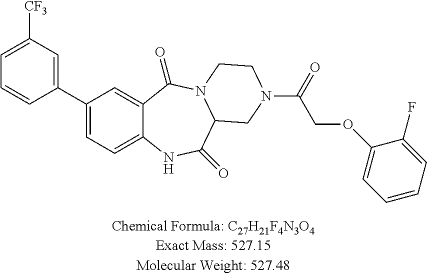 [SMILES: O=C1Nc2ccc(-c3cccc(C(F)(F)F)c3)cc2C(=O)N2CCN(C(=O)COc3ccccc3F)CC12]

This example is directed to the synthesis of 2-(2-(2-fluorophenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW008-079) in an aspect of the invention.

The compound was prepared following General Procedure G using 8-bromo-2-(2-(2-fluorophenoxy)acetyl)-1,3,4,12a-tetrahydrobenzo[e]pyrazino[1,2-a][1,4]diazepine-6,12 (2H, 11H)-dione (KJW008-073-1): ¹H NMR (400 MHz, Acetic Acid-d₄) δ 8.30 (d, J=2.2 Hz, 1H), 7.99 (s, 1H), 7.94 (t, J=10.1 Hz, 2H), 7.69 (dt, J=15.3, 7.7 Hz, 2H), 7.32 (d, J=8.4 Hz, 1H), 7.09 (dt, J=14.9, 9.1 Hz, 3H), 6.94 (p, J=7.2 Hz, 1H), 5.12 (s, 0.58×2H), 4.94 (q, J=14.9 Hz, 0.42×2H), 4.56 (dd, J=14.2, 4.8 Hz, 0.43×1H), 4.49 (q, J=4.9 Hz, 1H), 4.43 (dd, J=14.6, 4.1 Hz, 0.57×1H), 4.30-4.11 (m, 1H), 4.08-3.67 (m, 4H). ¹⁹F NMR (376 MHz, Acetic Acid-d₄) δ−63.40, −131.9, −-140.2 (m). LCMS RT (Method 1)=5.454 min, m/z 528.0 [M+H⁺].

### Example 39

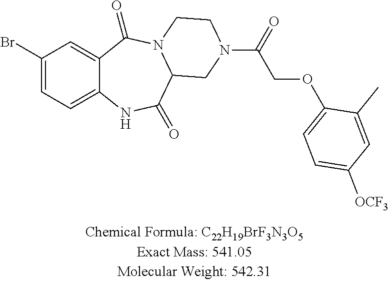 [SMILES: Cc1cc(OC(F)(F)F)ccc1OCC(=O)N1CCN2C(=O)c3cc(Br)ccc3NC(=O)C2C1]

This example is directed to the synthesis of 8-bromo-2-(2-(2-methyl-4-(trifluoromethoxy)phenoxy)acetyl)-1,3,4,12a-tetrahydrobenzo[e]pyrazino[1,2-a][1,4]diazepine-6,12 (2H, 11H)-dione (KJW011-032) in an aspect of the invention.

The compound was prepared following General Procedure D using 2-methyl-4-trifluoromethoxy phenol. ¹H NMR (400 MHz, DMSO-d₆) δ 10.74 (s, 0.68×1H), 10.65 (s, 0.32×1H), 7.86 (dd, J=2.5, 1.5 Hz, 1H), 7.73 (dt, J=8.7, 2.5 Hz, 1H), 7.17 (td, J=1.9, 1.0 Hz, 1H), 7.13-7.05 (m, 2H), 7.00-6.92 (m, 1H), 5.06 (q, J=15.0 Hz, 0.67×2H), 4.97-4.78 (m, 0.33×2H), 4.38-4.28 (m, 1H), 4.20-3.78 (m, 2H), 3.78-3.47 (m, 3H), 3.43-3.34 (m, 1H), 2.22 (s, 0.38×3H), 2.21 (s, 0.62×3H). ¹⁹F NMR (376 MHz, DMSO-d₆) δ−57.12. LCMS RT (Method 2)=3.258 min, m/z 544.1 [M+2H⁺].

### Example 40

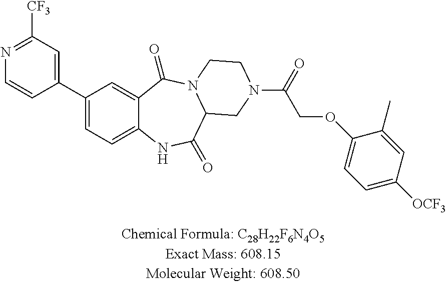 [SMILES: Cc1cc(OC(F)(F)F)ccc1OCC(=O)N1CCN2C(=O)c3cc(-c4ccnc(C(F)(F)F)c4)ccc3NC(=O)C2C1]

This example is directed to the synthesis of 2-(2-(2-methyl-4-(trifluoromethoxy)phenoxy)acetyl)-8-(2-(trifluoromethyl)pyridin-4-yl)-1,3,4,12a-tetrahydrobenzo[e]pyrazino[1,2-a][1,4]diazepine-6,12 (2H, 11H)-dione (KJW011-039) in an aspect of the invention.

The compound was prepared following General Procedure I using 8-bromo-2-(2-(2-methyl-4-(trifluoromethoxy)phenoxy)acetyl)-1,3,4,12a-tetrahydrobenzo[e]pyrazino[1,2-a][1,4]diazepine-6,12 (2H, 11H)-dione (KJW011-032) and (2-(trifluoromethyl)pyridin-4-yl)boronic acid. ¹H NMR (400 MHz, DMSO-d₆) δ 10.88 (s, 0.69×1H), 10.79 (s, 0.31×1H), 8.84 (d, J=5.1 Hz, 1H), 8.27 (dd, J=5.6, 2.3 Hz, 1H), 8.21 (d, J=8.6 Hz, 1H), 8.17-8.05 (m, 2H), 7.30 (d, J=8.5 Hz, 1H), 7.17 (t, J=4.5 Hz, 1H), 7.08 (dd, J=8.9, 2.9 Hz, 1H), 6.97 (dd, J=17.6, 9.0 Hz, 1H), 5.15-5.00 (m, 0.69×2H), 4.39 (q, J=5.7, 5.2 Hz, 0.31×2H), 4.26-4.09 (m, 2H), 3.77 (dd, J=14.8, 4.8 Hz, 1H), 3.75-3.63 (m, 1H), 3.57 (ddd, J=13.3, 8.7, 4.2 Hz, 1H), 3.44-3.36 (m, 2H), 2.22 (d, J=7.2 Hz, 3H). ¹⁹F NMR (376 MHz, DMSO-d₆) δ−57.13, −57.15, −66.40, −66.42. LCMS RT (Method 1)=5.472 min, m/z 609.1 [M+H].

### Example 41

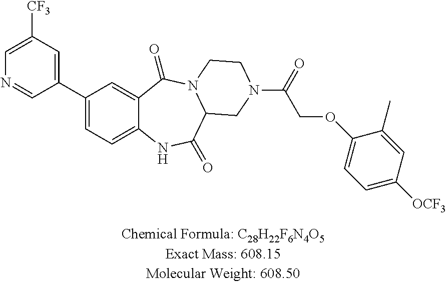 [SMILES: Cc1cc(OC(F)(F)F)ccc1OCC(=O)N1CCN2C(=O)c3cc(-c4cncc(C(F)(F)F)c4)ccc3NC(=O)C2C1]

This example is directed to the synthesis of 2-(2-(2-methyl-4-(trifluoromethoxy)phenoxy)acetyl)-8-(5-(trifluoromethyl)pyridin-3-yl)-1,3,4,12a-tetrahydrobenzo[e]pyrazino[1,2-a][1,4]diazepine-6,12 (2H, 11H)-dione (KJW011-037) in an aspect of the invention.

The compound was prepared following General Procedure I using 8-bromo-2-(2-(2-methyl-4-(trifluoromethoxy)phenoxy)acetyl)-1,3,4,12a-tetrahydrobenzo[e]pyrazino[1,2-a][1,4]diazepine-6,12 (2H, 11H)-dione (KJW011-032) and (5-(trifluoromethyl)pyridin-3-yl)boronic acid. LCMS RT (Method 1)=5.454 min, m/z 609.2 [M+H⁺].

### Example 42

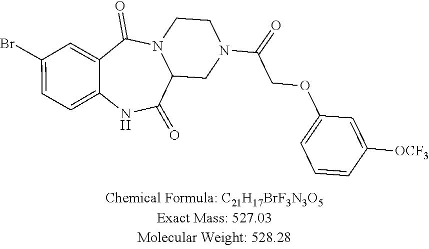 [SMILES: O=C1Nc2ccc(Br)cc2C(=O)N2CCN(C(=O)COc3cccc(OC(F)(F)F)c3)CC12]

This example is directed to the synthesis of 8-bromo-2-(2-(3-(trifluoromethoxy)phenoxy)acetyl)-1,3,4,12a-tetrahydrobenzo[e]pyrazino[1,2-a][1,4]diazepine-6,12 (2H, 11H)-dione (KJW008-096-1) in an aspect of the invention.

The compound was prepared following General Procedure D without purification using 3-trifluoromethoxyphenol. LCMS RT (Method 2)=3.169 min, m/z 609.2 [M+H⁺].

### Example 43

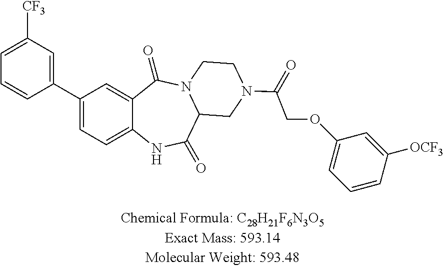 [SMILES: O=C1Nc2ccc(-c3cccc(C(F)(F)F)c3)cc2C(=O)N2CCN(C(=O)COc3cccc(OC(F)(F)F)c3)CC12]

This example is directed to the synthesis of 2-(2-(3-(trifluoromethoxy)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW008-099) in an aspect of the invention.

The compound was prepared following General Procedure G using 8-bromo-2-(2-(3-(trifluoromethoxy)phenoxy)acetyl)-1,3,4,12a-tetrahydrobenzo[e]pyrazino[1,2-a][1,4]diazepine-6,12 (2H, 11H)-dione (KJW008-096-1). ¹H NMR (400 MHz, Acetic Acid-d₄) δ 8.30 (d, J=2.3 Hz, 1H), 7.99 (d, J=4.4 Hz, 1H), 7.97-7.90 (m, 2H), 7.69 (dt, J=15.3, 7.8 Hz, 2H), 7.41-7.28 (m, 2H), 6.98 (ddd, J=10.5, 8.1, 2.3 Hz, 1H), 6.94-6.86 (m, 2H), 5.19-5.02 (m, 0.56×2H), 4.91 (q, J=15.0 Hz, 0.44×2H), 4.55 (dd, J=14.3, 4.9 Hz, 0.42×1H), 4.48 (dt, J=7.9, 4.5 Hz, 1H), 4.38 (dd, J=14.6, 3.9 Hz, 0.58×1H), 4.30-4.11 (m, 1H), 4.06-3.64 (m, 4H). ¹⁹F NMR (376 MHz, Acetic Acid-d₄) δ−58.18, −62.95 LCMS RT (Method 1)=5.884 min, m/z 530.0 [M+2H⁺].

### Example 44

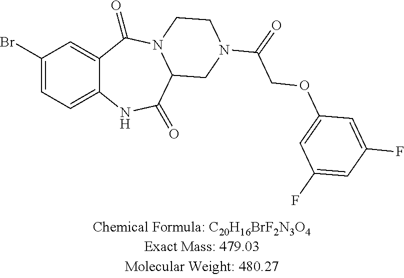 [SMILES: O=C1Nc2ccc(Br)cc2C(=O)N2CCN(C(=O)COc3cc(F)cc(F)c3)CC12]

This example is directed to the synthesis of 8-bromo-2-(2-(3,5-difluorophenoxy)acetyl)-1,3,4,12a-tetrahydrobenzo[e]pyrazino[1,2-a][1,4]diazepine-6,12 (2H, 11H)-dione (KJW008-96-2) in an aspect of the invention.

The compound was prepared following General Procedure D without purification using 3,5-difluoromethoxyphenol. LCMS RT (Method 2)=3.059 min, m/z 480.0 [M⁺].

### Example 45

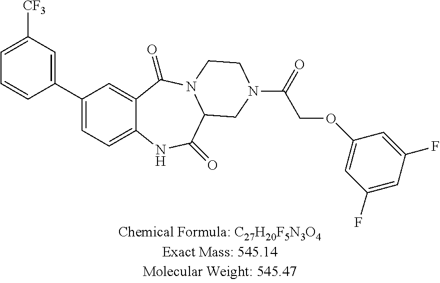 [SMILES: O=C1Nc2ccc(-c3cccc(C(F)(F)F)c3)cc2C(=O)N2CCN(C(=O)COc3cc(F)cc(F)c3)CC12]

This example is directed to the synthesis of 2-(2-(3,5-difluorophenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW008-100) in an aspect of the invention.

The compound was prepared following General Procedure G using 8-bromo-2-(2-(3,5-difluorophenoxy)acetyl)-1,3,4,12a-tetrahydrobenzo[e]pyrazino[1,2-a][1,4]diazepine-6,12 (2H, 11H)-dione (KJW008-96-2). ¹H NMR (400 MHz, Acetic Acid-d₄) δ 8.30 (dd, J=3.8, 2.3 Hz, 1H), 7.99 (d, J=4.5 Hz, 1H), 7.97-7.89 (m, 2H), 7.69 (dt, J=15.2, 7.8 Hz, 2H), 7.32 (d, J=8.4 Hz, 1H), 6.61 (ddd, J=8.5, 5.7, 2.2 Hz, 2H), 6.53 (tdd, J=9.1, 6.5, 2.3 Hz, 1H), 5.18-4.99 (m, 0.53×2H), 4.96-4.83 (m, 0.47×2H), 4.58-4.32 (m, 2H), 4.30-3.64 (m, 5H). ¹⁹F NMR (376 MHz, Acetic Acid-d₄) δ−63.40, −110.23 (dt, J=14.5, 8.8 Hz). LCMS RT (Method 1)=5.582 min. m/z 546.1 [M+H⁺].

### Example 46

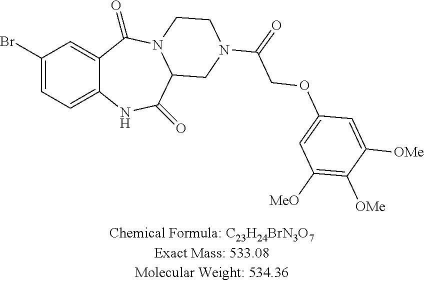 [SMILES: COc1cc(OCC(=O)N2CCN3C(=O)c4cc(Br)ccc4NC(=O)C3C2)cc(OC)c1OC]

This example is directed to the synthesis of 8-bromo-2-(2-(3,4,5-trimethoxyphenoxy)acetyl)-1,3,4,12a-tetrahydrobenzo[e]pyrazino[1,2-a][1,4]diazepine-6,12 (2H, 11H)-dione (KJW008-096-3) in an aspect of the invention.

The compound was prepared following General Procedure D without purification using 3,4,5-trimethoxyphenol. LCMS RT (Method 2)=3.014 min, m/z 534.1 [M⁺].

### Example 47

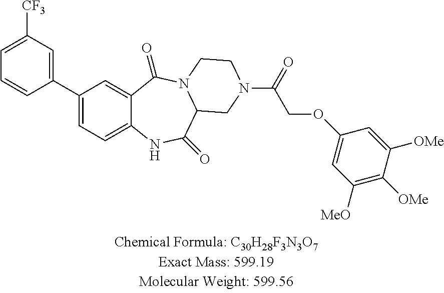 [SMILES: COc1cc(OCC(=O)N2CCN3C(=O)c4cc(-c5cccc(C(F)(F)F)c5)ccc4NC(=O)C3C2)cc(OC)c1OC]

This example is directed to the synthesis of 8-(3-(trifluoromethyl)phenyl)-2-(2-(3,4,5-trimethoxyphenoxy)acetyl)-1,3,4,12a-tetrahydrobenzo[e]pyrazino[1,2-a][1,4]diazepine-6,12 (2H, 11H)-dione (KJW009-002) in an aspect of the invention.

The compound was prepared following General Procedure G using 8-bromo-2-(2-(3,4,5-trimethoxyphenoxy)acetyl)-1,3,4,12a-tetrahydrobenzo[e]pyrazino[1,2-a][1,4]diazepine-6,12 (2H, 11H)-dione (KJW008-096-3). ¹H NMR (400 MHz, Acetic Acid-d₄) δ 8.28 (d, J=2.3 Hz, 1H), 8.06-7.88 (m, 3H), 7.69 (dt, J=15.4, 7.9 Hz, 2H), 7.44-7.20 (m, 1H), 6.31 (d, J=5.2 Hz, 2H), 5.02 (s, 0.59×2H), 4.85 (d, J=2.9 Hz, 0.41×2H), 3.82 (s, 0.43×6H), 3.79 (s, 0.57×6H), 3.72 (s, 0.44×3H), 3.70 (s, 0.56×3H). ¹⁹F NMR (376 MHz, Acetic Acid-d₄) δ−63.40. LCMS RT (Method 1)=5.240 min, m/z 600.2 [M+H⁺].

### Example 48

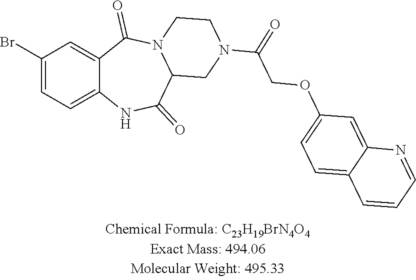 [SMILES: O=C1Nc2ccc(Br)cc2C(=O)N2CCN(C(=O)COc3ccc4cccnc4c3)CC12]

This example is directed to the synthesis of 8-bromo-2-(2-(quinolin-7-yloxy)acetyl)-1,3,4,12a-tetrahydrobenzo[e]pyrazino[1,2-a][1,4]diazepine-6,12 (2H, 11H)-dione (KJW008-096-4) in an aspect of the invention.

The compound was prepared following General Procedure D without purification using quinolin-7-ol. LCMS RT (Method 2)=497.1 min, m/z 2.528 [M+2H⁺].

### Example 49

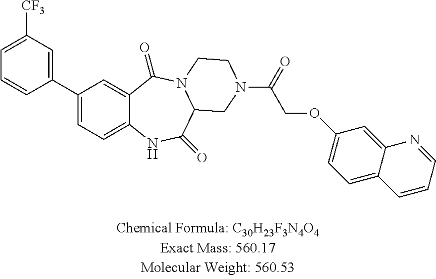 [SMILES: O=C1Nc2ccc(-c3cccc(C(F)(F)F)c3)cc2C(=O)N2CCN(C(=O)COc3ccc4cccnc4c3)CC12]

This example is directed to the synthesis of 2-(2-(quinolin-7-yloxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW009-003) in an aspect of the invention.

The compound was prepared following General Procedure G using 8-bromo-2-(2-(quinolin-7-yloxy)acetyl)-1,3,4,12a-tetrahydrobenzo[e]pyrazino[1,2-a][1,4]diazepine-6,12 (2H, 11H)-dione (KJW008-096-4). ¹H NMR (400 MHz, Acetic Acid-d₄) δ 9.18 (dd, J=18.0, 5.3 Hz, 1H), 8.92 (dd, J=8.0, 4.7 Hz, 1H), 8.31 (d, J=2.7 Hz, 1H), 8.17 (dd, J=9.2, 2.0 Hz, 1H), 8.02-7.89 (m, 4H), 7.86 (dtd, J=8.1, 5.0, 2.4 Hz, 1H), 7.76-7.61 (m, 3H), 7.34 (dd, J=8.5, 4.9 Hz, 1H), 5.54-5.28 (m, 0.59×2H), 5.26-5.16 (m, 0.41×2H), 4.65-4.39 (m, 2H), 4.37-4.18 (m, 1H), 4.12-3.68 (m, 4H). ¹⁹F NMR (376 MHz, Acetic Acid-d₄) δ−63.39. LCMS RT (Method 1)=4.453 min, m/z 561.2 [M+H⁺].

### Example 50

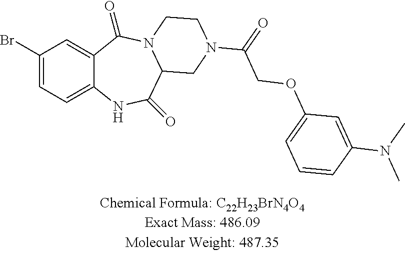 [SMILES: CN(C)c1cccc(OCC(=O)N2CCN3C(=O)c4cc(Br)ccc4NC(=O)C3C2)c1]

This example is directed to the synthesis of 8-bromo-2-(2-(3-(dimethylamino)phenoxy)acetyl)-1,3,4,12a-tetrahydrobenzo[e]pyrazino[1,2-a][1,4]diazepine-6,12 (2H, 11H)-dione (KJW009-008) in an aspect of the invention.

The compound was prepared following General Procedure D without purification using 3-(dimethylamino)phenol. LCMS RT (Method 2)=2.482 min, m/z 489.1 [M+2H⁺].

### Example 51

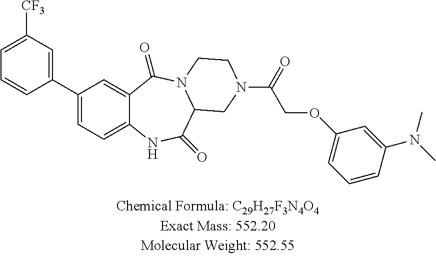 [SMILES: CN(C)c1cccc(OCC(=O)N2CCN3C(=O)c4cc(-c5cccc(C(F)(F)F)c5)ccc4NC(=O)C3C2)c1]

This example is directed to the synthesis of 2-(2-(3-(dimethylamino)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW009-012) in an aspect of the invention.

The compound was prepared following General Procedure G using 8-bromo-2-(2-(3-(dimethylamino)phenoxy)acetyl)-1,3,4,12a-tetrahydrobenzo[e]pyrazino[1,2-a][1,4]diazepine-6,12 (2H, 11H)-dione (KJW009-008). ¹H NMR (400 MHz, Acetic Acid-d₄) δ 8.30 (dd, J=3.7, 2.2 Hz, 1H), 8.01-7.91 (m, 3H), 7.75-7.61 (m, 4H), 7.33 (dd, J=8.5, 1.8 Hz, 1H), 7.20-7.10 (m, 2H), 5.30-5.03 (m, 0.57×2H), 5.02-4.87 (m, 0.43×2H), 4.58 (dd, J=14.4, 4.5 Hz, 0.43×1H), 4.50 (t, J=4.2 Hz, 1H), 4.38 (dd, J=14.6, 3.5 Hz, 0.57×1H), 4.30-3.65 (m, 5H), 3.28 (d, J=2.4 Hz, 6H). ¹⁹F NMR (376 MHz Acetic Acid-d₄) δ−63.40. LCMS RT (Method 1)=4.400 min, m/z 553.2 [M+H⁺].

### Example 52

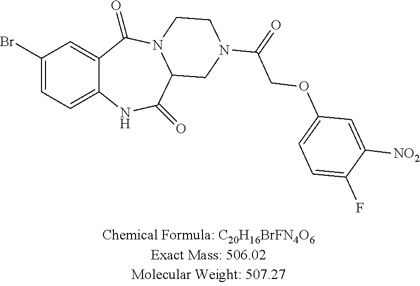 [SMILES: O=C1Nc2ccc(Br)cc2C(=O)N2CCN(C(=O)COc3ccc(F)c([N+](=O)[O-])c3)CC12]

This example is directed to the synthesis of 8-bromo-2-(2-(4-fluoro-3-nitrophenoxy)acetyl)-1,3,4,12a-tetrahydrobenzo[e]pyrazino[1,2-a][1,4]diazepine-6,12 (2H, 11H)-dione (KJW008-097-4) in an aspect of the invention.

The compound was prepared following General Procedure D without purification using 3-(dimethylamino)phenol. LCMS RT (Method 2)=3.049 min, m/z 508.8 [M+2H⁺].

### Example 53

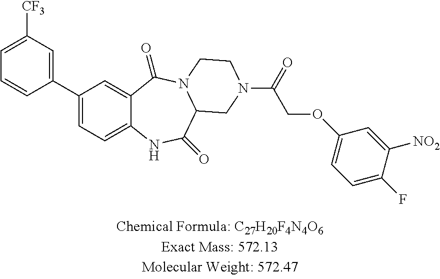 [SMILES: O=C1Nc2ccc(-c3cccc(C(F)(F)F)c3)cc2C(=O)N2CCN(C(=O)COc3ccc(F)c([N+](=O)[O-])c3)CC12]

This example is directed to the synthesis of 2-(2-(4-fluoro-3-nitrophenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW009-005) in an aspect of the invention.

The compound was prepared following General Procedure G using 8-bromo-2-(2-(4-fluoro-3-nitrophenoxy)acetyl)-1,3,4,12a-tetrahydrobenzo[e]pyrazino[1,2-a][1,4]diazepine-6,12 (2H, 11H)-dione (KJW008-097-4). ¹H NMR (400 MHz, Acetic Acid-d₄) δ 8.30 (t, J=2.7 Hz, 1H), 7.99 (s, 1H), 7.97-7.90 (m, 2H), 7.75-7.64 (m, 3H), 7.33 (ddd, J=11.6, 7.7, 3.3 Hz, 3H), 5.31-5.06 (m, 0.56×2H), 5.05-4.93 (m, 0.44×2H), 4.57 (dd, J=14.6, 4.6 Hz, 0.43×1H), 4.49 (t, J=4.4 Hz, 1H), 4.36 (dd, J=14.7, 3.8 Hz, 0.57×1H), 0.31-4.14 (m, 1H), 4.03-3.64 (m, 4H). ¹⁹F NMR (376 MHz, Acetic Acid-d₄) δ−63.41 (s, 3F), −128.99 (ddt, J=71.1, 10.2, 5.0 Hz, 1F) LCMS RT (Method 1)=5.433 min, m/z 573.1 [M+H⁺].

### Example 54

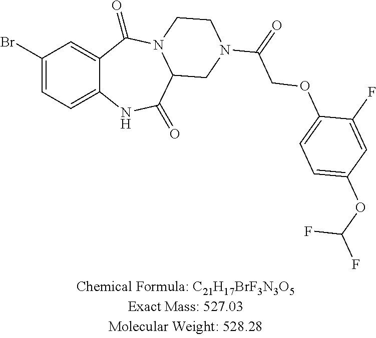 [SMILES: O=C1Nc2ccc(Br)cc2C(=O)N2CCN(C(=O)COc3ccc(OC(F)F)cc3F)CC12]

This example is directed to the synthesis of 8-bromo-2-(2-(4-(difluoromethoxy)-2-fluorophenoxy)acetyl)-1,3,4,12a-tetrahydrobenzo[e]pyrazino[1,2-a][1,4]diazepine-6,12 (2H, 11H)-dione (KJW009-032) in an aspect of the invention.

The compound was prepared following General Procedure D without purification using 3 4-(difluoromethoxy)-2-fluorophenol. LCMS RT (Method 3)=2.989 min, m/z 527.8 [M⁺].

### Example 55

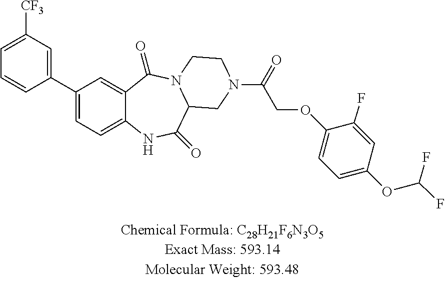 [SMILES: O=C1Nc2ccc(-c3cccc(C(F)(F)F)c3)cc2C(=O)N2CCN(C(=O)COc3ccc(OC(F)F)cc3F)CC12]

This example is directed to the synthesis of 2-(2-(4-(difluoromethoxy)-2-fluorophenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW009-036) in an aspect of the invention.

The compound was prepared following General Procedure G using 8-bromo-2-(2-(4-(difluoromethoxy)-2-fluorophenoxy)acetyl)-1,3,4,12a-tetrahydrobenzo[e]pyrazino[1,2-a][1,4]diazepine-6,12 (2H, 11H)-dione (KJW009-032). ¹H NMR (400 MHz, Acetic Acid-d₄) δ 8.30 (t, J=2.7 Hz, 1H), 7.99 (s, 1H), 7.97-7.90 (m, 2H), 7.69 (dt, J=15.3, 7.8 Hz, 2H), 7.32 (d, J=8.4 Hz, 1H), 7.13 (dt, J=25.7, 9.2 Hz, 1H), 7.00 (ddd, J=11.9, 9.3, 2.8 Hz, 1H), 6.92 (d, J=9.1 Hz, 1H), 6.65 (td, J=73.8, 6.0 Hz, 1H), 5.14 (q, J=14.7 Hz, 0.56×2H), 4.95 (q, J=15.0 Hz, 0.55×2H), 4.58 (dd, J=14.6, 4.5 Hz, 0.45×1H), 4.49 (q, J=4.3 Hz, 1H), 4.40 (dd, J=14.7, 3.8 Hz, 0.55×1H), 4.28-4.16 (m, 1H), 4.06-3.65 (m, 4H). ¹⁹F NMR (376 MHz, Acetic Acid-d₄) δ−63.41, −81.51-−84.27 (m), −131.18 (dt, J=95.4, 10.6 Hz). LCMS RT (Method 1)=5.633 min, m/z 594.2 [M+H⁺].

### Example 56

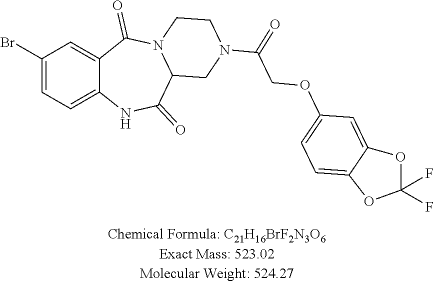 [SMILES: O=C1Nc2ccc(Br)cc2C(=O)N2CCN(C(=O)COc3ccc4c(c3)OC(F)(F)O4)CC12]

This example is directed to the synthesis of 8-bromo-2-(2-((2,2-difluorobenzo[d][1,3]dioxol-5-yl)oxy)acetyl)-1,3,4,12a-tetrahydrobenzo[e]pyrazino[1,2-a][1,4]diazepine-6,12 (2H, 11H)-dione (KJW009-088-3) in an aspect of the invention.

The compound was prepared following General Procedure D using 2,2-difluorobenzo[d][1,3]dioxol-5-ol. LCMS RT (Method 2)=3.204 min, m/z 524.0 [M⁺].

### Example 57

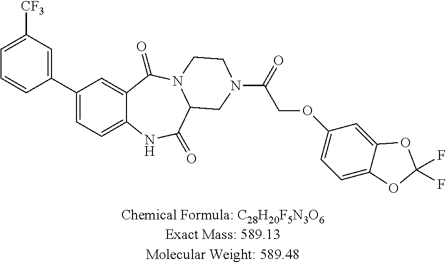 [SMILES: O=C1Nc2ccc(-c3cccc(C(F)(F)F)c3)cc2C(=O)N2CCN(C(=O)COc3ccc4c(c3)OC(F)(F)O4)CC12]

This example is directed to the synthesis of 2-(2-((2,2-difluorobenzo[d][1,3]dioxol-5-yl)oxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW009-093) in an aspect of the invention.

The compound was prepared following General Procedure G using 8-bromo-2-(2-(4-(difluoromethoxy)-2-fluorophenoxy)acetyl)-1,3,4,12a-tetrahydrobenzo[e]pyrazino[1,2-a][1,4]diazepine-6,12 (2H, 11H)-dione (KJW009-088-3). ¹H NMR (400 MHz, Acetic Acid-d₄) δ 8.30 (dd, J=4.7, 2.2 Hz, 1H), 8.01-7.97 (m, 1H), 7.97-7.90 (m, 2H), 7.73-7.65 (m, 2H), 7.32 (dd, J=8.5, 1.7 Hz, 1H), 7.05 (dd, J=8.8, 5.7 Hz, 1H), 6.92-6.89 m, 1H), 6.74 (ddd, J=9.3, 7.4, 2.5 Hz, 1H), 5.16-4.97 (m, 0.53×2H), 4.94-4.81 (m, 0.47×2H), 4.56 (dd, J=14.5, 4.6 Hz, 0.47×1H), 4.51-4.46 (m, 1H), 4.37 (dd, J=14.7, 3.7 Hz, 0.53×1H), 4.29-3.63 (m, 5H). ¹⁹F NMR (376 MHz, Acetic Acid-d₄) δ−51.31, −63.41. LCMS RT (Method 1)=5.773 min, m/z 590.1 [M+H⁺].

### Example 58

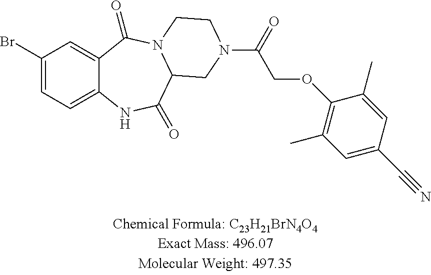 [SMILES: Cc1cc(C#N)cc(C)c1OCC(=O)N1CCN2C(=O)c3cc(Br)ccc3NC(=O)C2C1]

This example is directed to the synthesis of 4-(2-(8-bromo-6,12-dioxo-3,4,6,11,12,12a-hexahydrobenzo[e]pyrazino[1,2-a][1,4]diazepin-2 (1H)-yl)-2-oxoethoxy)-3,5-dimethylbenzonitrile (KJW009-088-4) in an aspect of the invention.

The compound was prepared following General Procedure D using 4-hydroxy-3,5-dimethylbenzonitrile. LCMS RT (Method 2)=2.997 min, m/z 499.1 [M+2H⁺].

### Example 59

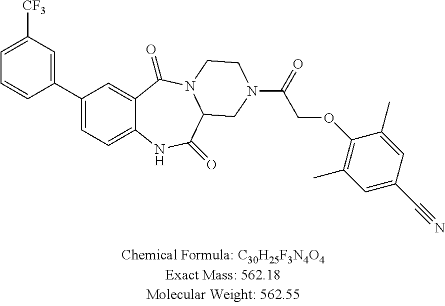 [SMILES: Cc1cc(C#N)cc(C)c1OCC(=O)N1CCN2C(=O)c3cc(-c4cccc(C(F)(F)F)c4)ccc3NC(=O)C2C1]

This example is directed to the synthesis of 4-(2-(6,12-dioxo-8-(3-(trifluoromethyl)phenyl)-3,4,6,11,12,12a-hexahydrobenzo[e]pyrazino[1,2-a][1,4]diazepin-2 (1H)-yl)-2-oxoethoxy)-3,5-dimethylbenzonitrile (KJW009-094) in an aspect of the invention.

The compound was prepared following General Procedure G using 4-(2-(8-bromo-6,12-dioxo-3,4,6,11,12,12a-hexahydrobenzo[e]pyrazino[1,2-a][1,4]diazepin-2 (1H)-yl)-2-oxoethoxy)-3,5-dimethylbenzonitrile (KJW009-088-4). LCMS RT (Method 1)=5.506 min, m/z 563.2 [M+H⁺].

### Example 60

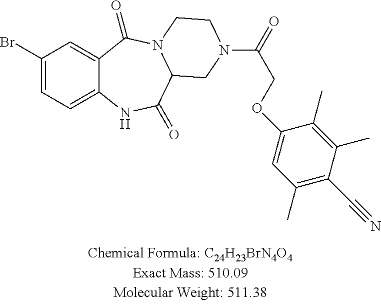 [SMILES: Cc1cc(OCC(=O)N2CCN3C(=O)c4cc(Br)ccc4NC(=O)C3C2)c(C)c(C)c1C#N]

4-(2-(8-bromo-6,12-dioxo-3,4,6,11,12,12a-hexahydrobenzo[e]pyrazino[1,2-a][1,4]diazepin-2 (1H)-yl)-2-oxoethoxy)-2,3,6-trimethylbenzonitrile (KJW009-095-1) in an aspect of the invention.

The compound was prepared following General Procedure D using 4-hydroxy-2,3,6-trimethylbenzonitrile. LCMS RT (Method 2)=3.154 min, m/z 511.1 [M⁺].

### Example 61

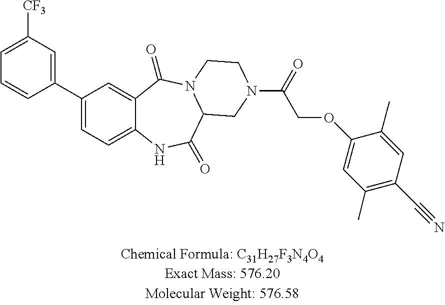 [SMILES: Cc1cc(OCC(=O)N2CCN3C(=O)c4cc(-c5cccc(C(F)(F)F)c5)ccc4NC(=O)C3C2)c(C)cc1C#N]

This example is directed to the synthesis of 4-(2-(6,12-dioxo-8-(3-(trifluoromethyl)phenyl)-3,4,6,11,12,12a-hexahydrobenzo[e]pyrazino[1,2-a][1,4]diazepin-2 (1H)-yl)-2-oxoethoxy)-2,3,6-trimethylbenzonitrile (KJW009-099) in an aspect of the invention.

The compound was prepared following General Procedure G using 4-(2-(8-bromo-6,12-dioxo-3,4,6,11,12,12a-hexahydrobenzo[e]pyrazino[1,2-a][1,4]diazepin-2 (1H)-yl)-2-oxoethoxy)-2,3,6-trimethylbenzonitrile (KJW009-095-1). ¹H NMR (400 MHz, Acetic Acid-d₄) δ 8.30 (dd, J=3.9, 2.2 Hz, 1H), 7.99 (s, 1H), 7.97-7.90 (m, 2H), 7.75-7.65 (m, 2H), 7.32 (dd, J=8.4, 1.9 Hz, 1H), 6.81 (s, 0.57×1H), 6.74 (s, 0.43×1H), 5.23-5.07 (m, 0.57×2H), 5.02-4.91 (m, 0.43×2H), 4.58 (dd, J=14.5, 4.4 Hz, 0.43×1H), 4.50 (t, J=4.7 Hz, 0.43×1H), 4.46 (t, J=4.1 Hz, 0.57×1H), 4.39 (dd, J=14.6, 3.9 Hz, 0.57×1H), 4.32-3.64 (m, 5H), 2.47-2.42 (m, 6H), 2.18 (d, J=8.7 Hz, 3H), 2.19 (s, 0.49×3H), 2.17 (s, 0.51×3H). ¹⁹F NMR (376 MHz, Acetic Acid-d₄) δ−63.41. LCMS RT (Method 1)=5.705 min, m/z 577.2 [M+H⁺].

### Example 62

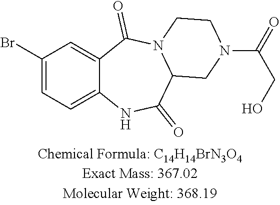 [SMILES: O=C1Nc2ccc(Br)cc2C(=O)N2CCN(C(=O)CO)CC12]

This example is directed to the synthesis of 8-bromo-2-(2-hydroxyacetyl)-1,3,4,12a-tetrahydrobenzo[e]pyrazino[1,2-a][1,4]diazepine-6,12 (2H, 11H)-dione (KJW010-047) in an aspect of the invention.

To 8-bromo-1,3,4,12a-tetrahydrobenzo[e]pyrazino[1,2-a][1,4]diazepine-6,12 (2H, 11H)-dione 2,2,2-trifluoroacetate (KJW008-042) in DMF was added HATU (1.3 eq.), (DIEA), and 2-hydroxyacetic acid (1.2 eq.). The reaction was allowed to stir for 2 hours and diluted with EtOAc. The result was washed with twice satd. NaHCO₃, dried (Na₂SO₄) and concentrated in vacuo. The residue was purified by preparative thin layer chromatography to afford the title compound. ¹H NMR (400 MHz, Acetic Acid-d₄) δ 8.24 (q, J=4.7, 3.3 Hz, 1H), 7.84 (dd, J=8.6, 2.3 Hz, 1H), 7.23 (d, J=8.7 Hz, 1H), 4.75-3.67 (m, 9H). LCMS RT (Method 2)=5.443 min. m/z 370 [M+2H⁺].

### Example 63

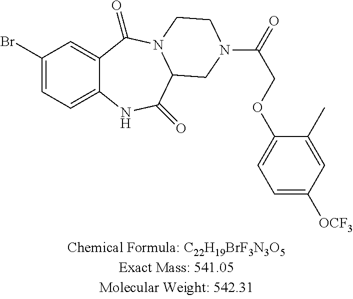 [SMILES: Cc1cc(OC(F)(F)F)ccc1OCC(=O)N1CCN2C(=O)c3cc(Br)ccc3NC(=O)C2C1]

This example is directed to the synthesis of 8-bromo-2-(2-(2-methyl-4-(trifluoromethoxy)phenoxy)acetyl)-1,3,4,12a-tetrahydrobenzo[e]pyrazino[1,2-a][1,4]diazepine-6,12 (2H, 11H)-dione (KJW010-043) in an aspect of the invention. See FIG. 6.

To 8-bromo-2-(2-hydroxyacetyl)-1,3,4,12a-tetrahydrobenzo[e]pyrazino[1,2-a][1,4]diazepine-6,12 (2H, 11H)-dione (KJW010-047) (1 eq.) in THF was added triphenylphosphane (1.2 eq.), (diisopropyl (E)-diazene-1,2-dicarboxylate (1.2 eq.) and 2-methyl-4-(trifluoromethoxy)phenol (1.1 eq.). The reaction was allowed to stir at room temperature overnight and diluted with EtOAc. The result was washed with twice satd. NaHCO₃, dried (Na₂SO₄) and concentrated in vacuo. The residue was filtered through a pad of silica gel with 10% MeOH-DCM and the residue was purified by flash column chromatography (silica gel, 0-5% MeOH-DCM) to afford the title compound. ¹H NMR (400 MHz, Acetic Acid-d₄) δ 8.13-8.06 (m, 1H), 7.71 (dt, J=8.7, 2.3 Hz, 1H), 7.16-6.82 (m, 4H), 5.17-5.03 (m, 0.56×2H), 4.97-4.84 (m, 0.44×2H), 4.64-4.32 (m, 2H), 4.16 (tq, J=14.2, 8.4, 7.3 Hz, 1H), 4.05-3.62 (m, 4H), 2.28 (s, 0.47×3H), 2.26 (s, 0.53×3H). ¹⁹F NMR (376 MHz, Acetic acid-d₄) 6-63.62. LCMS RT (Method 2)=3.33 min, m/z 542.0 [M⁺].

### Example 64

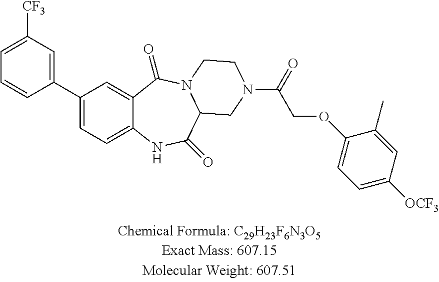 [SMILES: Cc1cc(OC(F)(F)F)ccc1OCC(=O)N1CCN2C(=O)c3cc(-c4cccc(C(F)(F)F)c4)ccc3NC(=O)C2C1]

This example is directed to the synthesis of 2-(2-(2-methyl-4-(trifluoromethoxy)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-049) in an aspect of the invention. See FIG. 6.

To 8-bromo-2-(2-(2-methyl-4-(trifluoromethoxy)phenoxy)acetyl)-1,3,4,12a-tetrahydrobenzo[e]pyrazino[1,2-a][1,4]diazepine-6,12 (2H, 11H)-dione (KJW010-043) (1 eq.) in dioxane was added 1M aq. K₃PO₄ (3.5 eq.), (2-dicyclohexylphosphino-2′,6′-diisopropoxy-1,1′-biphenyl)[2-(2′-amino-1,1′-biphenyl)]palladium(II) methanesulfonate (RuPhos Pd G3) (0.15 eq.), and (3-(trifluoromethyl)phenyl)boronic acid (1.6 eq.). The resultant slurry was heated to 95° C. for 18 hours, concentrated under vacuum. The residue was purified via standard reverse phase HPLC conditions using a gradient of 10-100% ACN in H₂O with 0.1% TFA to afford the title compound. ¹H NMR (400 MHz, Acetic Acid-d₄) δ 8.31 (d, J=2.3 Hz, 1H), 7.99 (d, J=4.2 Hz, 1H), 7.97-7.91 (m, 2H), 7.70 (dt, J=15.3, 7.8 Hz, 2H), 7.32 (d, J=8.4 Hz, 1H), 7.12-7.04 (m, 2H), 6.98-6.86 (m, 1H), 5.20-5.04 (m, 0.56×2H), 5.00-4.84 (m, 0.44×2H), 4.60 (dd, J=14.6, 4.4 Hz, 0.45×1H), 4.49 (dt, J=12.5, 4.4 Hz, 1H), 4.41 (dd, J=14.6, 3.7 Hz, 0.55×1H), 4.29-4.14 (m, 2H), 4.06-3.66 (m, 3H), 2.29 (s, 0.46×3H), 2.26 (s, 0.54×3H). ¹⁹F NMR (376 MHz, Acetic Acid-d₄) δ−59.06, −59.09, −63.41. LCMS RT (Method 1)=5.974 min, m/z 608.2 [M+H⁺].

### Example 65

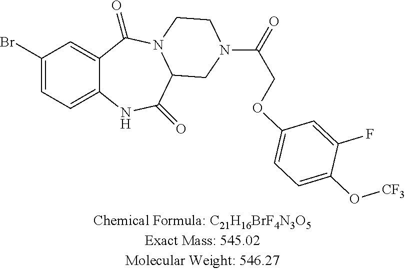 [SMILES: O=C1Nc2ccc(Br)cc2C(=O)N2CCN(C(=O)COc3ccc(OC(F)(F)F)c(F)c3)CC12]

This example is directed to the synthesis of 8-bromo-2-(2-(3-fluoro-4-(trifluoromethoxy)phenoxy)acetyl)-1,3,4,12a-tetrahydrobenzo[e]pyrazino[1,2-a][1,4]diazepine-6,12 (2H, 11H)-dione (KJW009-095-3) in an aspect of the invention.

The compound was prepared following General Procedure D using 3-fluoro-4-trifluoromethoxyphenol. LCMS RT (Method 2)=3.275 min, m/z 548.0 [M+2H⁺].

### Example 66

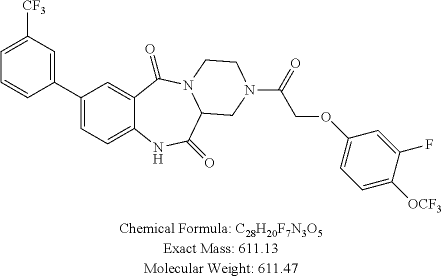 [SMILES: O=C1Nc2ccc(-c3cccc(C(F)(F)F)c3)cc2C(=O)N2CCN(C(=O)COc3ccc(OC(F)(F)F)c(F)c3)CC12]

This example is directed to the synthesis of 2-(2-(3-fluoro-4-(trifluoromethoxy)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-004) in an aspect of the invention.

The compound was prepared following General Procedure G using 8-bromo-2-(2-(3-fluoro-4-(trifluoromethoxy)phenoxy)acetyl)-1,3,4,12a-tetrahydrobenzo[e]pyrazino[1,2-a][1,4]diazepine-6,12 (2H, 11H)-dione (KJW009-095-3). ¹H NMR (400 MHz, Acetic Acid-d₄) δ 8.30 (dd, J=4.1, 2.1 Hz, 1H), 7.99 (d, J=4.0 Hz, 1H), 7.97-7.91 (m, 2H), 7.69 (dt, J=15.3, 7.7 Hz, 2H), 7.32 (d, J=8.5 Hz, 2H), 6.95 (ddd, J=11.8, 5.9, 2.8 Hz, 1H), 6.86 (dd, J=13.0, 9.5 Hz, 1H), 5.23-5.00 (m, 0.56×2H), 4.92 (q, J=15.1 Hz, 0.44×2H), 4.57 (dd, J=14.4, 4.6 Hz, 0.44×1H), 4.49 (m, 1H), 4.35 (dd, J=14.5, 3.6 Hz, 0.56×1H), 4.30-4.15 (m, 1H), 4.03-3.63 (m, 4H), −60.27 (dd, J=12.8, 5.1 Hz), −63.41, −76.87, −127.62 (m). LCMS RT (Method 1)=5.92 min, m/z 612.1 [M+H⁺].

### Example 67

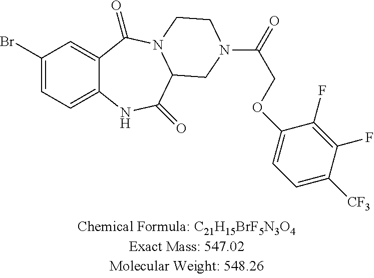 [SMILES: O=C1Nc2ccc(Br)cc2C(=O)N2CCN(C(=O)COc3ccc(C(F)(F)F)c(F)c3F)CC12]

This example is directed to the synthesis of 8-bromo-2-(2-(2,3-difluoro-4-(trifluoromethyl)phenoxy)acetyl)-1,3,4,12a-tetrahydrobenzo[e]pyrazino[1,2-a][1,4]diazepine-6,12 (2H, 11H)-dione (KJW009-095-4) in an aspect of the invention.

The compound was prepared following General Procedure D using 2,3-difluoro-4-(trifluoromethyl)phenol. LCMS RT (Method 2)=3.182 min, m/z 548.0 [M⁺].

### Example 68

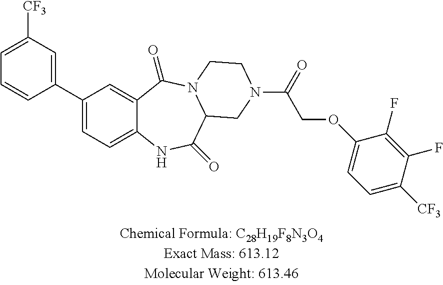 [SMILES: O=C1Nc2ccc(-c3cccc(C(F)(F)F)c3)cc2C(=O)N2CCN(C(=O)COc3ccc(C(F)(F)F)c(F)c3F)CC12]

This example is directed to the synthesis of 2-(2-(2,3-difluoro-4-(trifluoromethyl)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-005) in an aspect of the invention.

The compound was prepared following General Procedure G using 8-bromo-2-(2-(2,3-difluoro-4-(trifluoromethyl)phenoxy)acetyl)-1,3,4,12a-tetrahydrobenzo[e]pyrazino[1,2-a][1,4]diazepine-6,12 (2H, 11H)-dione (KJW009-095-4). ¹H NMR (400 MHz, Acetic Acid-d₄) δ 8.31 (dd, J=4.4, 2.2 Hz, 1H), 7.99 (s, 1H), 7.97-7.90 (m, 2H), 7.70 (dt, J=15.4, 7.8 Hz, 2H), 7.41 (q, J=7.8 Hz, 1H), 7.32 (dd, J=8.4, 3.3 Hz, 1H), 7.04 (dt, J=37.8, 8.2 Hz, 1H), 5.41-5.19 (m, 0.054×2H), 5.09 (q, J=15.3 Hz, 0.46×1H), 4.61 (dd, J=14.6, 4.2 Hz, 0.43×1H), 4.50 (t, J=4.1 Hz, 1H), 4.37 (dd, J=14.7, 3.4 Hz, 0.57×1H), 4.27-4.12 m, 1H), 4.03-3.62 (m, 3H). ¹⁹F NMR (376 MHz, Acetic Acid-d₄) δ−61.18, −61.22, −63.41, −140.08-−140.86 (m, 1F), −158.01 (ddd, J=26.8, 18.0, 6.9 Hz, 1F). LCMS RT (Method 1)=5.992 min, m/z 614.2 [M+H⁺).

### Example 69

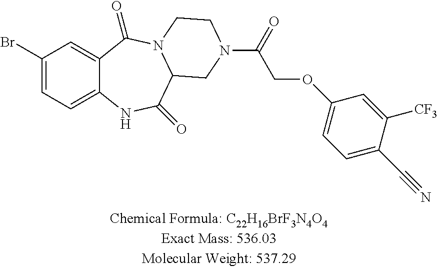 [SMILES: N#Cc1ccc(OCC(=O)N2CCN3C(=O)c4cc(Br)ccc4NC(=O)C3C2)cc1C(F)(F)F]

This example is directed to the synthesis of 4-(2-(8-bromo-6,12-dioxo-3,4,6,11,12,12a-hexahydrobenzo[e]pyrazino[1,2-a][1,4]diazepin-2 (1H)-yl)-2-oxoethoxy)-2-(trifluoromethyl)benzonitrile (KJW009-095-9) in an aspect of the invention.

The compound was prepared following General Procedure D using 4-hydroxy-2-(trifluoromethyl)benzonitrile. LCMS RT (Method 2)=3.042 min, m/z 537.0 [M⁺].

### Example 70

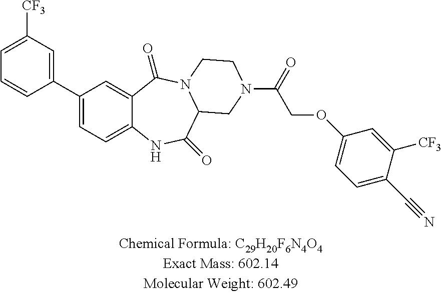 [SMILES: N#Cc1ccc(OCC(=O)N2CCN3C(=O)c4cc(-c5cccc(C(F)(F)F)c5)ccc4NC(=O)C3C2)cc1C(F)(F)F]

This example is directed to the synthesis of 4-(2-(6,12-dioxo-8-(3-(trifluoromethyl)phenyl)-3,4,6,11,12,12a-hexahydrobenzo[e]pyrazino[1,2-a][1,4]diazepin-2 (1H)-yl)-2-oxoethoxy)-2-(trifluoromethyl)benzonitrile (KJW010-010) in an aspect of the invention.

The compound was prepared following General Procedure G using 4-(2-(8-bromo-6,12-dioxo-3,4,6,11,12,12a-hexahydrobenzo[e]pyrazino[1,2-a][1,4]diazepin-2 (1H)-yl)-2-oxoethoxy)-2-(trifluoromethyl)benzonitrile (KJW009-095-9). ¹H NMR (400 MHz, Acetic Acid-d₄) δ 8.30 (dd, J=5.4, 2.2 Hz, 1H), 7.99 (s, 1H), 7.97-7.86 (m, 3H), 7.70 (dt, J=15.3, 7.8 Hz, 2H), 7.47 (t, J=3.1 Hz, 1H), 7.34 (m, 2H), 5.29 (m, 0.55×2H), 5.15-5.03 (m, 0.45×2H), 4.58 (dd, J=14.6, 4.4 Hz, 0.40×1H), 4.50 (t, J=4.2 Hz, 1H), 4.35 (dd, J=14.7, 3.4 Hz, 0.60×1H), 4.50 (t, J=4.2 Hz, 1H), 4.21 (tdd, J=22.4, 12.4, 6.7 Hz, 1H), 4.07-3.61 (m, 3H). ¹⁹F NMR (376 MHz, Acetic Acid-d₄)) δ−63.00, −63.41. LCMS RT (Method 1)=5.643 min, m/z 603.2 [M+H⁺].

### Example 71

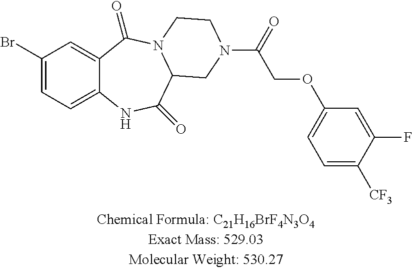 [SMILES: O=C1Nc2ccc(Br)cc2C(=O)N2CCN(C(=O)COc3ccc(C(F)(F)F)c(F)c3)CC12]

This example is directed to the synthesis of 8-bromo-2-(2-(3-fluoro-4-(trifluoromethyl)phenoxy)acetyl)-1,3,4,12a-tetrahydrobenzo[e]pyrazino[1,2-a][1,4]diazepine-6,12 (2H, 11H)-dione (KJW009-100-2) in an aspect of the invention.

The compound was prepared following General Procedure D using 3-fluoro-4-(trifluoromethyl)phenol. LCMS RT (Method 2) 3.225 min, m/z 530.0 [M⁺].

### Example 72

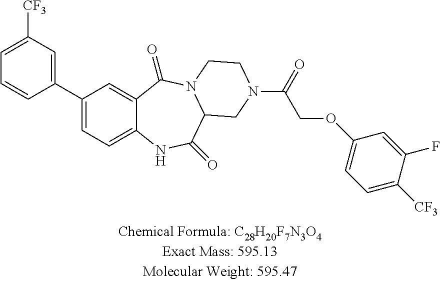 [SMILES: O=C1Nc2ccc(-c3cccc(C(F)(F)F)c3)cc2C(=O)N2CCN(C(=O)COc3ccc(C(F)(F)F)c(F)c3)CC12]

This example is directed to the synthesis of 2-(2-(3-fluoro-4-(trifluoromethyl)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-012) in an aspect of the invention.

The compound was prepared following General Procedure G using bromo-2-(2-(3-fluoro-4-(trifluoromethyl)phenoxy)acetyl)-1,3,4,12a-tetrahydrobenzo[e]pyrazino[1,2-a][1,4]diazepine-6,12 (2H, 11H)-dione (KJW009-100-2). ¹H NMR (400 MHz, Acetic Acid-d₄) δ 8.30 (dd, J=5.0, 2.1 Hz, 1H), 8.01-7.90 (m, 3H), 7.70 (dt, J=15.3, 7.8 Hz, 2H), 7.59 (q, J=8.0 Hz, 1H), 7.32 (dd, J=8.4, 2.7 Hz, 1H), 6.93 (dd, J=13.5, 8.8 Hz, 2H), 5.31-5.05 (m, 0.55×2H), 5.03-4.92 (m, 0.45×2H), 4.58 (dd, J=14.5, 4.6 Hz, 0.42×1H), 4.49 (m, 1H), 4.36 (dd, J=14.7, 3.5 Hz, 0.58×1H), 4.23 (m, 2H), 4.04-3.63 (m, 3H). ¹⁹F NMR (376 MHz, Acetic acid-d₄) 6-61.14, −61.17, −63.41, −76.87, −113.57 (q, J=12.2, 11.6 Hz). LCMS RT (Method 1)=5.866 min, m/z 596.1 [M+H⁺].

### Example 73

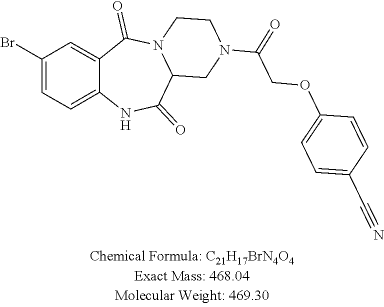 [SMILES: N#Cc1ccc(OCC(=O)N2CCN3C(=O)c4cc(Br)ccc4NC(=O)C3C2)cc1]

This example is directed to the synthesis of 4-(2-(8-bromo-6,12-dioxo-3,4,6,11,12,12a-hexahydrobenzo[e]pyrazino[1,2-a][1,4]diazepin-2 (1H)-yl)-2-oxoethoxy)benzonitrile (KJW009-095-7) in an aspect of the invention.

The compound was prepared following General Procedure D using 4-hydroxybenzonitrile. LCMS RT (Method 2)=2.824 min, m/z 469.0 [M⁺].

### Example 74

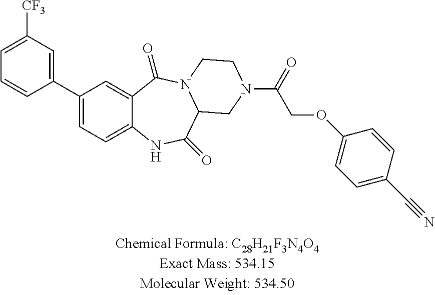 [SMILES: N#Cc1ccc(OCC(=O)N2CCN3C(=O)c4cc(-c5cccc(C(F)(F)F)c5)ccc4NC(=O)C3C2)cc1]

This example is directed to the synthesis of 4-(2-(6,12-dioxo-8-(3-(trifluoromethyl)phenyl)-3,4,6,11,12,12a-hexahydrobenzo[e]pyrazino[1,2-a][1,4]diazepin-2 (1H)-yl)-2-oxoethoxy)benzonitrile (KJW010-008) in an aspect of the invention.

The compound was prepared following General Procedure G using 4-(2-(8-bromo-6,12-dioxo-3,4,6,11,12,12a-hexahydrobenzo[e]pyrazino[1,2-a][1,4]diazepin-2 (1H)-yl)-2-oxoethoxy)benzonitrile (KJW009-095-7). ¹H NMR (400 MHz, Acetic Acid-d₄) δ 8.30 (dd, J=5.3, 2.1 Hz, 1H), 7.99 (s, 1H), 7.97-7.88 (m, 2H), 7.75-7.61 (m, 4H), 7.32 (dd, J=8.4, 3.3 Hz, 1H), 7.11 (dd, J=11.3, 8.5 Hz, 2H), 5.34-5.05 (m, 0.54×2H), 5.06-4.88 (m, 0.46×2H), 4.59 (dd, J=14.5, 4.4 Hz, 0.43×1H), 4.51-4.47 (m, 1H), 4.36 (dd, J=14.6, 3.5 Hz, 0.57×1H), 4.21 (dtd, J=12.6, 7.7, 3.7 Hz, 1H), 4.02-3.93 (m, 2H), 3.86 (ddd, J=12.4, 7.9, 4.8 Hz, 1H), 3.67 (ddd, J=13.4, 7.9, 4.2 Hz, 1H). ¹⁹F NMR (376 MHz, Acetic Acid-d₄) δ−63.40. LCMS RT (Method 1)=4.957 min. m/z 535.1 [M+H⁺]

### Example 75

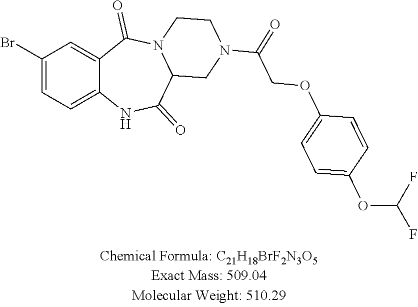 [SMILES: O=C1Nc2ccc(Br)cc2C(=O)N2CCN(C(=O)COc3ccc(OC(F)F)cc3)CC12]

This example is directed to the synthesis of 8-bromo-2-(2-(4-(difluoromethoxy)phenoxy)acetyl)-1,3,4,12a-tetrahydrobenzo[e]pyrazino[1,2-a][1,4]diazepine-6,12 (2H, 11H)-dione (KJW010-013-4) in an aspect of the invention.

The compound was prepared following General Procedure D using 4-(difluoromethoxy)phenol. LCMS RT (Method 2)=2.977 min, m/z 510.0 [M⁺].

### Example 76

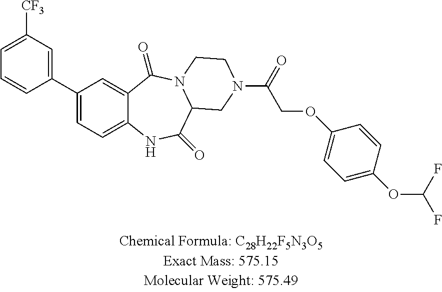 [SMILES: O=C1Nc2ccc(-c3cccc(C(F)(F)F)c3)cc2C(=O)N2CCN(C(=O)COc3ccc(OC(F)F)cc3)CC12]

This example is directed to the synthesis of 2-(2-(4-(difluoromethoxy)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-018) in an aspect of the invention.

The compound was prepared following General Procedure G using 8-bromo-2-(2-(4-(difluoromethoxy)phenoxy)acetyl)-1,3,4,12a-tetrahydrobenzo[e]pyrazino[1,2-a][1,4]diazepine-6,12 (2H, 11H)-dione (KJW010-013-4). ¹H NMR (400 MHz, Acetic Acid-d₄) δ 8.30 (t, J=2.6 Hz, 1H), 7.99 (d, J=3.7 Hz, 1H), 7.97-7.91 (m, 2H), 7.69 (dt, J=15.3, 7.7 Hz, 2H), 7.32 (d, J=8.4 Hz, 1H), 7.10 (t, J=8.4 Hz, 2H), 7.00 (dd, J=9.0, 6.6 Hz, 2H), 6.60 (td, J=74.4, 6.8 Hz, 1H), 5.13-4.98 (m, 0.56×2H), 4.93-4.81 (m, 0.44×2H), 4.58 (dd, J=14.5, 4.6 Hz, 0.44×1H), 4.48 (dt, J=8.2, 4.4 Hz, 1H), 4.39 (dd, J=14.5, 3.8 Hz, 0.56×1H), 4.28-4.21 (m, 0.43×1H), 4.18 (t, J=7.0 Hz, 1H), 4.05-3.83 (m, 3H), 3.70 (dt, J=10.8, 6.3 Hz, 0.57×1H). ¹⁹F NMR (376 MHz, Acetic acid-d₄) δ−63.41, −81.90 (dd, J=74.3, 34.6 Hz). LCMS RT (Method 1)=5.547 min, m/z 576.2 [M+H⁺].

### Example 77

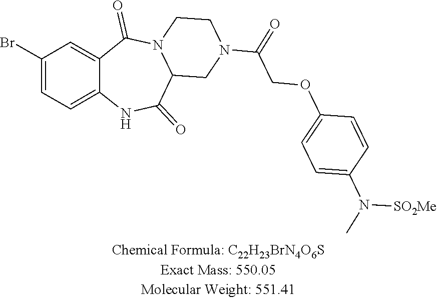 [SMILES: CN(c1ccc(OCC(=O)N2CCN3C(=O)c4cc(Br)ccc4NC(=O)C3C2)cc1)S(C)(=O)=O]

This example is directed to the synthesis of N-(4-(2-(8-bromo-6,12-dioxo-3,4,6,11,12,12a-hexahydrobenzo[e]pyrazino[1,2-a][1,4]diazepin-2 (1H)-yl)-2-oxoethoxy)phenyl)-N-methylmethanesulfonamide (KJW010-013-6) in an aspect of the invention.

The compound was prepared following General Procedure D using N-(4-hydroxyphenyl)-N-methylmethanesulfonamide. LCMS RT (Method 2)=2.723 min, m/z 551.1 [M⁺].

### Example 78

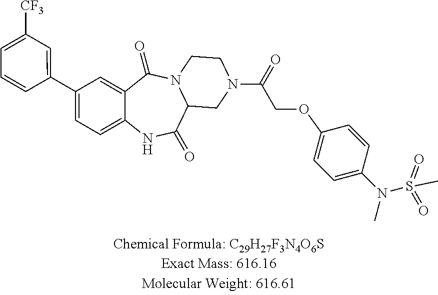 [SMILES: CN(c1ccc(OCC(=O)N2CCN3C(=O)c4cc(-c5cccc(C(F)(F)F)c5)ccc4NC(=O)C3C2)cc1)S(C)(=O)=O]

This example is directed to the synthesis of N-(4-(2-(6,12-dioxo-8-(3-(trifluoromethyl)phenyl)-3,4,6,11,12,12a-hexahydrobenzo[e]pyrazino[1,2-a][1,4]diazepin-2 (1H)-yl)-2-oxoethoxy)phenyl)-N-methylmethanesulfonamide (KJW010-020) in an aspect of the invention.

The compound was prepared following General Procedure G using N-(4-(2-(8-Bromo-6,12-dioxo-3,4,6,11,12,12a-hexahydrobenzo[e]pyrazino[1,2-a][1,4]diazepin-2 (1H)-yl)-2-oxoethoxy)phenyl)-N-methylmethanesulfonamide (KJW010-013-6). ¹H NMR (400 MHz, DMSO-d₆) δ 10.80 (s, 0.68×1H), 10.70 (s, 0.32×1H), 8.10 (t, J=3.3 Hz, 1H), 8.05-7.95 m, 3H), 7.78-7.70 (m, 2H), 7.32-7.25 m, 3H), 7.01-6.94 (m, 2H), 5.13-4.95 (m, 0.69×2H), 4.93-4.75 (m, 0.31×2H), 4.43-4.33 (m, 1H), 4.27-3.83 (m, 3H), 3.79-3.66 (m, 2H), 3.62-3.52 (m, 0.58×1H), 3.43-3.36 (m, 0.42×1H), 3.18 (s, 3H), 2.90 (s, 3H). ¹⁹F NMR (376 MHz, DMSO-d₆) δ−61.04. LCMS RT (Method 1)=5.115 min, m/z 617.2 [M+H⁺].

### Example 79

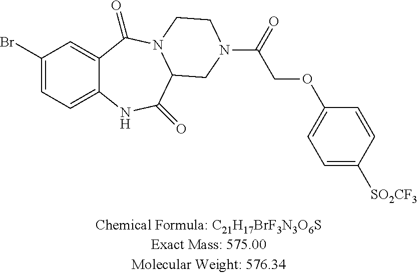

This example is directed to the synthesis of 8-bromo-2-(2-(4-((trifluoromethyl)sulfonyl)phenoxy)acetyl)-1,3,4,12a-tetrahydrobenzo[e]pyrazino[1,2-a][1,4]diazepine-6,12 (2H, 11H)-dione (KJW010-013-8) in an aspect of the invention.

The compound was prepared following General Procedure D using 4-((trifluoromethyl)sulfonyl)phenol. LCMS RT (Method 2)=3.104 min. m/z 576.0 [M⁺].

### Example 80

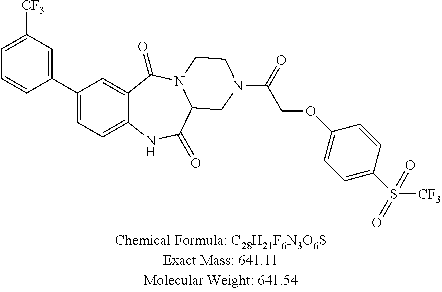 [SMILES: O=C1Nc2ccc(-c3cccc(C(F)(F)F)c3)cc2C(=O)N2CCN(C(=O)COc3ccc(S(=O)(=O)C(F)(F)F)cc3)CC12]

This example is directed to the synthesis of 8-(3-(trifluoromethyl)phenyl)-2-(2-(4-((trifluoromethyl)sulfonyl)phenoxy)acetyl)-1,3,4,12a-tetrahydrobenzo[e]pyrazino[1,2-a][1,4]diazepine-6,12 (2H, 11H)-dione (KJW010-022) in an aspect of the invention.

The compound was prepared following General Procedure G using 8-bromo-2-(2-(4-((trifluoromethyl)sulfonyl)phenoxy)acetyl)-1,3,4,12a-tetrahydrobenzo[e]pyrazino[1,2-a][1,4]diazepine-6,12 (2H, 11H)-dione (KJW010-013-8). ¹H NMR (400 MHz, DMSO-d₆) δ 8.31 (dd, J=5.4, 2.2 Hz, 1H), 8.04 (dd, J=9.0, 7.4 Hz, 2H), 7.99 (d, J=3.0 Hz, 1H), 7.97-7.91 (m, 2H), 7.70 (dt, J=15.3, 7.8 Hz, 2H), 7.35-7.26 (m, 3H), 5.45-5.14 (m, 0.55×2H), 5.08 (q, J=15.4 Hz, 0.45×2), 4.65 (dd, J=14.6, 4.1 Hz, 0.44×1H), 4.51 (m, 4.53-4.49, 1H), 4.36 (dd, J=14.6, 3.2 Hz, 0.56×1H), 4.31-4.10 (m, 1H), 4.06-3.61 (m, 3H). ¹⁹F NMR (376 MHz, DMSO-d₆) δ−62.94, −79.70, −79.71. LCMS RT (Method 1)=5.776 min, m/z 642.1 [M+H⁺].

### Example 81

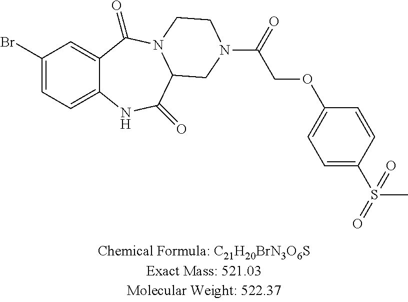 [SMILES: CS(=O)(=O)c1ccc(OCC(=O)N2CCN3C(=O)c4cc(Br)ccc4NC(=O)C3C2)cc1]

This example is directed to the synthesis of 8-bromo-2-(2-(4-(methylsulfonyl)phenoxy)acetyl)-1,3,4,12a-tetrahydrobenzo[e]pyrazino[1,2-a][1,4]diazepine-6,12 (2H, 11H)-dione (KJW010-13-7) in an aspect of the invention.

The compound was prepared following General Procedure D using 4-methylsulfonylphenol. LCMS RT (Method 2)=2.074 min, m/z 522.0 [M⁺].

### Example 82

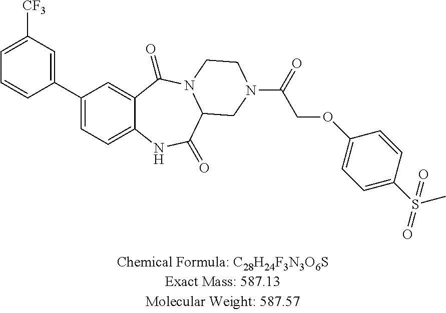 [SMILES: CS(=O)(=O)c1ccc(OCC(=O)N2CCN3C(=O)c4cc(-c5cccc(C(F)(F)F)c5)ccc4NC(=O)C3C2)cc1]

This example is directed to the synthesis of 2-(2-(4-(methylsulfonyl)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-021) in an aspect of the invention.

The compound was prepared following General Procedure G using 8-bromo-2-(2-(4-(methylsulfonyl)phenoxy)acetyl)-1,3,4,12a-tetrahydrobenzo[e]pyrazino[1,2-a][1,4]diazepine-6,12 (2H, 11H)-dione (KJW010-13-7). ¹H NMR (400 MHz, Acetic Acid-d₄) δ 8.31 (dd, J=4.6, 2.2 Hz, 1H), 8.02-7.87 (m, 5H), 7.76-7.50 (m, 3H), 7.33 (dd, J=8.4, 2.2 Hz, 1H), 7.23-7.12 (m, 2H), 5.34-5.08 (m, 0.56×2H), 5.07-4.94 (m, 0.44×2H), 4.61 (dd, J=14.5, 4.3 Hz, 0.44×1H), 4.50 (d, J=4.7 Hz, 1H), 4.45-4.32 (m, 0.56×1H), 4.28-3.56 (m, 5H), 3.10 (d, J=2.5 Hz, 3H). ¹⁹F NMR (376 MHz, Acetic Acid-d₄) δ−63.41, −63.56. LCMS RT (Method 1)=4.963 min. m/z 588.2 [M+H⁺].

### Example 83

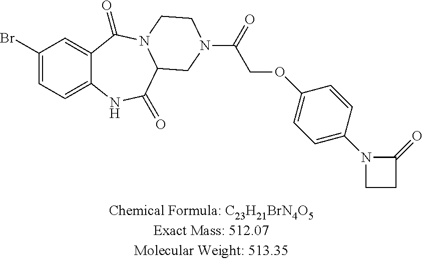 [SMILES: O=C1Nc2ccc(Br)cc2C(=O)N2CCN(C(=O)COc3ccc(N4CCC4=O)cc3)CC12]

This example is directed to the synthesis of 8-bromo-2-(2-(4-(2-oxoazetidin-1-yl)phenoxy)acetyl)-1,3,4,12a-tetrahydrobenzo[e]pyrazino[1,2-a][1,4]diazepine-6,12 (2H, 11H)-dione (KJW010-013-2) in an aspect of the invention.

The compound was prepared following General Procedure D using 1-(4-hydroxyphenyl)azetidin-2-one. LCMS RT (Method 2)=2.755 min, m/z 513.1 [M⁺].

### Example 84

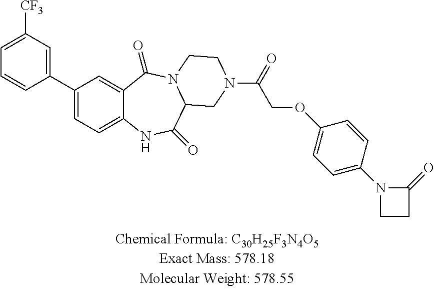 [SMILES: O=C1Nc2ccc(-c3cccc(C(F)(F)F)c3)cc2C(=O)N2CCN(C(=O)COc3ccc(N4CCC4=O)cc3)CC12]

This example is directed to the synthesis of 2-(2-(4-(2-oxoazetidin-1-yl)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-016) in an aspect of the invention.

The compound was prepared following General Procedure G using 8-bromo-2-(2-(4-(2-oxoazetidin-1-yl)phenoxy)acetyl)-1,3,4,12a-tetrahydrobenzo[e]pyrazino[1,2-a][1,4]diazepine-6,12 (2H, 11H)-dione (KJW010-013-2). ¹H NMR (400 MHz, Acetic Acid-d₄) δ 8.30 (d, J=2.2 Hz, 1H), 7.99 (s, 1H), 7.97-7.91 (m, 2H), 7.71 (td, J=8.9, 7.5, 2.6 Hz, 2H), 7.34 (dt, J=10.0, 8.5 Hz, 3H), 7.00 (dd, J=8.8, 5.9 Hz, 2H), 5.14-4.97 (m, 0.56×2H), 4.94-4.79 (m, 0.44×2H), 4.58 (dd, J=14.6, 4.4 Hz, 0.049×1H), 4.54-4.43 (m, 1H), 4.40 (d, J=15.0 Hz, 0.51×1H), 4.26-4.17 (m, 1H), 4.06-3.86 (m, 3H), 3.74-3.64 (m, 3H), 3.12 (dt, J=13.2, 4.4 Hz, 2H). ¹⁹F NMR (376 MHz, Acetic Acid-d₄) δ−63.40. LCMS RT (Method 1)=5.051 min, m/z 579.2 [M+H⁺].

### Example 85

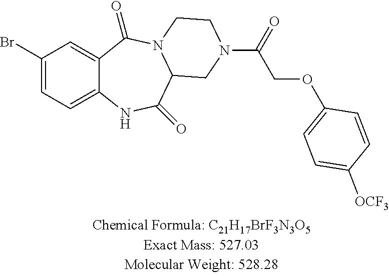 [SMILES: O=C1Nc2ccc(Br)cc2C(=O)N2CCN(C(=O)COc3ccc(OC(F)(F)F)cc3)CC12]

This example is directed to the synthesis of 8-bromo-2-(2-(4-(trifluoromethoxy)phenoxy)acetyl)-1,3,4,12a-tetrahydrobenzo[e]pyrazino[1,2-a][1,4]diazepine-6,12 (2H, 11H)-dione (KJW010-013-3) in an aspect of the invention.

The compound was prepared following General Procedure D using 4-trifluoromethoxyphenol. LCMS RT (Method 2)=3.155 min, m/z 528.0 [M⁺].

### Example 86

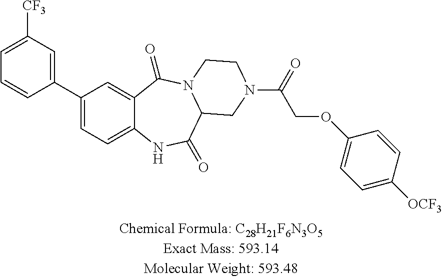 [SMILES: O=C1Nc2ccc(-c3cccc(C(F)(F)F)c3)cc2C(=O)N2CCN(C(=O)COc3ccc(OC(F)(F)F)cc3)CC12]

This example is directed to the synthesis of 2-(2-(4-(trifluoromethoxy)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-017) in an aspect of the invention.

The compound was prepared following General Procedure G using 8-bromo-2-(2-(4-(trifluoromethoxy)phenoxy)acetyl)-1,3,4,12a-tetrahydrobenzo[e]pyrazino[1,2-a][1,4]diazepine-6,12 (2H, 11H)-dione (KJW010-013-3). ¹H NMR (400 MHz, Acetic Acid-d₄) δ 8.30 (t, J=2.6 Hz, 1H), 8.01-7.89 (m, 3H), 7.69 (dt, J=15.3, 7.7 Hz, 2H), 7.32 (d, J=8.5 Hz, 1H), 7.22 (t, J=8.3 Hz, 2H), 7.04 (t, J=8.9 Hz, 2H), 4.96-4.83 (m, 0.55×1H), 4.59 (dd, J=14.6, 4.6 Hz, 0.45×2H), 4.49 (m, 1H), 4.38 (dd, J=14.7, 3.7 Hz, 0.57×1H), 4.21 (m, 2H), 4.05-3.63 (m, 3H). ¹⁹F NMR (376 MHz, Acetic Acid-d₄) δ−59.21, −59.24, −63.41. LCMS RT (Method 1)=5.854 min, m/z 594.0 [M+H⁺].

### Example 87

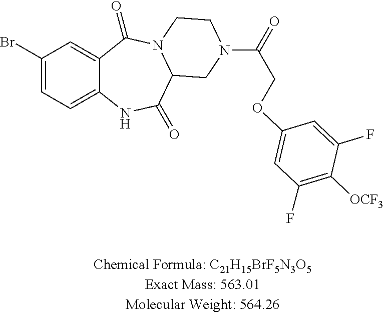 [SMILES: O=C1Nc2ccc(Br)cc2C(=O)N2CCN(C(=O)COc3cc(F)c(OC(F)(F)F)c(F)c3)CC12]

This example is directed to the synthesis of 8-bromo-2-(2-(3,5-difluoro-4-(trifluoromethoxy)phenoxy)acetyl)-1,3,4,12a-tetrahydrobenzo[e]pyrazino[1,2-a][1,4]diazepine-6,12 (2H, 11H)-dione KJW010-013-5) in an aspect of the invention.

The compound was prepared following General Procedure D using 3,5-difluoro-4-(trifluoromethoxy)phenol. LCMS RT (Method 2)=3.245 min, m/z 564.0 [M⁺].

### Example 88

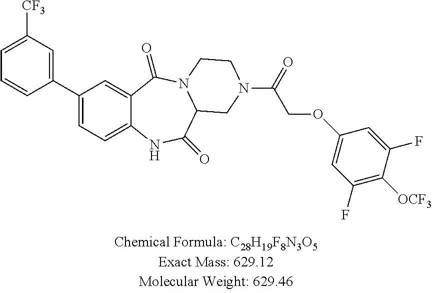 [SMILES: O=C1Nc2ccc(-c3cccc(C(F)(F)F)c3)cc2C(=O)N2CCN(C(=O)COc3cc(F)c(OC(F)(F)F)c(F)c3)CC12]

This example is directed to the synthesis of 2-(2-(3,5-difluoro-4-(trifluoromethoxy)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-019) in an aspect of the invention.

The compound was prepared following General Procedure G using 8-bromo-2-(2-(3,5-difluoro-4-(trifluoromethoxy)phenoxy)acetyl)-1,3,4,12a-tetrahydrobenzo[e]pyrazino[1,2-a][1,4]diazepine-6,12 (2H, 11H)-dione (KJW010-013-5). ¹H NMR (400 MHz, Acetic Acid-d₄) δ 8.30 (dd, J=4.9, 2.2 Hz, 1H), 7.99 (d, J=4.2 Hz, 1H), 7.97-7.90 (m, 2H), 7.69 (dt, J=15.3, 7.8 Hz, 2H), 7.32 (dd, J=8.5, 1.8 Hz, 1H), 6.85-6.76 (m, 2H), 5.26-5.01 (m, 0.55×2H), 5.00-4.87 (m, 0.45×2H), 4.55 (dd, J=14.4, 4.8 Hz, 0.45×1H), 4.49 (m, 1H), 4.32 (dd, J=14.6, 3.6 Hz, 0.55×1H), 4.30-4.10 (m, 1H), 4.06-3.61 (m, 4H). ¹⁹F NMR (376 MHz, Acetic Acid-d₄) δ−61.29 (dt, J=10.3, 6.9 Hz, 1F), −63.41, −76.88, −125.43-−125.53 (m, 1F). LCMS RT (Method 1)=6.078 min, m/z 630.2 [M+H⁺].

### Example 89

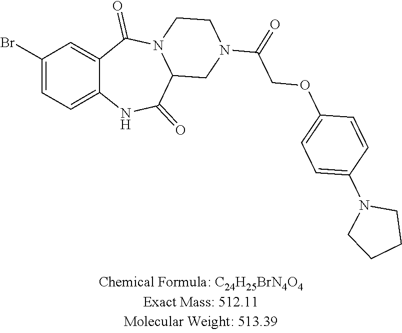 [SMILES: O=C1Nc2ccc(Br)cc2C(=O)N2CCN(C(=O)COc3ccc(N4CCCC4)cc3)CC12]

This example is directed to the synthesis of 8-bromo-2-(2-(4-(pyrrolidin-1-yl)phenoxy)acetyl)-1,3,4,12a-tetrahydrobenzo[e]pyrazino[1,2-a][1,4]diazepine-6,12 (2H, 11H)-dione (KJW010-013-1) in an aspect of the invention.

The compound was prepared following General Procedure D using 4-(pyrrolidin-1-yl)phenol. LCMS RT (Method 2)=2.564 min, m/z 515.1 [M+2H⁺].

### Example 90

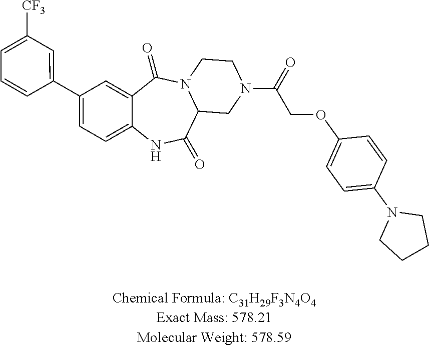 [SMILES: O=C1Nc2ccc(-c3cccc(C(F)(F)F)c3)cc2C(=O)N2CCN(C(=O)COc3ccc(N4CCCC4)cc3)CC12]

This example is directed to the synthesis of 2-(2-(4-(pyrrolidin-1-yl)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-015) in an aspect of the invention.

The compound was prepared following General Procedure G using 8-bromo-2-(2-(4-(pyrrolidin-1-yl)phenoxy)acetyl)-1,3,4,12a-tetrahydrobenzo[e]pyrazino[1,2-a][1,4]diazepine-6,12 (2H, 11H)-dione (KJW010-013-1). ¹H NMR (400 MHz, Acetic Acid-d₄) δ 8.30 (t, J=2.7 Hz, 1H), 8.01-7.90 (m, 4H), 7.74-7.65 (m, 3H), 7.54 (dd, J=9.2, 3.5 Hz, 1H), 7.33 (d, J=8.3 Hz, 1H), 7.11 (dd, J=11.7, 8.7 Hz, 1H), 5.24-5.02 (m, 0.58×2H), 4.99-4.87 (m, 0.42×2H), 4.57 (dd, J=14.4, 4.6 Hz, 0.44×1H), 4.50 (t, J=4.5 Hz, 1H), 4.42-4.35 (m, 0.56×1H), 4.27-3.86 (m, 5H), 3.76-3.65 (m, 4H), 2.38-2.20 (m, 4H). ¹⁹F NMR (376 MHz, acetic acid) 6-63.40. LCMS RT (Method 1)=4.596 min, m/z 579.2 [M+H⁺].

### Example 91

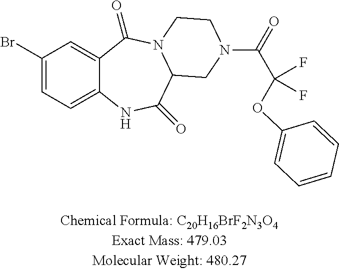 [SMILES: O=C1Nc2ccc(Br)cc2C(=O)N2CCN(C(=O)C(F)(F)Oc3ccccc3)CC12]

This example is directed to the synthesis of 8-bromo-2-(2,2-difluoro-2-phenoxyacetyl)-1,3,4,12a-tetrahydrobenzo[e]pyrazino[1,2-a][1,4]diazepine-6,12 (2H, 11H)-dione (KJW010-025) in an aspect of the invention.

The compound was prepared following General Procedure C using 8-bromo-1,3,4,12a-tetrahydrobenzo[e]pyrazino[1,2-a][1,4]diazepine-6,12 (2H, 11H)-dione 2,2,2-trifluoroacetate (KJW008-042) and sodium 2,2-difluoro-2-phenoxyacetate. ¹H NMR (400 MHz, Chloroform-d) δ 9.12 (s, 1H), 8.06 (dd, J=9.4, 2.4 Hz, 1H), 8.01 (s, 1H), 7.56 (ddd, J=8.5, 2.4, 1.4 Hz, 1H), 7.40-7.30 (m, 2H), 7.21-7.14 (m, 2H), 6.96 (dd, J=8.6, 3.9 Hz, 1H), 4.54-4.35 (m, 1H), 4.33-4.01 (m, 2H), 3.96-3.58 (m, 4H). LCMS RT (Method 2)=3.125 min, m/z 482.0 [M+2H⁺].

### Example 92

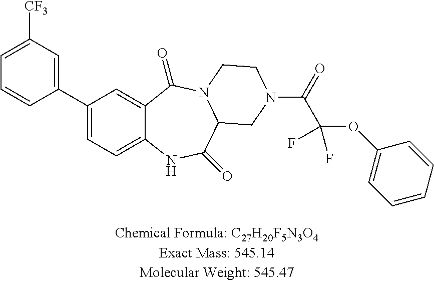 [SMILES: O=C1Nc2ccc(-c3cccc(C(F)(F)F)c3)cc2C(=O)N2CCN(C(=O)C(F)(F)Oc3ccccc3)CC12]

This example is directed to the synthesis of 2-(2,2-difluoro-2-phenoxyacetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-035) in an aspect of the invention.

The compound was prepared following General Procedure G using 8-bromo-2-(2,2-difluoro-2-phenoxyacetyl)-1,3,4,12a-tetrahydrobenzo[e]pyrazino[1,2-a][1,4]diazepine-6,12 (2H, 11H)-dione (KJW010-025). ¹H NMR (400 MHz, Acetic Acid-d₄) δ 8.31 (t, J=2.0 Hz, 1H), 8.00-7.89 (m, 3H), 7.76-7.66 (m, 2H), 7.46-7.22 (m, 6H), 4.68-4.48 (m, 2H), 4.45-4.35 (m, 1H), 4.32-3.61 (m, 4H). ¹⁹F NMR (376 MHz, Acetic Acid-d₄) δ−63.41, −72.10-−73.69 (m, 2F). LCMS RT (Method 1)=5.770 min, m/z 546.2 [M+H⁺].

### Example 93

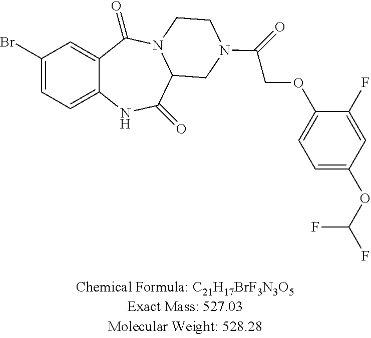 [SMILES: O=C1Nc2ccc(Br)cc2C(=O)N2CCN(C(=O)COc3ccc(OC(F)F)cc3F)CC12]

This example is directed to the synthesis of 8-bromo-2-(2-(4-(difluoromethoxy)-2-fluorophenoxy)acetyl)-1,3,4,12a-tetrahydrobenzo[e]pyrazino[1,2-a][1,4]diazepine-6,12 (2H, 11H)-dione (KJW009-100-1) in an aspect of the invention.

The compound was prepared following General Procedure D using 8-bromo-2-(2-bromoacetyl)-1,3,4,12a-tetrahydrobenzo[e]pyrazino[1,2-a][1,4]diazepine-6,12 (2H, 11H)-dione (KJW009-015) and 4-(difluoromethoxy)-2-fluorophenol. LCMS RT (Method 2)=3.023 min, m/z 528.0 [M⁺].

### Example 94

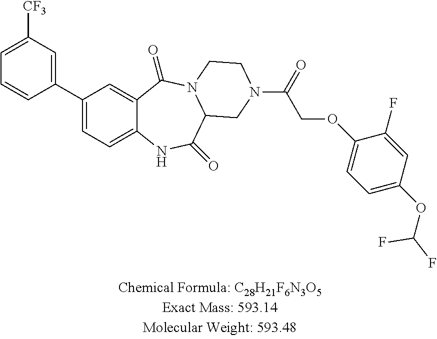 [SMILES: O=C1Nc2ccc(-c3cccc(C(F)(F)F)c3)cc2C(=O)N2CCN(C(=O)COc3ccc(OC(F)F)cc3F)CC12]

This example is directed to the synthesis of 2-(2-(4-(difluoromethoxy)-2-fluorophenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-011) in an aspect of the invention.

The compound was prepared following General Procedure G using 8-bromo-2-(2-(4-(difluoromethoxy)-2-fluorophenoxy)acetyl)-1,3,4,12a-tetrahydrobenzo[e]pyrazino[1,2-a][1,4]diazepine-6,12 (2H, 11H)-dione (KJW009-100-1). ¹H NMR (400 MHz, Acetic Acid-d₄) δ 8.30 (t, J=2.7 Hz, 1H), 7.99 (s, 1H), 7.94 (t, J=8.8 Hz, 2H), 7.69 (dt, J=15.3, 7.8 Hz, 2H), 7.32 (d, J=8.4 Hz, 1H), 7.13 (dt, J=25.7, 9.1 Hz, 1H), 7.04-6.88 (m, 2H), 6.65 (td, J=73.8, 6.0 Hz, 1H), 5.14 (q, J=14.7 Hz, 0.57×2H), 4.95 (q, J=15.0 Hz, 0.43×2H), 4.58 (dd, J=14.5, 4.5 Hz, 0.42×1H), 4.49 (q, J=4.3 Hz, 1H),), 4.40 (dd, J=14.7, 3.7 Hz, 0.58×1H), 4.28-4.21 (m, 0.48×1H), 4.21-4.16 (m, 1H), 4.04-3.65 (m, 4H). ¹⁹F NMR (376 MHz, Acetic Acid-d₄) δ−63.41, −81.51-−85.17 (m), −131.18 (dt, J=95.8, 10.4 Hz). LCMS RT (Method 1)=5.637 min, m/z 594.2 [M+H⁺].

### Example 95

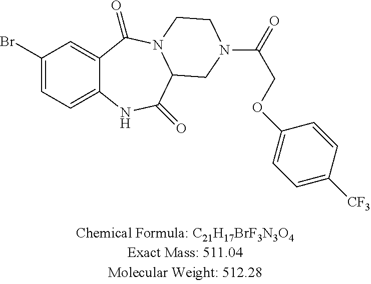 [SMILES: O=C1Nc2ccc(Br)cc2C(=O)N2CCN(C(=O)COc3ccc(C(F)(F)F)cc3)CC12]

This example is directed to the synthesis of 8-bromo-2-(2-(4-(trifluoromethyl)phenoxy)acetyl)-1,3,4,12a-tetrahydrobenzo[e]pyrazino[1,2-a][1,4]diazepine-6,12 (2H, 11H)-dione (KJW010-032) in an aspect of the invention.

The compound was prepared following General Procedure D using 8-bromo-2-(2-bromoacetyl)-1,3,4,12a-tetrahydrobenzo[e]pyrazino[1,2-a][1,4]diazepine-6,12 (2H, 11H)-dione (KJW009-015) and 4-trifluoromethylphenol. LCMS RT (Method 2)=3.188 min, m/z 514.0 [M+2H⁺].

### Example 96

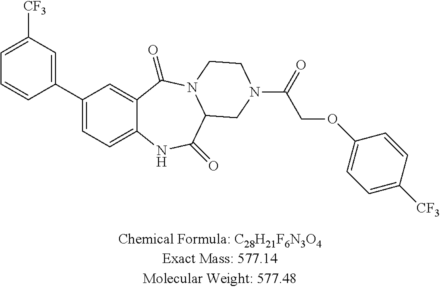 [SMILES: O=C1Nc2ccc(-c3cccc(C(F)(F)F)c3)cc2C(=O)N2CCN(C(=O)COc3ccc(C(F)(F)F)cc3)CC12]

This example is directed to the synthesis of 2-(2-(4-(trifluoromethyl)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-033) in an aspect of the invention.

The compound was prepared following General Procedure G using 8-bromo-2-(2-(4-(trifluoromethyl)phenoxy)acetyl)-1,3,4,12a-tetrahydrobenzo[e]pyrazino[1,2-a][1,4]diazepine-6,12 (2H, 11H)-dione (KJW010-032). ¹H NMR (400 MHz, Acetic Acid-d₄) δ 8.31 (dd, J=4.2, 2.2 Hz, 1H), 7.99 (d, J=3.6 Hz, 1H), 7.98-7.91 (m, 2H), 7.69 (dt, J=15.3, 7.8 Hz, 2H), 7.61 (dd, J=8.7, 6.8 Hz, 2H), 7.32 (dd, J=8.5, 1.9 Hz, 1H), 7.12 (dd, J=11.7, 8.6 Hz, 2H), 5.28-5.04 (m, 0.53×2H), 5.03-4.88 (m, 0.47×2H), 4.60 (dd, J=14.5, 4.4 Hz, 0.45×1H), 4.52-4.47 (m, 1H), 4.39 (dd, J=14.5, 3.6 Hz, 0.55×1H), 4.29-3.64 (m, 5H). ¹⁹F NMR (376 MHz, Acetic Acid-d₄) δ−62.37, −63.41. LCMS RT (Method 1)=5.75 min, m/z 578.0 [M+H⁺].

### Example 97

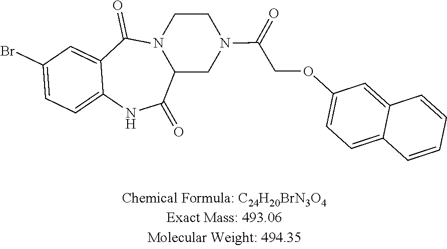 [SMILES: O=C1Nc2ccc(Br)cc2C(=O)N2CCN(C(=O)COc3ccc4ccccc4c3)CC12]

This example is directed to the synthesis of 8-bromo-2-(2-(naphthalen-2-yloxy)acetyl)-1,3,4,12a-tetrahydrobenzo[e]pyrazino[1,2-a][1,4]diazepine-6,12 (2H, 11H)-dione (KJW010-052) in an aspect of the invention.

The compound was prepared following General Procedure D using 8-bromo-2-(2-bromoacetyl)-1,3,4,12a-tetrahydrobenzo[e]pyrazino[1,2-a][1,4]diazepine-6,12 (2H, 11H)-dione (KJW009-015) and 2-hydroxynapthalene. LCMS RT (Method 2)=3.454 min, m/z 496.1 [M+2H⁺].

### Example 98

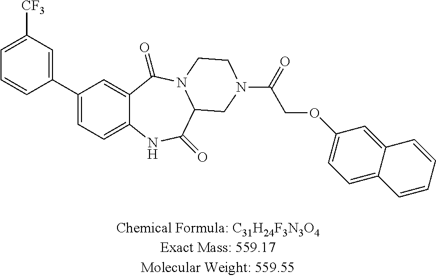 [SMILES: O=C1Nc2ccc(-c3cccc(C(F)(F)F)c3)cc2C(=O)N2CCN(C(=O)COc3ccc4ccccc4c3)CC12]

This example is directed to the synthesis of 2-(2-(naphthalen-2-yloxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-054) in an aspect of the invention.

The compound was prepared following General Procedure G using 8-bromo-2-(2-(naphthalen-2-yloxy)acetyl)-1,3,4,12a-tetrahydrobenzo[e]pyrazino[1,2-a][1,4]diazepine-6,12 (2H, 11H)-dione (KJW010-052). ¹H NMR (400 MHz, Acetic Acid-d₄) δ 8.28 (dd, J=11.1, 2.3 Hz, 1H), 8.02-7.87 (m, 3H), 7.82-7.64 (m, 5H), 7.42 (ddd, J=8.1, 6.3, 4.0 Hz, 1H), 7.38-7.18 (m, 4H), 5.17 (q, J=14.5 Hz, 0.59×2H), 5.08-4.93 (m, 0.41×2H), 4.61-4.43 (m, 2H), 4.27 (dd, J=12.0, 6.4 Hz, 1H), 4.21-3.68 (m, 4H). ¹⁹F NMR (376 MHz, Acetic Acid-d₄) δ−63.40. LCMS RT (Method 1)=5.739 min, m/z 560.2 [M+H⁺].

### Example 99

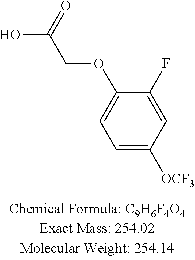 [SMILES: O=C(O)COc1ccc(OC(F)(F)F)cc1F]

This example is directed to the synthesis of 2-(2-fluoro-4-(trifluoromethoxy)phenoxy)-acetic acid (KJW013-010) in an aspect of the invention.

The compound was prepared following General Procedure E using 2-fluoro-4-trifluoromethoxyphenol. ¹H NMR (400 MHz, DMSO-d₆) δ 13.16 (s, 1H), 7.66-7.30 (m, 1H), 7.35-7.03 (m, 2H), 4.82 (d, J=1.1 Hz, 2H). ¹⁹F NMR (376 MHz, DMSO-d₆) δ−57.55, −130.45.

### Example 100

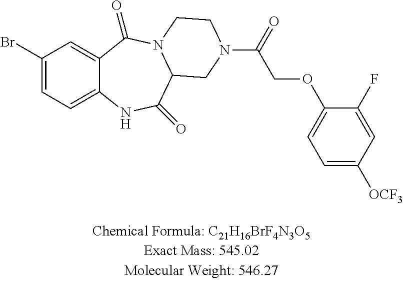 [SMILES: O=C1Nc2ccc(Br)cc2C(=O)N2CCN(C(=O)COc3ccc(OC(F)(F)F)cc3F)CC12]

This example is directed to the synthesis of 8-bromo-2-(2-(2-fluoro-4-(trifluoromethoxy)phenoxy)acetyl)-1,3,4,12a-tetrahydrobenzo[e]pyrazino[1,2-a][1,4]diazepine-6,12 (2H, 11H)-dione (KJW010-064) in an aspect of the invention.

The compound was prepared following General Procedure C using 8-bromo-1,3,4,12a-tetrahydrobenzo[e]pyrazino[1,2-a][1,4]diazepine-6,12 (2H, 11H)-dione and 2-(2-fluoro-4-(trifluoromethoxy)phenoxy)-acetic acid (KJW013-010). LCMS RT (Method 2)=3.474 min, m/z 547.6 [M+H⁺].

### Example 101

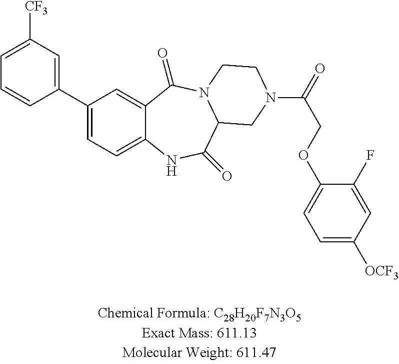 [SMILES: O=C1Nc2ccc(-c3cccc(C(F)(F)F)c3)cc2C(=O)N2CCN(C(=O)COc3ccc(OC(F)(F)F)cc3F)CC12]

This example is directed to the synthesis of 2-(2-(2-fluoro-4-(trifluoromethoxy)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-068) in an aspect of the invention.

The compound was prepared following General Procedure G using 8-bromo-2-(2-(2-fluoro-4-(trifluoromethoxy)phenoxy)acetyl)-1,3,4,12a-tetrahydrobenzo[e]pyrazino[1,2-a][1,4]diazepine-6,12 (2H, 11H)-dione (KJW010-064) and 3-trifluoromethylphenylboronic acid. ¹H NMR (600 MHz, Acetic Acid-d₄) δ 8.32 (dd, J=5.4, 2.3 Hz, 1H), 8.00 (dd, J=5.9, 1.9 Hz, 1H), 7.96 (ddt, J=12.7, 8.4, 1.8 Hz, 2H), 7.76-7.66 (m, 2H), 7.33 (dd, J=8.4, 1.6 Hz, 1H), 7.24-7.04 (m, 3H), 5.29-5.12 (m, 0.56×2H), 5.08-4.89 (m, 0.44×2H), 4.61 (dd, J=14.6, 4.4 Hz, 0.43×1H), 4.54-4.47 (m, 1H), 4.39 (dd, J=14.7, 3.6 Hz, 0.57×1H), 4.27-4.14 (m, 1H), 4.04-3.93 (m, 3H), 3.90 (ddd, J=12.5, 8.3, 4.3 Hz, 0.43×1H), 3.69 (ddd, J=14.9, 8.0, 4.3 Hz, 0.57×1H). ¹⁹F NMR (564 MHz, DMSO-d₆) δ−57.52, −57.53, −61.03, −61.04. LCMS RT (Method 1)=5.903 min, m/z 612.2 [M+H⁺].

### Example 102

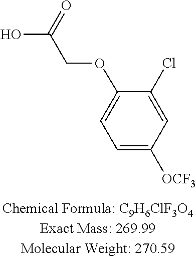 [SMILES: O=C(O)COc1ccc(OC(F)(F)F)cc1Cl]

This example is directed to the synthesis of 2-(2-chloro-4-(trifluoromethoxy)phenoxy)acetic acid (KJW010-072) in an aspect of the invention.

The compound was prepared following General Procedure E using 2-chloro-4-(trifluoromethoxy)phenol. ¹H NMR (400 MHz, Chloroform-d) δ 7.30 (d, J=2.6 Hz, 1H), 7.13-7.05 (m, 1H), 6.85 (dd, J=9.1, 1.9 Hz, 1H), 4.72 (s 2H), 3.82 (s, 3H). ¹⁹F NMR (376 MHz, Chloroform-d) 6-58.46. LCMS RT (Method 2)=3.565 min, m/z 284.9 [M+H⁺].

### Example 103

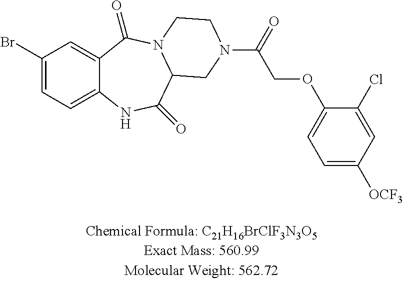 [SMILES: O=C1Nc2ccc(Br)cc2C(=O)N2CCN(C(=O)COc3ccc(OC(F)(F)F)cc3Cl)CC12]

This example is directed to the synthesis of 8-bromo-2-(2-(2-chloro-4-(trifluoromethoxy)phenoxy)acetyl)-1,3,4,12a-tetrahydrobenzo[e]pyrazino[1,2-a][1,4]diazepine-6,12 (2H, 11H)-dione (KJW010-070) in an aspect of the invention.

To a slurry of 8-bromo-1,3,4,12a-tetrahydrobenzo[e]pyrazino[1,2-a][1,4]diazepine-6,12 (2H, 11H)-dione 2,2,2-trifluoroacetate (KJW008-042) (1 eq.) and methyl 2-(2-chloro-4-(trifluoromethoxy)phenoxy)acetate (KJW010-072) (1.4 eq.) in toluene was added AlMe₃ (3 eq.) as a 2M solution in toluene. The result was allowed to stir at 95° C. for 3 hours. The reaction was cooled in an ice-bath and quenched with saturated aq. Rochelle's salt and allowed to warm to room temperature. The reaction was poured into EtOAc and washed twice with NaHCO₃, dried (Na₂SO₄), filtered through CELITE™ and concentrated under vacuum. The residue was purified by flash column chromatography (silica gel, 0-5% MeOH-DCM) to afford the title compound. LCMS RT (Method 2)=3.273 min. m/z 562.1 [M⁺].

### Example 104

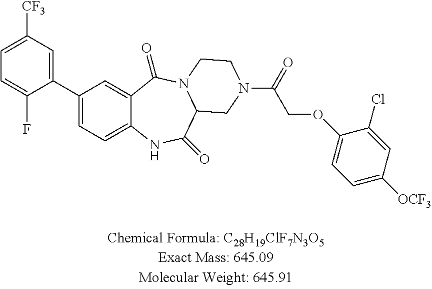 [SMILES: O=C1Nc2ccc(-c3cc(C(F)(F)F)ccc3F)cc2C(=O)N2CCN(C(=O)COc3ccc(OC(F)(F)F)cc3Cl)CC12]

This example is directed to the synthesis of 2-(2-(2-chloro-4-(trifluoromethoxy)phenoxy)acetyl)-8-(2-fluoro-5-(trifluoromethyl)phenyl)-1,3,4,12a-tetrahydrobenzo[e]pyrazino[1,2-a][1,4]diazepine-6,12 (2H, 11H)-dione (KJW010-100) in an aspect of the invention.

The compound was prepared following General Procedure I using 8-bromo-2-(2-(2-chloro-4-(trifluoromethoxy)phenoxy)acetyl)-1,3,4,12a-tetrahydrobenzo[e]pyrazino[1,2-a][1,4]diazepine-6,12 (2H, 11H)-dione (KJW010-070) and 2-fluoro-5-(trifluoromethyl)-phenyl)boronic acid. ¹H NMR (400 MHz, Acetic Acid-d₄) δ 8.23 (s, 1H), 7.92-7.84 (m, 2H), 7.76 (d, J=8.6 Hz, 1H), 7.42 (t, J=9.4 Hz, 1H), 7.39-7.30 (m, 2H), 7.25-7.05 (m, 2H), 5.27-5.14 (m, 0.58×2H), 5.01 (q, J=15.2 Hz, 0.42×2H), 4.66-4.57 (m, 0.35×1H), 4.51 (t, J=4.4 Hz, 1H), 4.41 (dd, J=14.5, 4.1 Hz, 0.65×1H), 4.31-4.14 (m, 1H), 4.10-3.64 (m, 4H). ¹⁹F NMR (376 MHz, Acetic Acid-d₄) δ−59.35, −59.41, −113.35-−133.42 (m). LCMS RT (Method 1)=6.042 min, m/z 646.1 [M+H⁺].

### Example 105

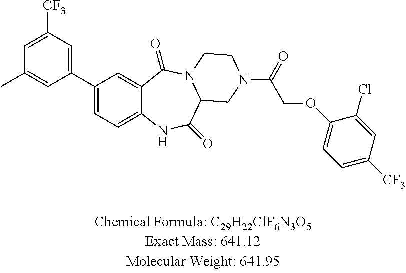 [SMILES: Cc1cc(-c2ccc3c(c2)C(=O)N2CCN(C(=O)COc4ccc(C(F)(F)F)cc4Cl)CC2C(=O)N3)cc(C(F)(F)F)c1]

This example is directed to the synthesis of 2-(2-(2-chloro-4-(trifluoromethoxy)phenoxy)acetyl)-8-(3-methyl-5-(trifluoromethyl)phenyl)-1,3,4,12a-tetrahydrobenzo[e]pyrazino[1,2-a][1,4]diazepine-6,12 (2H, 11H)-dione (KJW011-001) in an aspect of the invention.

The compound was prepared following General Procedure I using 8-bromo-2-(2-(2-chloro-4-(trifluoromethoxy)phenoxy)acetyl)-1,3,4,12a-tetrahydrobenzo[e]pyrazino[1,2-a][1,4]diazepine-6,12 (2H, 11H)-dione (KJW010-070) and 3-methyl-5-(trifluoromethyl)-phenyl)boronic acid. ¹H NMR (400 MHz, Acetic Acid-d₄) δ 8.28 (d, J=2.2 Hz, 1H), 7.91 (dd, J=8.4, 2.2 Hz, 1H), 7.79-7.75 (m, 2H), 7.53 (s, 1H), 7.39-7.27 (m, 2H), 7.24-7.05 (m, 2H), 5.26-5.11 (m, 0.58×2H), 5.01 (q, J=15.1 Hz, 0.42×2H), 4.61 (dd, J=14.5, 4.2 Hz, 0.40×1H), 4.49 (t, J=4.4 Hz, 1H), 4.41 (dd, J=14.8, 4.1 Hz, 0.60×1H), 4.32-4.14 (m, 1H), 4.09-3.66 (m, 4H), 2.51 (s, 3H). ¹⁹F NMR (376 MHz, Acetic acid-d₄) δ−59.35, −59.40, −63.29. LCMS RT (Method 1)=6.257 min, m/z 642.1 [M+H⁺].

### Example 106

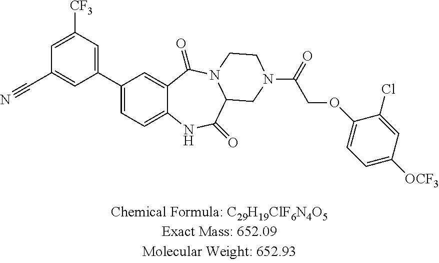 [SMILES: N#Cc1cc(-c2ccc3c(c2)C(=O)N2CCN(C(=O)COc4ccc(OC(F)(F)F)cc4Cl)CC2C(=O)N3)cc(C(F)(F)F)c1]

This example is directed to the synthesis of 3-(2-(2-(2-chloro-4-(trifluoromethoxy)phenoxy)acetyl)-6,12-dioxo-1,2,3,4,6,11,12,12a-octahydrobenzo[e]pyrazino[1,2-a]1[1,4]diazepin-8-yl)-5-(trifluoromethyl)benzonitrile (KJW011-002) in an aspect of the invention.

The compound was prepared following General Procedure I using 8-bromo-2-(2-(2-chloro-4-(trifluoromethoxy)phenoxy)acetyl)-1,3,4,12a-tetrahydrobenzo[e]pyrazino[1,2-a][1,4]diazepine-6,12 (2H, 11H)-dione (KJW010-070) and 3-cyano-5-(trifluoromethyl)-phenylboronic acid. ¹H NMR (400 MHz, Acetic Acid-d₄) δ 8.28 (d, J=2.2 Hz, 1H), 7.91 (dd, J=8.4, 2.2 Hz, 1H), 7.77 (d, J=5.8 Hz, 2H), 7.53 (s, 1H), 7.39-7.27 (m, 2H), 7.25-7.04 (m, 2H), 5.25-5.13 (m, 0.58×2H), 5.01 (q, J=15.1 Hz, 0.42×2H), 4.61 (dd, J=14.5, 4.2 Hz, 0.40×2H), 4.49 (t, J=4.4 Hz, 1H), 4.41 (dd, J=14.8, 4.1 Hz, 0.60×1H), 4.33-4.14 (m, 1H), 4.09-3.66 (m, 4H). ¹⁹F NMR (376 MHz, Acetic Acid-d₄) δ−59.35, −59.40, −63.30. LCMS RT (Method 1)=5.906 min, m/z 653.2 [M+H⁺].

### Example 107

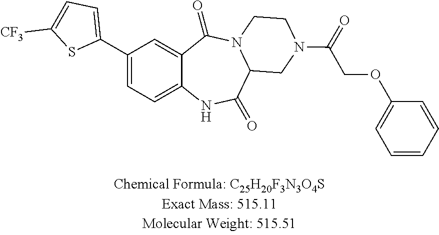 [SMILES: O=C1Nc2ccc(-c3ccc(C(F)(F)F)s3)cc2C(=O)N2CCN(C(=O)COc3ccccc3)CC12]

This example is directed to the synthesis of 2-(2-phenoxyacetyl)-8-(5-(trifluoromethyl)thiophen-2-yl)-1,3,4,12a-tetrahydrobenzo[e]pyrazino[1,2-a][1,4]diazepine-6,12 (2H, 11H)-dione (KJW011-010) in an aspect of the invention.

The compound was prepared following General Procedure I using 8-bromo-2-(2-phenoxyacetyl)-1,3,4,12a-tetrahydrobenzo[e]pyrazino[1,2-a][1,4]diazepine-6,12 (2H, 11H)-dione (KJW011-004) and 5-(trifluoromethyl)thiophen-2-yl)-boronic acid. ¹H NMR (400 MHz, DMSO-d₆) δ 10.82 (s, 0.69×1H), 10.74 (s, 0.31×1H), 8.06 (t, J=2.7 Hz, 1H), 7.94 (dd, J=8.4, 2.3 Hz, 1H), 7.79-7.72 (m, 1H), 7.65 (ddd, J=6.9, 3.3, 1.4 Hz, 1H), 7.30-7.20 (m, 3H), 7.00-6.89 (m, 3H), 5.07-4.91 (m, 0.69×2H), 4.88-4.70 (m, 0.31×1H), 4.42-4.34 (m, 1H), 4.23-3.94 (m, 2H), 3.91-3.35 (m, 3H). ¹⁹F NMR (376 MHz, DMSO-d₆) δ−53.93. LCMS RT (Method 1)=5.435 min. m/z 516.1 [M+H⁺].

### Example 108

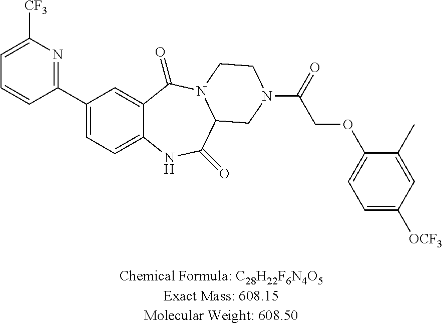 [SMILES: Cc1cc(OC(F)(F)F)ccc1OCC(=O)N1CCN2C(=O)c3cc(-c4cccc(C(F)(F)F)n4)ccc3NC(=O)C2C1]

This example is directed to the synthesis of 2-(2-(2-methyl-4-(trifluoromethoxy)phenoxy)-acetyl)-8-(6-(trifluoromethyl)pyridin-2-yl)-1,3,4,12a-tetrahydrobenzo[e]pyrazino[1,2-a][1,4]diazepine-6,12 (2H, 11H)-dione (KJW011-045) in an aspect of the invention.

The compound was prepared following General Procedure I using 8-bromo-2-(2-(2-methyl-4-(trifluoromethoxy)phenoxy)acetyl)-1,3,4,12a-tetrahydrobenzo[e]pyrazino[1,2-a][1,4]diazepine-6,12 (2H, 11H)-dione and 3-(trifluoromethyl)-phenylboronic acid. H¹ NMR (400 MHz, Acetic Acid-d₄) δ 8.70 (dd, J=9.3, 2.2 Hz, 1H), 8.43 (ddd, J=7.9, 5.5, 2.2 Hz, 1H), 8.19 (d, J=7.4 Hz, 1H), 8.10 (t, J=7.9 Hz, 1H), 7.72 (dd, J=22.3, 7.4 Hz, 2H), 7.12-6.87 (m, 3H), 5.20-5.01 (m, 0.58×2H), 5.00-4.83 (m, 0.42×2H), 4.60 (dd, J=14.4, 4.6 Hz, 0.42×1H), 4.49 (dt, J=13.5, 4.4 Hz, 1H), 4.41 (dd, J=14.7, 3.9 Hz, 0.58×1H), 4.28-4.15 (m, 1H), 4.07-3.63 (m, 4H), 2.29 (s, 0.49×3H), 2.26 (s, 0.51×3H). ¹⁹F NMR (376 MHz, acetic acid) 6-59.06, −59.10, −68.87. LCMS RT (Method 1)=5.762 min, m/z 609.2 [M+H⁺].

### Example 109

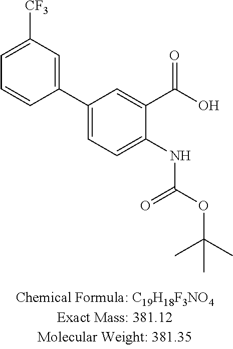 [SMILES: CC(C)(C)OC(=O)Nc1ccc(-c2cccc(C(F)(F)F)c2)cc1C(=O)O]

This example is directed to the synthesis of 4-((tert-butoxycarbonyl)amino)-3′-(trifluoromethyl)-[1,1′-biphenyl]-3-carboxylic acid (KJW010-066) in an aspect of the invention.

The compound was prepared following General Procedure K using 3-trifluorophenylboronic acid. ¹H NMR (400 MHz, DMSO-d₆) δ 13.83 (s, 1H), 10.56 (s, 1H), 8.40 (d, J=8.8 Hz, 1H), 8.25 (d, J=2.4 Hz, 1H), 8.04-7.91 (m, 3H), 7.76-7.65 (m, 2H), 1.50 (s, 9H). ¹⁹F NMR (376 MHz, DMSO-d₆) δ−61.00. LCMS RT (Method 2)=3.760 min, m/z 282.1 [M-BOC+H⁺].

### Example 110

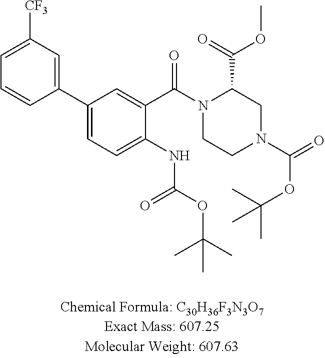 [SMILES: COC(=O)[C@@H]1CN(C(=O)OC(C)(C)C)CCN1C(=O)c1cc(-c2cccc(C(F)(F)F)c2)ccc1NC(=O)OC(C)(C)C]

This example is directed to the synthesis of 1-(tert-butyl)-3-methyl (S)-4-(4-((tert-butoxycarbonyl)amino)-3′-(trifluoromethyl)-[1,1′-biphenyl]-3-carbonyl)piperazine-1,3-dicarboxylate (KJW011-022-2) in an aspect of the invention.

The compound was prepared following General Procedure A using (S)-1-(tert-butyl) 3-methyl piperazine-1,3-dicarboxylate and 4-((tert-butoxycarbonyl)amino)-3′-(trifluoromethyl)-[1,1′-biphenyl]-3-carboxylic acid (KJW010-066). [α]_{D}²⁰=−0.92° (c=1, CHCl₃). ¹H NMR (400 MHz, Acetic Acid-d₄) δ 8.09-7.82 (m, 3H), 7.77 (dt, J=7.7, 3.8 Hz, 1H), 7.70-7.59 (m, 3H), 5.44 (bs, 1H), 4.75-4.50 (m, 2H), 4.24-2.90 (m, 7H), 1.54 (s, 9H), 1.46 (s, 9H). ¹⁹F NMR (376 MHz, Acetonitrile-d₃) δ−63.09. LCMS RT (Method 2)=3.852 min. m/z 607.8 [M⁺].

### Example 111

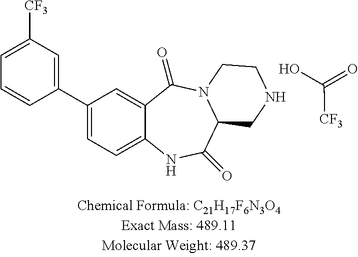

This example is directed to the synthesis of (S)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW010-088) in an aspect of the invention.

The compound was prepared following General Procedure B using 1-(tert-butyl)-3-methyl (S)-4-(4-((tert-butoxycarbonyl)amino)-3′-(trifluoromethyl)-[1,1′-biphenyl]-3-carbonyl)piperazine-1,3-dicarboxylate (KJW011-022-2). [α]_{D}²⁰=+16.4 (c=1, MeOH). ¹H NMR (400 MHz, DMSO-d₆) δ 10.95 (s, 1H), 9.39 (bs, 0.62×1H), 8.68 (bs, 0.38×1H), 8.07 (d, J=2.3 Hz, 1H), 8.04-7.96 (m, 3H), 7.79-7.70 (m, 2H), 7.28 (d, J=8.5 Hz, 1H), 4.57 (dd, J=5.3, 2.9 Hz, 1H), 4.23 (dt, J=14.5, 4.4 Hz, 1H), 3.69 (dd, J=13.7, 3.0 Hz, 1H), 3.53-3.18 (m, 4H). ¹⁹F NMR (376 MHz, DMSO-d₆) δ−61.06, −73.63. LCMS RT (Method 2)=2.998 min, m/z 376.2 [M+H⁺].

### Example 112

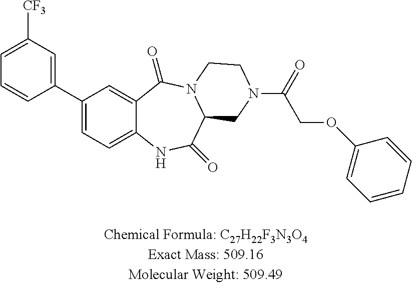 [SMILES: O=C1Nc2ccc(-c3cccc(C(F)(F)F)c3)cc2C(=O)N2CCN(C(=O)COc3ccccc3)C[C@@H]12]

This example is directed to the synthesis of (S)-2-(2-phenoxyacetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-083) in an aspect of the invention.

The compound was prepared following General Procedure C using (S)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW010-088) and 2-phenoxyacetic acid. [α]_{D}²⁰=+17.6° (c=1, DMSO). ¹H NMR (400 MHz, Acetic Acid-d₄) δ 8.29 (d, J=2.4 Hz, 1H), 7.99 (d, J=4.0 Hz, 1H), 7.97-7.89 (m, 2H), 7.75-7.64 (m, 2H), 7.28 (dt, J=19.7, 8.2 Hz, 3H), 7.01-6.90 (m, 3H), 5.10-4.98 (m, 0.56×2H), 4.94-4.79 (m, 0.44×2H), 4.60-4.37 (m, 2H), 4.29-3.65 (m, 5H). ¹⁹F NMR (376 MHz, DMSO-d₆) δ−61.04, −61.06. LCMS RT (Method 1)=5.235 min, m/z 510.1 [M+H⁺].

### Example 113

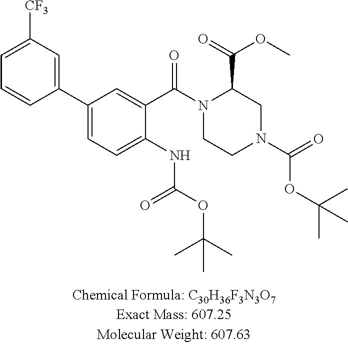 [SMILES: COC(=O)[C@H]1CN(C(=O)OC(C)(C)C)CCN1C(=O)c1cc(-c2cccc(C(F)(F)F)c2)ccc1NC(=O)OC(C)(C)C]

This example is directed to the synthesis of 1-(tert-butyl) 3-methyl (R)-4-(4-((tert-butoxycarbonyl)amino)-3′-(trifluoromethyl)-[1,1′-biphenyl]-3-carbonyl)piperazine-1,3-dicarboxylate (KJW010-087) in an aspect of the invention.

The compound was prepared following General Procedure A using (R)-1-(tert-butyl) 3-methyl piperazine-1,3-dicarboxylate and 4-((tert-butoxycarbonyl)amino)-3′-(trifluoromethyl)-[1,1′-biphenyl]-3-carboxylic acid (KJW010-066). [α]_{D}²⁰=+0.64° (c=1, CHCl₃). ¹H NMR (400 MHz, Acetic Acid-d₄) δ 8.09-7.82 (m, 3H), 7.77 (dt, J=7.7, 3.8 Hz, 1H), 7.70-7.59 (m, 3H), 5.44 (bs, 1H), 4.75-4.50 (m, 2H), 4.24-2.90 (m, 7H), 1.54 (s, 9H), 1.46 (s, 9H). ¹⁹F NMR (376 MHz, Acetic Acid-d₄) δ−63.34, −63.37. LCMS RT (Method 2)=3.852 min. m/z 607.8 [M⁺].

### Example 114

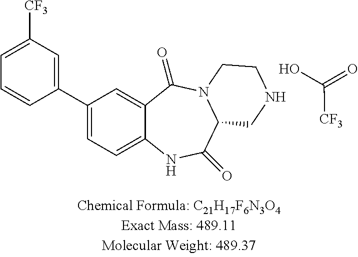

This example is directed to the synthesis of (R)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW010-079) in an aspect of the invention.

The compound was prepared following General Procedure B using 1-(tert-butyl) 3-methyl (R)-4-(4-((tert-butoxycarbonyl)amino)-3′-(trifluoromethyl)-[1,1′-biphenyl]-3-carbonyl)piperazine-1,3-dicarboxylate (KJW011-022-2). [α]_{D}²⁰=−16.4 (c=1, MeOH). ¹H NMR (400 MHz, DMSO-d₆) δ 10.95 (s, 1H), 9.39 (bs, 0.62×1H), 8.68 (bs, 0.38×1H), 8.07 (d, J=2.3 Hz, 1H), 8.04-7.96 (m, 3H), 7.79-7.70 (m, 2H), 7.28 (d, J=8.5 Hz, 1H), 4.57 (dd, J=5.3, 2.9 Hz, 1H), 4.23 (dt, J=14.5, 4.4 Hz, 1H), 3.69 (dd, J=13.7, 3.0 Hz, 1H), 3.53-3.18 (m, 4H). ¹⁹F NMR (376 MHz, DMSO-d₆) δ−61.06, −73.63. LCMS RT (Method 2)=2.998 min, m/z 376.2 [M+H⁺].

### Example 115

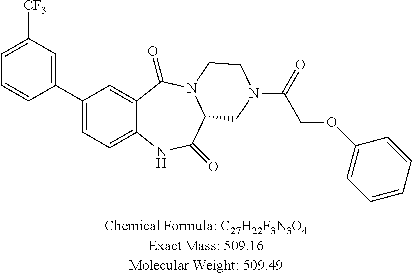 [SMILES: O=C1Nc2ccc(-c3cccc(C(F)(F)F)c3)cc2C(=O)N2CCN(C(=O)COc3ccccc3)C[C@H]12]

This example is directed to the synthesis of (R)-2-(2-phenoxyacetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-082) in an aspect of the invention.

The compound was prepared following General Procedure C using (R)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW010-079) and 2-phenoxyacetic acid. [α]_{D}²⁰=−17.6° (c=1, DMSO). ¹H NMR (400 MHz, Acetic Acid-d₄) δ 8.29 (d, J=2.4 Hz, 1H), 7.99 (d, J=4.0 Hz, 1H), 7.97-7.89 (m, 2H), 7.75-7.64 (m, 2H), 7.28 (dt, J=19.7, 8.2 Hz, 3H), 7.01-6.90 (m, 3H), 5.10-4.98 (m, 0.56×2H), 4.94-4.79 (m, 0.44×2H), 4.60-4.37 (m, 2H), 4.29-3.65 (m, 5H). ¹⁹F NMR (376 MHz, DMSO-d₆) δ−61.04, −61.06. LCMS RT (Method 1)=5.235 min, m/z 510.1 [M+H⁺].

### Example 116

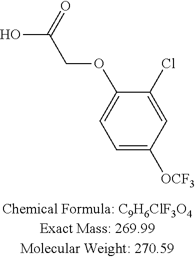 [SMILES: O=C(O)COc1ccc(OC(F)(F)F)cc1Cl]

This example is directed to the synthesis of 2-(2-chloro-4-(trifluoromethoxy)phenoxy)acetic acid (KJW010-093) in an aspect of the invention.

The compound was prepared following General Procedure E using 2-chloro-4-trifluoromethoxyphenol. 1H NMR (400 MHz, DMSO-d₆) δ 13.17 (bs, 1H), 7.65-7.51 (m, 1H), 7.33 (ddq, J=9.1, 3.0, 1.0 Hz, 1H), 7.14 (d, J=9.2 Hz, 1H), 4.86 (s, 2H). ¹⁹F NMR (376 MHz, DMSO-d₆) δ−57.48.

### Example 117

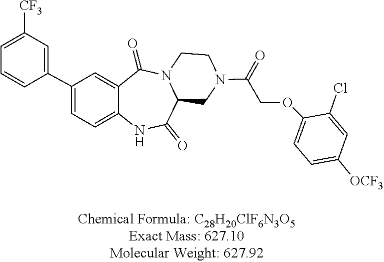 [SMILES: O=C1Nc2ccc(-c3cccc(C(F)(F)F)c3)cc2C(=O)N2CCN(C(=O)COc3ccc(OC(F)(F)F)cc3Cl)C[C@@H]12]

This example is directed to the synthesis of (S)-2-(2-(2-chloro-4-(trifluoromethoxy)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-095) in an aspect of the invention.

The compound was prepared following General Procedure C using (S)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW010-088) and 2-(2-chloro-4-(trifluoromethoxy)phenoxy)acetic acid (KJW010-093). ¹H NMR (400 MHz, DMSO-d₆) δ 10.81 (s, 0.69×1H), 10.70 (s, 0.31×1H). 8.10 (dd, J=3.3, 2.3 Hz, 1H), 8.05-7.93 (m, 3H), 7.78-7.70 (m, 2H), 7.56 (ddd, J=6.1, 2.9, 0.8 Hz, 1H), 7.32-7.13 (m, 3H), 5.28-5.14 (m, 0.70×2H), 5.12-4.94 (m, 0.30×2H), 4.42 (t, J=4.6 Hz, 0.70×1H), 4.36 (t, J=4.9 Hz, 0.30×1H), 4.24-3.84 (m, 2H), 3.78-3.66 (m, 2H), 3.56 (m, 0.45×1H), 3.39 (m, 0.55×1H). ¹⁹F NMR (376 MHz, DMSO-d₆) δ−57.45, −57.47, −61.05, −61.06. LCMS RT (Method 1)=6.524 min, m/z 627.6 [M⁺].

### Example 118

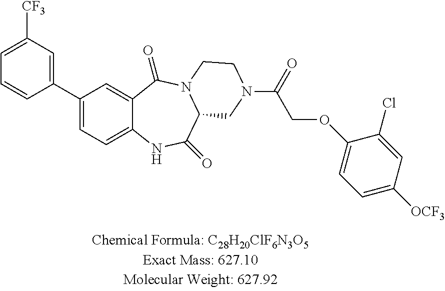 [SMILES: O=C1Nc2ccc(-c3cccc(C(F)(F)F)c3)cc2C(=O)N2CCN(C(=O)COc3ccc(OC(F)(F)F)cc3Cl)C[C@H]12]

This example is directed to the synthesis of (R)-2-(2-(2-chloro-4-(trifluoromethoxy)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW010-096) in an aspect of the invention.

The compound was prepared following General Procedure C using (R)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW010-079) and 2-(2-chloro-4-(trifluoromethoxy)-phenoxy)acetic acid (KJW010-093). ¹H NMR (400 MHz, DMSO-d₆) δ 10.81 (s, 0.69×1H), 10.70 (s, 0.31×1H). 8.10 (dd, J=3.3, 2.3 Hz, 1H), 8.05-7.93 (m, 3H), 7.78-7.70 (m, 2H), 7.56 (ddd, J=6.1, 2.9, 0.8 Hz, 1H), 7.32-7.13 (m, 3H), 5.28-5.14 (m, 0.70×2H), 5.12-4.94 (m, 0.30×2H), 4.42 (t, J=4.6 Hz, 0.70×1H), 4.36 (t, J=4.9 Hz, 0.30×1H), 4.24-3.84 (m, 2H), 3.78-3.66 (m, 2H), 3.56 (m, 0.45×1H), 3.39 (m, 0.55×1H). ¹⁹F NMR (376 MHz, DMSO-d₆) δ−57.45, −57.47, −61.05, −61.06. LCMS RT (Method 1)=6.524 min, m/z 627.6 [M⁺].

### Example 119

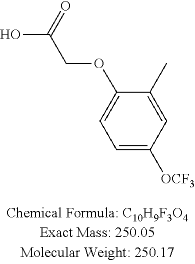 [SMILES: Cc1cc(OC(F)(F)F)ccc1OCC(=O)O]

This example is directed to the synthesis of 2-(2-methyl-4-(trifluoromethoxy)-phenoxy)acetic acid (KJW011-028) in an aspect of the invention.

The compound was prepared following General Procedure E using 2-methyl-4-trifluoromethoxyphenol. ¹H NMR (400 MHz, DMSO-d₆) δ 13.18 (s, 1H), 7.57 (dt, J=2.8, 0.8 Hz, 1H), 7.32 (ddq, J=9.1, 3.0, 1.0 Hz, 1H), 7.14 (d, J=9.2 Hz, 1H), 4.86 (s, 2H). LCMS RT (Method 4)=1.556 min, m/z 249.0 [M−H]⁻.

### Example 120

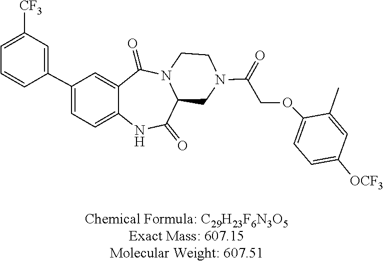 [SMILES: Cc1cc(OC(F)(F)F)ccc1OCC(=O)N1CCN2C(=O)c3cc(-c4cccc(C(F)(F)F)c4)ccc3NC(=O)[C@@H]2C1]

This example is directed to the synthesis of (S)-2-(2-(2-methyl-4-(trifluoromethoxy)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW011-005) in an aspect of the invention.

The compound was prepared following General Procedure C using (S)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW010-088) and 2-(2-methyl-4-(trifluoromethoxy)-phenoxy)-acetic acid (KJW011-028). ¹H NMR (400 MHz, Acetic Acid-d₄) δ 8.31 (d, J=2.2 Hz, 1H), 7.99 (d, J=4.1 Hz, 1H), 7.97-7.90 (m, 2H), 7.70 (dt, J=15.3, 7.8 Hz, 2H), 7.32 (d, J=8.4 Hz, 1H), 7.11-7.03 (m, 2H), 6.97-6.87 (m, 1H), 5.19-5.03 (m, 0.55×2H), 5.00-4.84 (m, 0.45×2H), 4.61 (dd, J=14.6, 4.4 Hz, 0.46×1H), 4.49 (dt, J=12.4, 4.4 Hz, 1H), 4.41 (dd, J=14.5, 3.7 Hz, 0.54×1H), 4.29-3.66 (m, 5H), 2.29 (s, 0.45×3H), 2.26 (s, 0.55×3H). ¹⁹F NMR (376 MHz, Acetic Acid-d₄) δ−59.06, −59.09, −63.41. LCMS RT (Method 1)=5.974 min, m/z 608.1 [M+H⁺].

### Example 121

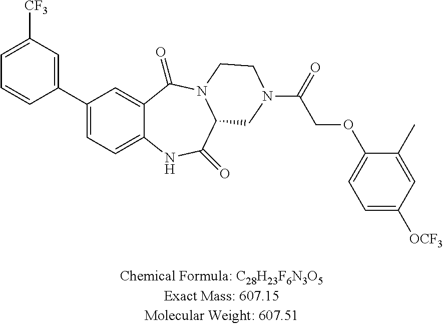 [SMILES: Cc1cc(OC(F)(F)F)ccc1OCC(=O)N1CCN2C(=O)c3cc(-c4cccc(C(F)(F)F)c4)ccc3NC(=O)[C@H]2C1]

This example is directed to the synthesis of (R)-2-(2-(2-methyl-4-(trifluoromethoxy)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW011-006) in an aspect of the invention.

The compound was prepared following General Procedure C using (R)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW010-079) and 2-(2-methyl-4-(trifluoromethoxy)-phenoxy)acetic acid (KJW011-028). ¹H NMR (400 MHz, Acetic Acid-d₄) δ 8.31 (d, J=2.2 Hz, 1H), 7.99 (d, J=4.1 Hz, 1H), 7.97-7.90 (m, 2H), 7.70 (dt, J=15.3, 7.8 Hz, 2H), 7.32 (d, J=8.4 Hz, 1H), 7.11-7.03 (m, 2H), 6.97-6.87 (m, 1H), 5.19-5.03 (m, 0.55×2H), 5.00-4.84 (m, 0.45×2H), 4.61 (dd, J=14.6, 4.4 Hz, 0.46×1H), 4.49 (dt, J=12.4, 4.4 Hz, 1H), 4.41 (dd, J=14.5, 3.7 Hz, 0.54×1H), 4.29-3.66 (m, 5H), 2.29 (s, 0.45×3H), 2.26 (s, 0.55×3H). ¹⁹F NMR (376 MHz, Acetic Acid-d₄) δ−59.06, −59.09, −63.41. LCMS RT (Method 1)=5.974 min, m/z 608.1 [M+H⁺].

### Example 122

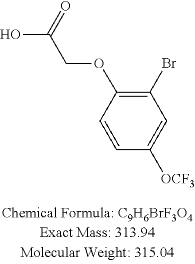 [SMILES: O=C(O)COc1ccc(OC(F)(F)F)cc1Br]

This example is directed to the synthesis of 2-(2-bromo-4-(trifluoromethoxy)phenoxy)acetic acid (KJW011-027) in an aspect of the invention.

The compound was prepared following General Procedure E using 2-bromo-14-trifluoromethoxyphenol. ¹H NMR (400 MHz, DMSO-d₆) δ 13.18 (s, 1H), 7.69 (dd, J=2.9, 0.9 Hz, 1H), 7.37 (ddq, J=9.1, 2.9, 1.0 Hz, 1H), 7.10 (d, J=9.1 Hz, 1H), 4.85 (s, 2H). ¹⁹F NMR (376 MHz, DMSO-d₆) δ−57.45. LCMS RT (Method 4)=1.987 min, m/z 314.9 [M−H]⁻.

### Example 123

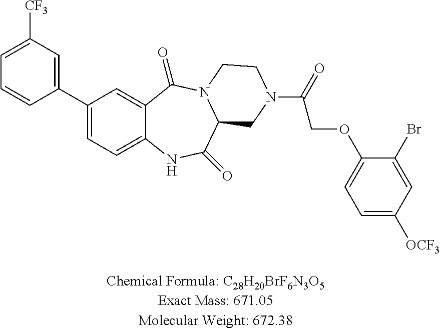 [SMILES: O=C1Nc2ccc(-c3cccc(C(F)(F)F)c3)cc2C(=O)N2CCN(C(=O)COc3ccc(OC(F)(F)F)cc3Br)C[C@@H]12]

This example is directed to the synthesis of (S)-2-(2-(2-bromo-4-(trifluoromethoxy)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW011-042) in an aspect of the invention.

The compound was prepared following General Procedure C using (S)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW008-042) and 2-(2-bromo-4-(trifluoromethoxy)-phenoxy)-acetic acid (KJW011-027). ¹H NMR (400 MHz, Acetic Acid-d₄) δ 8.30 (d, J=2.4 Hz, 1H), 7.99 (d, J=3.2 Hz, 1H), 7.97-7.90 (m, 2H), 7.69 (dt, J=15.3, 7.8 Hz, 2H), 7.54-7.47 (m, 1H), 7.32 (dd, J=8.4, 2.5 Hz, 1H), 7.29-7.23 (m, 1H), 7.14-7.04 (m, 1H). ¹⁹F NMR (376 MHz Acetic Acid-d₄) δ−59.34, −59.40, −63.40. LCMS RT (Method 1)=5.956 min, m/z 672.1 [M⁺].

### Example 124

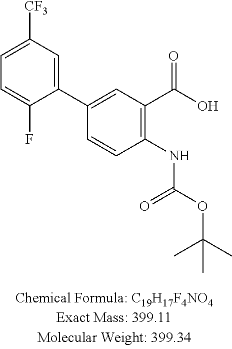 [SMILES: CC(C)(C)OC(=O)Nc1ccc(-c2cc(C(F)(F)F)ccc2F)cc1C(=O)O]

This example is directed to the synthesis of 4-((tert-butoxycarbonyl)amino)-2′-fluoro-5′-(trifluoromethyl)-[1,1′-biphenyl]-3-carboxylic acid (KJW011-038) in an aspect of the invention.

The compound was prepared following General Procedure K using (2-fluoro-5-(trifluoromethyl)-phenyl)-boronic acid. ¹H NMR (400 MHz, DMSO-d₆) δ 10.60 (s, 1H), 8.41 (d, J=8.8 Hz, 1H), 8.20-8.15 (m, 1H), 7.94-7.77 (m, 3H), 7.57 (ddd, J=10.7, 8.7, 0.9 Hz, 1H), 1.50 (s, 9H). ¹⁹F NMR (376 MHz, DMSO-d₆) δ−60.35, −112.43. LCMS RT (Method 4)=2.723 min, m/z 398.1 [M−H]⁻.

### Example 125

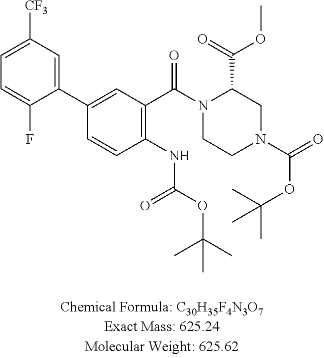 [SMILES: COC(=O)[C@@H]1CN(C(=O)OC(C)(C)C)CCN1C(=O)c1cc(-c2cc(C(F)(F)F)ccc2F)ccc1NC(=O)OC(C)(C)C]

This example is directed to the synthesis of 1-(tert-butyl) 3-methyl (S)-4-(4-((tert-butoxycarbonyl)amino)-2′-fluoro-5′-(trifluoromethyl)-[1,1′-biphenyl]-3-carbonyl)piperazine-1,3-dicarboxylate (KJW011-043) in an aspect of the invention.

The compound was prepared following General Procedure A using 4-((tert-butoxycarbonyl)amino)-2′-fluoro-5′-(trifluoromethyl)-[1,1′-biphenyl]-3-carboxylic acid (KJW011-038) and (S)-1-(tert-butyl) 3-methyl piperazine-1,3-dicarboxylate. ¹H NMR (400 MHz, Chloroform-d) δ 8.31 (d, J=8.7 Hz, 1H), 7.83 (s, 1H), 7.72-7.64 (m, 1H), 7.62-7.55 (m, 3H), 7.46 (s, 1H), 7.26 (d, J=3.1 Hz, 2H), 5.36 (s, 1H), 4.71 (d, J=13.9 Hz, 1H), 3.83 (s, 3H), 3.69-3.40 (m, 2H), 3.21 (d, J=13.9 Hz, 1H), 2.99-2.81 (m, 2H), 1.53 (s, 9H), 1.45 (s, 9H). ¹⁹F NMR (376 MHz, Chloroform-d) δ−61.98, −112.62. LCMS RT (Method 2)=3.599 min, m/z 625.8 [M⁺].

### Example 126

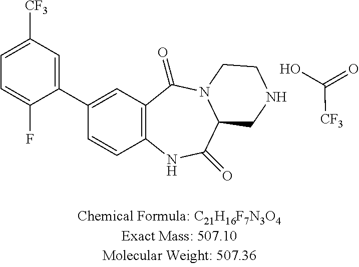

This example is directed to the synthesis of (S)-8-(2-fluoro-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-044) in an aspect of the invention.

The compound was prepared following General Procedure B using 1-(tert-butyl) 3-methyl (S)-4-(4-((tert-butoxycarbonyl)amino)-2′-fluoro-5′-(trifluoromethyl)-[1,1′-biphenyl]-3-carbonyl)piperazine-1,3-dicarboxylate (KJW011-043). ¹H NMR (400 MHz, DMSO-d₆) δ 10.99 (s, 1H), 9.41 (s, 1H), 8.66 (s, 1H), 7.98 (t, J=1.9 Hz, 1H), 7.94-7.80 (m, 3H), 7.65-7.57 (m, 1H), 7.29 (d, J=8.5 Hz, 1H), 4.57 (dd, J=5.2, 2.9 Hz, 1H), 4.21 (dt, J=14.4, 4.4 Hz, 1H), 3.81-3.61 (m, 2H), 3.56-3.20 (m, 3H). ¹⁹F NMR (376 MHz, DMSO-d₆) δ−60.39, −73.72, −112.23. LCMS RT (Method 1)=4.080 min, m/z 393.8 [M+H⁺].

### Example 127

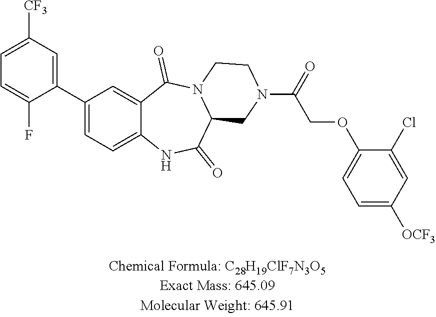 [SMILES: O=C1Nc2ccc(-c3cc(C(F)(F)F)ccc3F)cc2C(=O)N2CCN(C(=O)COc3ccc(OC(F)(F)F)cc3Cl)C[C@@H]12]

This example is directed to the synthesis of (S)-2-(2-(2-chloro-4-(trifluoromethoxy)phenoxy)acetyl)-8-(2-fluoro-5-(trifluoromethyl)phenyl)-1,3,4,12a-tetrahydrobenzo[e]pyrazino[1,2-a][1,4]diazepine-6,12 (2H, 11H)-dione (KJW011-046) in an aspect of the invention.

The compound was prepared following General Procedure C using (S)-8-(2-fluoro-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-044) and 2-(2-chloro-4-(trifluoromethoxy)-phenoxy)-acetic acid (KJW010-030). ¹H NMR (400 MHz, DMSO-d₆) δ 10.86 (s, 0.66×1H), 10.75 (s, 0.34×1H), 8.01 (t, J=1.9 Hz, 1H), 7.97-7.88 (m, 2H), 7.87-7.78 (m, 1H), 7.67-7.52 (m, 2H), 7.34-7.10 (m, 3H), 5.29-5.15 (m, 0.68×2H), 5.13-4.94 (m, 0.32×2H), 4.42 (t, J=4.5 Hz, 0.75×1H), 4.39-4.34 (m, 0.25×1H), 4.25-3.96 (m, 2H), 3.94-3.62 (m, 2H), 3.62-3.50 (m, 0.41×1H), 3.39 (ddd, J=12.6, 8.5, 4.4 Hz, 0.59×1H). ¹⁹F NMR (376 MHz, DMSO-d₆) δ−57.47, −57.49, −60.39, −112.20. LCMS RT (Method 1)=6.000 min, m/z 645.6 [M⁺].

### Example 128

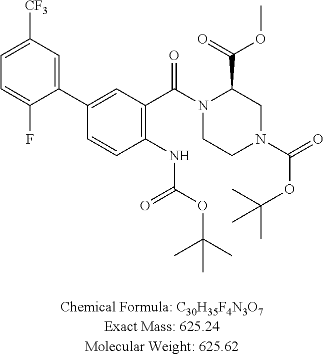 [SMILES: COC(=O)[C@H]1CN(C(=O)OC(C)(C)C)CCN1C(=O)c1cc(-c2cc(C(F)(F)F)ccc2F)ccc1NC(=O)OC(C)(C)C]

This example is directed to the synthesis of 1-(tert-butyl) 3-methyl (R)-4-(4-((tert-butoxycarbonyl)amino)-2′-fluoro-5′-(trifluoromethyl)-[1,1′-biphenyl]-3-carbonyl)piperazine-1,3-dicarboxylate (KJW011-050) in an aspect of the invention.

The compound was prepared following General Procedure A using 4-((tert-butoxycarbonyl)amino)-2′-fluoro-5′-(trifluoromethyl)-[1,1′-biphenyl]-3-carboxylic acid (KJW011-038) and (R)-1-(tert-butyl) 3-methyl piperazine-1,3-dicarboxylate. ¹H NMR (400 MHz, Chloroform-d) δ 8.31 (d, J=8.7 Hz, 1H), 7.83 (s, 1H), 7.72-7.64 (m, 1H), 7.62-7.55 (m, 2H), 7.46 (s, 1H), 7.26 (d, J=3.1 Hz, 1H), ¹H NMR (400 MHz, Chloroform-d) δ 8.31 (d, J=8.7 Hz, 1H), 7.83 (s, 1H), 7.72-7.64 (m, 1H), 7.62-7.55 (m, 3H), 7.46 (s, 1H), 7.26 (d, J=3.1 Hz, 2H), 5.36 (s, 1H), 4.71 (d, J=13.9 Hz, 1H), 3.83 (s, 3H), 3.69-3.40 (m, 2H), 3.21 (d, J=13.9 Hz, 1H), 2.99-2.81 (m, 2H), 1.53 (s, 9H), 1.45 (s, 9H). ¹⁹F NMR (376 MHz, Chloroform-d) δ−61.98, −112.62. LCMS RT (Method 2)=3.599 min, m/z 625.8 [M⁺].

### Example 129

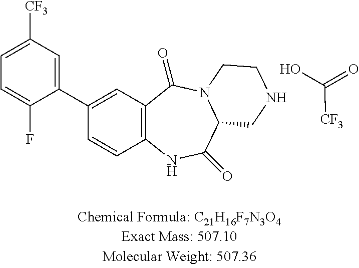

This example is directed to the synthesis of (R)-8-(2-fluoro-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-057) in an aspect of the invention.

The compound was prepared following General Procedure B using 1-(tert-butyl)-3-methyl (R)-4-(4-((tert-butoxycarbonyl)amino)-2′-fluoro-5′-(trifluoromethyl)-[1,1′-biphenyl]-3-carbonyl)piperazine-1,3-dicarboxylate (KJW011-050). ¹H NMR (400 MHz, DMSO-d₆) δ 10.99 (s, 1H), 9.41 (s, 1H), 8.66 (s, 1H), 7.98 (t, J=1.9 Hz, 1H), 7.94-7.80 (m, 3H), 7.65-7.57 (m, 1H), 7.29 (d, J=8.5 Hz, 1H), 4.57 (dd, J=5.2, 2.9 Hz, 1H), 4.21 (dt, J=14.4, 4.4 Hz, 1H), 3.81-3.61 (m, 2H), 3.56-3.20 (m, 3H). ¹⁹F NMR (376 MHz, DMSO-d₆) δ−60.39, −73.72, −112.23. LCMS RT (Method 1)=4.080 min, m/z 393.8 [M+H⁺].

### Example 130

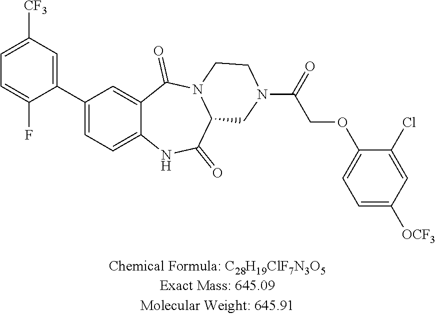 [SMILES: O=C1Nc2ccc(-c3cc(C(F)(F)F)ccc3F)cc2C(=O)N2CCN(C(=O)COc3ccc(OC(F)(F)F)cc3Cl)C[C@H]12]

This example is directed to the synthesis of (R)-2-(2-(2-chloro-4-(trifluoromethoxy)phenoxy)acetyl)-8-(2-fluoro-5-(trifluoromethyl)phenyl)-1,3,4,12a-tetrahydrobenzo[e]pyrazino[1,2-a][1,4]diazepine-6,12 (2H, 11H)-dione (KJW011-063) in an aspect of the invention.

The compound was prepared following General Procedure C using (R)-8-(2-fluoro-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-057) and 2-(2-chloro-4-(trifluoromethoxy)-phenoxy)-acetic acid (KJW010-030). ¹H NMR (400 MHz, DMSO-d₆) δ 10.86 (s, 0.66×1H), 10.75 (s, 0.34×1H), 8.01 (t, J=1.9 Hz, 1H), 7.97-7.88 (m, 2H), 7.87-7.78 (m, 1H), 7.67-7.52 (m, 2H), 7.34-7.10 (m, 3H), 5.29-5.15 (m, 0.68×2H), 5.13-4.94 (m, 0.32×2H), 4.42 (t, J=4.5 Hz, 0.75×1H), 4.39-4.34 (m, 0.25×1H), 4.25-3.96 (m, 2H), 3.94-3.62 (m, 2H), 3.62-3.50 (m, 0.41×1H), 3.39 (ddd, J=12.6, 8.5, 4.4 Hz, 0.59×1H). ¹⁹F NMR (376 MHz, DMSO-d₆) δ−57.47, −57.49, −60.39, −112.20. LCMS RT (Method 1)=6.000 min, m/z 645.6 [M⁺].

### Example 131

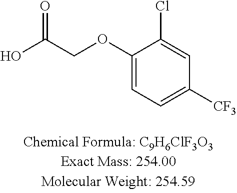 [SMILES: O=C(O)COc1ccc(C(F)(F)F)cc1Cl]

This example is directed to the synthesis of 2-(2-chloro-4-(trifluoromethyl)phenoxy)acetic acid (KJW011-036-2) in an aspect of the invention.

The compound was prepared following General Procedure E using 2-chloro-4-trifluoromethylphenol. ¹H NMR (400 MHz, DMSO-d₆) δ 13.33 (bs, 1H), 7.84 (dt, J=2.4, 0.8 Hz, 1H), 7.66 (ddq, J=8.7, 2.3, 0.8 Hz, 1H), 7.23 (dd, J=8.8, 0.9 Hz, 1H), 4.93 (s, 2H). ¹⁹F NMR (376 MHz, DMSO-d₆) δ−60.17. LCMS RT (Method 4)=1.932 min, 253.0 [M−H]⁻.

### Example 132

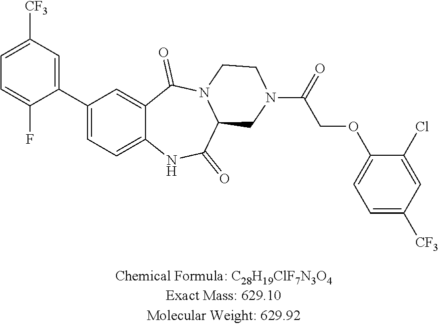 [SMILES: O=C1Nc2ccc(-c3cc(C(F)(F)F)ccc3F)cc2C(=O)N2CCN(C(=O)COc3ccc(C(F)(F)F)cc3Cl)C[C@@H]12]

This example is directed to the synthesis of (S)-2-(2-(2-chloro-4-(trifluoromethyl)phenoxy)acetyl)-8-(2-fluoro-5-(trifluoromethyl)phenyl)-1,3,4,12a-tetrahydrobenzo[e]pyrazino[1,2-a][1,4]diazepine-6,12 (2H, 11H)-dione (KJW011-047) in an aspect of the invention.

The compound was prepared following General Procedure C using (S)-8-(2-fluoro-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-044) and 2-(2-chloro-4-(trifluoromethyl)-phenoxy)-acetic acid (KJW011-036-2). ¹H NMR (400 MHz, DMSO-d₆) δ 10.86 (s, 0.67×1H), 10.75 (s, 0.33×H), 8.01 (t, J=1.9 Hz, 1H), 7.92 (td, J=6.5, 5.7, 2.4 Hz, 1H), 7.88-7.79 (m, 2H), 7.65-7.57 (m, 2H), 7.32-7.23 (m, 3H), 5.40-5.23 (m, 0.62×2H), 5.22-5.02 (m, 0.38×2H), 4.43 (t, J=4.4 Hz, 0.68×1H), 4.37 (t, J=4.9 Hz, 0.32×1H), 4.25-3.84 (m, 2H), 3.83-3.62 (m, 2H), 3.56 (ddd, J=13.4, 8.8, 4.4 Hz, 0.52×1H), 3.46-3.34 (m, 0.48×1H). ¹⁹F NMR (376 MHz, DMSO-d₆) δ−60.05, −60.06, −60.39, −112.20. LCMS RT (Method 1)=5.893 min, m/z 629.6 [M⁺].

### Example 133

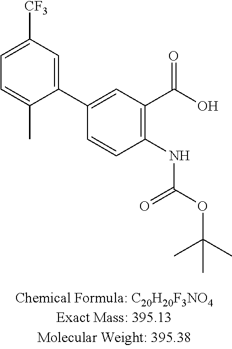 [SMILES: Cc1ccc(C(F)(F)F)cc1-c1ccc(NC(=O)OC(C)(C)C)c(C(=O)O)c1]

This example is directed to the synthesis of 4-((tert-butoxycarbonyl)amino)-2′-methyl-5′-(trifluoromethyl)-[1,1′-biphenyl]-3-carboxylic acid (KJW011-048) in an aspect of the invention.

The compound was prepared following General Procedure K using 2-methyl-5-trifluoromethylphenylboronic acid. 1H NMR (400 MHz, Chloroform-d) δ 10.07 (s, 1H), 8.56 (d, J=8.8 Hz, 1H), 8.07 (d, J=2.2 Hz, 1H), 7.57-7.45 (m, 3H), 7.44-7.35 (m, 1H), 2.32 (s, 3H), 1.56 (s, 9H). ¹⁹F NMR (376 MHz, Chloroform-d) δ−62.29. LCMS RT (Method 3)=2.886 min. m/z 295.9 [M-BOC+H⁺].

### Example 134

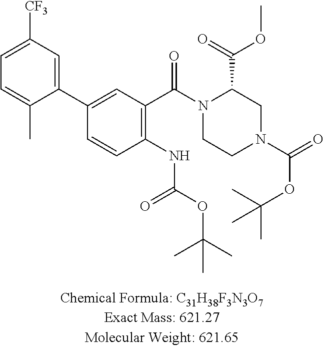 [SMILES: COC(=O)[C@@H]1CN(C(=O)OC(C)(C)C)CCN1C(=O)c1cc(-c2cc(C(F)(F)F)ccc2C)ccc1NC(=O)OC(C)(C)C]

This example is directed to the synthesis of 1-(tert-butyl)-3-methyl (S)-4-(4-((tert-butoxycarbonyl)amino)-2′-methyl-5′-(trifluoromethyl)-[1,1′-biphenyl]-3-carbonyl)piperazine-1,3-dicarboxylate (KJW011-048-3) in an aspect of the invention.

The compound was prepared following General Procedure A using 4-((tert-butoxycarbonyl)amino)-2′-methyl-5′-(trifluoromethyl)-[1,1′-biphenyl]-3-carboxylic acid (KJW011-048) and (S)-1-(tert-butyl) 3-methyl piperazine-1,3-dicarboxylate. ¹H NMR (400 MHz, Chloroform-d) δ 8.30-8.18 (m, 1H), 7.73 (s, 1H), 7.49 (dd, J=8.0, 2.0 Hz, 1H), 7.44 (s, 1H), 7.39-7.31 (m, 3H), 7.19 (s, 1H), 5.35 (s, 1H), 4.70 (d, J=13.9 Hz, 1H), 4.09-3.90 (m, 1H), 3.81 (s, 3H), 3.74-3.41 (m, 1H), 3.25-2.78 (m, 3H), 1.53 (s, 9H), 1.44 (s, 9H). ¹⁹F NMR (376 MHz, Chloroform-d) δ−62.29. LCMS RT (Method 3)=2.857 min, m/z 644.3 [M+Na⁺].

### Example 135

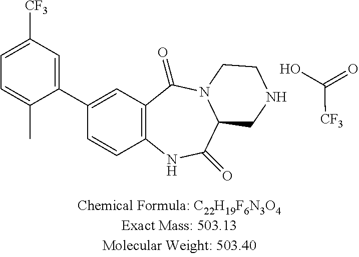

This example is directed to the synthesis of (S)-8-(2-methyl-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-052-1) in an aspect of the invention.

The compound was prepared following General Procedure B using 1-(tert-butyl)-3-methyl (S)-4-(4-((tert-butoxycarbonyl)amino)-2′-methyl-5′-(trifluoromethyl)-[1,1′-biphenyl]-3-carbonyl)piperazine-1,3-dicarboxylate (KJW011-048-3). ¹H NMR (400 MHz, DMSO-d₆) δ 10.94 (s, 1H), 9.36 (bs, 1H), 8.66 (bs, 1H), 7.72 (d, J=2.2 Hz, 1H), 7.66 (ddd, J=12.3, 8.3, 2.1 Hz, 2H), 7.58 (d, J=8.0 Hz, 1H), 7.49 (d, J=2.0 Hz, 1H), 7.25 (d, J=8.4 Hz, 1H), 4.58 (dd, J=5.2, 2.9 Hz, 1H), 4.21 (dt, J=14.5, 4.4 Hz, 1H), 3.78-3.63 (m, 1H), 3.64-3.02 (m, 4H), 2.33 (s, 3H). ¹⁹F NMR (376 MHz, DMSO-d₆) δ−60.77, −73.60. LCMS RT (Method 2)=3.204 min, m/z 390.1 [M+H⁺].

### Example 136

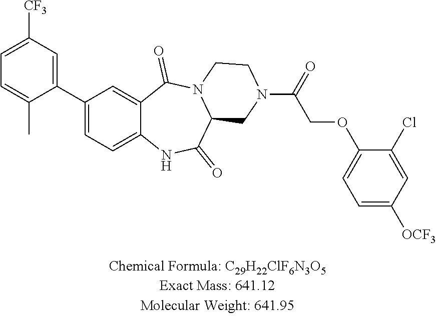 [SMILES: Cc1ccc(C(F)(F)F)cc1-c1ccc2c(c1)C(=O)N1CCN(C(=O)COc3ccc(OC(F)(F)F)cc3Cl)C[C@H]1C(=O)N2]

This example is directed to the synthesis of (S)-2-(2-(2-Chloro-4-(trifluoromethoxy)phenoxy)acetyl)-8-(2-methyl-5-(trifluoromethyl)phenyl)-1,3,4,12a-tetrahydrobenzo[e]pyrazino[1,2-a][1,4]diazepine-6,12 (2H, 11H)-dione (KJW011-053) in an aspect of the invention.

The compound was prepared following General Procedure C using (S)-8-(2-methyl-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-052-1) and 2-(2-chloro-4-(trifluoromethoxy)phenoxy)acetic acid (KJW010-093). ¹H NMR (400 MHz, Acetic Acid-d₄) δ 7.99 (t, J=2.7 Hz, 1H), 7.62 (ddd, J=9.3, 4.5, 2.1 Hz, 2H), 7.56 (t, J=2.8 Hz, 1H), 7.49 (d, J=8.0 Hz, 1H), 7.36 (dd, J=10.7, 2.7 Hz, 1H), 7.30 (dd, J=8.3, 2.2 Hz, 1H), 7.24-7.05 (m, 2H), 5.27-5.12 (m, 0.58×2H), 5.01 (q, J=15.1 Hz, 0.42×2H), 4.62 (dd, J=14.6, 4.2 Hz, 0.41×1H), 4.53 (dt, J=8.9, 4.4 Hz, 1H), 4.41 (dd, J=14.7, 4.0 Hz, 0.59×1H), 4.34-4.13 (m, 1H), 4.09-3.64 (m, 4H). ¹⁹F NMR (376 MHz, Acetic Acid-d₄) δ−59.35, −59.39, -63.07. LCMS RT (Method 1)=6.199 min, m/z 642.0 [M+H⁺].

### Example 137

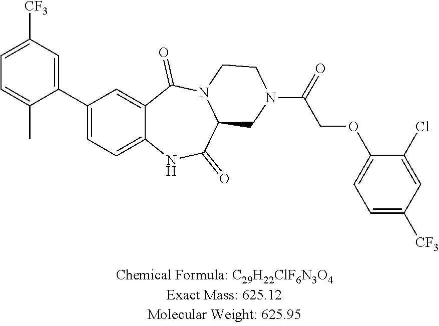 [SMILES: Cc1ccc(C(F)(F)F)cc1-c1ccc2c(c1)C(=O)N1CCN(C(=O)COc3ccc(C(F)(F)F)cc3Cl)C[C@H]1C(=O)N2]

This example is directed to the synthesis of (S)-2-(2-(2-chloro-4-(trifluoromethyl)phenoxy)acetyl)-8-(2-methyl-5-(trifluoromethyl)phenyl)-1,3,4,12a-tetrahydrobenzo[e]pyrazino[1,2-a][1,4]diazepine-6,12 (2H, 11H)-dione (KJW011-060) in an aspect of the invention.

The compound was prepared following General Procedure C using (S)-8-(2-methyl-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-036-2) and 2-(2-chloro-4-(trifluoromethyl)-phenoxy)-acetic acid (KJW010-093). ¹H NMR (400 MHz, Acetic Acid-d₄) δ 7.99 (dd, J=4.1, 2.0 Hz, 1H), 7.70 (dd, J=9.3, 2.2 Hz, 1H), 7.64-7.53 (m, 4H), 7.49 (d, J=8.0 Hz, 1H), 7.30 (dd, J=8.3, 4.4 Hz, 1H), 7.20 (dd, J=38.7, 8.7 Hz, 1H), 5.35-5.20 (m, 0.57×2H), 5.15-5.02 (m, 0.43×2H), 4.62 (dd, J=14.5, 4.2 Hz, 0.42×1H), 4.56-4.51 (m, 1H), 4.42 (dd, J=14.7, 3.9 Hz, 0.58×1H), 4.29-4.12 (m, 1H), 4.09-3.62 (m, 4H), 2.36 (s, 3H). ¹⁹F NMR (376 MHz, Acetic Acid-d₄) δ−62.63, −62.64, −63.07. LCMS RT (Method 1)=6.140 min, m/z 626.1 [M+H⁺].

### Example 138

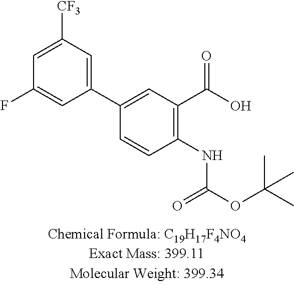 [SMILES: CC(C)(C)OC(=O)Nc1ccc(-c2cc(F)cc(C(F)(F)F)c2)cc1C(=O)O]

This example is directed to the synthesis of 4-((tert-butoxycarbonyl)amino)-3′-fluoro-5′-(trifluoromethyl)-[1,1′-biphenyl]-3-carboxylic acid (KJW011-048-2) in an aspect of the invention.

The compound was prepared following General Procedure K using (3-fluoro-5-(trifluoromethyl)phenyl)boronic acid). ¹H NMR (400 MHz, Chloroform-d) δ 10.09 (s, 1H), 8.62 (dd, J=8.9, 1.3 Hz, 1H), 8.34 (dd, J=2.4, 1.3 Hz, 1H), 7.79 (ddd, J=8.9, 2.4, 1.3 Hz, 1H), 7.62 (d, J=1.9 Hz, 1H), 7.47 (dt, J=9.5, 1.9 Hz, 1H), 7.34-7.24 (m, 1H), 1.57 (s, 9H). ¹⁹F NMR (376 MHz, Chloroform-d) δ−62.75, −110.26 (t, J=8.9 Hz). LCMS RT (Method 3)=2.859 min, m/z 299.8 [M-BOC+H⁺].

### Example 139

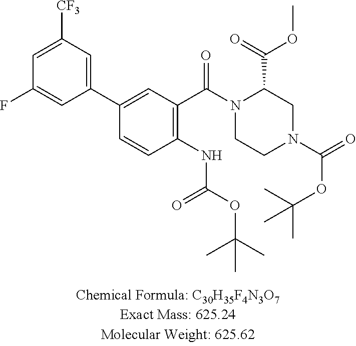 [SMILES: COC(=O)[C@@H]1CN(C(=O)OC(C)(C)C)CCN1C(=O)c1cc(-c2cc(F)cc(C(F)(F)F)c2)ccc1NC(=O)OC(C)(C)C]

This example is directed to the synthesis of 1-(tert-butyl) 3-methyl (S)-4-(4-((tert-butoxycarbonyl)amino)-3′-fluoro-5′-(trifluoromethyl)-[1,1′-biphenyl]-3-carbonyl)piperazine-1,3-dicarboxylate (KJW011-048-4) in an aspect of the invention.

The compound was prepared following General Procedure A using 4-((tert-butoxycarbonyl)amino)-3′-fluoro-5′-(trifluoromethyl)-[1,1′-biphenyl]-3-carboxylic acid (KJW011-048-2) and (S)-1-(tert-butyl) 3-methyl piperazine-1,3-dicarboxylate. ¹H NMR (400 MHz, Chloroform-d) δ 8.31 (dd, J=8.6, 4.7 Hz, 1H), 7.76 (s, 1H), 7.61 (dd, J=8.7, 2.3 Hz, 1H), 7.58 (s, 1H), 7.42 (dd, J=13.8, 4.7 Hz, 2H), 7.32-7.27 (m, 1H), 5.38 (bs, 1H), 4.73 (d, J=14.0 Hz, 1H), 3.84 (m, 4H), 3.50 (d, J=13.0 Hz, 1H), 3.29-2.73 (m, 2H), 1.53 (s, 9H), 1.45 (s, 9H). ¹⁹F NMR (376 MHz, Chloroform-d) δ−62.78, −107.92-−113.38 (m). LCMS RT (Method 4)=3.754 min, m/z 624.2 [M−H]⁻.

### Example 140

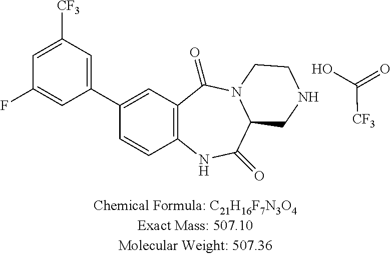

This example is directed to the synthesis of (S)-8-(3-fluoro-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-052-2) in an aspect of the invention.

The compound was prepared following General Procedure B using 1-(tert-butyl)-3-methyl (S)-4-(4-((tert-butoxycarbonyl)amino)-3′-fluoro-5′-(trifluoromethyl)-[1,1′-biphenyl]-3-carbonyl)piperazine-1,3-dicarboxylate (KJW011-048-4). ¹H NMR (400 MHz, DMSO-d₆) δ 10.98 (s, 1H), 9.48 (bs, 1H), 8.66 (bs, 1H), 8.13 (d, J=2.3 Hz, 1H), 8.04 (dd, J=8.5, 2.4 Hz, 1H), 7.94 (dt, J=10.1, 2.1 Hz, 1H), 7.89 (td, J=1.6, 0.8 Hz, 1H), 7.75-7.68 (m, 1H), 7.27 (d, J=8.5 Hz, 1H), 4.56 (dd, J=5.2, 2.8 Hz, 1H), 4.25 (t, J=4.4 Hz, 0.48×1H), 4.21 (t, J=4.4 Hz, 0.52×1H), 3.69 (dd, J=13.7, 2.9 Hz, 1H), 3.45 (ddd, J=14.2, 9.9, 3.8 Hz, 1H), 3.40-3.30 (m, 2H), 3.24 (ddd, J=13.2, 9.8, 4.0 Hz, 1H). LCMS RT (Method 3)=3.140 min, m/z 394.1 [M+H⁺].

### Example 141

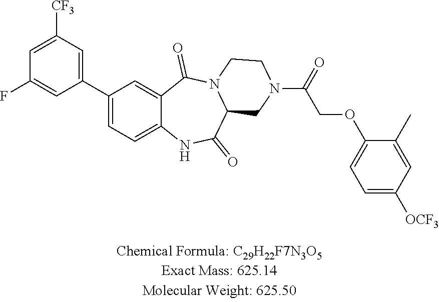 [SMILES: Cc1cc(OC(F)(F)F)ccc1OCC(=O)N1CCN2C(=O)c3cc(-c4cc(F)cc(C(F)(F)F)c4)ccc3NC(=O)[C@@H]2C1]

This example is directed to the synthesis of (S)-8-(3-fluoro-5-(trifluoromethyl)phenyl)-2-(2-(2-methyl-4-(trifluoromethoxy)phenoxy)acetyl)-1,3,4,12a-tetrahydrobenzo[e]pyrazino[1,2-a][1,4]diazepine-6,12 (2H, 11H)-dione (KJW011-055) in an aspect of the invention.

The compound was prepared following General Procedure C using (S)-8-(3-fluoro-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-052-2) and 2-(2-methyl-4-(trifluoromethoxy)-phenoxy)acetic acid (KJW011-028). ¹H NMR (400 MHz, Acetic Acid-d₄) δ 8.32 (t, J=2.6 Hz, 1H), 7.93 (dt, J=8.5, 2.2 Hz, 1H), 7.84 (d, J=3.7 Hz, 1H), 7.72 (d, J=9.7 Hz, 1H), 7.47 (d, J=8.4 Hz, 1H), 7.34 (d, J=8.4 Hz, 1H), 7.12-7.02 (m, 2H), 6.92 (dd, J=33.0, 9.1 Hz, 1H), 5.20-5.03 (m, 0.55×2H), 5.00-4.85 (m, 0.45×2H), 4.60 (dd, J=14.6, 4.5 Hz, 0.44×1H), 4.56-4.46 (m, 1H), 4.41 (dd, J=14.6, 3.8 Hz, 0.56×1H), 4.27-4.18 (m, 1H), 4.07-3.65 (m, 4H), 2.29 (s, 0.47×3H), 2.26 (s, 0.53×3H). ¹⁹F NMR (376 MHz, Acetic Acid-d₄) δ−59.06, −59.10, −63.52, −111.02 (q, J=8.5 Hz). LCMS RT (Method 1)=6.199 min, m/z 626.1 [M+H⁺].

### Example 142

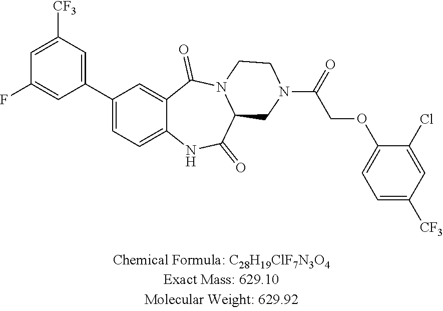 [SMILES: O=C1Nc2ccc(-c3cc(F)cc(C(F)(F)F)c3)cc2C(=O)N2CCN(C(=O)COc3ccc(C(F)(F)F)cc3Cl)C[C@@H]12]

This example is directed to the synthesis of (S)-2-(2-(2-chloro-4-(trifluoromethyl)phenoxy)acetyl)-8-(3-fluoro-5-(trifluoromethyl)phenyl)-1,3,4,12a-tetrahydrobenzo[e]pyrazino[1,2-a][1,4]diazepine-6,12 (2H, 11H)-dione (KJW011-059) in an aspect of the invention.

The compound was prepared following General Procedure C using (S)-8-(3-fluoro-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-052-2) and 2-(2-chloro-4-(trifluoromethyl)-phenoxy)-acetic acid (KJW010-093). ¹H NMR (400 MHz, Acetic Acid-d₄) δ 8.32 (t, J=2.4 Hz, 1H), 7.94 (dd, J=8.4, 2.2 Hz, 1H), 7.86-7.82 (m, 1H), 7.74-7.66 (m, 2H), 7.63-7.53 (m, 1H), 7.47 (d, J=8.4 Hz, 1H), 7.34 (dd, J=8.5, 4.1 Hz, 1H), 7.25-7.14 (m, 1H), 5.35-5.19 (m, 0.54×2H), 5.16-5.00 (m, 0.46×2H), 4.61 (dd, J=14.6, 4.2 Hz, 0.39×1H), 4.52-4.49 (m, 1H), 4.42 (dd, J=14.7, 3.9 Hz, 1H), 4.31-4.15 (m, 1H), 4.09-3.64 (m, 4H). ¹⁹F NMR (376 MHz, Acetic Acid-d₄) δ−62.63, −62.65, −63.52, −110.96-−111.04 m). LCMS RT (Method 1)=6.166 min, m/z 630.1 [M+H⁺].

### Example 143

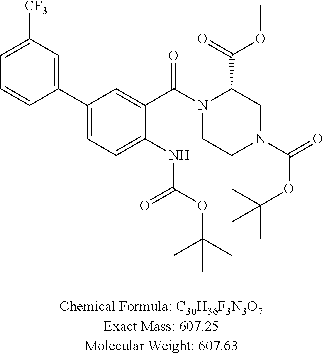 [SMILES: COC(=O)[C@@H]1CN(C(=O)OC(C)(C)C)CCN1C(=O)c1cc(-c2cccc(C(F)(F)F)c2)ccc1NC(=O)OC(C)(C)C]

This example is directed to the synthesis of 1-(tert-butyl) 3-methyl-(S)-4-(4-((tert-butoxycarbonyl)amino)-3′-(trifluoromethyl)-[1,1′-biphenyl]-3-carbonyl)piperazine-1,3-dicarboxylate (KJW011-022-2) in an aspect of the invention.

The compound was prepared following General Procedure A using 4-((tert-butoxycarbonyl)amino)-3′-(trifluoromethyl)-[1,1′-biphenyl]-3-carboxylic acid (KJW010-066) and 1-(tert-butyl) 3-methyl (S)-piperazine-1,3-dicarboxylate. ¹H NMR (400 MHz, Chloroform-d) δ 8.29 (d, J=8.7 Hz, 1H), 7.80-7.45 (m, 7H), 5.38 (s, 1H), 4.73 (d, J=13.9 Hz, 2H), 3.84 (s, 3H), 3.72 (s, 1H), 3.52 (dd, J=34.5, 10.1 Hz, 2H), 3.21 (d, J=14.1 Hz, 1H). ¹⁹F NMR (376 MHz, CDCl₃) δ−62.64. LCMS RT (Method 2)=3.852 min, m/z 607.8 [M⁺].

### Example 144

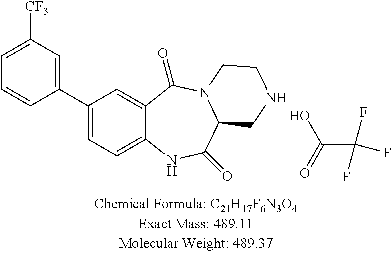

This example is directed to the synthesis of (S)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-030) in an aspect of the invention.

The compound was prepared following General Procedure B using 1-(tert-butyl) 3-methyl (S)-4-(4-((tert-butoxycarbonyl)amino)-3′-(trifluoromethyl)-[1,1′-biphenyl]-3-carbonyl)piperazine-1,3-dicarboxylate (KJW011-022-2). ¹H NMR (400 MHz, Acetic Acid-d₄) δ 8.27 (d, J=2.2 Hz, 1H), 7.98 (s, 1H), 7.97-7.91 (m, 2H), 7.70 (dt, J=15.3, 7.8 Hz, 2H), 7.33 (d, J=8.4 Hz, 1H), 4.74 (dd, J=5.0, 2.8 Hz, 1H), 4.44 (dt, J=14.8, 4.7 Hz, 1H), 4.09 (dd, J=14.1, 2.9 Hz, 1H), 3.89 (ddd, J=14.7, 8.8, 4.1 Hz, 1H), 3.79 (dd, J=14.1, 5.0 Hz, 1H), 3.75-3.62 (m, 2H). ¹⁹F NMR (376 MHz, Acetic acid-d₄) δ−63.43, −76.62. LCMS RT (Method 2)=2.777 min. m/z 376.1 [M+H⁺].

### Example 145

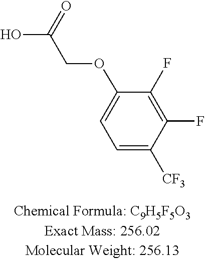 [SMILES: O=C(O)COc1ccc(C(F)(F)F)c(F)c1F]

This example is directed to the synthesis of 2-(2,3-difluoro-4-(trifluoromethyl)phenoxy)acetic acid (KJW011-064) in an aspect of the invention.

The compound was prepared following General Procedure E using 2,3-difluoro-4-trifluoromethylphenol. ¹H NMR (400 MHz, DMSO-d₆) δ 13.31 (bs, 1H), 7.53 (dddd, J=8.8, 7.8, 2.4, 0.9 Hz, 1H), 7.18-7.09 (m, 1H), 4.95 (s, 2H). ¹⁹F NMR (376 MHz, DMSO-d₆) δ-58.94, −58.97, −140.41-−140.71 (m), −157.80 (ddd, J=20.4, 7.5, 2.3 Hz). LCMS RT (Method 4)=0.925 min, m/z 255.0 [M−H]⁻.

### Example 146

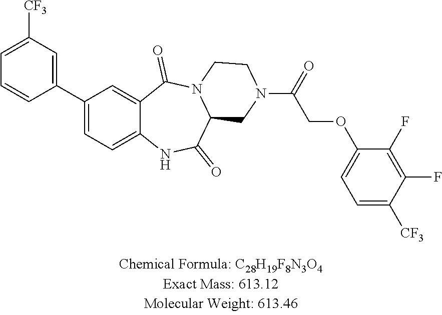 [SMILES: O=C1Nc2ccc(-c3cccc(C(F)(F)F)c3)cc2C(=O)N2CCN(C(=O)COc3ccc(C(F)(F)F)c(F)c3F)C[C@@H]12]

This example is directed to the synthesis of (S)-2-(2-(2,3-difluoro-4-(trifluoromethyl)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW011-066) in an aspect of the invention.

The compound was prepared following General Procedure C using 2-(2,3-difluoro-4-(trifluoromethyl)phenoxy)acetic acid (KJW011-064) and (S)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-030). ¹H NMR (400 MHz, Acetic Acid-d₄) δ 8.31 (dd, J=4.3, 2.1 Hz, 1H), 7.99 (d, J=3.3 Hz, 1H), 7.96-7.91 (m, 2H), 7.70 (dt, J=15.3, 7.8 Hz, 2H), 7.41 (q, J=7.9 Hz, 1H), 7.32 (dd, J=8.4, 3.5 Hz, 1H), 7.11-6.97 (m, 1H), 5.42-5.18 (m, 0.56×2H), 5.09 (q, J=15.4 Hz, 0.44×2H), 4.61 (dd, J=14.6, 4.2 Hz, 0.42×1H), 4.50 (t, J=4.1 Hz, 1H), 4.37 (dd, J=14.7, 3.4 Hz, 0.58×1H), 4.27-4.12 (m, 1H), 4.03-3.59 (m, 4H). ¹⁹F NMR (376 MHz, Acetic Acid-d₄) δ−61.19 (d, J=12.2 Hz), −63.41, −140.38 (dddd, J=25.7, 19.4, 12.6, 7.6 Hz), −158.01 (ddd, J=27.1, 18.4, 7.4 Hz). LCMS RT (Method 1)=5.695 min, m/z 614.2 [M+H⁺].

### Example 147

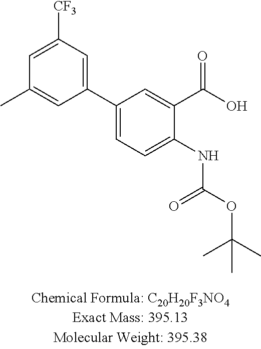 [SMILES: Cc1cc(-c2ccc(NC(=O)OC(C)(C)C)c(C(=O)O)c2)cc(C(F)(F)F)c1]

This example is directed to the synthesis of 4-((tert-butoxycarbonyl)amino)-3′-methyl-5′-(trifluoromethyl)-[1,1′-biphenyl]-3-carboxylic acid (KJW011-058) in an aspect of the invention.

The compound was prepared following General Procedure K using (3-methyl-5-(trifluoromethyl)phenyl)boronic acid. ¹H NMR (400 MHz, Chloroform-d) δ 10.06 (s, 1H), 8.59 (d, J=8.8 Hz, 1H), 8.33 (d, J=2.3 Hz, 1H), 7.80 (dd, J=8.8, 2.4 Hz, 1H), 7.68-7.46 (m, 1H), 7.42 (tt, J=1.6, 0.8 Hz, 1H), 2.49 (d, J=0.8 Hz, 3H). ¹⁹F NMR (376 MHz, DMSO-d₄) δ−61.21. LCMS RT (Method 4)=2.87 min. m/z 394.1 [M−H]⁻.

### Example 148

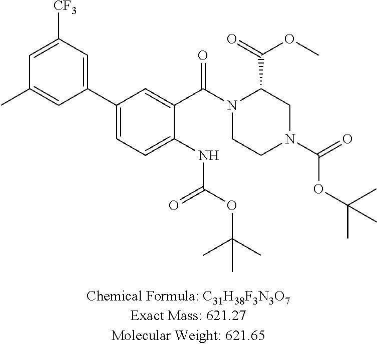 [SMILES: COC(=O)[C@@H]1CN(C(=O)OC(C)(C)C)CCN1C(=O)c1cc(-c2cc(C)cc(C(F)(F)F)c2)ccc1NC(=O)OC(C)(C)C]

This example is directed to the synthesis of 1-(tert-butyl)-3-methyl (S)-4-(4-((tert-butoxycarbonyl)amino)-3′-methyl-5′-(trifluoromethyl)-[1,1′-biphenyl]-3-carbonyl)piperazine-1,3-dicarboxylate (KJW011-065) in an aspect of the invention.

The compound was prepared following General Procedure A using 4-((tert-butoxycarbonyl)amino)-3′-methyl-5′-(trifluoromethyl)-[1,1′-biphenyl]-3-carboxylic acid (KJW011-058) and 1-(tert-butyl) 3-methyl (S)-piperazine-1,3-dicarboxylate. ¹H NMR (400 MHz, Chloroform-d) δ 8.27 (s, 1H), 7.71 (s, 1H), 7.62 (dd, J=8.7, 2.2 Hz, 1H), 7.57 (s, 1H), 7.52 (d, J=1.8 Hz, 1H), 7.45 (s, 1H), 7.40 (td, J=1.7, 0.9 Hz, 1H), 5.38 (s, 1H), 4.72 (d, J=13.9 Hz, 1H), 3.84 (s, 3H), 3.77-3.41 (m, 3H), 3.21 (d, J=14.3 Hz, 1H), 2.85 (bs, 1H), 2.47 (s, 3H), 1.53 (s, 9H), 1.45 (s, 9H). ¹⁹F NMR (376 MHz, Acetonitrile-d₃) δ−62.99. LCMS RT (Method 3)=3.809 min, m/z 622.3 [M+H⁺].

### Example 149

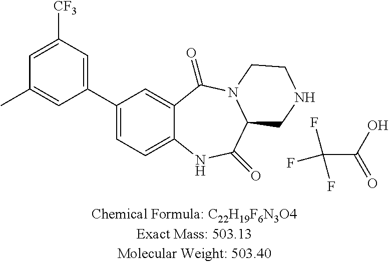

This example is directed to the synthesis of (S)-8-(3-methyl-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-087) in an aspect of the invention.

The compound was prepared following General Procedure B using 1-(tert-butyl) 3-methyl (S)-4-(4-((tert-butoxycarbonyl)amino)-3′-methyl-5′-(trifluoromethyl)-[1,1′-biphenyl]-3-carbonyl)piperazine-1,3-dicarboxylate (KJW011-065). ¹H NMR (400 MHz, Methanol-d₄) δ 8.12 (dd, J=2.3, 0.5 Hz, 1H), 7.91 (dd, J=8.4, 2.3 Hz, 1H), 7.80-7.71 (m, 2H), 7.53 (dq, J=1.6, 0.8 Hz, 1H), 7.29 (dd, J=8.5, 0.5 Hz, 1H), 4.69 (dd, J=5.0, 2.1 Hz, 1H), 4.38 (ddd, J=14.8, 5.4, 3.9 Hz, 1H), 3.96-3.84 (m, 1H), 3.69 (ddd, J=14.9, 9.7, 3.8 Hz, 1H), 3.61-3.47 (m, 2H), 3.41 (ddd, J=13.3, 9.7, 3.9 Hz, 1H), 2.52 (s, 3H). ¹⁹F NMR (376 MHz, Methanol-d₄) δ−64.11, −77.10. LCMS RT (Method 2)=2.846 min, m/z 390.1 [M+H⁺].

### Example 150

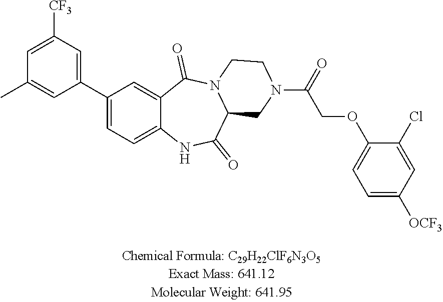 [SMILES: Cc1cc(-c2ccc3c(c2)C(=O)N2CCN(C(=O)COc4ccc(OC(F)(F)F)cc4Cl)C[C@H]2C(=O)N3)cc(C(F)(F)F)c1]

This example is directed to the synthesis of (S)-2-(2-(2-chloro-4-(trifluoromethoxy)phenoxy)acetyl)-8-(3-methyl-5-(trifluoromethyl)phenyl)-1,3,4,12a-tetrahydrobenzo[e]pyrazino[1,2-a][1,4]diazepine-6,12 (2H, 11H)-dione (KJW011-067) in an aspect of the invention.

The compound was prepared following General Procedure C using (S)-8-(3-Methyl-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-087) and 2-(2-chloro-4-(trifluoromethoxy)phenoxy)acetic acid (KJW010-093). ¹H NMR (400 MHz, Acetic Acid-d₄) δ 8.28 (d, J=2.2 Hz, 1H), 7.91 (dd, J=8.4, 2.2 Hz, 1H), 7.77 (d, J=6.0 Hz, 2H), 7.53 (s, 1H), 7.39-7.28 (m, 2H), 7.25-7.05 (m, 2H), 5.25-5.13 (m, 0.58×2H), 5.01 (q, J=15.1 Hz, 0.42×2H), 4.61 (dd, J=14.7, 4.3 Hz, 0.29×1H), 4.50 (t, J=4.4 Hz, 1H), 4.41 (dd, J=14.7, 4.1 Hz, 0.71×1H), 4.32-4.16 (m, 1H), 4.10-3.65 (m, 4H), 2.51 (s, 3H). ¹⁹F NMR (376 MHz, Acetic Acid-d₄) δ−59.35, −59.40, −63.29. LCMS RT (Method 1)=6.092 min, m/z 642.2 [M+H⁺].

### Example 151

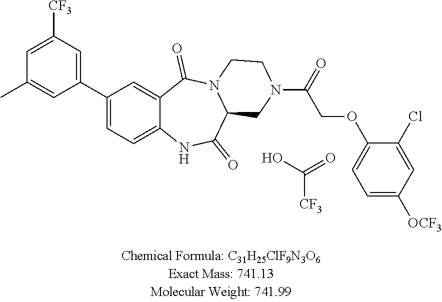

This example is directed to the synthesis of (R)-2-(2-(2-chloro-4-(trifluoromethoxy)phenoxy)acetyl)-8-(3-methyl-5-(trifluoromethyl)phenyl)-1,3,4,11,12,12a-hexahydrobenzo[e]pyrazino[1,2-a][1,4]diazepin-6 (2H)-one-2,2,2-trifluoroacetate (KJW011-074) in an aspect of the invention. See FIG. 10.

To (S)-2-(2-(2-chloro-4-(trifluoromethoxy)phenoxy)acetyl)-8-(3-methyl-5-(trifluoromethyl)phenyl)-1,3,4,12a-tetrahydrobenzo[e]pyrazino[1,2-a][1,4]diazepine-6,12 (2H, 11H)-dione (KJW011-067) (1 eq.) in THF was added borane-dimethylsulfide complex (2 eq., 2 M in THF), and the resultant solution was heated to 60° C. for 2.5 hours. The reaction was cooled and quenched with 3 mL of MeOH. The reaction was concentrated under vacuum and the residue was purified via reverse phase HPLC conditions using a gradient of 10-100% ACN in H₂O with 0.1% TFA to afford the product as a TFA salt. ¹H NMR (400 MHz, Acetic Acid-d₄) δ 8.24 (d, J=2.2 Hz, 1H), 7.91 (dd, J=8.4, 2.2 Hz, 1H), 7.76 (d, J=5.2 Hz, 2H), 7.53 (s, 1H), 7.39 (d, J=2.8 Hz, 1H), 7.31 (dd, J=8.5, 1.9 Hz, 1H), 7.29-7.24 (m, 1H), 7.19 (d, J=9.1 Hz, 1H), 4.91 (t, J=4.7 Hz, 1H), 4.69-4.58 (m, 3H), 4.35 (dd, J=13.7, 4.0 Hz, 1H), 4.13-3.82 (m, 5H), 2.51 (s, 3H). ¹⁹F NMR (376 MHz, Acetic Acid-d₄) δ−59.42, −63.31, −76.69. LCMS RT (Method 1)=5.611 min. m/z 628.2 [M+H⁺].

### Example 152

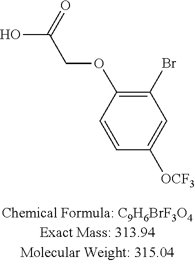 [SMILES: O=C(O)COc1ccc(OC(F)(F)F)cc1Br]

This example is directed to the synthesis of 2-(2-bromo-4-(trifluoromethoxy)phenoxy)acetic acid (KJW011-027) in an aspect of the invention.

The compound was prepared following General Procedure E using 2-bromo-4-trifluoromethoxypheniol. ¹H NMR (400 MHz, DMSO-d₆) δ 13.18 (bs, 1H), 7.69 (dd, J=2.9, 0.9 Hz, 1H), 7.37 (ddq, J=9.1, 2.9, 1.0 Hz, 1H), 7.10 (d, J=9.1 Hz, 1H), 4.85 (s, 2H). ¹⁹F NMR (376 MHz, DMSO-d₆) δ−57.45. LCMS RT (Method 4)=1.987 min, m/z 628.9 [2M−H]⁻

### Example 153

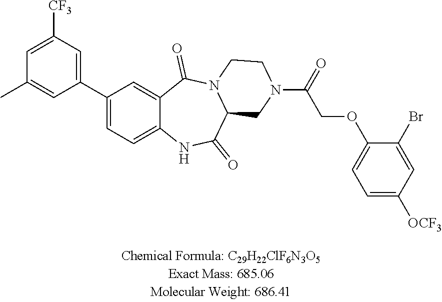 [SMILES: Cc1cc(-c2ccc3c(c2)C(=O)N2CCN(C(=O)COc4ccc(OC(F)(F)F)cc4Br)C[C@H]2C(=O)N3)cc(C(F)(F)F)c1]

This example is directed to the synthesis of (S)-2-(2-(2-bromo-4-(trifluoromethoxy)phenoxy)acetyl)-8-(3-methyl-5-(trifluoromethyl)phenyl)-1,3,4,12a-tetrahydrobenzo[e]pyrazino[1,2-a][1,4]diazepine-6,12 (2H, 11H)-dione (KJW011-078; Compound 4340) in an aspect of the invention.

The compound was prepared following General Procedure C using (S)-8-(3-methyl-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-087) and 2-(2-bromo-4-(trifluoromethoxy)phenoxy)acetic acid (KJW011-027). ¹H NMR (400 MHz, Acetic Acid-d₄) δ 8.28 (d, J=2.0 Hz, 1H), 7.91 (dd, J=8.5, 2.2 Hz, 1H), 7.77 (d, J=5.8 Hz, 2H), 7.56-7.45 (m, 2H), 7.34-7.22 (m, 2H), 7.15-7.04 (m, 1H), 5.18 (s, 0.56×2H), 5.0.1 (q, J=15.1 Hz, 0.44×2H), 4.61 (dd, J=14.7, 4.3 Hz, 0.41×1H), 4.49 (d, J=4.5 Hz, 1H), 4.41 (dd, J=14.6, 4.3 Hz, 0.59×1H), 4.33-4.14 (m, 1H), 4.11-3.67 (m, 4H), 2.51 (s, 3H). ¹⁹F NMR (376 MHz, Acetic Acid-d₄) δ−59.34, −59.40, −63.30. LCMS RT (Method 1)=6.087 min, m/z 685.5 [M⁺].

### Example 154

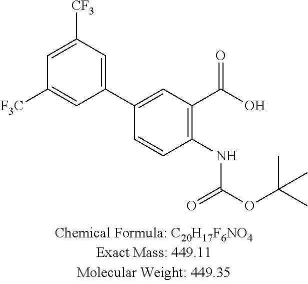 [SMILES: CC(C)(C)OC(=O)Nc1ccc(-c2cc(C(F)(F)F)cc(C(F)(F)F)c2)cc1C(=O)O]

This example is directed to the synthesis of 4-((tert-butoxycarbonyl)amino)-3′,5′-bis(trifluoromethyl)-[1,1′-biphenyl]-3-carboxylic acid (KJW011-072) in an aspect of the invention.

The compound was prepared following General Procedure K using (3,5-bis(trifluoromethyl)-phenyl)boronic acid. ¹H NMR (400 MHz, DMSO-d₆) δ 13.80 (s, 1H), 10.63 (s, 1H), 8.41 (d, J=8.9 Hz, 1H), 8.33-8.31 (m, 1H), 8.31-8.28 (m, 2H), 8.09-8.03 (m, 2H). ¹⁹F NMR (376 MHz, DMSO-d₆) δ−61.21. LCMS RT (Method 3)=2.911 min, m/z 349.8 [M-BOC+H⁺].

### Example 155

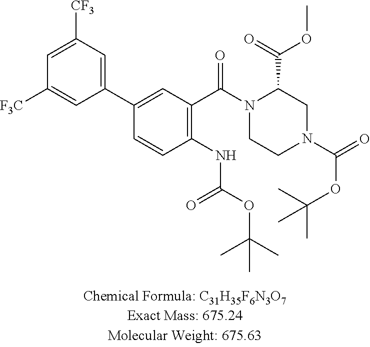 [SMILES: COC(=O)[C@@H]1CN(C(=O)OC(C)(C)C)CCN1C(=O)c1cc(-c2cc(C(F)(F)F)cc(C(F)(F)F)c2)ccc1NC(=O)OC(C)(C)C]

This example is directed to the synthesis of 1-(tert-butyl)-3-methyl (S)-4-(4-((tert-butoxycarbonyl)amino)-3′,5′-bis(trifluoromethyl)-[1,1′-biphenyl]-3-carbonyl)piperazine-1,3-dicarboxylate (KJW012-044) in an aspect of the invention.

The compound was prepared following General Procedure A using 4-((tert-butoxycarbonyl)amino)-3′,5′-bis(trifluoromethyl)-[1,1′-biphenyl]-3-carboxylic acid (KJW011-072) and 1-(tert-butyl)-3-methyl (S)-piperazine-1,3-dicarboxylate. ¹H NMR (400 MHz, Chloroform-d) δ 8.35 (d, J=8.1 Hz, 1H), 7.96 (s, 2H), 7.84-7.83 (m, 1H), 7.78 (s, 1H), 7.64 (dd, J=8.7, 2.3 Hz, 1H), 7.49-7.45 (m, 1H), 5.38 (bs, 1H), 4.73 (d, J=13.9 Hz, 1H), 4.01 (bs, 1H), 3.84 (s, 3H), 3.50 (s, 2H), 3.22 (d, J=13.7 Hz, 1H), 1.54 (s, 9H), 1.45 (s, 9H). LCMS RT (Method 3)=3.828 min, m/z 698.2 [M+Na⁺].

### Example 156

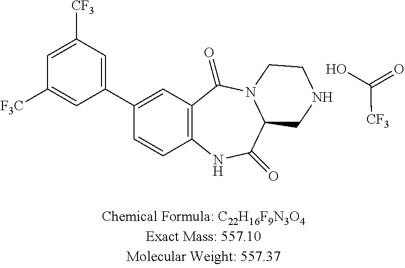

This example is directed to the synthesis of (S)-8-(3,5-bis(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-047) in an aspect of the invention.

The compound was prepared following General Procedure B using 1-(tert-butyl)-3-methyl (S)-4-(4-((tert-butoxycarbonyl)amino)-3′,5′-bis(trifluoromethyl)-[1,1′-biphenyl]-3-carbonyl)piperazine-1,3-dicarboxylate (KJW0112-044). ¹H NMR (400 MHz, DMSO-d₆) δ 10.99 (s, 1H), 9.39 (s, 1H), 8.35 (dt, J=1.4, 0.7 Hz, 2H), 8.21 (d, J=2.4 Hz, 1H), 8.15-8.07 (m, 2H), 7.29 (d, J=8.5 Hz, 1H), 4.55 (dd, J=5.2, 2.8 Hz, 1H), 4.23 (dt, J=14.4, 4.3 Hz, 1H), 3.69 (dd, J=13.7, 2.9 Hz, 1H), 3.46 (ddd, J=14.2, 9.9, 3.8 Hz, 1H), 3.36 (dd, J=13.4, 6.2 Hz, 2H), 3.29-3.18 (m, 1H). ¹⁹F NMR (376 MHz, DMSO-d₆) δ−61.19, −73.60. LCMS RT (Method 3)=3.021 min, m/z 444.0 [M+H⁺].

### Example 157

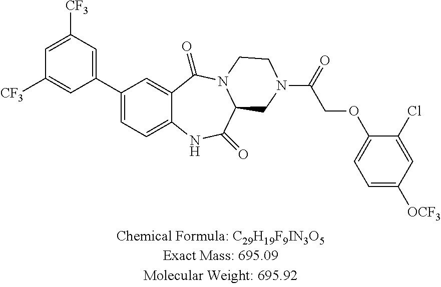 [SMILES: O=C1Nc2ccc(-c3cc(C(F)(F)F)cc(C(F)(F)F)c3)cc2C(=O)N2CCN(C(=O)COc3ccc(OC(F)(F)F)cc3Cl)C[C@@H]12]

This example is directed to the synthesis of (S)-8-(3,5-Bis(trifluoromethyl)phenyl)-2-(2-(2-chloro-4-(trifluoromethoxy)phenoxy)acetyl)-1,3,4,12a-tetrahydrobenzo[e]pyrazino[1,2-a][1,4]diazepine-6,12 (2H, 11H)-dione (KJW011-079)n in an aspect of the invention.

The compound was prepared following General Procedure C using (S)-8-(3,5-bis(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-047) and 2-(2-chloro-4-(trifluoromethoxy)-phenoxyacetic acid (KJW010-093). ¹H NMR (400 MHz, DMSO-d₆) δ 10.86 (s, 0.69×1H), 10.75 (s, 0.31×1H), 8.39-8.32 (m, 2H), 8.21 (dd, J=8.3, 2.4 Hz, 1H), 8.11 (td, J=1.7, 0.9 Hz, 1H), 8.08 (ddd, J=8.5, 4.1, 2.4 Hz, 1H), 7.60-7.52 (m, 1H), 7.32-7.24 (m, 2H), 7.17 (dd, J=16.7, 9.2 Hz, 1H), 5.28-5.14 (m, 0.69×2H), 5.13-4.95 (m, 0.31×2H), 4.40 (t, J=4.7 Hz, 0.71×1H), 4.35 (t, J=4.9 Hz, 0.29×1H), 4.25-3.84 (m, 2H), 3.81-3.64 (m, 2H), 3.55 (ddd, J=13.6, 9.0, 4.3 Hz, 1H), 3.40 (ddd, J=13.2, 8.9, 4.6 Hz, 1H). ¹⁹F NMR (376 MHz, DMSO-d₆) δ−57.48, −57.50, −61.20, −61.21. LCMS RT (Method 1)=6.203 min, m/z 696.2 [M+H⁺].

### Example 158

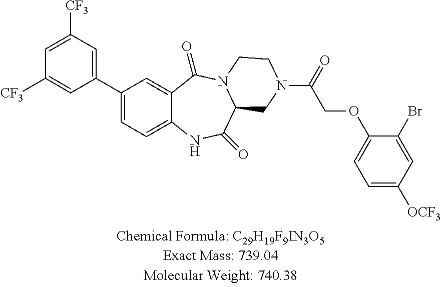 [SMILES: O=C1Nc2ccc(-c3cc(C(F)(F)F)cc(C(F)(F)F)c3)cc2C(=O)N2CCN(C(=O)COc3ccc(OC(F)(F)F)cc3Br)C[C@@H]12]

This example is directed to the synthesis of (S)-8-(3,5-Bis(trifluoromethyl)phenyl)-2-(2-(2-bromo-4-(trifluoromethoxy)phenoxy)acetyl)-1,3,4,12a-tetrahydrobenzo[e]pyrazino[1,2-a][1,4]diazepine-6,12 (2H, 11H)-dione (KJW011-080; Compound 4337) in an aspect of the invention.

The compound was prepared following General Procedure C using (S)-8-(3,5-bis(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-047) and 2-(2-bromo-4-(trifluoromethoxy)phenoxy)acetic acid (KJW011-027). ¹H NMR (400 MHz, DMSO-d₆) δ 10.86 (s, 0.70×1H), 10.75 (s, 0.30×1H), 8.38-8.33 (m, 2H), 8.21 (dd, J=7.4, 2.4 Hz, 1H), 8.11 (tt, J=1.6, 0.8 Hz, 1H), 8.08 (ddd, J=8.5, 4.3, 2.4 Hz, 1H), 7.71-7.61 (m, 1H), 7.36-7.24 (m, 2H), 7.13 (dd, J=15.9, 9.2 Hz, 1H), 5.29-5.13 (m, 0.70×2H), 5.12-4.92 (m, 0.30×2H), 4.40 (t, J=4.7 Hz, 0.70×1H), 4.35 (t, J=4.9 Hz, 0.30×1H), 4.25-3.84 (m, 2H), 3.81-3.63 (m, 2H), 3.55 (ddd, J=13.5, 9.0, 4.3 Hz, 1H), 3.40 (ddd, J=12.9, 8.9, 4.4 Hz, 1H). ¹⁹F NMR (376 MHz, DMSO) δ−57.46, −57.50, −61.20, −61.21. LCMS RT (Method 1)=6.427 min, m/z 740.0 [M⁺].

### Example 159

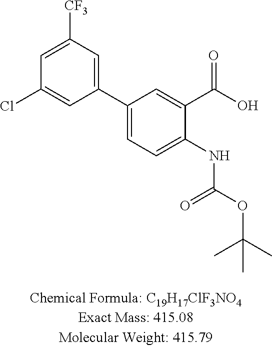 [SMILES: CC(C)(C)OC(=O)Nc1ccc(-c2cc(Cl)cc(C(F)(F)F)c2)cc1C(=O)O]

This example is directed to the synthesis of 4-((tert-butoxycarbonyl)amino)-3′-chloro-5′-(trifluoromethyl)-[1,1′-biphenyl]-3-carboxylic acid (KJW011-089) in an aspect of the invention.

The compound was prepared following General Procedure K using 3-chloro-5-trifluoromethylboronic acid. ¹H NMR (400 MHz, DMSO-d₆) δ 13.85 (bs, 1H), 10.58 (s, 1H), 8.40 (d, J=8.9 Hz, 1H), 8.28-8.25 (m, 1H), 8.07-8.04 (m, 1H), 8.02 (dd, J=8.8, 2.4 Hz, 1H), 7.94 (tt, J=1.7, 0.8 Hz, 1H), 7.82 (tt, J=1.6, 0.7 Hz, 1H), 1.50 (s, 9H). ¹⁹F NMR (376 MHz, DMSO-d₆) δ−61.18. LCMS RT (Method 2)=3.89 min, m/z 316.0 [M-BOC+H⁺].

### Example 160

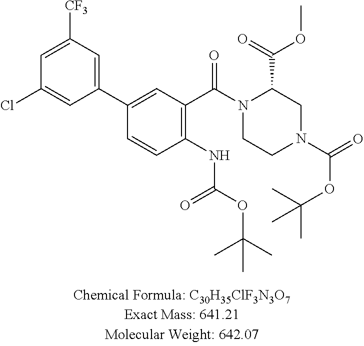 [SMILES: COC(=O)[C@@H]1CN(C(=O)OC(C)(C)C)CCN1C(=O)c1cc(-c2cc(Cl)cc(C(F)(F)F)c2)ccc1NC(=O)OC(C)(C)C]

This example is directed to the synthesis of 1-(tert-butyl)-3-methyl (S)-4-(4-((tert-butoxycarbonyl)amino)-3′-chloro-5′-(trifluoromethyl)-[1,1′-biphenyl]-3-carbonyl)piperazine-1,3-dicarboxylate (KJW011-090) in an aspect of the invention.

The compound was prepared following General Procedure A using 4-((tert-butoxycarbonyl)amino)-3′-chloro-5′-(trifluoromethyl)-[1,1′-biphenyl]-3-carboxylic acid (KJW011-089) and 1-(tert-butyl) 3-methyl (S)-piperazine-1,3-dicarboxylate. ¹H NMR (400 MHz, Chloroform-d) δ 8.33-8.31 (m, 1H), 7.75 (s, 1H), 7.69 (bs, 1H), 7.65 (bs, 1H), 7.63-7.59 (m, 1H), 7.57 (d, J=1.8 Hz, 1H), 7.44 (bs, 1H), 5.37 (s, 1H), 4.73 (d, J=13.9 Hz, 1H), 4.00 (d, J=17.0 Hz, 1H), 3.84 (s, 3H), 3.51 (s, 1H), 3.22 (d, J=13.8 Hz, 1H), 2.83 (bs, 1H), 1.53 (s, 9H), 1.45 (s, 9H). ¹⁹F NMR (376 MHz, Chloroform-d) δ−62.83. LCMS RT (Method 4)=3.915 min, m/z 640.2 [M−H]⁻.

### Example 161

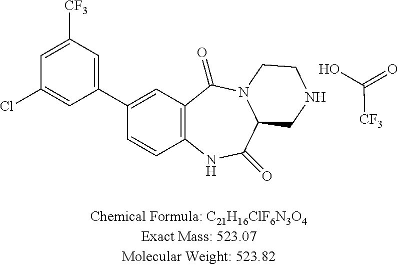

This example is directed to the synthesis of (S)-8-(3-chloro-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-091) in an aspect of the invention.

The compound was prepared following General Procedure B using 1-(tert-butyl) 3-methyl (S)-4-(4-((tert-butoxycarbonyl)amino)-3′-chloro-5′-(trifluoromethyl)-[1,1′-biphenyl]-3-carbonyl)piperazine-1,3-dicarboxylate (KJW011-090). ¹H NMR (400 MHz, DMSO-d₆) δ 10.98 (s, 1H), 8.13 (d, J=2.4 Hz, 1H), 8.13-8.11 (m, 1H), 8.04 (dd, J=8.5, 2.4 Hz, 1H), 7.99 (tt, J=1.7, 0.8 Hz, 1H), 7.89 (tt, J=1.6, 0.7 Hz, 1H), 7.27 (d, J=8.5 Hz, 1H), 4.55 (dd, J=5.2, 2.7 Hz, 1H), 4.22 (dt, J=14.5, 4.4 Hz, 1H), 3.69 (dd, J=13.6, 2.9 Hz, 1H), 3.45 (ddd, J=14.2, 9.9, 3.9 Hz, 1H), 3.35 (dt, J=12.9, 4.7 Hz, 1H), 3.29-3.17 (m, 1H). ¹⁹F NMR (376 MHz, DMSO-d₆) δ−61.18, −73.67. LCMS RT (Method 1)=4.240 min. m/z 410.1 [M+H⁺].

### Example 162

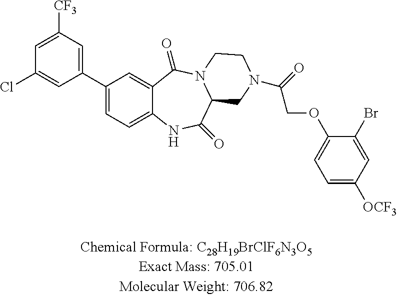 [SMILES: O=C1Nc2ccc(-c3cc(Cl)cc(C(F)(F)F)c3)cc2C(=O)N2CCN(C(=O)COc3ccc(OC(F)(F)F)cc3Br)C[C@@H]12]

This example is directed to the synthesis of (S)-2-(2-(2-Bromo-4-(trifluoromethoxy)phenoxy)acetyl)-8-(3-chloro-5-(trifluoromethyl)phenyl)-1,3,4,12a-tetrahydrobenzo[e]pyrazino[1,2-a][1,4]diazepine-6,12 (2H, 11H)-dione (KJW011-092) in an aspect of the invention.

The compound was prepared following General Procedure C using (S)-8-(3-chloro-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-091) and 2-(2-bromo-4-(trifluoromethoxy)phenoxy)acetic acid (KJW011-027). ¹H NMR (400 MHz, DMSO-d₆) δ 10.84 (s, 0.69×1H), 10.73 (s, 0.31×1H), 8.15 (d, J=2.4 Hz, 1H), 8.14-8.09 (m, 1H), 8.04-7.97 (m, 2H), 7.87 (tt, J=1.5, 0.7 Hz, 1H), 7.70-7.63 (m, 1H), 7.37-7.29 (m, 1H), 7.26 (d, J=8.5 Hz, 1H), 7.13 (dd, J=17.1, 9.2 Hz, 1H), 5.26-5.14 (m, 0.69×2H), 5.11-4.94 (m, 0.31×2H), 4.40 (t, J=4.7 Hz, 0.70×1H), 4.35 (t, J=4.9 Hz, 0.30×1H), 4.24-3.84 (m, 2H), 3.79-3.66 (m, 2H), 3.55 (ddd, J=13.4, 9.0, 4.2 Hz, 1H), 3.40 (ddd, J=13.3, 9.0, 4.6 Hz, 1H). ¹⁹F NMR (376 MHz, DMSO-d₆) δ−57.45, −57.47, −61.19, −61.20. LCMS RT (Method 1)=6.237 min, m/z 706.0 [M+H⁺].

### Example 163

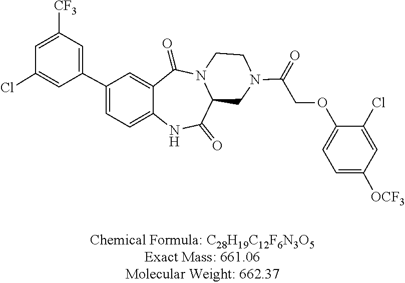 [SMILES: O=C1Nc2ccc(-c3cc(Cl)cc(C(F)(F)F)c3)cc2C(=O)N2CCN(C(=O)COc3ccc(OC(F)(F)F)cc3Cl)C[C@@H]12]

This example is directed to the synthesis of (S)-2-(2-(2-chloro-4-(trifluoromethoxy)phenoxy)acetyl)-8-(3-chloro-5-(trifluoromethyl)phenyl)-1,3,4,12a-tetrahydrobenzo[e]pyrazino[1,2-a][1,4]diazepine-6,12 (2H, 11H)-dione (KJW011-093) in an aspect of the invention.

The compound was prepared following General Procedure C using (S)-8-(3-chloro-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-091) and 2-(2-chloro-4-(trifluoromethoxy)phenoxy)acetic acid (KJW010-093). ¹H NMR (400 MHz, DMSO-d₆) δ 10.84 (s, 0.68×1H), 10.73 (s, 0.32×1H), 8.17-8.09 (m, 2H), 8.04-7.97 (m, 2H), 7.87 (tt, J=1.6, 0.8 Hz, 1H), 7.58-7.53 (m, 1H), 7.33-7.24 (m, 2H), 7.17 (dd, J=17.8, 9.2 Hz, 1H), 5.21 (q, J=15.3 Hz, 0.71×2H), 5.12-4.95 (m, 0.29×2H), 4.40 (t, J=4.6 Hz, 0.68×1H), 4.35 (t, J=4.8 Hz, 0.32×1H), 4.25-3.85 (m, 2H), 3.79-3.64 (m, 2H), 3.55 (ddd, J=13.5, 8.9, 4.2 Hz, 1H), 3.39 (ddd, J=13.1, 8.9, 4.5 Hz, 1H). ¹⁹F NMR (376 MHz, dmso) δ−57.45, −57.47, −61.18, −61.19. LCMS RT (Method 1)=6.175 min, m/z 662.1 [M⁺].

### Example 164

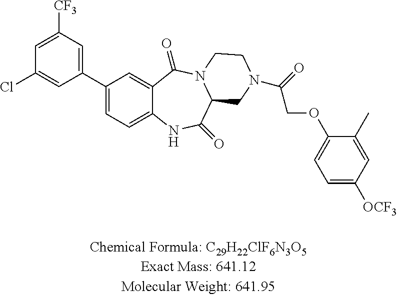 [SMILES: Cc1cc(OC(F)(F)F)ccc1OCC(=O)N1CCN2C(=O)c3cc(-c4cc(Cl)cc(C(F)(F)F)c4)ccc3NC(=O)[C@@H]2C1]

This example is directed to the synthesis of (S)-8-(3-chloro-5-(trifluoromethyl)phenyl)-2-(2-(2-methyl-4-(trifluoromethoxy)phenoxy)acetyl)-1,3,4,12a-tetrahydrobenzo[e]pyrazino[1,2-a][1,4]diazepine-6,12 (2H, 11H)-dione (KJW011-094) in an aspect of the invention.

The compound was prepared following General Procedure C using (S)-8-(3-chloro-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-091) and 2-(2-methyl-4-(trifluoromethoxy)-phenoxy)acetic acid (KJW011-028). ¹H NMR (400 MHz, DMSO-d₆) δ 10.82 (s, 0.70×1H), 10.73 (s, 0.30×1H), 8.17-8.09 (m, 2H), 8.04-7.97 (m, 2H), 7.87 (td, J=1.7, 0.7 Hz, 1H), 7.26 (d, J=8.5 Hz, 1H), 7.19-7.15 (m, 1H), 7.11-7.06 (m, 1H), 6.97 (dd, J=17.6, 9.0 Hz, 1H), 5.16-5.00 (m, 0.67×2H), 4.98-4.80 (m, 0.33×2H), 4.26-3.83 (m, 2H), 3.82-3.63 (m, 2H), 3.56 (ddd, J=13.4, 8.8, 4.2 Hz, 1H), 3.40 (ddd, J=11.8, 8.1, 3.8 Hz, 1H), 2.23 (s, 0.31×3H), 2.21 (s, 0.69×3H). ¹⁹F NMR (376 MHz, DMSO-d₆) δ−57.13, −57.15, −61.20, −61.21. LCMS RT (Method 1)=6.152 min, m/z 642.2 [M+H⁺].

### Example 165

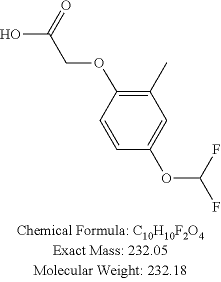 [SMILES: Cc1cc(OC(F)F)ccc1OCC(=O)O]

This example is directed to the synthesis of 2-(4-(difluoromethoxy)-2-methylphenoxy)acetic acid (KJW011-096-2) in an aspect of the invention.

The compound was prepared following General Procedure E using 4-(difluoromethoxy)-2-methylphenol. ¹H NMR (400 MHz, DMSO-d₆) δ 13.01 (s, 1H), 7.07 (t, J=76 Hz, 1H), 7.02 (dd, J=3.0, 0.8 Hz, 1H), 6.94 (ddq, J=8.8, 3.0, 0.6 Hz, 1H), 6.84 (d, J=8.9 Hz, 1H), 4.70 (s, 2H), 2.19 (s, 3H). ¹⁹F NMR (376 MHz, DMSO-d₆) δ−81.03, −81.23. LCMS RT (Method 3)=2.121 min, m/z 249.9 [M+H₂O]⁺.

### Example 166

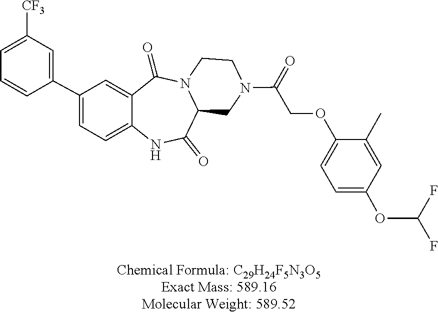 [SMILES: Cc1cc(OC(F)F)ccc1OCC(=O)N1CCN2C(=O)c3cc(-c4cccc(C(F)(F)F)c4)ccc3NC(=O)[C@@H]2C1]

This example is directed to the synthesis of (S)-2-(2-(4-(difluoromethoxy)-2-methylphenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW011-099) in an aspect of the invention.

The compound was prepared following General Procedure C using (S)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW010-088) and 2-(4-(difluoromethoxy)-2-methylphenoxy)acetic acid (KJW011-096-2). ¹H NMR (400 MHz, Acetic Acid-d₄) δ 8.30 (d, J=2.1 Hz, 1H), 7.99 (d, J=4.0 Hz, 1H), 7.97-7.90 (m, 2H), 7.69 (dt, J=15.3, 7.8 Hz, 2H), 7.32 (d, J=8.5 Hz, 1H), 7.02-6.81 (m, 3H), 6.58 (td, J=74.6, 7.6 Hz, 1H), 5.14-5.02 (m, 0.55×2H), 4.96-4.82 (m, 0.45×2H), 4.59 (dd, J=14.5, 4.4 Hz, 0.50×1H), 4.51-4.44 (m, 1H), 4.42 (dd, J=14.5, 3.8 Hz, 0.50×1H), 4.21-4.17 (m, 1H), 4.07-3.86 (m, 3H), 3.75-3.68 (m, 1H). 2.27 (s, 0.46×3H), 2.24 (s, 0.54×3H). ¹⁹F NMR (376 MHz, Acetic Acid-d₄) δ−63.39, −81.41−−81.86 (m). LCMS RT (Method 1)=5.349 min, m/z 589.7 [M+H⁺].

### Example 167

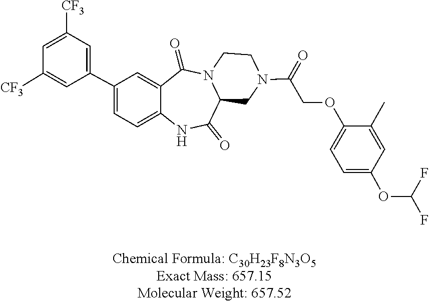 [SMILES: Cc1cc(OC(F)F)ccc1OCC(=O)N1CCN2C(=O)c3cc(-c4cc(C(F)(F)F)cc(C(F)(F)F)c4)ccc3NC(=O)[C@@H]2C1]

This example is directed to the synthesis of (S)-2-(2-(4-(difluoromethoxy)-2-methylphenoxy)acetyl)-8-(3-(trifluoromethyl)-5-(trifluoromethyl)phenyl)-1,3,4,12a-tetrahydrobenzo[e]pyrazino[1,2-a][1,4]diazepine-6,12 (2H, 11H)-dione (KJW011-100) in an aspect of the invention.

The compound was prepared following General Procedure C using (S)-8-(3,5-bis(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-047) and 2-(4-(difluoromethoxy)-2-methylphenoxy)acetic acid (KJW011-096-2). ¹H NMR (400 MHz, DMSO-d₆) δ 10.78 (s, 0.69×1H), 10.71 (s, 0.31×1H), 8.11-8.08 (m, 1H), 8.04-7.97 (m, 2H), 7.96 (t, J=2.1 Hz, 1H), 7.78-7.70 (m, 2H), 7.26 (d, J=8.6 Hz, 1H), 7.05 (t, J=74.8 Hz, 1H), 7.00 (d, J=3.1 Hz, 1H), 6.98-6.89 (m, 1H), 5.09-4.97 (m, 0.68×2H), 4.91-4.77 (m, 0.32×2H), 4.40-4.36 (m, 1H), 4.24-3.83 (m, 2H), 3.79-3.65 (m, 2H), 3.57 (ddd, J=13.4, 8.6, 4.2 Hz, 1H), 3.39 (ddd, J=12.9, 8.6, 4.3 Hz, 1H), 2.21 (s, 0.31×3H), 2.20-2.18 (m, 0.69×3H). ¹⁹F NMR (376 MHz, DMSO-d₆)) 6-61.05, −61.06, −80.96-−81.21 (m). LCMS RT (Method 2)=3.454 min, m/z 657.7 [M+H⁺].

### Example 168

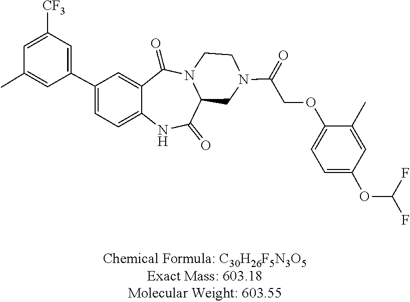 [SMILES: Cc1cc(-c2ccc3c(c2)C(=O)N2CCN(C(=O)COc4ccc(OC(F)F)cc4C)C[C@H]2C(=O)N3)cc(C(F)(F)F)c1]

This example is directed to the synthesis of (S)-2-(2-(4-(Difluoromethoxy)-2-methylphenoxy)acetyl)-8-(3-methyl-5-(trifluoromethyl)phenyl)-1,3,4,12a-tetrahydrobenzo[e]pyrazino[1,2-a][1,4]diazepine-6,12 (2H, 11H)-dione (KJW012-001) in an aspect of the invention.

The compound was prepared following General Procedure C using (S)-8-(3-methyl-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW011-087) and 2-(4-(difluoromethoxy)-2-methylphenoxy)acetic acid (KJW011-096-2). ¹H NMR (400 MHz, DMSO-d₆) δ 10.77 (s, 0.69×1H), 10.69 (s, 0.31×1H), 8.08 (t, J=2.8 Hz, 1H), 7.95 (dt, J=8.4, 2.3 Hz, 1H), 7.84 (d, J=6.4 Hz, 1H), 7.77 (d, J=6.8 Hz, 1H), 7.57 (s, 1H), 7.27-7.23 (m, 1H), 7.05 (t, J=72.0 Hz, 1H), 7.00 (d, J=3.1 Hz, 1H), 6.97-6.88 (m, 2H), 5.12-4.94 (m, 0.68×2H), 4.94-4.76 (m, 0.32×2H), 4.39-4.34 (m, 1H), 4.24-3.82 (m, 2H), 3.81-3.64 (m, 2H), 3.56 (ddd, J=13.3, 8.6, 4.1 Hz, 1H), 3.45-3.36 (m, 1H), 2.48 (s, 3H), 2.21 (s, 0.31×3H), 2.19 (d, 0.69×3H). ¹⁹F NMR (376 MHz, DMSO-d₆) δ−60.98, −60.99, −80.96, −81.01, −81.16, −81.21. LCMS RT (Method 1)=5.590 min, m/z 603.7 [M+H⁺].

### Example 169

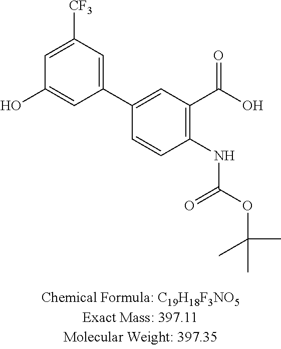 [SMILES: CC(C)(C)OC(=O)Nc1ccc(-c2cc(O)cc(C(F)(F)F)c2)cc1C(=O)O]

This example is directed to the synthesis of 4-((tert-butoxycarbonyl)amino)-3′-hydroxy-5′-(trifluoromethyl)-[1,1′-biphenyl]-3-carboxylic acid (KJW011-097) in an aspect of the invention.

The compound was prepared following General Procedure K using 3-hydroxy-5-trifluoromethyl-phenylboronic acid. LCMS RT (Method 4)=2.612 min, m/z 396.1 [M−H]⁻.

### Example 170

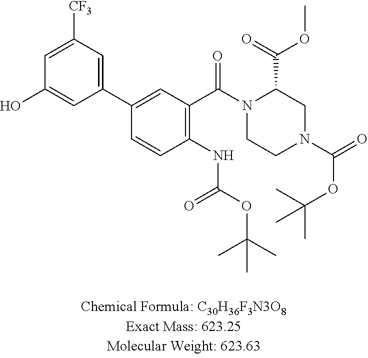 [SMILES: COC(=O)[C@@H]1CN(C(=O)OC(C)(C)C)CCN1C(=O)c1cc(-c2cc(O)cc(C(F)(F)F)c2)ccc1NC(=O)OC(C)(C)C]

This example is directed to the synthesis of 1-(tert-butyl) 3-methyl (S)-4-(4-((tert-butoxycarbonyl)amino)-3′-hydroxy-5′-(trifluoromethyl)-[1,1′-biphenyl]-3-carbonyl)piperazine-1,3-dicarboxylate (KJW011-098) in an aspect of the invention.

The compound was prepared following General Procedure A using 4-((tert-butoxycarbonyl)amino)-3′-hydroxy-5′-(trifluoromethyl)-[1,1′-biphenyl]-3-carboxylic acid (KJW011-097) and 1-(tert-butyl) 3-methyl (S)-piperazine-1,3-dicarboxylate. ¹H NMR (400 MHz, Chloroform-d) δ 8.16 (s, 1H), 7.69 (s, 1H), 7.53-7.46 (m, 1H), 7.42-7.38 (m, 1H), 7.23 (s, 2H), 7.06-7.00 (m, 2H), 5.39 (s, 1H), 4.73 (d, J=14.0 Hz, 1H), 3.84 (s, 3H), 3.76-2.80 (m, 4H), 1.53 (s, 9H), 1.46 (s, 9H). ¹⁹F NMR (376 MHz, Chloroform-d) δ−62.72. LCMS RT (Method 3)=3.553 min, m/z 646.2 [M+Na⁺].

### Example 171

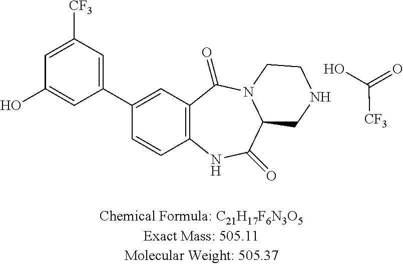

This example is directed to the synthesis of (S)-8-(3-Hydroxy-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-002) in an aspect of the invention.

The compound was prepared following General Procedure B using 1-(tert-butyl)-3-methyl (S)-4-(4-((tert-butoxycarbonyl)amino)-3′-hydroxy-5′-(trifluoromethyl)-[1,1′-biphenyl]-3-carbonyl)piperazine-1,3-dicarboxylate (KJW011-098). ¹H NMR (400 MHz, DMSO-d₆) δ 10.95 (s, 1H), 10.41 (s, 1H), 9.38 (s, 1H), 7.98 (d, J=2.3 Hz, 1H), 7.92 (dd, J=8.4, 2.3 Hz, 1H), 7.38 (dt, J=2.2, 1.1 Hz, 1H), 7.32 (t, J=2.0 Hz, 1H), 7.25 (d, J=8.5 Hz, 1H), 7.08 (t, J=1.9 Hz, 1H), 4.55 (dd, J=5.2, 2.9 Hz, 1H), 4.22 (dt, J=14.4, 4.4 Hz, 1H), 3.68 (dd, J=13.6, 3.0 Hz, 1H), 3.49-3.21 (m, 4H). ¹⁹F NMR (376 MHz, DMSO-d₆) δ−61.30, −73.60. LCMS RT (Method 1)=3.518 min, m/z 391.8 [M+H⁺].

### Example 172

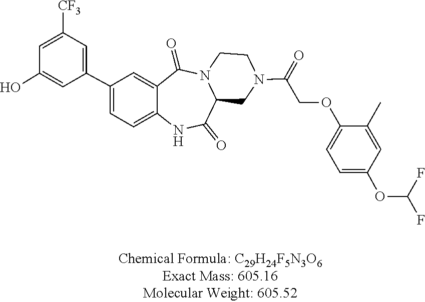 [SMILES: Cc1cc(OC(F)F)ccc1OCC(=O)N1CCN2C(=O)c3cc(-c4cc(O)cc(C(F)(F)F)c4)ccc3NC(=O)[C@@H]2C1]

This example is directed to the synthesis of (S)-2-(2-(4-difluoromethoxy)-2-methylphenoxy)acetyl)-8-(3-hydroxy-5-(trifluoromethyl)phenyl)-1,3,4,12a-tetrahydrobenzo[e]pyrazino[1,2-a][1,4]diazepine-6,12 (2H, 11H)-dione (KJW012-004) in an aspect of the invention.

The compound was prepared following General Procedure C using (S)-8-(3-hydroxy-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-002) and 2-(4-(difluoromethoxy)-2-methylphenoxy)acetic acid (KJW011-096-2). ¹H NMR (600 MHz, DMSO-d₆) δ 10.80 (s, 0.69×1H), 10.72 (s, 0.31×1H), 10.36 (s, 0.31×1H), 10.35 (s, 0.69×1H),), 8.01 (t, J=2.6 Hz, 1H), 7.89 (dt, J=8.5, 2.3 Hz, 1H), 7.41-7.36 (m, 1H), 7.32 (dt, J=4.4, 1.9 Hz, 1H), 7.23 (dd, J=8.5, 1.4 Hz, 1H), 7.20-7.05 (m, 2H), 7.00 (d, J=3.3 Hz, 1H), 6.96-6.93 (m, 1H), 6.92-6.89 (m, 1H), 5.10-4.96 (m, 0.69×2H), 4.91-4.77 (m, 0.31×2H), 4.38 (t, J=4.4 Hz, 0.73×1H), 4.36 (t, J=4.8 Hz, 0.27×1H), 4.22-3.85 (m, 2H), 3.80-3.61 (m, 2H), 3.61-3.54 (m, 1H), 3.41-3.37 (m, 1H), 2.20 (s, 0.31×3H), 2.19 (s, 0.69×3H). ¹⁹F NMR (376 MHz, DMSO-d₆) δ−61.28, −61.30, −80.95, −81.00, −81.15, −81.20. LCMS RT (Method 1)=5.024 min, m/z 605.7 [M+H⁺].

### Example 173

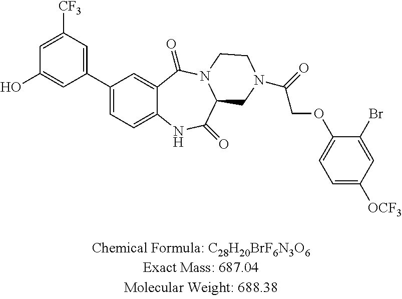 [SMILES: O=C1Nc2ccc(-c3cc(O)cc(C(F)(F)F)c3)cc2C(=O)N2CCN(C(=O)COc3ccc(OC(F)(F)F)cc3Br)C[C@@H]12]

This example is directed to the synthesis of (S)-2-(2-(2-Bromo-4-(trifluoromethoxy)phenoxy)acetyl)-8-(3-hydroxy-5-(trifluoromethyl)phenyl)-1,3,4,12a-tetrahydrobenzo[e]pyrazino[1,2-a][1,4]diazepine-6,12 (2H, 11H)-dione (KJW012-005) in an aspect of the invention.

The compound was prepared following General Procedure C using (S)-8-(3-hydroxy-5-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-002) and 2-(2-bromo-4-(trifluoromethoxy)-phenoxyacetic acid (KJW011-027). ¹H NMR (600 MHz, DMSO-d₆) δ 10.84 (s, 0.69×1H), 10.73 (s, 0.31×1H), 10.36 (s, 0.30×1H), 10.36 (s, 0.70×1H), 8.01 (d, J=2.3 Hz, 1H), 7.90 (ddd, J=8.6, 5.0, 2.4 Hz, 1H), 7.74-7.64 (m, 1H), 7.39 (dt, J=11.0, 1.7 Hz, 1H), 7.35-7.30 (m, 2H), 7.24 (dd, J=8.6, 1.1 Hz, 1H), 7.17-7.10 (m, 1H), 7.06 (d, J=2.0 Hz, 1H), 5.29-5.15 (m, 0.69×2H), 5.12-4.93 (m, 0.31×2H), 4.41 (t, J=4.5 Hz, 0.68×1H), 4.35 (t, J=4.9 Hz, 0.32×1H), 4.22-4.11 (m, 1H), 4.11-3.86 (m, 1H), 3.78-3.71 (m, 1H), 3.71-3.62 (m, 1H), 3.56 (ddd, J=13.5, 8.7, 4.4 Hz, 1H), 3.39 (ddd, J=13.4, 8.9, 4.6 Hz, 1H). ¹⁹F NMR (376 MHz, DMSO-d₆) δ−57.45, −57.46, −61.29, −61.30. LCMS RT (Method 1)=5.374 min, m/z 687.5 [M⁺1]

### Example 174

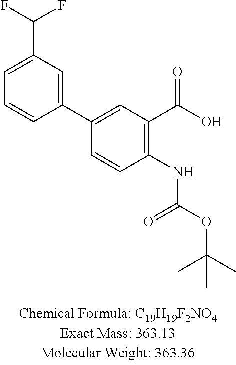 [SMILES: CC(C)(C)OC(=O)Nc1ccc(-c2cccc(C(F)F)c2)cc1C(=O)O]

This example is directed to the synthesis of 4-((tert-butoxycarbonyl)amino)-3′-(difluoromethyl)-[1,1′-biphenyl]-3-carboxylic acid (KJW012-006) in an aspect of the invention.

The compound was prepared following General Procedure K using (3-(difluoromethyl)phenyl)-boronic acid. ¹H NMR (400 MHz, DMSO-d₆) δ 13.82 (s, 1H), 10.55 (s, 1H), 8.41 (d, J=8.8 Hz, 1H), 8.26-8.23 (m, 1H), 7.95 (dd, J=8.8, 2.4 Hz, 1H), 7.88-7.82 (m, 2H), 7.66-7.54 (m, 3H), 7.11 (t, J=55.8 Hz, 1H), 1.50 (s, 9H). ¹⁹F NMR (376 MHz, DMSO-d₆) δ−109.38, −109.53. LCMS RT (Method 2)=3.525 min, m/z 386.8 [M+Na⁺].

### Example 175

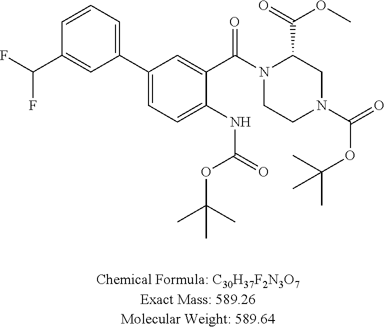 [SMILES: COC(=O)[C@@H]1CN(C(=O)OC(C)(C)C)CCN1C(=O)c1cc(-c2cccc(C(F)F)c2)ccc1NC(=O)OC(C)(C)C]

This example is directed to the synthesis of 1-(tert-butyl) 3-methyl —(S)-4-(4-((tert-butoxycarbonyl)amino)-3′-(difluoromethyl)-[1,1′-biphenyl]-3-carbonyl)piperazine-1,3-dicarboxylate (KJW012-007) in an aspect of the invention.

The compound was prepared following General Procedure A using 4-((tert-butoxycarbonyl)amino)-3′-(difluoromethyl)-[1,1′-biphenyl]-3-carboxylic acid (KJW012-006) and 1-(tert-butyl)-3-methyl (S)-piperazine-1,3-dicarboxylate. ¹H NMR (400 MHz, Chloroform-d) δ 8.24 (dd, J=16.0, 8.7 Hz, 1H), 7.75-7.59 (m, 4H), 7.56-7.45 (m, 3H), 6.70 (t, J=56.4 Hz, 1H), 5.38 (s, 1H), 4.72 (d, J=13.8 Hz, 1H), 3.83 (s, 3H), 3.57 (d, J=12.7 Hz, 1H), 3.47 (bs, 1H), 3.21 (d, J=14.0 Hz, 1H), 3.06 (bs, 1H), 2.85 (bs, 1H), 1.53 (s, 9H), 1.45 (s, 9H). ¹⁹F NMR (376 MHz, Chloroform-d) δ−110.69, −110.84. LCMS RT (Method 2)=3.645 min, m/z 589.8 [M+H⁺].

### Example 176

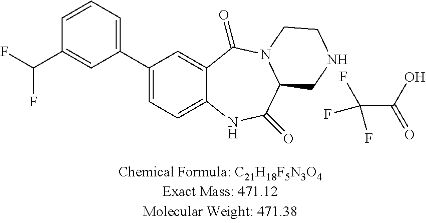

This example is directed to the synthesis of (S)-8-(3-(difluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-008) in an aspect of the invention.

The compound was prepared following General Procedure B using 1-(tert-butyl) 3-methyl (S)-4-(4-((tert-butoxycarbonyl)amino)-3′-(difluoromethyl)-[1,1′-biphenyl]-3-carbonyl)piperazine-1,3-dicarboxylate (KJW012-007). ¹H NMR (400 MHz, DMSO-d₆) δ 10.95 (s, 1H), 9.47 (s, 1H), 8.73 (s, 1H), 8.04 (d, J=2.3 Hz, 1H), 7.95 (dd, J=8.4, 2.3 Hz, 1H), 7.90-7.85 (m, 2H), 7.68-7.58 (m, 2H), 7.28 (d, J=8.5 Hz, 1H), 7.11 (t, J=55.8 Hz, 1H), 4.57 (dd, J=5.2, 2.9 Hz, 1H), 4.23 (dt, J=14.4, 4.4 Hz, 1H), 3.68 (dd, J=13.7, 3.0 Hz, 1H), 3.46 (ddd, J=14.2, 9.8, 3.9 Hz, 1H), 3.41-3.29 (m, 2H), 3.30-3.19 (m, 1H). ¹⁹F NMR (376 MHz, DMSO-d₆) δ−73.63, 109.59 (dd, J=55.9, 10.2 Hz). LCMS RT (Method 2)=2.620 min, m/z 358.1 [M+H⁺].

### Example 177

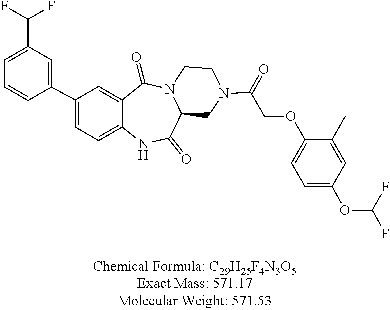 [SMILES: Cc1cc(OC(F)F)ccc1OCC(=O)N1CCN2C(=O)c3cc(-c4cccc(C(F)F)c4)ccc3NC(=O)[C@@H]2C1]

This example is directed to the synthesis of (S)-2-(2-(4-(difluoromethoxy)-2-methylphenoxy)acetyl)-8-(3-(difluoromethyl)phenyl)-1,3,4,12a-tetrahydrobenzo[e]pyrazino[1,2-a][1,4]diazepine-6,12 (2H, 11H)-dione (KJW012-015) in an aspect of the invention.

The compound was prepared following General Procedure C using (S)-8-(3-(difluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-008) and 2-(4-(difluoromethoxy)-2-methylphenoxy)acetic acid (KJW011-096-2). ¹H NMR (600 MHz, DMSO-d₆) δ 10.79 (s, 0.68×1H), 10.71 (s, 0.32×H), 8.07-8.05 (m, 1H), 7.95-7.82 (m, 3H), 7.69-7.57 (m, 2H), 7.25 (dd, J=8.5, 1.1 Hz, 1H), 7.21-6.92 (m, 4H), 6.92-6.89 (m, 1H), 5.13-4.98 (m, 0.66×2H), 4.95-4.78 (m, 0.34×2H), 4.39 (t, J=4.5 Hz, 0.70×1H), 4.37 (t, J=4.8 Hz, 0.30×1H), 4.22-4.09 (m, 2H), 4.00-3.73 (m, 2H), 3.72-3.63 (m, 1H), 3.57 (ddd, J=13.4, 8.6, 4.3 Hz, 1H), 2.20 (s, 0.30×3H), 2.19 (s, 0.70×3H). ¹⁹F NMR (376 MHz, DMSO-d₆) δ−80.97, −81.02, −81.11, −81.17, −81.22, −81.3-−109.56 (ddd, J=55.8, 18.4, 10.0 Hz). LCMS RT (Method 1)=5.214 min, m/z 572.2 [M+H⁺].

### Example 178

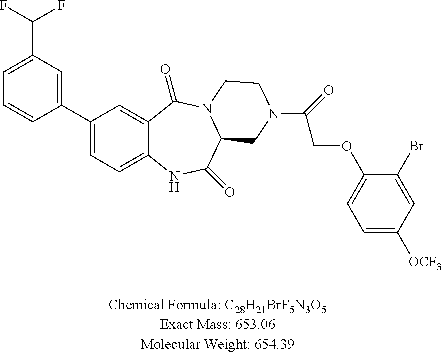 [SMILES: O=C1Nc2ccc(-c3cccc(C(F)F)c3)cc2C(=O)N2CCN(C(=O)COc3ccc(OC(F)(F)F)cc3Br)C[C@@H]12]

This example is directed to the synthesis of (S)-2-(2-(2-Bromo-4-(trifluoromethoxy)phenoxy)acetyl)-8-(3-(difluoromethyl)phenyl)-1,3,4,12a-tetrahydrobenzo[e]pyrazino[1,2-a][1,4]diazepine-6,12 (2H, 11H)-dione (KJW012-016) in an aspect of the invention.

The compound was prepared following General Procedure C using (S)-8-(3-(difluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-008) and 2-(2-bromo-4-(trifluoromethoxy)-phenoxyacetic acid (KJW011-027). ¹H NMR (600 MHz, DMSO-d₆) δ 10.83 (s, 0.69×1H), 10.72 (s, 0.31×1H), 8.07 (d, J=2.3 Hz, 1H), 7.96-7.84 (m, 3H), 7.71-7.67 (m, 1H), 7.64 (t, J=8.0 Hz, 1H), 7.60 (dt, J=7.7, 1.4 Hz, 1H), 7.32 (tdd, J=11.9, 2.9, 1.0 Hz, 1H), 7.26 (dd, J=8.5, 1.4 Hz, 1H), 7.16 (d, J=9.2 Hz, 1H), 7.11 (td, J=54.0, 2.5 Hz, 1H), 5.29-5.13 (m, 0.67×2H), 5.11-4.94 (m, 0.33×2H), 4.42 (t, J=4.6 Hz, 0.70×1H), 4.36 (t, J=4.9 Hz, 0.30×1H), 4.22-4.05 (m, 2H), 4.04-3.63 (m, 3H), 3.61-3.52 (m, 1H). ¹⁹F NMR (376 MHz, DMSO-d₆) δ−57.44, −57.46-−109.57 (ddd, J=55.8, 22.2, 11.1 Hz). LCMS RT (Method 1)=5.662 min, m/z 654.1 [M].

### Example 179

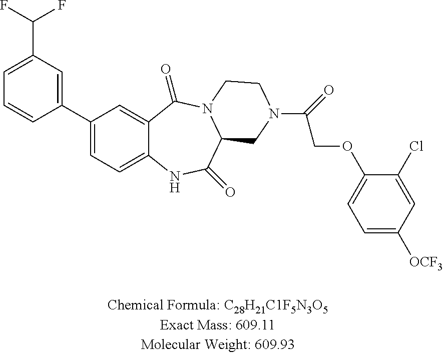 [SMILES: O=C1Nc2ccc(-c3cccc(C(F)F)c3)cc2C(=O)N2CCN(C(=O)COc3ccc(OC(F)(F)F)cc3Cl)C[C@@H]12]

This example is directed to the synthesis of (S)-2-(2-(2-chloro-4-(trifluoromethoxy)phenoxy)acetyl)-8-(3-(difluoromethyl)phenyl)-1,3,4,12a-tetrahydrobenzo[e]pyrazino[1,2-a][1,4]diazepine-6,12 (2H, 11H)-dione (KJW012-017) in an aspect of the invention.

The compound was prepared following General Procedure C using (S)-8-(3-(difluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-008) and 2-(2-chloro-4-(trifluoromethoxy)-phenoxyacetic acid (KJW011-017). ¹H NMR (400 MHz, DMSO-d₆) δ 10.82 (s, 0.69×1H), 10.71 (s, 0.31×1H), 8.07 (d, J=2.3 Hz, 1H), 7.97-7.84 (m, 3H), 7.69-7.52 (m, 3H), 7.32-6.95 (m, 4H), 5.29-5.14 (m, 0.69×2H), 5.12-4.95 (m, 0.31×2H), 4.42 (t, J=4.5 Hz, 0.69×1H), 4.37 (t, J=4.9 Hz, 0.31×1H), 4.24-3.85 (m, 2H), 3.71 (ddt, J=25.8, 12.7, 6.4 Hz, 2H), 3.60-3.34 (m, 1H). ¹⁹F NMR (376 MHz, DMSO-d₆) δ−57.48, 57.49, −109.49 (dd, J=21.8, 10.9 Hz), −109.64 (dd, J=21.7, 10.9 Hz). LCMS RT (Method 1)=5.607 min, m/z 610.2 [M+H⁺].

### Example 180

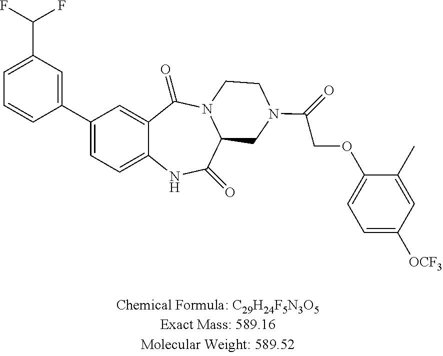 [SMILES: Cc1cc(OC(F)(F)F)ccc1OCC(=O)N1CCN2C(=O)c3cc(-c4cccc(C(F)F)c4)ccc3NC(=O)[C@@H]2C1]

This example is directed to the synthesis of (S)-8-(3-(difluoromethyl)phenyl)-2-(2-(2-methyl-4-(trifluoromethoxy)phenoxy)acetyl)-1,3,4,12a-tetrahydrobenzo[e]pyrazino[1,2-a][1,4]diazepine-6,12 (2H, 11H)-dione (KJW012-018) in an aspect of the invention.

The compound was prepared following General Procedure C using (S)-8-(3-(difluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-008) and 2-(2-methyl-4-(trifluoromethoxy)-phenoxyacetic acid (KJW011-028). ¹H NMR (400 MHz, DMSO-d₆) δ 10.77 (s, 0.68×1H), 10.69 (s, 0.32×1H), 7.96-7.81 (m, 3H), 7.62 (dt, J=15.5, 7.8 Hz, 2H), 7.26 (d, J=8.3 Hz, 1H), 7.17 (d, J=3.2 Hz, 1H), 7.13-6.91 (m, 3H), 5.08 (q, J=15.1 Hz, 0.68×2H), 4.98-4.81 (m, 0.32×2H), 4.38 (dt, J=10.3, 4.7 Hz, 1H), 4.24-4.07 (m, 2H), 4.04-3.63 (m, 3H), 3.48 (m, 1H), 2.22 (d, J=5.4 Hz, 3H). ¹⁹F NMR (376 MHz, DMSO-d₆) δ −57.12, −57.14, −109.59 (ddd, J=55.9, 18.7, 10.2 Hz)). LCMS RT (Method 2)=3.443 min, m/z 590.2 [M+H⁺].

### Example 181

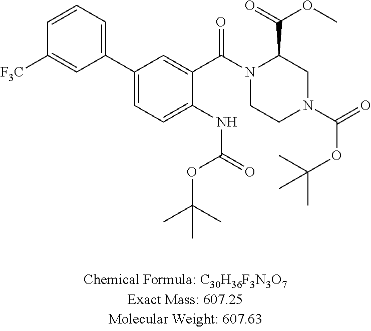 [SMILES: COC(=O)[C@H]1CN(C(=O)OC(C)(C)C)CCN1C(=O)c1cc(-c2cccc(C(F)(F)F)c2)ccc1NC(=O)OC(C)(C)C]

This example is directed to the synthesis of 1-(tert-butyl) 3-methyl (R)-4-(4-((tert-butoxycarbonyl)amino)-3′-(trifluoromethyl)-[1,1′-biphenyl]-3-carbonyl)piperazine-1,3-dicarboxylate (KJW012-011) in an aspect of the invention.

The compound was prepared following General Procedure A using 4-((tert-butoxycarbonyl)amino)-3′-(trifluoromethyl)-[1,1′-biphenyl]-3-carboxylic acid (KJW010-066) and 1-(tert-butyl) 3-methyl (R)-piperazine-1,3-dicarboxylate. [α]_{D}²⁰=+0.64° (c=1, CHCl₃). ¹H NMR (400 MHz, Acetic Acid-d₄) δ 8.09-7.82 (m, 3H), 7.77 (dt, J=7.7, 3.8 Hz, 1H), 7.70-7.59 (m, 3H), 5.44 (bs, 1H), 4.75-4.50 (m, 2H), 4.24-2.90 (m, 7H), 1.54 (s, 9H), 1.46 (s, 9H). ¹⁹F NMR (376 MHz, Acetonitrile-d₃) δ−63.09. LCMS RT (Method 2)=3.852 min, m/z 607.8 [M+H⁺].

### Example 182

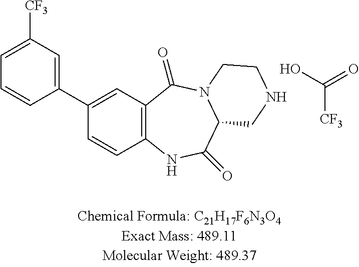

This example is directed to the synthesis of (R)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-012) in an aspect of the invention.

The compound was prepared following General Procedure B using 1-(tert-butyl) 3-methyl (R)-4-(4-((tert-butoxycarbonyl)amino)-3′-(trifluoromethyl)-[1,1′-biphenyl]-3-carbonyl)piperazine-1,3-dicarboxylate (KJW012-011). ¹H NMR (400 MHz, Acetic Acid-d₄) δ 8.27 (d, J=2.2 Hz, 1H), 7.98 (s, 1H), 7.97-7.91 (m, 2H), 7.70 (dt, J=15.3, 7.8 Hz, 2H), 7.33 (d, J=8.4 Hz, 1H), 4.74 (dd, J=5.0, 2.8 Hz, 1H), 4.44 (dt, J=14.8, 4.7 Hz, 1H), 4.09 (dd, J=14.1, 2.9 Hz, 1H), 3.89 (ddd, J=14.7, 8.8, 4.1 Hz, 1H), 3.79 (dd, J=14.1, 5.0 Hz, 1H), 3.75-3.62 (m, 2H). ¹⁹F NMR (376 MHz, Acetic acid-d₄) δ−63.43, −76.62. LCMS RT (Method 2)=2.998 min, m/z 376.1 [M+H⁺].

### Example 183

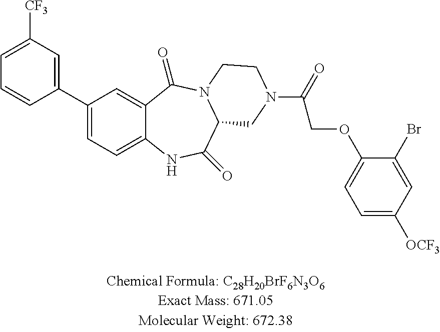 [SMILES: O=C1Nc2ccc(-c3cccc(C(F)(F)F)c3)cc2C(=O)N2CCN(C(=O)COc3ccc(OC(F)(F)F)cc3Br)C[C@H]12]

This example is directed to the synthesis of (R)-2-(2-(2-bromo-4-(trifluoromethoxy)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW012-013) in an aspect of the invention.

The compound was prepared following General Procedure C using 1-(tert-butyl) 3-methyl (R)-4-(4-((tert-butoxycarbonyl)amino)-3′-(trifluoromethyl)-[1,1′-biphenyl]-3-carbonyl)piperazine-1,3-dicarboxylate (KJW012-011) and 2-(2-bromo-4-(trifluoromethoxy)phenoxy)acetic acid (KJW011-027). ¹H NMR (400 MHz, Acetic Acid-d₄) δ 8.30 (d, J=2.4 Hz, 1H), 7.99 (d, J=3.2 Hz, 1H), 7.97-7.90 (m, 2H), 7.69 (dt, J=15.3, 7.8 Hz, 2H), 7.54-7.47 (m, 1H), 7.32 (dd, J=8.4, 2.5 Hz, 1H), 7.29-7.23 (m, 1H), 7.14-7.04 (m, 1H). ¹⁹F NMR (376 MHz Acetic Acid-d₄) δ−59.34, −59.40, −63.40. LCMS RT (Method 1)=5.956 min, m/z 672.1 [M⁺].

### Example 184

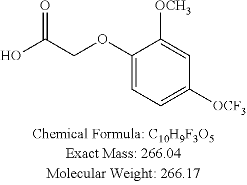 [SMILES: COc1cc(OC(F)(F)F)ccc1OCC(=O)O]

This example is directed to the synthesis of 2-(2-methoxy-4-(trifluoromethoxy)phenoxy)acetic acid (KJW012-039-2) in an aspect of the invention.

The compound was prepared following General Procedure K using 2-methoxy-4-(trifluoromethoxy)phenol. ¹H NMR (400 MHz, DMSO-d₆) δ 6.99 (dd, J=2.8, 0.8 Hz, 1H), 6.92 (d, J=8.9 Hz, 1H), 6.88-6.83 (m, 1H), 4.68 (s, 2H), 3.80 (s, 3H). ¹⁹F NMR (376 MHz, DMSO-d₆) δ−57.07. LCMS RT (Method 2)=2.216 min, m/z 288.8 [M+Na⁺].

### Example 185

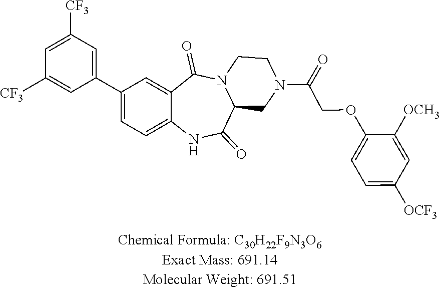 [SMILES: COc1cc(OC(F)(F)F)ccc1OCC(=O)N1CCN2C(=O)c3cc(-c4cc(C(F)(F)F)cc(C(F)(F)F)c4)ccc3NC(=O)[C@@H]2C1]

This example is directed to the synthesis of (S)-8-(3,5-bis(trifluoromethyl)phenyl)-2-(2-(2-methoxy-4-(trifluoromethoxy)phenoxy)acetyl)-1,3,4,12a-tetrahydrobenzo[e]pyrazino[1,2-a][1,4]diazepine-6,12 (2H, 11H)-dione (KJW012-050) in an aspect of the invention.

The compound was prepared following General Procedure C using (S)-8-(3,5-bis(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-047) and 2-(2-methoxy-4-(trifluoromethoxy)phenoxy)acetic acid (KJW012-039-2). ¹H NMR (400 MHz, DMSO-d₆) δ 10.85 (s, 0.69×1H), 10.75 (s, 0.31×1H), 8.38-8.32 (m, 2H), 8.21 (dd, J=7.2, 2.4 Hz, 1H), 8.12-8.04 (m, 2H), 7.28 (d, J=8.5 Hz, 1H), 7.04-6.92 (m, 2H), 6.81 (dtd, J=8.8, 2.7, 1.3 Hz, 1H), 5.01 (m, 0.71×2H), 4.93-4.75 (m, 0.29×2H), 4.38-4.34 (m, 1H), 4.25-3.83 (m, 2H), 3.81 (s, 0.32×3H), 3.76-3.73 (m, 1H), 3.75 (s, 0.68×3H), 3.69 (ddd, J=12.5, 9.0, 4.5 Hz, 1H), 3.56 (ddd, J=13.5, 9.0, 4.2 Hz, 1H), 3.41 (ddd, J=13.2, 8.9, 4.6 Hz, 1H). ¹⁹F NMR (376 MHz, dmso) δ−57.09, −57.13, −61.22, −61.23. LCMS RT (Method 1)=5.899 min, m/z 692.1 [M+H⁺].

### Example 186

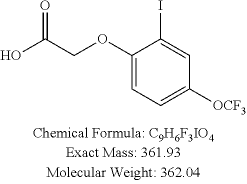 [SMILES: O=C(O)COc1ccc(OC(F)(F)F)cc1I]

This example is directed to the synthesis of 2-(2-iodo-4-(trifluoromethoxy)phenoxy)acetic acid (KJW012-049) in an aspect of the invention.

The compound was prepared following General Procedure E using 2-iodo-4-trifluoromethoxyphenol. ¹H NMR (400 MHz, Methanol-d₄) δ 7.69 (dq, J=2.8, 0.9 Hz, 1H), 7.26 (ddq, J=9.0, 2.9, 1.0 Hz, 1H), 6.91 (d, J=9.1 Hz, 1H), 4.77 (s, 2H). ¹⁹F NMR (376 MHz, Methanol-d₄) δ−60.09. LCMS RT (Method 2)=3.366 min, m/z 384.6 [M+Na⁺].

### Example 187

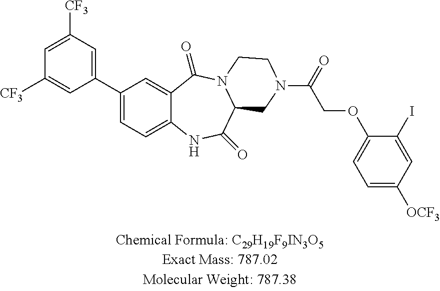 [SMILES: O=C1Nc2ccc(-c3cc(C(F)(F)F)cc(C(F)(F)F)c3)cc2C(=O)N2CCN(C(=O)COc3ccc(OC(F)(F)F)cc3I)C[C@@H]12]

This example is directed to the synthesis of (S)-8-(3,5-bis(trifluoromethyl)phenyl)-2-(2-(2-iodo-4-(trifluoromethoxy)phenoxy)acetyl)-1,3,4,12a-tetrahydrobenzo[e]pyrazino[1,2-a][1,4]diazepine-6,12 (2H, 11H)-dione (KJW012-055) in an aspect of the invention.

The compound was prepared following General Procedure C using (S)-8-(3,5-bis(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-047) and 2-(2-iodo-4-(trifluoromethoxy)phenoxy)acetic acid (KJW012-049). ¹H NMR (400 MHz, DMSO-d₆) δ 10.85 (s, 0.71×1H), 10.75 (s, 0.29×1H), 8.39-8.30 (m, 2H), 8.20 (dd, J=3.7, 2.4 Hz, 1H), 8.14-8.03 (m, 2H), 7.81-7.71 (m, 1H), 7.37-7.23 (m, 2H), 7.01 (dd, J=13.8, 9.2 Hz, 1H), 5.21-5.11 (m, 0.70×2H), 5.07-4.92 (m, 0.30×2H), 4.37 (dt, J=11.9, 4.8 Hz, 1H), 4.26-3.84 (m, 3H), 3.83-3.63 (m, 2H), 3.48 (m, 1H). ¹⁹F NMR (376 MHz, DMSO-d₆) δ−57.41, −57.46, −61.22, −61.23. LCMS RT (Method 1)=6.242 min, m/z 788.0 [M+H⁺].

### Example 188

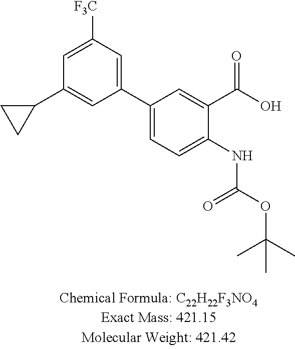 [SMILES: CC(C)(C)OC(=O)Nc1ccc(-c2cc(C3CC3)cc(C(F)(F)F)c2)cc1C(=O)O]

This example is directed to the synthesis of 4-((tert-butoxycarbonyl)amino)-3′-cyclopropyl-5′-(trifluoromethyl)-[1,1′-biphenyl]-3-carboxylic acid (KJW012-042) in an aspect of the invention.

The compound was prepared following General Procedure K using 3-cyclopropyl-5-trifluoromethylphenol. ¹H NMR (400 MHz, DMSO-d₆) δ 13.82 (s, 1H), 10.55 (s, 1H), 8.39 (d, J=8.8 Hz, 1H), 8.22 (d, J=2.4 Hz, 1H), 7.96 (dd, J=8.8, 2.4 Hz, 1H), 7.73-7.60 (m, 2H), 7.37 (t, J=1.6 Hz, 1H), 2.14 (tt, J=8.4, 5.1 Hz, 1H), 1.50 (s, 9H), 1.08-1.01 (m, 2H), 0.89-0.80 (m, 2H). ¹⁹F NMR (376 MHz, DMSO-d₆) δ−60.96. LCMS RT (Method 3)=2.947 min, m/z 444.1 [M+Na⁺].

### Example 189

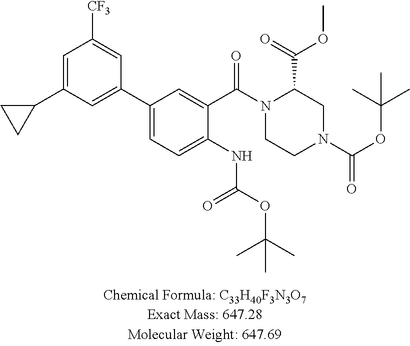 [SMILES: COC(=O)[C@@H]1CN(C(=O)OC(C)(C)C)CCN1C(=O)c1cc(-c2cc(C3CC3)cc(C(F)(F)F)c2)ccc1NC(=O)OC(C)(C)C]

This example is directed to the synthesis of 1-(tert-butyl) 3-methyl (S)-4-(4-((tert-butoxycarbonyl)amino)-3′-cyclopropyl-5′-(trifluoromethyl)-[1,1′-biphenyl]-3-carbonyl)piperazine-1,3-dicarboxylate (KJW012-045) in an aspect of the invention.

The compound was prepared following General Procedure A using (S)-8-(3,5-bis(trifluoromethyl)phenyl)-2-(2-(2-iodo-4-(trifluoromethoxy)phenoxy)acetyl)-1,3,4,12a-tetrahydrobenzo[e]pyrazino[1,2-a][1,4]diazepine-6,12 (2H, 11H)-dione (KJW012-055) and 1-(tert-butyl) 3-methyl (S)-piperazine-1,3-dicarboxylate. ¹H NMR (400 MHz, Methanol-d₄) δ 7.87 (d, J=9.9 Hz, 1H), 7.78-7.71 (m, 1H), 7.65 (s, 1H), 7.62-7.51 (m, 2H), 7.35 (d, J=2.0 Hz, 1H), 5.36-5.26 (m, 1H), 4.72-4.45 (m, 2H), 3.86 (s, 3H), 3.63-3.44 (m, 2H), 3.25-2.86 (m, 1H), 2.11 (qd, J=9.0, 5.2 Hz, 1H), 1.55 (s, 9H), 1.48 (s, 9H), 1.17-1.04 (m, 2H), 0.90-0.80 (m, 2H). ¹⁹F NMR (376 MHz, Methanol-d₄) δ−64.06. LCMS RT (Method 3)=3.831 min. m/z 648.1 [M+H⁺].

### Example 190

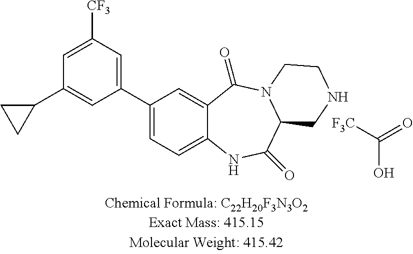

This example is directed to the synthesis of (S)-8-(3-cyclopropyl-5-(trifluoromethyl)phenyl)-1,3,4,12a-tetrahydrobenzo[e]pyrazino[1,2-a][1,4]diazepine-6,12 (2H, 11H)-dione (KJW012-048) in an aspect of the invention.

The compound was prepared following General Procedure B using 1-(tert-butyl) 3-methyl (S)-4-(4-((tert-butoxycarbonyl)amino)-3′-cyclopropyl-5′-(trifluoromethyl)-[1,1′-biphenyl]-3-carbonyl)piperazine-1,3-dicarboxylate (KJW012-045). ¹H NMR (400 MHz, DMSO-d₆) δ 10.93 (s, 1H), 8.05 (d, J=2.3 Hz, 1H), 7.98 (dd, J=8.4, 2.3 Hz, 1H), 7.71 (td, J=1.7, 0.8 Hz, 1H), 7.64 (d, J=1.7 Hz, 1H), 7.44 (d, J=1.6 Hz, 1H), 7.26 (d, J=8.5 Hz, 1H), 4.55 (dd, J=5.2, 2.9 Hz, 1H), 4.23 (dt, J=14.4, 4.4 Hz, 1H), 3.69 (dd, J=13.7, 3.0 Hz, 1H), 3.46 (ddd, J=14.2, 9.9, 3.9 Hz, 1H), 3.35 (dq, J=12.6, 4.7, 4.3 Hz, 2H), 3.24 (ddd, J=13.1, 9.7, 4.0 Hz, 1H), 2.15 (tt, J=8.4, 5.1 Hz, 1H), 1.13-1.00 (m, 2H), 0.96-0.72 (m, 2H). ¹⁹F NMR (376 MHz, DMSO-d₆) δ−61.00, −73.83. LCMS RT (Method 3)=3.010 min, m/z 416.1 [M+H⁺].

### Example 190

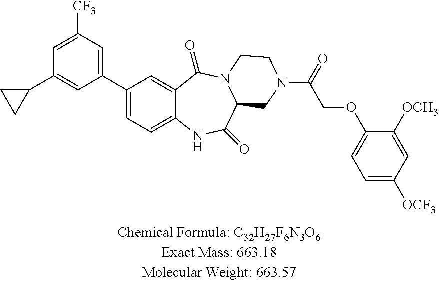 [SMILES: COc1cc(OC(F)(F)F)ccc1OCC(=O)N1CCN2C(=O)c3cc(-c4cc(C5CC5)cc(C(F)(F)F)c4)ccc3NC(=O)[C@@H]2C1]

This example is directed to the synthesis of (S)-8-(3-cyclopropyl-5-(trifluoromethyl)phenyl)-2-(2-(2-methoxy-4-(trifluoromethoxy)phenoxy)acetyl)-1,3,4,12a-tetrahydrobenzo[e]pyrazino[1,2-a][1,4]diazepine-6,12 (2H, 11H)-dione (KJW012-056) in an aspect of the invention.

The compound was prepared following General Procedure C using (S)-8-(3-cyclopropyl-5-(trifluoromethyl)phenyl)-1,3,4,12a-tetrahydrobenzo[e]pyrazino[1,2-a][1,4]diazepine-6,12 (2H, 11H)-dione (KJW012-048) and 2-(2-methoxy-4-(trifluoromethoxy)phenoxy)acetic acid (KJW012-039-2). ¹H NMR (400 MHz, DMSO-d₆) δ 10.79 (s, 1H), 10.69 (s, OH), 8.07 (dd, J=5.1, 2.4 Hz, 1H), 7.95 (ddd, J=8.5, 3.2, 2.3 Hz, 1H), 7.76-7.61 (m, 2H), 7.43 (d, J=1.7 Hz, 1H), 7.25 (d, J=8.5 Hz, 1H), 7.04-6.94 (m, 2H), 6.81 (dtq, J=7.7, 2.8, 1.1 Hz, 1H), 5.07-4.96 (m, 0.70×2H), 4.93-4.75 (m, 0.30×2H), 4.36 (dt, J=8.9, 4.8 Hz, 1H), 4.29-3.85 (m, 2H), 3.79-3.38 (m, 4H), 2.15 (tt, J=8.4, 5.1 Hz, 1H), 1.11-0.98 (m, 2H), 0.98-0.81 (m, 2H). ¹⁹F NMR (376 MHz, DMSO-d₆) δ−57.07, −57.10, −60.99, −61.00. LCMS RT (Method 1)=6.335 min, m/z 663.7 [M+H⁺].

### Example 191

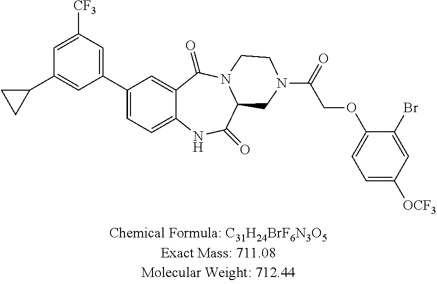 [SMILES: O=C1Nc2ccc(-c3cc(C4CC4)cc(C(F)(F)F)c3)cc2C(=O)N2CCN(C(=O)COc3ccc(OC(F)(F)F)cc3Br)C[C@@H]12]

This example is directed to the synthesis of (S)-2-(2-(2-bromo-4-(trifluoromethoxy)phenoxy)acetyl)-8-(3-cyclopropyl-5-(trifluoromethyl)phenyl)-1,3,4,12a-tetrahydrobenzo[e]pyrazino[1,2-a][1,4]diazepine-6,12 (2H, 11H)-dione (KJW012-057) in an aspect of the invention.

The compound was prepared following General Procedure C using (S)-8-(3-cyclopropyl-5-(trifluoromethyl)phenyl)-1,3,4,12a-tetrahydrobenzo[e]pyrazino[1,2-a][1,4]diazepine-6,12 (2H, 11H)-dione (KJW012-048) and 2-(2-bromo-4-(trifluoromethoxy)phenoxy)acetic acid (KJW011-027). ¹H NMR (400 MHz, DMSO-d₆) δ 10.81 (s, 0.65×1H), 10.73 (s, 0.35×1H), 8.07 (dd, J=4.6, 2.3 Hz, 1H), 7.95 (ddd, J=8.4, 3.7, 2.3 Hz, 1H), 7.74-7.60 (m, 4H), 7.43 (d, J=2.0 Hz, 1H), 7.37-7.28 (m, 1H), 7.25 (d, J=8.4 Hz, 1H), 7.14 (dd, J=18.8, 9.2 Hz, 1H), 5.20 (q, J=15.3 Hz, 0.69×2H), 5.12-4.94 (m, 0.31×2H), 4.41 (t, J=4.7 Hz, 0.70×1H), 4.35 (t, J=4.9 Hz, 0.30×1H), 4.24-3.61 (m, 5H), 3.47 (dddd, J=61.7, 13.2, 8.9, 4.5 Hz, 1H), 2.15 (tt, J=8.4, 5.1 Hz, 1H), 1.09-1.00 (m, 2H), 0.87 (ddd, J=7.0, 4.8, 1.9 Hz, 2H). ¹⁹F NMR (376 MHz, DMSO-d₆) δ−57.47, −57.49, −57.51, −61.00. LCMS RT (Method 1)=6.222 min, m/z 712.0 [M⁺].

### Example 192

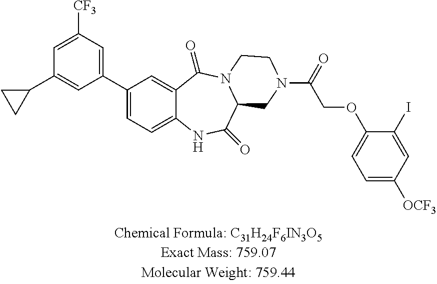 [SMILES: O=C1Nc2ccc(-c3cc(C4CC4)cc(C(F)(F)F)c3)cc2C(=O)N2CCN(C(=O)COc3ccc(OC(F)(F)F)cc3I)C[C@@H]12]

This example is directed to the synthesis of (S)-8-(3-cyclopropyl-5-(trifluoromethyl)phenyl)-2-(2-(2-iodo-4-(trifluoromethoxy)phenoxy)acetyl)-1,3,4,12a-tetrahydrobenzo[e]pyrazino[1,2-a][1,4]diazepine-6,12 (2H, 11H)-dione (KJW012-058) in an aspect of the invention.

The compound was prepared following General Procedure C using (S)-8-(3-cyclopropyl-5-(trifluoromethyl)phenyl)-1,3,4,12a-tetrahydrobenzo[e]pyrazino[1,2-a][1,4]diazepine-6,12 (2H, 11H)-dione (KJW012-048) and 2-(2-iodo-4-(trifluoromethoxy)phenoxy)acetic acid (KJW012-049). ¹H NMR (400 MHz, DMSO-d₆) δ 10.82 (s, 0.71×1H), 10.71 (s, 0.29×1H), 8.07 (t, J=2.2 Hz, 1H), 7.95 (ddd, J=8.5, 3.4, 2.3 Hz, 1H), 7.80-7.69 (m, 2H), 7.64 (dt, J=6.8, 1.8 Hz, 1H), 7.43 (d, J=2.0 Hz, 1H), 7.32 (dddt, J=7.9, 6.0, 3.0, 1.0 Hz, 1H), 7.25 (d, J=8.4 Hz, 1H), 7.01 (dd, J=17.3, 9.2 Hz, 1H), 5.26-5.09 (m, 0.68×2H), 5.09-4.89 (m, 0.32×2H), 4.40 (t, J=4.7 Hz, 0.07×1H), 4.35 (t, J=4.9 Hz, 0.30×1H), 4.25-3.62 (m, 4H), 3.47 (dddd, J=60.5, 13.0, 8.9, 4.2 Hz, 2H), 2.15 (tt, J=8.4, 5.0 Hz, 1H), 1.11-0.98 (m, 2H), 0.94-0.79 (m, 2H). ¹⁹F NMR (376 MHz, DMSO-d₆) δ−57.40 (0.32×3F), −57.42 (0.68×3F), −60.99 (0.67×3F), −61.00 (0.33×3F). LCMS RT (Method 1)=6.299 min, m/z 760.0 [M+H⁺].

### Example 193

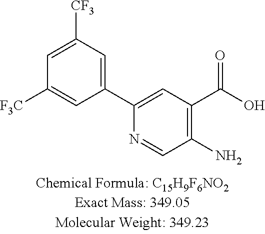 [SMILES: Nc1cnc(-c2cc(C(F)(F)F)cc(C(F)(F)F)c2)cc1C(=O)O]

This example is directed to the synthesis of 4-amino-3′,5′-bis(trifluoromethyl)-[1,1′-biphenyl]-3-carboxylic acid (KJW012-065) in an aspect of the invention.

The compound was prepared following General Procedure K using 5-amino-2-bromoisonicotinic acid and (3,5-bis(trifluoromethyl)phenyl)boronic acid and was used without further purification in KWJ012-066. LCMS RT (Method 2)=3.471 min, m/z 350.8 [M+H].

### Example 194

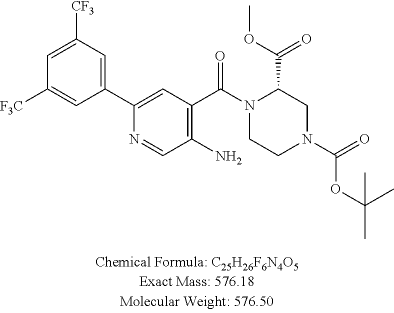 [SMILES: COC(=O)[C@@H]1CN(C(=O)OC(C)(C)C)CCN1C(=O)c1cc(-c2cc(C(F)(F)F)cc(C(F)(F)F)c2)ncc1N]

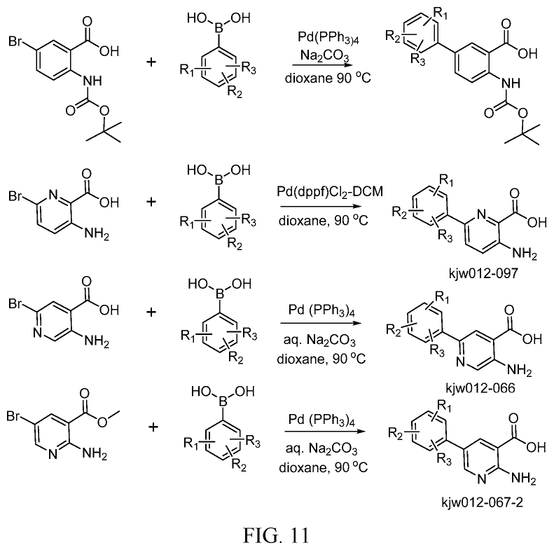

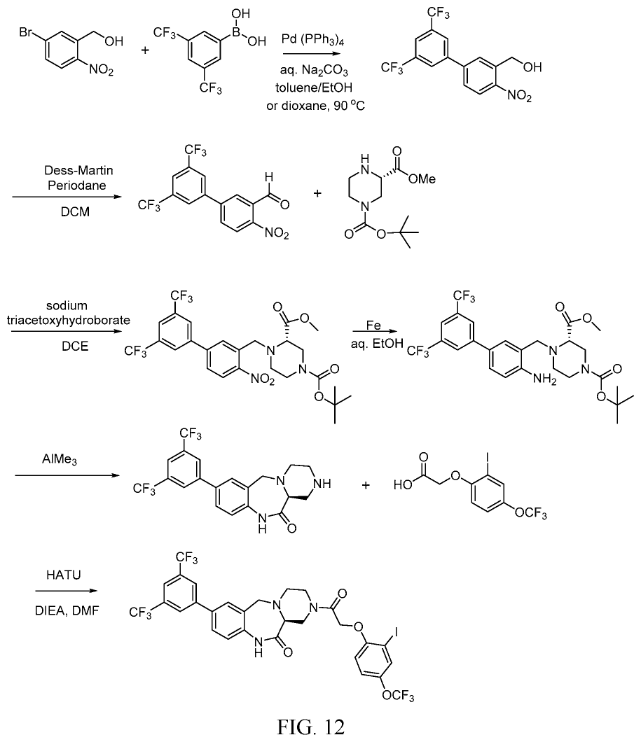

This example is directed to the synthesis of 1-(tert-butyl) 3-methyl (S)-4-(5-amino-2-(3,5-bis(trifluoromethyl)phenyl)isonicotinoyl)piperazine-1,3-dicarboxylate (KJW012-066) in an aspect of the invention. See FIG. 11.

The compound was prepared following General Procedure A using 4-amino-3′,5′-bis(trifluoromethyl)-[1,1′-biphenyl]-3-carboxylic acid (KJW012-065) and 1-(tert-butyl) 3-methyl (S)-piperazine-1,3-dicarboxylate. ¹H NMR (400 MHz, Chloroform-d) δ 8.35 (s, 2H), 8.23 (s, 1H), 7.80 (s, 1H), 7.58 (s, 1H), 5.40-5.37 (m, 1H), 4.78-4.71 (m, 3H), 3.83 (s, 3H), 3.66-3.45 (m, 2H), 3.21 (dd, J=14.0, 4.6 Hz, 1H), 1.44 (s, 9H). ¹⁹F NMR (376 MHz, Chloroform-d) δ−62.88. LCMS RT (Method 2)=3.723 min, m/z 576.7 [M+H⁺].

### Example 195

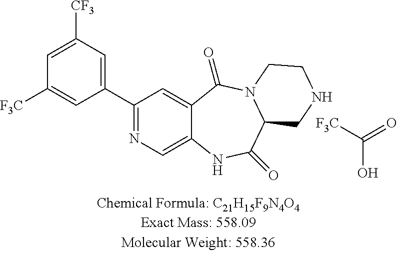

This example is directed to the synthesis of (S)-8-(3,5-bis(trifluoromethyl)phenyl)-1,3,4,12a-tetrahydropyrazino[1,2-a]pyrido[3,4-e][1,4]diazepine-6,12 (2H, 11H)-dione 2,2,2-trifluoroacetate (KJW012-079) in an aspect of the invention.

The compound was prepared following General Procedure B using 1-(tert-butyl) 3-methyl (S)-4-(5-amino-2-(3,5-bis(trifluoromethyl)phenyl)isonicotinoyl)piperazine-1,3-dicarboxylate (KJW012-066). ¹H NMR (400 MHz, DMSO-d₆) δ 11.27 (s, 1H), 9.51 (bs, 1H), 8.78 (s, 3H), 8.62 (d, J=11.4 Hz, 2H), 8.19 (s, 1H), 4.63-4.59 (m, 1H), 4.22 (dt, J=14.6, 4.5 Hz, 1H), 3.71 (dd, J=13.7, 3.1 Hz, 1H), 3.51 (ddd, J=14.2, 9.6, 3.9 Hz, 1H), 3.55-3.48 (m, 2H), 3.25 (ddd, J=13.0, 9.6, 4.0 Hz, 1H). ¹⁹F NMR (376 MHz, DMSO-d₆) δ−61.30, −73.81. LCMS RT (Method 2)=3.044 min, m/z 444.8 [M+H⁺].

### Example 196

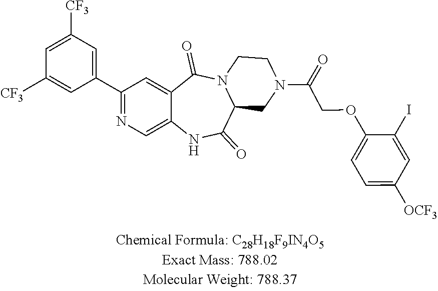 [SMILES: O=C1Nc2cnc(-c3cc(C(F)(F)F)cc(C(F)(F)F)c3)cc2C(=O)N2CCN(C(=O)COc3ccc(OC(F)(F)F)cc3I)C[C@@H]12]

This example is directed to the synthesis of (S)-8-(3,5-bis(trifluoromethyl)phenyl)-2-(2-(2-iodo-4-(trifluoromethoxy)phenoxy)acetyl)-1,3,4,12a-tetrahydropyrazino[1,2-a]pyrido[3,4-e][1,4]diazepine-6,12 (2H, 11H)-dione (KJW012-080) in an aspect of the invention.

The compound was prepared following General Procedure C using (S)-8-(3,5-bis(trifluoromethyl)phenyl)-1,3,4,12a-tetrahydropyrazino[1,2-a]pyrido[3,4-e][1,4]diazepine-6,12 (2H, 11H)-dione 2,2,2-trifluoroacetate (KJW012-079) and 2-(2-iodo-4-(trifluoromethoxy)phenoxy)acetic acid (KJW012-049). ¹H NMR (400 MHz, DMSO-d₆) δ 11.12 (s, 0.70×1H), 11.02 (s, 0.30×1H), 8.76 (d, J=9.9 Hz, 2H), 8.66-8.50 (m, 2H), 8.18 (s, 1H), 7.77 (dd, J=16.5, 2.9 Hz, 1H), 7.33 (dt, J=8.2, 3.9 Hz, 1H), 7.00 (dd, J=11.4, 9.1 Hz, 1H), 5.15 (m, 0.66×2H), 5.08-4.91 (m, 0.33×2H), 4.49-4.38 (m, 1H), 4.26-3.54 (m, 5H), 3.44 (ddd, J=12.2, 8.4, 4.8 Hz, 1H). ¹⁹F NMR (376 MHz, DMSO-d₆) δ−57.41, −57.46, −61.32. LCMS RT (Method 1)=6.143 min, m/z 788.7 [M+H⁺].

### Example 197

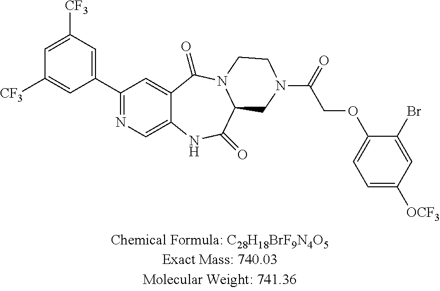 [SMILES: O=C1Nc2cnc(-c3cc(C(F)(F)F)cc(C(F)(F)F)c3)cc2C(=O)N2CCN(C(=O)COc3ccc(OC(F)(F)F)cc3Br)C[C@@H]12]

This example is directed to the synthesis of (S)-8-(3,5-bis(trifluoromethyl)phenyl)-2-(2-(2-bromo-4-(trifluoromethoxy)phenoxy)acetyl)-1,3,4,12a-tetrahydropyrazino[1,2-a]pyrido[3,4-e][1,4]diazepine-6,12 (2H, 11H)-dione (KJW012-081) in an aspect of the invention.

The compound was prepared following General Procedure C using (S)-8-(3,5-bis(trifluoromethyl)phenyl)-1,3,4,12a-tetrahydropyrazino[1,2-a]pyrido[3,4-e][1,4]diazepine-6,12 (2H, 11H)-dione 2,2,2-trifluoroacetate (KJW012-079) and 2-(2-bromo-4-(trifluoromethoxy)phenoxy)acetic acid (KJW011-027). ¹H NMR (400 MHz, DMSO-d₆) δ 11.13 (s, 0.68×1H), 11.02 (s, 0.32×1H), 8.76 (d, J=10.4 Hz, 2H), 8.67-8.48 (m, 2H), 8.18 (s, 1H), 7.67 (dd, J=11.0, 2.9 Hz, 1H), 7.33 (td, J=7.7, 6.5, 2.8 Hz, 1H), 7.12 (dd, J=13.5, 9.1 Hz, 1H), 5.25-5.12 (m, 0.68×2H), 5.11-4.94 (m, 0.32×2H), 4.46 (t, J=4.8 Hz, 0.67×1H), 4.43 (t, J=5.0 Hz, 0.33×1H), 4.28-3.86 (m, 2H), 3.86-3.54 (m, 2H), 3.52-3.27 (m, 2H). ¹⁹F NMR (376 MHz, DMSO-d₆) δ−57.47, −57.50, −61.31. LCMS RT (Method 1)=6.087 min, m/z 740.8 [M⁺].

### Example 198

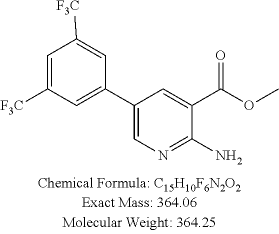 [SMILES: COC(=O)c1cc(-c2cc(C(F)(F)F)cc(C(F)(F)F)c2)cnc1N]

This example is directed to the synthesis of methyl 2-amino-5-(3,5-bis(trifluoromethyl)phenyl)nicotinate (KJW012-067-1) in an aspect of the invention.

The compound was prepared following General Procedure K using methyl 2-amino-5-bromonicotinate and (3,5-bis(trifluoromethyl)phenyl)boronic acid. ¹H NMR (400 MHz, Chloroform-d) δ 8.50 (d, J=2.5 Hz, 1H), 8.37 (d, J=2.5 Hz, 1H), 7.94 (s, 2H), 7.84 (s, 1H), 3.96 (d, J=1.1 Hz, 3H). ¹⁹F NMR (376 MHz, Chloroform-d) δ−62.86. LCMS RT (Method 2)=3.318 min, m/z 365.0 [M⁺].

### Example 199

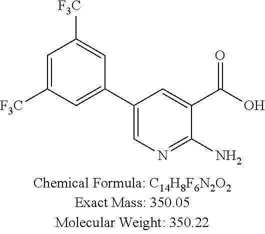 [SMILES: Nc1ncc(-c2cc(C(F)(F)F)cc(C(F)(F)F)c2)cc1C(=O)O]

This example is directed to the synthesis of 2-amino-5-(3,5-bis(trifluoromethyl)phenyl)nicotinic acid (KJW012-067-2) in an aspect of the invention. See FIG. 11.

To a stirring solution of methyl 2-amino-5-(3,5-bis(trifluoromethyl)phenyl)nicotinate (KJW012-067-1) (1 eq) in 1:1 MeOH-THF was added 4M NaOH (1.4 eq.) and the resulting mixture was heated to 60° C. for 6 hours. The reaction was allowed to cool and then acidified with 2M HCl (1.6 eq.) to pH˜5, diluted with water and extracted twice with 10% MeOH-DCM. The layers were separated and the organic layer was dried (Na₂SO₄) and concentrated under vacuum to afford the title compound. ¹H NMR (400 MHz, DMSO-d6) δ 8.54 (d, J=2.6 Hz, 1H), 8.38 (d, J=2.7 Hz, 1H), 8.22 (s, 2H), 7.95 (s, 1H). ¹⁹F NMR (376 MHz, DMSO-d₆) δ−61.25. LCMS RT (Method 3)=2.550 min, m/z 350.8 [M⁺].

### Example 200

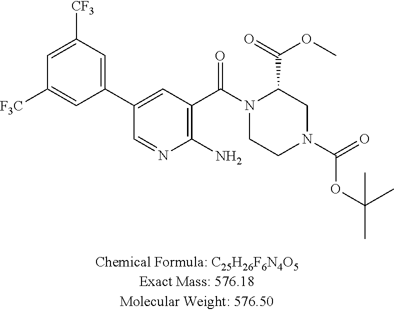 [SMILES: COC(=O)[C@@H]1CN(C(=O)OC(C)(C)C)CCN1C(=O)c1cc(-c2cc(C(F)(F)F)cc(C(F)(F)F)c2)cnc1N]

This example is directed to the synthesis of 1-(tert-butyl) 3-methyl (S)-4-(2-amino-5-(3,5-bis(trifluoromethyl)phenyl)nicotinoyl)piperazine-1,3-dicarboxylate (KJW012-072) in an aspect of the invention.

The compound was prepared following General Procedure A using 2-amino-5-(3,5-bis(trifluoromethyl)phenyl)nicotinic acid (KJW012-067) and 1-(tert-butyl) 3-methyl (S)-piperazine-1,3-dicarboxylate. LCMS RT (Method 3)=3.471 min, m/z 576.9 [M+H⁺].

### Example 201

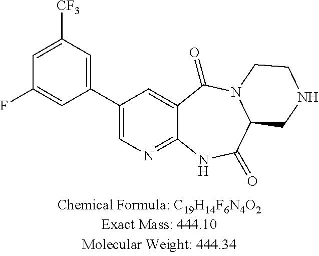 [SMILES: O=C1Nc2ncc(-c3cc(F)cc(C(F)(F)F)c3)cc2C(=O)N2CCNC[C@@H]12]

This example is directed to the synthesis of (S)-3-(3,5-bis(trifluoromethyl)phenyl)-8,9,10,10a-tetrahydropyrazino[1,2-a]pyrido[2,3-e][1,4]diazepine-5,11 (7H, 12H)-dione (KJW012-092) in an aspect of the invention.

To a slurry 1-(tert-butyl)-3-methyl (S)-4-(2-amino-5-(3,5-bis(trifluoromethyl)phenyl)nicotinoyl)piperazine-1,3-dicarboxylate (KJW012-072) (1 eq.) in toluene was added AlMe₃ (3 eq., 2M in toluene). The result was allowed to stir at 95° C. for 18 hours. The reaction was cooled in an ice-bath, quenched with saturated aq. Rochelle's salt and allowed to warm to room temperature. The reaction was poured into EtOAc and washed twice with NaHCO₃, dried (Na₂SO₄), filtered through CELITE™, and concentrated under vacuum. The residue was purified by flash column chromatography (silica gel, 0-5% MeOH-DCM) to afford the title compound. ¹H NMR (400 MHz, DMSO-d₆) δ 11.08 (s, 1H), 9.07 (d, J=2.5 Hz, 1H), 8.63 (d, J=2.5 Hz, 1H), 8.49 (s, 2H), 8.14 (s, 1H), 4.23-4.12 (m, 1H), 4.07 (d, J=4.8 Hz, 1H), 3.11-3.00 (m, 1H), 2.88-2.79 (m, 1H), 2.79-2.71 (m, 2H), 2.69-2.60 (m, 1H). ¹⁹F NMR (376 MHz, DMSO-d₆) δ−61.17. LCMS RT (Method 2)=2.836 min, m/z 445.0 [M+H⁺].

### Example 202

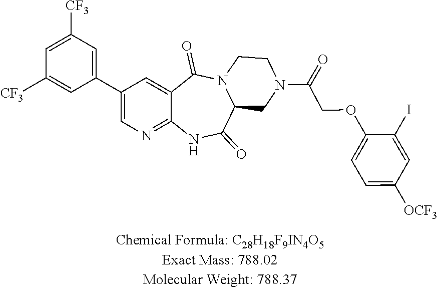 [SMILES: O=C1Nc2ncc(-c3cc(C(F)(F)F)cc(C(F)(F)F)c3)cc2C(=O)N2CCN(C(=O)COc3ccc(OC(F)(F)F)cc3I)C[C@@H]12]

This example is directed to the synthesis of (S)-3-(3,5-bis(trifluoromethyl)phenyl)-9-(2-(2-iodo-4-(trifluoromethoxy)phenoxy)acetyl)-8,9,10,10a-tetrahydropyrazino[1,2-a]pyrido[2,3-e][1,4]diazepine-5,11 (7H, 12H)-dione (KJW012-093) in an aspect of the invention.

The compound was prepared following General Procedure C using ((S)-3-(3,5-bis(trifluoromethyl)phenyl)-8,9,10,10a-tetrahydropyrazino[1,2-a]pyrido[2,3-e][1,4]diazepine-5,11 (7H, 12H)-dione (KJW012-092) and 2-(2-iodo-4-(trifluoromethoxy)phenoxy)acetic acid (KJW012-049). ¹H NMR (400 MHz, DMSO-d₆) δ 9.00 (d, J=2.4 Hz, 1H), 8.86 (dd, J=4.0, 2.4 Hz, 1H), 8.32 (s, 2H), 8.07 (s, 1H), 7.70 (dd, J=23.2, 2.8 Hz, 1H), 7.29 (dt, J=8.3, 3.4 Hz, 1H), 7.01 (dd, J=31.4, 9.1 Hz, 1H), 5.21-5.13 (m, 0.60×2H), 5.06-4.92 (m, 0.40×2H), 4.64-4.42 (m, 2H), 4.33-4.08 (m, 2H), 4.08-3.69 (m, 3H). ¹⁹F NMR (376 MHz, DMSO-d₆) δ−58.87, −58.92, −63.17, −63.17. LCMS RT (Method 1)=5.952 min, m/z 789.0 [M+H⁺].

### Example 203

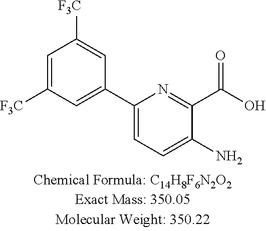 [SMILES: Nc1ccc(-c2cc(C(F)(F)F)cc(C(F)(F)F)c2)nc1C(=O)O]

This example is directed to the synthesis of 3-amino-6-(3,5-bis(trifluoromethyl)phenyl)picolinic acid (KJW012-097) in an aspect of the invention. See FIG. 11.

The compound was prepared following General Procedure K substituting Pd(dppf)Cl₂-DCM ([1,1′-bis(diphenylphosphino)ferrocene]dichloropalladium(II), complex with dichloromethane) for tetrakis(triphenylphosphine)palladium(0) and using, 3-amino-6-bromopicolinic acid and (3,5-bis(trifluoromethyl)phenyl)boronic acid. ¹H NMR (400 MHz, Chloroform-d) δ 11.09 (s, 1H), 8.27 (s, 2H), 7.89 (s, 1H), 7.84 (d, J=8.7 Hz, 1H), 7.28 (d, J=8.7 Hz, 1H), 6.11 (bs, 2H). LCMS RT (Method 2)=3.386 min, m/z 351.0 [M+H⁺].

### Example 204

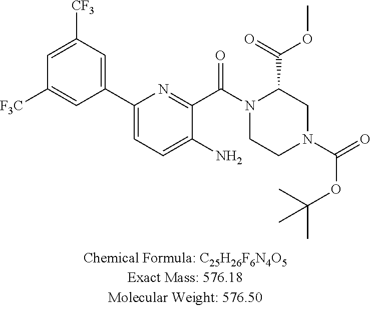 [SMILES: COC(=O)[C@@H]1CN(C(=O)OC(C)(C)C)CCN1C(=O)c1nc(-c2cc(C(F)(F)F)cc(C(F)(F)F)c2)ccc1N]

This example is directed to the synthesis of 1-(tert-butyl) 3-methyl (S)-4-(3-amino-6-(3,5-bis(trifluoromethyl)phenyl)picolinoyl)piperazine-1,3-dicarboxylate (KJW012-098) in an aspect of the invention.

The compound was prepared following General Procedure A using 3-amino-6-(3,5-bis(trifluoromethyl)phenyl)picolinic acid (KJW012-097) and 1-(tert-butyl) 3-methyl (S)-piperazine-1,3-dicarboxylate. ¹H NMR (400 MHz, Chloroform-d) δ 8.35 (s, 1H), 8.24 (s, 1H), 7.81 (s, 1H), 7.67 (dd, J=15.6, 8.6 Hz, 1H), 7.16 (d, J=8.6 Hz, 1H), 5.29 (t, J=3.1 Hz, 1H), 4.70 (d, J=13.8 Hz, 1H), 4.56 (t, J=14.5 Hz, 1H), 4.21 (d, J=12.5 Hz, 2H), 3.82 (s, 3H), 3.63 (s, 2H), 3.34 (dd, J=13.9, 4.7 Hz, 1H), 1.46 (d, J=4.0 Hz, 9H). ¹⁹F NMR (376 MHz, Chloroform-d) δ−62.86, −62.93. LCMS RT (Method 2)=3.625 min, m/z 577.2 [M+H⁺].

### Example 205

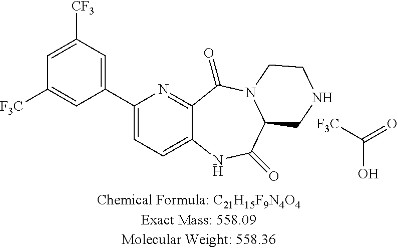

This example is directed to the synthesis of (S)-2-(3,5-bis(trifluoromethyl)phenyl)-7,8,9,10-tetrahydropyrazino[1,2-a]pyrido[3,2-e][1,4]diazepine-6,12 (5H, 6aH)-dione 2,2,2-trifluoroacetate (KJW012-099) in an aspect of the invention.

The compound was prepared following General Procedure B using 1-(tert-butyl) 3-methyl (S)-4-(3-amino-6-(3,5-bis(trifluoromethyl)phenyl)picolinoyl)piperazine-1,3-dicarboxylate (KJW012-098). LCMS RT (Method 3)=3.213 min, m/z 445.1 [M+H⁺].

### Example 206

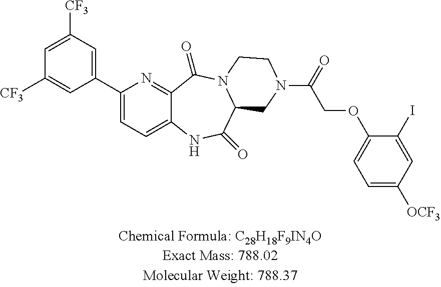 [SMILES: O=C1Nc2ccc(-c3cc(C(F)(F)F)cc(C(F)(F)F)c3)nc2C(=O)N2CCN(C(=O)COc3ccc(OC(F)(F)F)cc3I)C[C@@H]12]

This example is directed to the synthesis of (S)-2-(3,5-bis(trifluoromethyl)phenyl)-8-(2-(2-iodo-4-(trifluoromethoxy)phenoxy)acetyl)-7,8,9,10-tetrahydropyrazino[1,2-a]pyrido[3,2-e][1,4]diazepine-6,12 (5H, 6aH)-dione (KJW012-100) in an aspect of the invention.

The compound was prepared following General Procedure C using (S)-2-(3,5-bis(trifluoromethyl)phenyl)-7,8,9,10-tetrahydropyrazino[1,2-a]pyrido[3,2-e][1,4]diazepine-6,12 (5H, 6aH)-dione 2,2,2-trifluoroacetate (KJW012-099) and 2-(2-iodo-4-(trifluoromethoxy)phenoxy)acetic acid (KJW012-049). ¹H NMR (400 MHz, Methanol-d₄) δ 8.78 (d, J=3.8 Hz, 2H), 8.28 (dd, J=8.6, 3.3 Hz, 1H), 8.04 (s, 1H), 7.76-7.57 (m, 2H), 7.24 (td, J=9.7, 9.2, 2.8 Hz, 1H), 7.08-7.00 (m, 1H), 5.18 (s, 0.67×2H), 5.06-4.89 (m, 0.33×2H), 4.60-4.27 (m, 2H), 4.26-3.82 (m, 3H), 3.76 (ddd, J=13.5, 8.8, 4.2 Hz, 1H), 3.54 (ddd, J=13.1, 8.7, 4.4 Hz, 1H). ¹⁹F NMR (376 MHz, Methanol-d₄) δ−60.13, −60.18, −64.32, −64.34. LCMS RT (Method 1)=5.984 min, m/z 789.0 [M+H⁺].

### Example 207

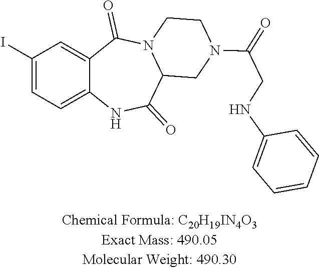 [SMILES: O=C1Nc2ccc(I)cc2C(=O)N2CCN(C(=O)CNc3ccccc3)CC12]

This example is directed to the synthesis of 8-iodo-2-(phenylglycyl)-1,3,4,12a-tetrahydrobenzo[e]pyrazino[1,2-a][1,4]diazepine-6,12 (2H, 11H)-dione (KJW006-061-1) in an aspect of the invention.

To a solution 2-(2-bromoacetyl)-8-iodo-1,3,4,12a-tetrahydrobenzo[e]pyrazino[1,2-a][1,4]diazepine-6,12 (2H, 11H)-dione (KJW006-059) (1 eq.) in DMF was added disopropylethylamine (2.5 eq.) followed by aniline (1.5 eq). The reaction was heated to 60° C. for 3 hours, diluted with EtOAc and washed twice with water, dried (Na₂SO₄), and concentrated in vacuo. The residue was purified by flash column chromatography (silica gel, 0-10% MeOH-DCM) to afford the title compound. ¹H NMR (400 MHz, DMSO-d₆) δ 10.61 (s, 0.64×1H), 10.53 (s, 0.36×1H), 7.60 (ddd, J=8.3, 3.7, 1.6 Hz, 1H), 7.53 (dd, J=8.3, 1.2 Hz, 2H), 7.12-7.02 (m, 2H), 6.65 (dq, J=7.7, 1.0 Hz, 2H), 6.59-6.48 (m, 1H), 4.31 (dt, J=11.3, 4.7 Hz, 1H), 4.25-3.90 (m, 3H), 3.89-3.24 (m, 6H). LCMS RT (Method 1)=4.119 min, m/z 491.1 [M+H⁺).

### Example 208

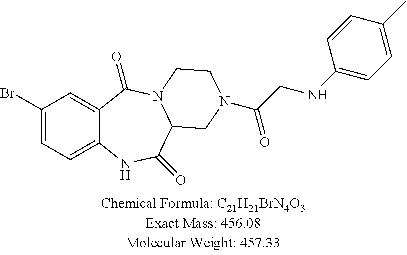 [SMILES: Cc1ccc(NCC(=O)N2CCN3C(=O)c4cc(Br)ccc4NC(=O)C3C2)cc1]

This example is directed to the synthesis of 8-bromo-2-(p-tolylglycyl)-1,3,4,12a-tetrahydrobenzo[e]pyrazino[1,2-a][1,4]diazepine-6,12 (2H, 11H)-dione (KJW009-022) in an aspect of the invention.

The compound was prepared following General Procedure C, substituting p-tolylglycine (1.3 eq.) for the 2-phenoxyacetic acid. LCMS RT (Method 3)=2.87 min, m/z 457.1 [M⁺).

### Example 209

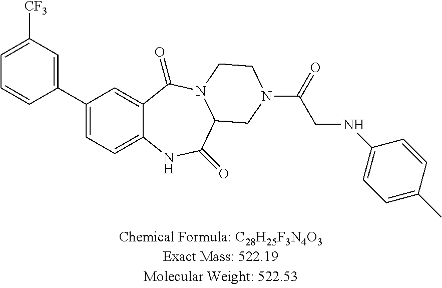 [SMILES: Cc1ccc(NCC(=O)N2CCN3C(=O)c4cc(-c5cccc(C(F)(F)F)c5)ccc4NC(=O)C3C2)cc1]

This example is directed to the synthesis of 2-(p-tolylglycyl)-8-(3-(trifluoromethyl)phenyl)-1,3,4,12a-tetrahydrobenzo[e]pyrazino[1,2-a][1,4]diazepine-6,12 (2H, 11H)-dione (KJW009-023) in an aspect of the invention.

The compound was prepared following General Procedure G using 8-bromo-2-(p-tolylglycyl)-1,3,4,12a-tetrahydrobenzo[e]pyrazino[1,2-a][1,4]diazepine-6,12 (2H, 11H)-dione (Example 208) and (3-(trifluoromethyl)phenyl)boronic acid. ¹H NMR (400 MHz, DMSO-d₆) δ 10.76 (s, 0.67×1H), 10.68 (s, 0.33×1H), 8.09 (d, J=2.3 Hz, 1H), 8.06-7.90 (m, 3H), 7.73 (d, J=8.1 Hz, 2H), 7.26 (d, J=8.5 Hz, 1H), 6.88 (dd, J=12.2, 8.0 Hz, 2H), 6.57 (t, J=7.2 Hz, 2H), 4.36 (dd, J=10.4, 5.3 Hz, 2H), 4.28-3.54 (m, 7H),) 2.15 (s, 0.36×3H), 2.13 (s, 0.64×3H). ¹⁹F NMR (376 MHz, DMSO-d₆) δ−61.04, −61.06. LCMS RT (Method 1)=5.094 min, m/z 523.1 [M+H⁺).

### Example 210

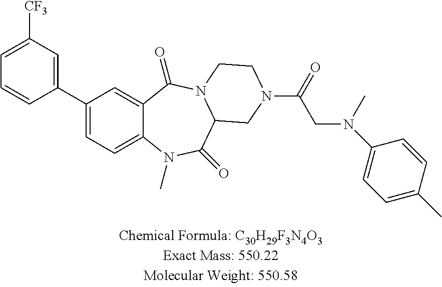 [SMILES: Cc1ccc(N(C)CC(=O)N2CCN3C(=O)c4cc(-c5cccc(C(F)(F)F)c5)ccc4N(C)C(=O)C3C2)cc1]

This example is directed to the synthesis of 11-methyl-2-(N-methyl-N-(p-tolyl)glycyl)-8-(3-(trifluoromethyl)phenyl)-1,3,4,12a-tetrahydrobenzo[e]pyrazino[1,2-a][1,4]diazepine-6,12 (2H, 11H)-dione (KJW009-025) in an aspect of the invention.

To a slurry of 2-(p-tolylglycyl)-8-(3-(trifluoromethyl)phenyl)-1,3,4,12a-tetrahydrobenzo[e]pyrazino[1,2-a][1,4]diazepine-6,12 (2H, 11H)-dione (KJW009-023) in DMF was added K₂CO₃ (3 eq.) followed by methyl iodide (3 eq.), and the resulting mixture was allowed to stir 18 hours at RT. The reaction mixture was diluted with EtOAc and washed twice with NaHCO₃. The EtOAc was dried (Na₂SO₄) and concentrated under vacuum. The residue was purified by flash column chromatography (silica gel, 0-10% MeOH-DCM) to afford the title compound. ¹⁹F NMR (376 MHz, DMSO-d₆) δ−61.02, −61.03. LCMS RT (Method 1)=5.526 min, m/z 551.1 [M+H⁺).

### Example 211

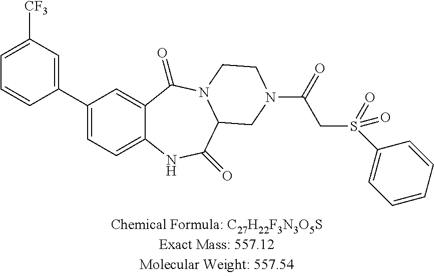 [SMILES: O=C1Nc2ccc(-c3cccc(C(F)(F)F)c3)cc2C(=O)N2CCN(C(=O)CS(=O)(=O)c3ccccc3)CC12]

This example is directed to the synthesis of 2-(2-(phenylsulfonyl)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW009-049) in an aspect of the invention.

The compound was prepared following General Procedure C, substituting 2-(phenylsulfonyl)acetic acid (1.3 eq.) for the 2-phenoxyacetic acid. The product 8-bromo-2-(2-(phenylsulfonyl)acetyl)-1,3,4,12a-tetrahydrobenzo[e]pyrazino[1,2-a][1,4]diazepine-6,12 (2H, 11H)-dione was used without purification and allowed to react (3-(trifluoromethyl)phenyl)boronic acid using General Procedure G. The reaction mixture was purified by flash column chromatography (silica gel, 0-10% MeOH-DCM) to afford the title compound. ¹H NMR (400 MHz, DMSO-d₆) δ 10.76 (s, 0.65×1H), 10.69 (s, 0.35×1H), 8.08 (dd, J=7.2, 2.3 Hz, 1H), 8.06-7.99 (m, 2H), 7.96 (d, J=8.3 Hz, 2H), 7.94-7.89 (m, 1H), 7.77-7.68 (m, 3H), 7.63 (t, J=7.6 Hz, 2H), 7.25 (dd, J=8.4, 2.4 Hz, 1H), 4.87-4.60 (m, 2H), 4.36-4.06 (m, 3H), 3.96-3.65 (m, 2H), 3.58 (ddt, J=13.8, 8.7, 5.0 Hz, 1H), 3.52-3.34 (m, 1H). ¹⁹F NMR (376 MHz, DMSO-d₆) δ−61.04. LCMS RT (Method 2)=3.145 min, m/z 558.1 [M+H⁺).

### Example 212

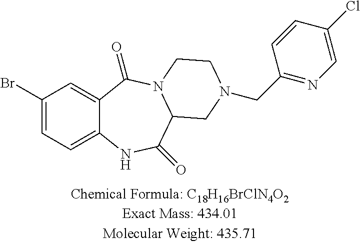 [SMILES: O=C1Nc2ccc(Br)cc2C(=O)N2CCN(Cc3ccc(Cl)cn3)CC12]

This example is directed to the synthesis of 8-bromo-2-((5-chloropyridin-2-yl)methyl)-1,3,4,12a-tetrahydrobenzo[e]pyrazino[1,2-a][1,4]diazepine-6,12 (2H, 11H)-dione (KJW008-051-1) in an aspect of the invention.

The compound was prepared following General Procedure F using 4-chloro-2-(chloromethyl)pyridine. ¹H NMR (400 MHz, Chloroform-d) δ 8.72 (s, 1H), 8.42 (dd, J=5.4, 0.6 Hz, 1H), 8.04-7.93 (m, 2H), 7.54 (dd, J=8.6, 2.4 Hz, 1H), 7.19 (dd, J=5.4, 2.1 Hz, 1H), 6.90 (d, J=8.6 Hz, 1H), 4.54 (ddd, J=13.6, 3.6, 2.0 Hz, 1H), 4.08 (dd, J=5.1, 1.6 Hz, 1H), 3.94-3.63 (m, 2H), 3.38 (dt, J=12.3, 1.9 Hz, 1H), 3.27 (ddd, J=13.6, 12.2, 4.0 Hz, 1H), 3.00 (ddt, J=11.5, 4.0, 2.0 Hz, 1H), 2.50-2.32 (m, 2H). LCMS RT (Method 2)=2.567 min, m/z 435.0 [M⁺].

### Example 213

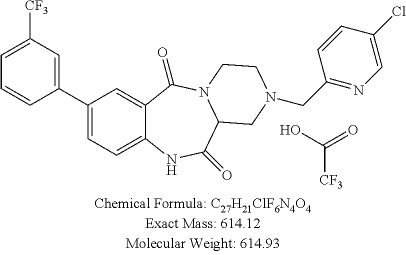

This example is directed to the synthesis of 2-((5-chloropyridin-2-yl)methyl)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate(KJW008-060) in an aspect of the invention.

The compound was prepared following General Procedure G using 8-bromo-2-((5-chloropyridin-2-yl)methyl)-1,3,4,12a-tetrahydrobenzo[e]pyrazino[1,2-a][1,4]diazepine-6,12 (2H, 11H)-dione (KJW008-051-1). ¹H NMR (600 MHz, DMSO-d₆) δ 10.85 (s, 1H), 8.61 (d, J=5.4 Hz, 1H), 8.06 (d, J=2.4 Hz, 1H), 8.03-7.95 (m, 4H), 7.77-7.70 (m, 2H), 7.62-7.58 (dt, J=6.6, 3.4 Hz, 1H), 7.23 (d, J=8.4 Hz, 1H), 4.59-4.35 (m, 3H), 4.34-4.23 (m, 1H), 3.62 (bs, 1H), 3.49-3.29 (m, 2H), 3.22-2.93 (m, 2H). ¹⁹F NMR (564 MHz, DMSO-d₆) δ−61.03, −74.07. LCMS RT (Method 1)=4.523 min, m/z 501.1 [M+H⁺].

### Example 214

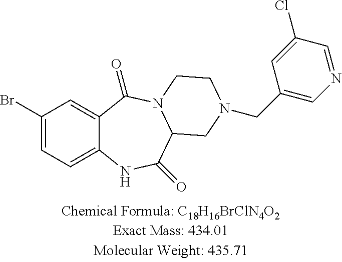 [SMILES: O=C1Nc2ccc(Br)cc2C(=O)N2CCN(Cc3cncc(Cl)c3)CC12]

This example is directed to the synthesis of 8-bromo-2-((5-chloropyridin-3-yl)methyl)-1,3,4,12a-tetrahydrobenzo[e]pyrazino[1,2-a][1,4]diazepine-6,12 (2H, 11H)-dione (KJW-008-051-3) in an aspect of the invention.

The compound was prepared following General Procedure F using 3-chloro-5-(chloromethyl)pyridine. ¹H NMR (400 MHz, DMSO-d₆) δ 10.49 (s, 1H), 8.62-8.45 (m, 2H), 8.22 (dd, J=2.5, 1.8 Hz, 1H), 7.79 (d, J=2.4 Hz, 1H), 7.67 (dd, J=8.6, 2.5 Hz, 1H), 7.06-6.98 (m, 1H), 4.34-4.26 (m, 1H), 4.22-4.17 (m, 1H), 3.82 (d, J=14.8 Hz, 1H), 3.52 (d, J=14.8 Hz, 1H), 3.15-3.08 (m, 1H), 3.08-2.88 (m, 2H), 2.29-2.10 (m, 2H). LCMS RT (Method 2)=2.653 min, m/z 434.8 [M+H⁺].

### Example 215

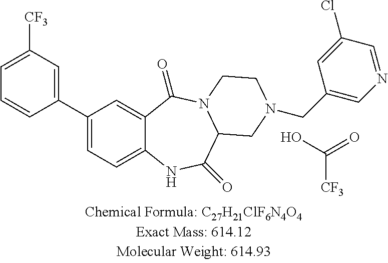

This example is directed to the synthesis of 2-((5-chloropyridin-3-yl)methyl)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW008-062) in an aspect of the invention.

The compound was prepared following General Procedure G using 8-bromo-2-((5-chloropyridin-3-yl)methyl)-1,3,4,12a-tetrahydrobenzo[e]pyrazino[1,2-a][1,4]diazepine-6,12 (2H, 11H)-dione (KJW-008-051-3). ¹H NMR (400 MHz, DMSO-d₆) δ 10.54 (s, 1H), 8.64-8.59 (m, 1H), 8.55 (s, 1H), 8.24 (s, 1H), 8.11-7.97 (m, 4H), 7.93 (d, J=8.5 Hz, 1H), 7.73 (d, J=8.1 Hz, 2H), 7.21 (d, J=8.5 Hz, 1H), 4.47-4.14 (m, 2H), 3.89-3.81 (m, 1H), 3.14-2.18 (m, 5H). ¹⁹F NMR (376 MHz, DMSO-d₆) δ−61.04, −73.65. LCMS RT (Method 1)=4.538 min, m/z 501.1 [M+H].

### Example 216

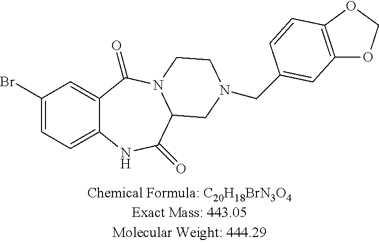 [SMILES: O=C1Nc2ccc(Br)cc2C(=O)N2CCN(Cc3ccc4c(c3)OCO4)CC12]

This example is directed to the synthesis of 2-(benzo[d][1,3]dioxol-5-ylmethyl)-8-bromo-1,3,4,12a-tetrahydrobenzo[e]pyrazino[1,2-a][1,4]diazepine-6,12 (2H, 11H)-dione (KJW008-076) in an aspect of the invention.

To a slurry of 8-bromo-1,3,4,12a-tetrahydrobenzo[e]pyrazino[1,2-a][1,4]diazepine-6,12 (2H, 11H)-dione 2,2,2-trifluoroacetate (1 eq.) (KJW008-042) in dichloroethane (DCE) was added benzo[d][1,3]dioxole-5-carbaldehyde (2 eq.) followed by sodium cyanotrihydroborate (2.2 eq.). The reaction was allowed to stir for 18 hours, heated to 45° C. for 4 hours and then stirred another 18 hours. The reaction was diluted with 10% MeOH-DCM, filtered thru CELITE™, concentrated under vacuum, and purified by silica gel chromatography (0-5% MeOH DCM) to afford the title compound. LCMS RT (Method 2)=2.651 min, m/z 445.0 (M+H⁺].

### Example 217

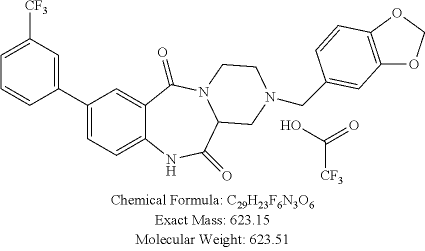

This example is directed to the synthesis of 2-(benzo[d][1,3]dioxol-5-ylmethyl)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate(KJW008-081) in an aspect of the invention.

The compound was prepared following General Procedure G using 2-(benzo[d][1,3]dioxol-5-ylmethyl)-8-bromo-1,3,4,12a-tetrahydrobenzo[e]pyrazino[1,2-a][1,4]diazepine-6,12 (2H, 11H)-dione (KJW008-076). LCMS RT (Method 1)=4.474 min, m/z 510.1 [M+H⁺].

### Example 218

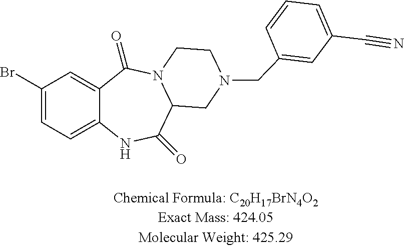 [SMILES: N#Cc1cccc(CN2CCN3C(=O)c4cc(Br)ccc4NC(=O)C3C2)c1]

This example is directed to the synthesis of 3-((8-Bromo-6,12-dioxo-3,4,6,11,12,12a-hexahydrobenzo[e]pyrazino[1,2-a][1,4]diazepin-2 (1H)-yl)methyl)benzonitrile (KJW008-077) in an aspect of the invention.

To a slurry of 8-bromo-1,3,4,12a-tetrahydrobenzo[e]pyrazino[1,2-a][1,4]diazepine-6,12 (2H, 11H)-dione 2,2,2-trifluoroacetate (1 eq.) (KJW008-042) in dichloroethane (DCE) was added 3-formylbenzonitrilecarbaldehyde (2 eq.) followed by sodium cyanotrihydroborate (2.2 eq.). The reaction was allowed to stir for 18 hours, heated to 45° C. for 4 hours and then stirred another 18 hours. The reaction was diluted with 10% MeOH-DCM, filtered thru CELITE™, concentrated under vacuum, and purified by silica gel chromatography (0-5% MeOH DCM) to afford the title compound. ¹H NMR (400 MHz, DMSO-d₆) δ 10.47 (s, 1H), 8.15-8.09 (m, 1H), 7.79 (d, J=2.4 Hz, 1H), 7.77 (dt, J=7.6, 1.4 Hz, 1H), 7.72 (dt, J=7.8, 1.4 Hz, 1H), 7.67 (dd, J=8.7, 2.4 Hz, 1H), 7.54 (td, J=7.7, 0.6 Hz, 1H), 7.08-6.93 (m, 1H), 4.36-4.27 (m, 1H), 4.24-4.14 (m, 1H), 3.66 (dd, J=110.7, 14.7 Hz, 2H), 3.17-3.10 (m, 1H), 3.02 (td, J=12.8, 3.9 Hz, 1H), 2.95-2.88 (m, 1H), 2.21 (td, J=11.7, 3.5 Hz, 1H), 2.14 (dd, J=12.2, 5.2 Hz, 1H). LCMS RT (Method 2)=2.623 min, m/z 425.9 [M+H⁺].

### Example 219

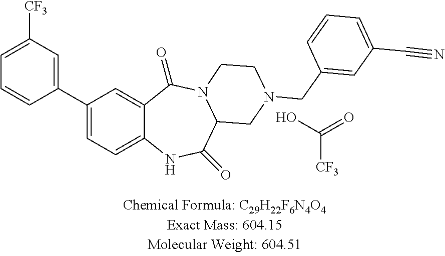

This example is directed to the synthesis of 3-((6,12-dioxo-8-(3-(trifluoromethyl)phenyl)-3,4,6,11,12,12a-hexahydrobenzo[e]pyrazino[1,2-a][1,4]diazepin-2 (1H)-yl)methyl)benzonitrile 2,2,2-trifluoroacetate (KJW008-082) in an aspect of the invention.

The compound was prepared following General Procedure G using 3-((8-bromo-6,12-dioxo-3,4,6,11,12,12a-hexahydrobenzo[e]pyrazino[1,2-a][1,4]diazepin-2 (1H)-yl)methyl)benzonitrile (KJW008-077). ¹⁹F NMR (564 MHz, DMSO-d₆) δ−61.02, −73.96. LCMS RT (Method 1)=4.443 min, m/z 491.1 [M+H⁺].

### Example 220

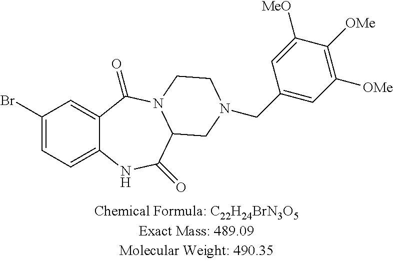 [SMILES: COc1cc(CN2CCN3C(=O)c4cc(Br)ccc4NC(=O)C3C2)cc(OC)c1OC]

This example is directed to the synthesis of 8-bromo-2-(3,4,5-trimethoxybenzyl)-1,3,4,12a-tetrahydrobenzo[e]pyrazino[1,2-a][1,4]diazepine-6,12 (2H, 11H)-dione (KJW008-086) in an aspect of the invention.

To a slurry of 8-bromo-1,3,4,12a-tetrahydrobenzo[e]pyrazino[1,2-a][1,4]diazepine-6,12 (2H, 11H)-dione 2,2,2-trifluoroacetate (1 eq.) (KJW008-042) in dichloroethane (DCE) was added 3,4,5-trimethoxybenzaldehyde (2 eq.) followed by sodium cyanotrihydroborate (2.2 eq.). The reaction was allowed to stir for 18 hours, heated to 45° C. for 4 hours and then stirred another 18 hours. The reaction was diluted with 10% MeOH-DCM, filtered thru CELITE™, concentrated under vacuum, and purified by silica gel chromatography (0-5% MeOH DCM) to afford the title compound. LCMS RT (Method 2)=2.654 min, m/z 490.1 [M+H⁺].

### Example 221

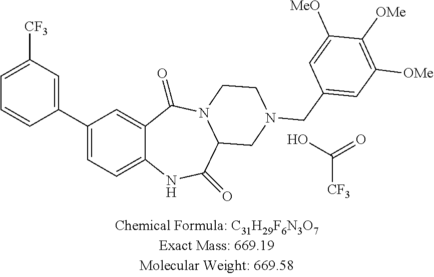

This example is directed to the synthesis of 8-(3-(trifluoromethyl)phenyl)-2-(3,4,5-trimethoxybenzyl)-1,3,4,12a-tetrahydrobenzo[e]pyrazino[1,2-a][1,4]diazepine-6,12 (2H, 11H)-dione 2,2,2-trifluoroacetate (KJW008-090) in an aspect of the invention.

The compound was prepared following General Procedure G using 8-bromo-2-(3,4,5-trimethoxybenzyl)-1,3,4,12a-tetrahydrobenzo[e]pyrazino[1,2-a][1,4]diazepine-6,12 (2H, 11H)-dione (KJW008-086). ¹H NMR (400 MHz, DMSO-d₆) δ 10.48 (s, 1H), 7.79 (d, J=2.5 Hz, 1H), 7.67 (dd, J=8.7, 2.5 Hz, 1H), 7.02 (d, J=8.7 Hz, 1H), 6.89 (s, 2H), 6.70-6.51 (m, 2H), 5.14 (t, J=5.8 Hz, 1H), 4.42 (dt, J=5.8, 0.7 Hz, 2H), 4.32 (d, J=13.2 Hz, 1H), 4.19 (d, J=4.8 Hz, 1H), 3.82 (s, 3H), 3.76 (s, 3H), 3.65 (s, 3H), 3.58 (m, 3H), 3.30-2.93 (m, 3H). LCMS RT (Method 1)=4.691 min, m/z 556.2 [M+H⁺].

### Example 222

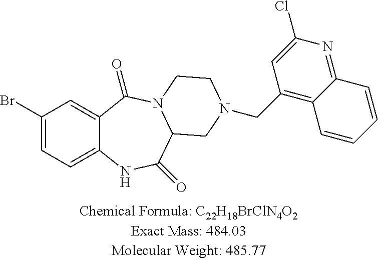 [SMILES: O=C1Nc2ccc(Br)cc2C(=O)N2CCN(Cc3cc(Cl)nc4ccccc34)CC12]

This example is directed to the synthesis of 8-bromo-2-((2-chloroquinolin-4-yl)methyl)-1,3,4,12a-tetrahydrobenzo[e]pyrazino[1,2-a][1,4]diazepine-6,12 (2H, 11H)-dione (KJW008-051-2) in an aspect of the invention.

The compound was prepared following General Procedure F using 2-chloro-4-(chloromethyl)quinoline. ¹H NMR (400 MHz, DMSO-d₆) δ 10.60 (s, 1H), 8.35 (d, J=1.0 Hz, 1H), 8.27 (ddd, J=8.5, 1.5, 0.7 Hz, 1H), 7.96 (ddd, J=8.5, 1.4, 0.6 Hz, 1H), 7.88-7.74 (m, 2H), 7.73-7.60 (m, 2H), 7.04 (d, J=8.7 Hz, 1H), 4.37-4.23 (m, 2H), 4.22-4.02 (m, 2H), 3.08-2.95 (m, 2H), 2.89 (d, J=0.5 Hz, 1H), 2.73 (d, J=0.7 Hz, 1H), 2.48-2.23 (m, 1H). LCMS RT (Method 2)=3.034 min. m/z 485.0 [M⁺].

### Example 223

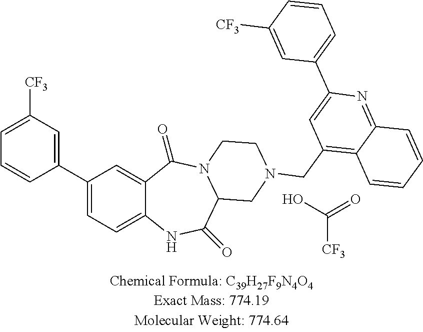

This example is directed to the synthesis of 8-(3-(trifluoromethyl)phenyl)-2-((2-(3-(trifluoromethyl)phenyl)quinolin-4-yl)methyl)-1,3,4,12a-tetrahydrobenzo[e]pyrazino[1,2-a][1,4]diazepine-6,12 (2H, 11H)-dione 2,2,2-trifluoroacetate (KJW008-061) in an aspect of the invention.

The compound was prepared following General Procedure G using 8-bromo-2-((2-chloroquinolin-4-yl)methyl)-1,3,4,12a-tetrahydrobenzo[e]pyrazino[1,2-a][1,4]diazepine-6,12 (2H, 11H)-dione (KJW008-051-2). ¹H NMR (400 MHz, DMSO-d₆) δ 10.52 (s, 1H), 8.84 (s, 1H), 8.80 (d, J=7.9 Hz, 1H), 8.76 (s, 1H), 8.34 (d, J=8.4 Hz, 1H), 8.16 (d, J=8.4 Hz, 1H), 8.05 (d, J=2.2 Hz, 1H), 8.04-7.99 (m, 3H), 7.96-7.87 (m, 3H), 7.81 (t, J=7.8 Hz, 2H), 7.72 (d, J=7.8 Hz, 2H), 7.64 (t, J=7.6 Hz, 1H), 7.25 (d, J=8.4 Hz, 1H), 4.47-4.39 (m, 1H), 4.33 (d, J=4.8 Hz, 1H), 4.28-4.12 (m, 2H), 3.49 (d, J=12.3 Hz, 1H), 3.17-3.03 (m, 2H), 2.47-2.29 (m, 2H). ¹⁹F NMR (376 MHz, DMSO-d₆) δ−60.96, −61.04, −73.48. LCMS RT (Method 1)=6.152 min, m/z 661.2 [M+H⁺].

### Example 224

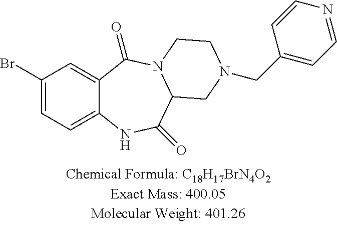 [SMILES: O=C1Nc2ccc(Br)cc2C(=O)N2CCN(Cc3ccncc3)CC12]

This example is directed to the synthesis of 8-bromo-2-(pyridin-4-ylmethyl)-1,3,4,12a-tetrahydrobenzo[e]pyrazino[1,2-a][1,4]diazepine-6,12 (2H, 11H)-dione (KJW008-051-4) in an aspect of the invention.

The compound was prepared following General Procedure F using 4-(bromomethyl)pyridine hydrobromide. ¹H NMR (400 MHz, DMSO-d₆) δ 10.44 (s, 1H), 8.54-8.46 (m, 2H), 7.79 (d, J=2.5 Hz, 1H), 7.68 (dd, J=8.6, 2.4 Hz, 1H), 7.57-7.51 (m, 2H), 7.10-7.01 (m, 1H), 4.34-4.26 (m, 1H), 4.24-4.18 (m, 1H), 3.74 (d, J=15.2 Hz, 1H), 3.52 (d, J=15.2 Hz, 1H), 3.21-3.13 (m, 1H), 3.00 (td, J=12.8, 3.8 Hz, 1H), 2.92-2.84 (m, 1H), 2.17 (td, J=12.1, 11.6, 4.1 Hz, 2H). LCMS RT (Method 2)=2.519 min, m/z 400.9 [M+H⁺].

### Example 225

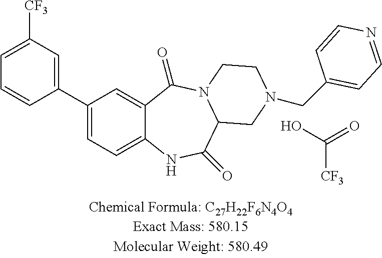

This example is directed to the synthesis of 2-(pyridin-4-ylmethyl)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW008-063) in an aspect of the invention.

The compound was prepared following General Procedure G using 8-bromo-2-(pyridin-4-ylmethyl)-1,3,4,12a-tetrahydrobenzo[e]pyrazino[1,2-a][1,4]diazepine-6,12 (2H, 11H)-dione (KJW008-051-4). ¹⁹F NMR (376 MHz, DMSO-d₆) δ−61.04, −73.80, −73.81. LCMS RT (Method 1)=4.275 min, m/z 467.2 [M+H⁺].

### Example 226

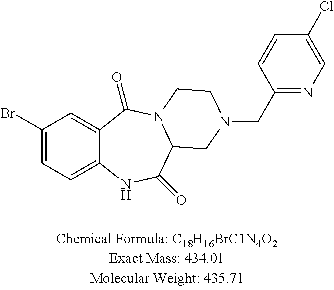 [SMILES: O=C1Nc2ccc(Br)cc2C(=O)N2CCN(Cc3ccc(Cl)cn3)CC12]

This example is directed to the synthesis of 8-bromo-2-((5-chloropyridin-2-yl)methyl)-1,3,4,12a-tetrahydrobenzo[e]pyrazino[1,2-a][1,4]diazepine-6,12 (2H, 11H)-dione (KJW008-051-1) in an aspect of the invention.

The compound was prepared following General Procedure F using 5-chloro-2-(chloromethyl)pyridine. ¹H NMR (400 MHz, Chloroform-d) δ 8.72 (s, 1H), 8.42 (dd, J=5.4, 0.6 Hz, 1H), 8.04-7.95 (m, 2H), 7.54 (dd, J=8.6, 2.4 Hz, 1H), 7.19 (dd, J=5.4, 2.1 Hz, 1H), 6.90 (d, J=8.6 Hz, 1H), 4.54 (ddd, J=13.6, 3.6, 2.0 Hz, 1H), 4.08 (dd, J=5.1, 1.6 Hz, 1H), 3.90 (d, J=15.1 Hz, 1H), 3.71 (d, J=15.1 Hz, 1H), 3.38 (dt, J=12.3, 1.9 Hz, 1H), 3.27 (ddd, J=13.6, 12.2, 4.0 Hz, 1H), 3.06-2.98 (m, 1H), 2.95 (d, J=0.5 Hz, 1H), 2.88 (d, J=0.6 Hz, 1H), 2.49-2.34 (m, 2H). LCMS RT (Method 2)=2.567 min, m/z 435.0 [M⁺].

### Example 227

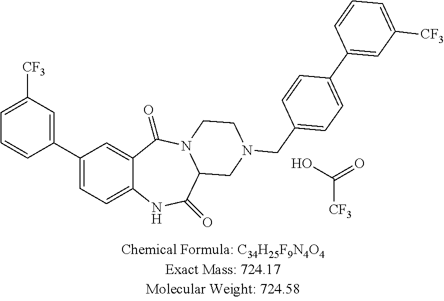

This example is directed to the synthesis of 8-(3-(trifluoromethyl)phenyl)-2-((5-(3-(trifluoromethyl)phenyl)pyridin-2-yl)methyl)-1,3,4,12a-tetrahydrobenzo[e]pyrazino[1,2-a][1,4]diazepine-6,12 (2H, 11H)-dione 2,2,2-trifluoroacetate (KJW008-064) in an aspect of the invention.

The compound was prepared following General Procedure G using 8-bromo-2-((5-chloropyridin-2-yl)methyl)-1,3,4,12a-tetrahydrobenzo[e]pyrazino[1,2-a][1,4]diazepine-6,12 (2H, 11H)-dione (KJW008-051-1). LCMS RT (Method 1)=5.298 min, m/z 611.2 [M+H].

### Example 228

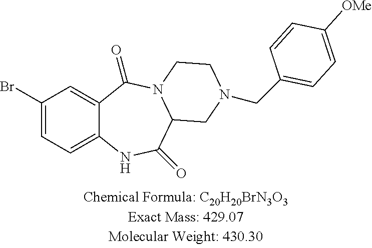 [SMILES: COc1ccc(CN2CCN3C(=O)c4cc(Br)ccc4NC(=O)C3C2)cc1]

This example is directed to the synthesis of 8-bromo-2-(4-methoxybenzyl)-1,3,4,12a-tetrahydrobenzo[e]pyrazino[1,2-a][1,4]diazepine-6,12 (2H, 11H)-dione (KJW008-070) in an aspect of the invention.

To a slurry of 8-bromo-1,3,4,12a-tetrahydrobenzo[e]pyrazino[1,2-a][1,4]diazepine-6,12 (2H, 11H)-dione 2,2,2-trifluoroacetate (1 eq.) (KJW008-042) in dichloroethane (DCE) was added 4-methoxybenzaldehyde (2 eq.) followed by sodium cyanotrihydroborate (2.2 eq.). The reaction was allowed to stir for 18 hours, heated to 45° C. for 4 hours and then stirred another 18 hours. The reaction was diluted with 10% MeOH-DCM, filtered thru CELITE™, concentrated under vacuum, and purified by silica gel chromatography (0-5% MeOH DCM) to afford the title compound. ¹H NMR (400 MHz, Chloroform-d) δ 9.00 (s, 1H), 8.01 (d, J=2.4 Hz, 1H), 7.59-7.49 (m, 1H), 7.39-7.30 (m, 2H), 6.89-6.80 (m, 3H), 4.50 (ddd, J=13.6, 3.7, 2.0 Hz, 1H), 4.03 (dd, J=5.3, 1.7 Hz, 1H), 3.78 (s, 3H), 3.68 (d, J=13.2 Hz, 1H), 3.54-3.46 (m, 1H), 3.35 (dt, J=12.1, 1.9 Hz, 1H), 3.21 (ddd, J=13.5, 12.1, 4.1 Hz, 1H), 3.02-2.93 (m, 1H), 2.24 (td, J=11.8, 4.3 Hz, 2H). LCMS RT (Method 2)=2.687 min, m/z 429.9 [M⁺].

### Example 229

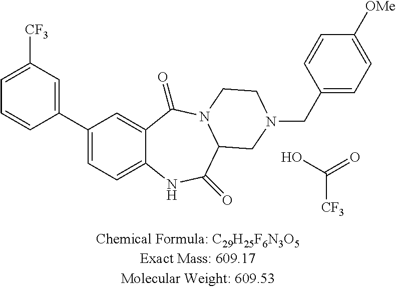

This example is directed to the synthesis of 2-(4-methoxybenzyl)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW008-075) in an aspect of the invention.

The compound was prepared following General Procedure G using 8-bromo-2-(4-methoxybenzyl)-1,3,4,12a-tetrahydrobenzo[e]pyrazino[1,2-a][1,4]diazepine-6,12 (2H, 11H)-dione (KJW008-070). ¹⁹F NMR (376 MHz, DMSO-d₆) δ−61.04, −73.61. LCMS RT (Method 1)=4.522 min, m/z 496.1 [M+H⁺].

### Example 230

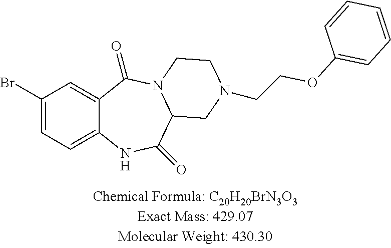 [SMILES: O=C1Nc2ccc(Br)cc2C(=O)N2CCN(CCOc3ccccc3)CC12]

This example is directed to the synthesis of 8-bromo-2-(2-phenoxyethyl)-1,3,4,12a-tetrahydrobenzo[e]pyrazino[1,2-a][1,4]diazepine-6,12 (2H, 11H)-dione (KJW011-007) in an aspect of the invention.

The compound was prepared following General Procedure F using 2-bromoethoxy-benzene. ¹H NMR (400 MHz, Chloroform-d) δ 8.94 (s, 1H), 8.01 (d, J=2.6 Hz, 1H), 7.42 (dq, J=9.0, 2.1 Hz, 1H), 7.28 (dt, J=9.0, 4.5 Hz, 3H), 6.96 (t, J=7.5 Hz, 1H), 6.88 (ddd, J=18.8, 8.6, 2.3 Hz, 4H), 4.51 (dd, J=13.9, 3.2 Hz, 1H), 4.23 (dt, J=7.3, 3.6 Hz, 2H), 4.10-4.00 (m, 1H), 3.57 (d, J=12.3 Hz, 1H), 3.24 (tt, J=13.5, 3.4 Hz, 1H), 3.12-2.98 (m, 2H), 2.98-2.84 (m, 1H), 2.48 (tq, J=12.2, 3.2 Hz, 3H). LCMS RT (Method 1)=4.863 min, m/z 429.7 [M⁺].

### Example 231

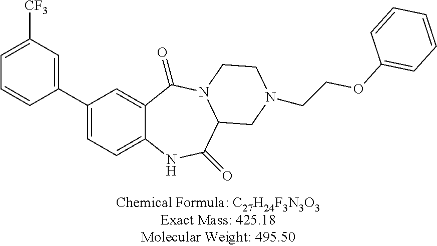 [SMILES: O=C1Nc2ccc(-c3cccc(C(F)(F)F)c3)cc2C(=O)N2CCN(CCOc3ccccc3)CC12]

This example is directed to the synthesis of 2-(2-phenoxyethyl)-8-(3-(trifluoromethyl)phenyl)-1,3,4,12a-tetrahydrobenzo[e]pyrazino[1,2-a][1,4]diazepine-6,12 (2H, 11H)-dione (KJW011-016) in an aspect of the invention.

The compound was prepared following General Procedure G, except purification was done by flash column chromatography (silica gel, 0-10% MeOH-DCM) to afford the title compound. ¹H NMR (400 MHz, DMSO-d₆) δ 10.49 (s, 1H), 8.06-7.97 (m, 3H), 7.95-7.88 (m, 1H), 7.72 (d, J=8.1 Hz, 2H), 7.29 (t, J=7.5 Hz, 2H), 7.21 (d, J=8.4 Hz, 1H), 6.94 (dd, J=15.8, 7.8 Hz, 3H), 4.32 (d, J=13.1 Hz, 1H), 4.27-4.20 (m, 1H), 4.21-4.07 (m, 2H), 3.32 (m, 1H), 3.02 (d, J=11.4 Hz, 2H), 2.84 (d, J=6.1 Hz, 2H), 2.38 (dd, J=12.6, 5.2 Hz, 1H), 2.34-2.21 (m, 1H). ¹⁹F NMR (376 MHz, DMSO-d₆) δ−61.02. LCMS RT (Method 1)=4.655 min, m/z 496.2 [M+H⁺].

### Example 232

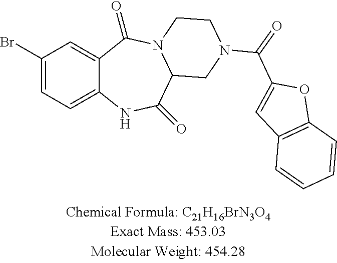 [SMILES: O=C1Nc2ccc(Br)cc2C(=O)N2CCN(C(=O)c3cc4ccccc4o3)CC12]

This example is directed to the synthesis of 2-(benzofuran-2-carbonyl)-8-bromo-1,3,4,12a-tetrahydrobenzo[e]pyrazino[1,2-a][1,4]diazepine-6,12 (2H, 11H)-dione (KJW010-051-2) in an aspect of the invention.

The compound was prepared following General Procedure H using benzofuran-2-carboxylic acid. ¹H NMR (400 MHz, DMSO-d₆) δ 10.62 (d, J=28.8 Hz, 1H), 7.87 (d, J=2.4 Hz, 1H), 7.79-7.65 (m, 3H), 7.54-7.42 (m, 2H), 7.38-7.30 (m, 1H), 7.07 (s, 1H), 4.58-3.55 (m, 7H). LCMS RT (Method 2)=3.032 min, m/z 456.0 [M+2H⁺].

### Example 233

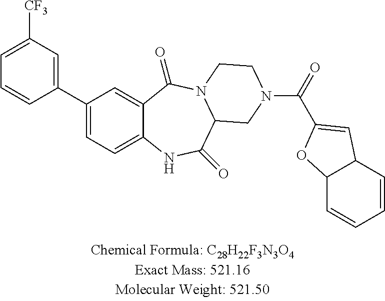 [SMILES: O=C1Nc2ccc(-c3cccc(C(F)(F)F)c3)cc2C(=O)N2CCN(C(=O)C3=CC4C=CC=CC4O3)CC12]

This example is directed to the synthesis of 2-(3a, 7a-dihydrobenzofuran-2-carbonyl)-8-(3-(trifluoromethyl)phenyl)-1,3,4,12a-tetrahydrobenzo[e]pyrazino[1,2-a][1,4]diazepine-6,12 (2H, 11H)-dione (KJW013-012) in an aspect of the invention.

The compound was prepared following General Procedure I using 2-(benzofuran-2-carbonyl)-8-bromo-1,3,4,12a-tetrahydrobenzo[e]pyrazino[1,2-a][1,4]diazepine-6,12 (2H, 11H)-dione (KJW010-051-2) and 3-trifluoromethylphenylboronic acid. ¹H NMR (400 MHz, Acetic Acid-d₄) δ 8.32 (d, J=2.3 Hz, 1H), 7.99 (s, 1H), 7.94 (t, J=9.8 Hz, 2H), 7.70 (dq, J=15.3, 8.2, 7.7 Hz, 3H), 7.60 (s, 2H), 7.50-7.42 (m, 1H), 7.31 (q, J=11.8, 9.6 Hz, 2H), 4.94-4.11 (m, 6H), 3.97 (s, 1H). ¹⁹F NMR (376 MHz, Acetic Acid-d₄)) δ−62.94. LCMS RT (Method 1)=5.444 min, m/z 521.1 [M⁺].

### Example 234

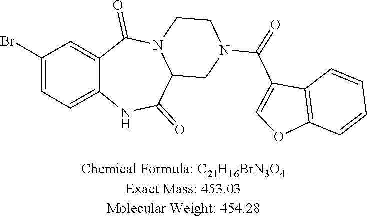 [SMILES: O=C1Nc2ccc(Br)cc2C(=O)N2CCN(C(=O)c3coc4ccccc34)CC12]

This example is directed to the synthesis of 2-(benzofuran-3-carbonyl)-8-bromo-1,3,4,12a-tetrahydrobenzo[e]pyrazino[1,2-a][1,4]diazepine-6,12 (2H, 11H)-dione (KJW010-051-1) in an aspect of the invention.

The compound was prepared following General Procedure H using benzofuran-3-carboxylic acid. ¹H NMR (400 MHz, DMSO-d₆) δ 10.62 (s, 1H), 8.43 (s, 1H), 7.87 (d, J=2.4 Hz, 1H), 7.82 (s, 1H), 7.75-7.69 (m, 1H), 7.66 (dt, J=8.2, 1.0 Hz, 1H), 7.43-7.29 (m, 2H), 7.07 (s, 1H), 4.37-3.51 (m, 7H). LCMS RT (Method 2)=2.972 min, m/z 456.0 [M+2H⁺).

### Example 235

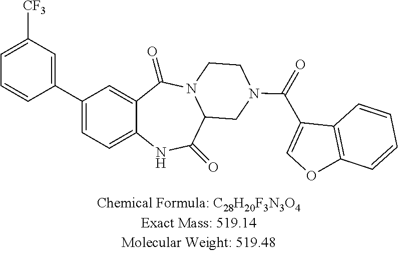 [SMILES: O=C1Nc2ccc(-c3cccc(C(F)(F)F)c3)cc2C(=O)N2CCN(C(=O)c3coc4ccccc34)CC12]

This example is directed to the synthesis of 2-(benzofuran-3-carbonyl)-8-(3-(trifluoromethyl)phenyl)-1,3,4,12a-tetrahydrobenzo[e]pyrazino[1,2-a][1,4]diazepine-6,12 (2H, 11H)-dione (KJW013-013) in an aspect of the invention.

The compound was prepared following General Procedure I using 2-(benzofuran-3-carbonyl)-8-bromo-1,3,4,12a-tetrahydrobenzo[e]pyrazino[1,2-a][1,4]diazepine-6,12 (2H, 11H)-dione (KJW010-051-1) and 3-(trifluoromethyl)phenyl)boronic acid. ¹H NMR (400 MHz, Acetic Acid-d₄) δ 8.31 (d, J=2.3 Hz, 1H), 8.19 (d, J=1.9 Hz, 1H), 7.98 (s, 1H), 7.93 (dd, J=13.9, 7.9 Hz, 2H), 7.85 (d, J=7.5 Hz, 1H), 7.69 (dt, J=15.3, 7.7 Hz, 2H), 7.56 (d, J=8.2 Hz, 1H), 7.43-7.30 (m, 3H), 4.57 (s, 2H), 4.17 (dd, J=14.2, 4.9 Hz, 3H), 4.01 (t, J=6.0 Hz, 2H). ¹⁹F NMR (376 MHz, Acetic Acid-d₄) δ−62.94. LCMS RT (Method 3)=3.300 min, m/z 520.2 [M+H⁺).

### Example 236

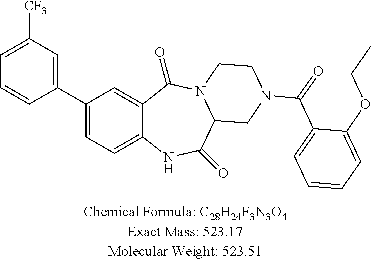 [SMILES: CCOc1ccccc1C(=O)N1CCN2C(=O)c3cc(-c4cccc(C(F)(F)F)c4)ccc3NC(=O)C2C1]

This example is directed to the synthesis of 2-(2-ethoxybenzoyl)-8-(3-(trifluoromethyl)phenyl)-1,3,4,12a-tetrahydrobenzo[e]pyrazino[1,2-a][1,4]diazepine-6,12 (2H, 11H)-dione (KJW008-056) in an aspect of the invention.

The compound was prepared following General Procedure I using 8-bromo-2-(2-ethoxybenzoyl)-1,3,4,12a-tetrahydrobenzo[e]pyrazino[1,2-a][1,4]diazepine-6,12 (2H, 11H)-dione (KJW008-054) and 3-trifluorophenylboronic acid. ¹H NMR (400 MHz, Acetic Acid-d₄) δ 8.31 (dd, J=11.2, 2.2 Hz, 1H), 8.02-7.85 (m, 3H), 7.75-7.64 (m, 2H), 7.55-7.20 (m, 3H), 7.10-6.90 (m, 2H), 4.67 (dd, J=14.6, 4.1 Hz, 1H), 4.37 (t, J=5.1 Hz, 1H), 4.24 (d, J=8.4 Hz, 1H), 4.14 (q, J=7.3 Hz, 2H), 4.08-3.97 (m, 3H), 3.92 (d, J=11.8 Hz, 1H), 1.23 (t, J=7.0 Hz, 3H). ¹⁹F NMR (376 MHz, Acetic Acid-d₄) δ−63.41. LCMS RT (Method 1)=5.396 min, m/z 524.2 [M+H⁺).

### Example 237

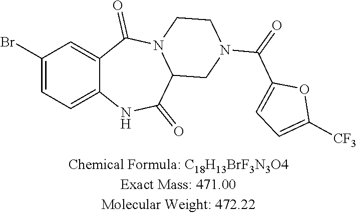 [SMILES: O=C1Nc2ccc(Br)cc2C(=O)N2CCN(C(=O)c3ccc(C(F)(F)F)o3)CC12]

This example is directed to the synthesis of 8-bromo-2-(5-(trifluoromethyl)furan-2-carbonyl)-1,3,4,12a-tetrahydrobenzo[e]pyrazino[1,2-a][1,4]diazepine-6,12 (2H, 11H)-dione (KJW008-068) in an aspect of the invention.

The compound was prepared following General Procedure H using 5-(trifluoromethyl)furan-2-carboxylic acid. ¹H NMR (400 MHz, DMSO-d₆) δ 10.68 (s, 0.45×1H), 10.62 (s, 0.55×1H), 7.86 (d, J=2.5 Hz, 1H), 7.72 (d, J=8.3 Hz, 1H), 7.42 (s, 1H), 7.25 (s, 1H), 7.08 (dd, J=19.2, 8.7 Hz, 1H), 4.45-4.15 (m, 3H), 4.07-3.75 (m, 3H), 3.60 (bs, 1H). ¹⁹F NMR (376 MHz, DMSO-d₆) δ−62.77 (s, 0.60×3F), −62.82 (s, 0.40×3F). LCMS RT (Method 2)=3.024 min, m/z 474.0 [M+2H⁺].

### Example 238

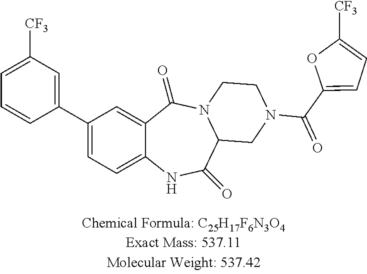 [SMILES: O=C1Nc2ccc(-c3cccc(C(F)(F)F)c3)cc2C(=O)N2CCN(C(=O)c3ccc(C(F)(F)F)o3)CC12]

This example is directed to the synthesis of 2-(5-(trifluoromethyl)furan-2-carbonyl)-8-(3-(trifluoromethyl)phenyl)-1,3,4,12a-tetrahydrobenzo[e]pyrazino[1,2-a][1,4]diazepine-6,12 (2H, 11H)-dione (KJW008-071) in an aspect of the invention.

The compound was prepared following General Procedure I using 8-bromo-2-(5-(trifluoromethyl)furan-2-carbonyl)-1,3,4,12a-tetrahydrobenzo[e]pyrazino[1,2-a][1,4]diazepine-6,12 (2H, 11H)-dione (KJW008-068) and 3-trifluoromethylphenylboronic acid. ¹H NMR (400 MHz, Acetic Acid-d₄) δ 8.31 (s, 1H), 7.98 (s, 1H), 7.93 (dd, J=12.6, 7.5 Hz, 2H), 7.73-7.65 (m, 2H), 7.31 (d, J=3.7 Hz, 2H), 7.09 (d, J=3.7 Hz, 1H), 4.79-4.45 (m, 2H), 4.37-4.10 (m, 4H), 3.97-3.90 (m, 1H). ¹⁹F NMR (376 MHz, Acetic Acid-d₄) δ−73.61, −61.04. LCMS RT (Method 1)=5.617 min, m/z 538.1 [M+H⁺].

### Example 239

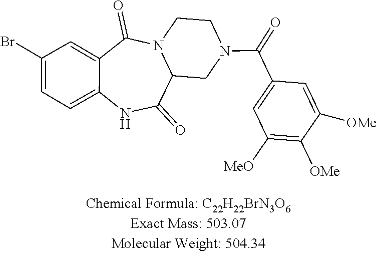 [SMILES: COc1cc(C(=O)N2CCN3C(=O)c4cc(Br)ccc4NC(=O)C3C2)cc(OC)c1OC]

This example is directed to the synthesis of 8-bromo-2-(3,4,5-trimethoxybenzoyl)-1,3,4,12a-tetrahydrobenzo[e]pyrazino[1,2-a][1,4]diazepine-6,12 (2H, 11H)-dione (KJW008-069) in an aspect of the invention.

The compound was prepared following General Procedure H using 3,4,5-trimethoxybenzoic acid. ¹H NMR (400 MHz, Chloroform-d) δ 8.14 (s, 1H), 8.09 (d, J=2.4 Hz, 1H), 7.60 (dd, J=8.6, 2.4 Hz, 1H), 6.88 (d, J=8.6 Hz, 1H), 6.76 (s, 2H), 4.35-4.03 (m, 4H), 3.85 (s, 3H), 3.69 (s, 6H), 3.75-3.69 (m, 3H). LCMS RT (Method 2)=2.915 min, m/z 504.9 [M+H⁺].

### Example 240

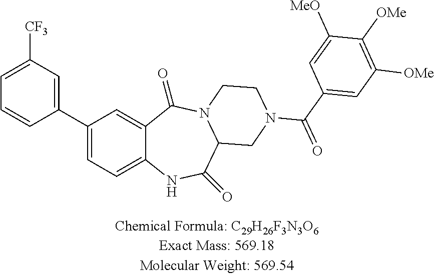 [SMILES: COc1cc(C(=O)N2CCN3C(=O)c4cc(-c5cccc(C(F)(F)F)c5)ccc4NC(=O)C3C2)cc(OC)c1OC]

This example is directed to the synthesis of 8-(3-(trifluoromethyl)phenyl)-2-(3,4,5-trimethoxybenzoyl)-1,3,4,12a-tetrahydrobenzo[e]pyrazino[1,2-a][1,4]diazepine-6,12 (2H, 11H)-dione (KJW008-072) in an aspect of the invention.

The compound was prepared following General Procedure I using 8-bromo-2-(5-(trifluoromethyl)furan-2-carbonyl)-1,3,4,12a-tetrahydrobenzo[e]pyrazino[1,2-a][1,4]diazepine-6,12 (2H, 11H)-dione (KJW008-068) and 3-trifluoromethylphenylboronic acid. ¹H NMR (400 MHz, Acetic Acid-d₄) δ 8.30 (d, J=2.2 Hz, 1H), 8.01-7.84 (m, 3H), 7.73-7.65 (m, 2H), 7.28 (s, 1H), 6.90 (s, 2H), 4.64-3.89 (m, 9H), 3.87 (s, 6H), 3.83 (s, 3H). ¹⁹F NMR (376 MHz, Acetic Acid-d₄) δ−57.30. LCMS RT (Method 1)=5.151 min, m/z 570.1 [M+H⁺).

### Example 241

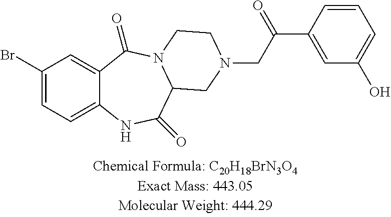 [SMILES: O=C(CN1CCN2C(=O)c3cc(Br)ccc3NC(=O)C2C1)c1cccc(O)c1]

This example is directed to the synthesis of 8-bromo-2-(2-(3-hydroxyphenyl)-2-oxoethyl)-1,3,4,12a-tetrahydrobenzo[e]pyrazino[1,2-a][1,4]diazepine-6,12 (2H, 11H)-dione (KJW008-091) in an aspect of the invention.

The compound was prepared following General Procedure J using 2-bromo-1-(3-hydroxyphenyl)ethan-1-one. LCMS RT (Method 2)=2.419 min, m/z 446.0 [M+2H⁺).

### Example 242

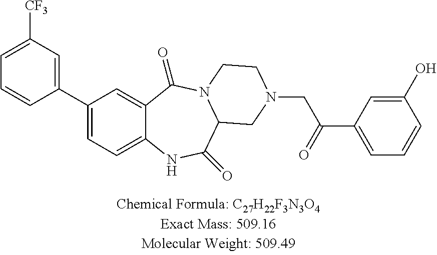 [SMILES: O=C(CN1CCN2C(=O)c3cc(-c4cccc(C(F)(F)F)c4)ccc3NC(=O)C2C1)c1cccc(O)c1]

This example is directed to the synthesis of 2-(2-(3-hydroxyphenyl)-2-oxoethyl)-8-(3-(trifluoromethyl)phenyl)-1,3,4,12a-tetrahydrobenzo[e]pyrazino[1,2-a][1,4]diazepine-6,12 (2H, 11H)-dione (KJW008-093) in an aspect of the invention.

The compound was prepared following General Procedure J using 8-bromo-2-(2-(3-hydroxyphenyl)-2-oxoethyl)-1,3,4,12a-tetrahydrobenzo[e]pyrazino[1,2-a][1,4]diazepine-6,12 (2H, 11H)-dione (KJW008-091). ¹H NMR (400 MHz, DMSO-d₆) δ 10.51 (s, 1H), 9.74 (s, 1H), 8.07-7.90 (m, 4H), 7.72 (d, J=8.0 Hz, 2H), 7.55 (d, J=7.7 Hz, 1H), 7.43-7.16 (m, 3H), 7.02 (dd, J=8.1, 2.4 Hz, 1H), 4.38-4.16 (m, 0.56×2H), 4.14-3.97 (m, 0.44×2H), 4.29-4.09 (m, 2H), 4.10-3.85 (m, 4H), 3.71 (dt, J=10.3, 5.9 Hz, 1H). ¹⁹F NMR (376 MHz, DMSO-d₆) δ−61.03. LCMS RT (Method 1)=4.332 min, m/z 510.1 [M+H⁺).

### Example 243

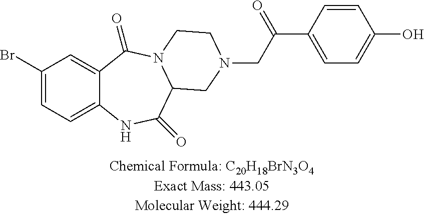 [SMILES: O=C(CN1CCN2C(=O)c3cc(Br)ccc3NC(=O)C2C1)c1ccc(O)cc1]

This example is directed to the synthesis of 8-Bromo-2-(2-(4-hydroxyphenyl)-2-oxoethyl)-1,3,4,12a-tetrahydrobenzo[e]pyrazino[1,2-a][1,4]diazepine-6,12 (2H, 11H)-dione (KJW008-092) in an aspect of the invention.

The compound was prepared following General Procedure J using 2-bromo-1-(4-hydroxyphenyl)ethan-1-one. LCMS RT (Method 2)=2.436 min, m/z 446.0 [M+2H⁺).

### Example 244

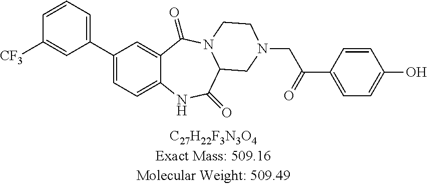 [SMILES: O=C(CN1CCN2C(=O)c3cc(-c4cccc(C(F)(F)F)c4)ccc3NC(=O)C2C1)c1ccc(O)cc1]

This example is directed to the synthesis of 2-(2-(4-hydroxyphenyl)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW008-095) in an aspect of the invention.

The compound was prepared following General Procedure J using 8-bromo-2-(2-(4-hydroxyphenyl)-2-oxoethyl)-1,3,4,12a-tetrahydrobenzo[e]pyrazino[1,2-a][1,4]diazepine-6,12 (2H, 11H)-dione (KJW008-092). ¹H NMR (400 MHz, DMSO-d₆) δ 10.51 (s, 1H), 10.33 (s, 1H), 8.07-7.96 (m, 5H), 7.93 (dt, J=8.6, 1.9 Hz, 1H), 7.78-7.66 (m, 2H), 7.22 (d, J=8.5 Hz, 1H), 6.87-6.75 (m, 2H), 4.34-4.20 (m, 2H), 4.04 (d, J=15.9 Hz, 1H), 3.69 (d, J=15.9 Hz, 1H), 3.36 (d, J=12.4 Hz, 1H), 3.02-2.87 (m, 2H), 2.59 (dd, J=12.6, 5.4 Hz, 1H), 2.39 (d, J=10.6 Hz, 1H). ¹⁹F NMR (376 MHz, DMSO-d₆) δ−61.04. LCMS RT (Method 1)=4.491 min. m/z 509.9 [M+H⁺).

### Example 245

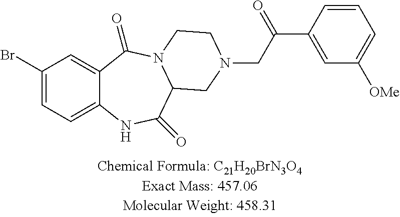 [SMILES: COc1cccc(C(=O)CN2CCN3C(=O)c4cc(Br)ccc4NC(=O)C3C2)c1]

This example is directed to the synthesis of 8-bromo-2-(2-(3-methoxyphenyl)-2-oxoethyl)-1,3,4,12a-tetrahydrobenzo[e]pyrazino[1,2-a][1,4]diazepine-6,12 (2H, 11H)-dione (KJW009-034-1) in an aspect of the invention.

The compound was prepared following General Procedure J using 2-bromo-1-(3-methoxyphenyl)ethan-1-one. LCMS RT (Method 2)=2.725 min, m/z 449.9 [M+2H⁺).

### Example 246

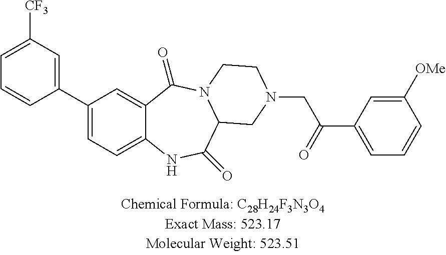 [SMILES: COc1cccc(C(=O)CN2CCN3C(=O)c4cc(-c5cccc(C(F)(F)F)c5)ccc4NC(=O)C3C2)c1]

This example is directed to the synthesis of 2-(2-(3-methoxyphenyl)-2-oxoethyl)-8-(3-(trifluoromethyl)phenyl)-1,3,4,12a-tetrahydrobenzo[e]pyrazino[1,2-a][1,4]diazepine-6,12 (2H, 11H)-dione (KJW009-037) in an aspect of the invention.

The compound was prepared following General Procedure J using 8-bromo-2-(2-(3-methoxyphenyl)-2-oxoethyl)-1,3,4,12a-tetrahydrobenzo[e]pyrazino[1,2-a][1,4]diazepine-6,12 (2H, 11H)-dione (KJW009-034-1). ¹H NMR (400 MHz, DMSO-d₆) δ 8.14-7.94 (m, 4H), 7.83-7.69 (m, 2H), 7.64-7.48 (m, 3H), 7.35-7.27 (m, 2H), 4.68 (s, 2H), 4.57 (d, J=4.6 Hz, 1H), 4.50-4.19 (m, 1H), 3.85 (s, 3H), 3.83-3.75 (m, 1H), 3.72-3.65 (m, 1H), 3.51-3.25 (m, 3H). ¹⁹F NMR (376 MHz, DMSO-d₆) δ−61.05. LCMS RT (Method 1)=4.732 min, m/z 524.2 [M+H⁺].

### Example 247

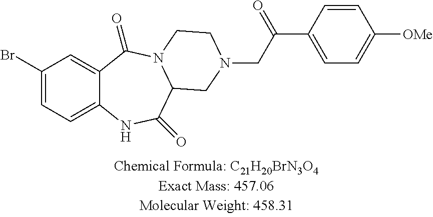 [SMILES: COc1ccc(C(=O)CN2CCN3C(=O)c4cc(Br)ccc4NC(=O)C3C2)cc1]

This example is directed to the synthesis of 8-bromo-2-(2-(4-methoxyphenyl)-2-oxoethyl)-1,3,4,12a-tetrahydrobenzo[e]pyrazino[1,2-a][1,4]diazepine-6,12 (2H, 11H)-dione (KJW034-1) in an aspect of the invention.

The compound was prepared following General Procedure J using 2-bromo-1-(4-methoxyphenyl)ethan-1-one. LCMS RT (Method 2)=2.710 min, m/z 459.9 [M+2H⁺).

### Example 248

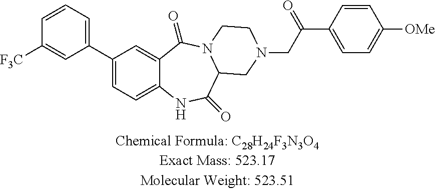 [SMILES: COc1ccc(C(=O)CN2CCN3C(=O)c4cc(-c5cccc(C(F)(F)F)c5)ccc4NC(=O)C3C2)cc1]

This example is directed to the synthesis of 2-(2-(4-methoxyphenyl)-2-oxoethyl)-8-(3-(trifluoromethyl)phenyl)-1,3,4,12a-tetrahydrobenzo[e]pyrazino[1,2-a][1,4]diazepine-6,12 (2H, 11H)-dione (KJW009-038) in an aspect of the invention.

The compound was prepared following General Procedure J using 8-bromo-2-(2-(4-methoxyphenyl)-2-oxoethyl)-1,3,4,12a-tetrahydrobenzo[e]pyrazino[1,2-a][1,4]diazepine-6,12 (2H, 11H)-dione (KJW034-1). LCMS RT (Method 1)=3.247 min, m/z 547.2 [M+Na⁺].

### Example 249

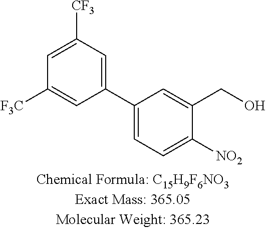 [SMILES: O=[N+]([O-])c1ccc(-c2cc(C(F)(F)F)cc(C(F)(F)F)c2)cc1CO]

This example is directed to the synthesis of (4-nitro-3′,5′-bis(trifluoromethyl)-[1,1′-biphenyl]-3-yl)methanol (KJW012-082) in an aspect of the invention.

To a stirring solution of (5-bromo-2-nitrophenyl)methanol (1 eq.) and (3,5-bis(trifluoromethyl)phenyl)boronic acid (1.4 eq.) was added aq. 2M sodium carbonate (5 eq.) followed by tetakis(triphenylphosphine)paladium(0) (0.12 eq.). The resulting mixture was heated to 95° C. for 16 hours, cooled, and the solvent removed under vacuum. The residue was slurried in CH₂Cl₂ and washed twice with brine. The organic layer was dried (Na₂SO₄) and concentrated in vacuo. The crude product was purified by flash column chromatography (silica gel, 0-25% EtOAc-hexane) to afford the title compound. ¹H NMR (400 MHz, Chloroform-d) δ 8.27 (d, J=8.5 Hz, 1H), 8.05 (d, J=9.0 Hz, 3H), 7.96 (s, 1H), 7.70 (dd, J=8.5, 2.1 Hz, 1H), 5.12 (d, J=5.2 Hz, 2H), 2.52 (t, J=6.1 Hz, 1H). ¹⁹F NMR (376 MHz, Chloroform-d) δ−62.88.

### Example 250

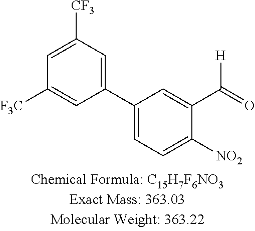 [SMILES: O=Cc1cc(-c2cc(C(F)(F)F)cc(C(F)(F)F)c2)ccc1[N+](=O)[O-]]

This example is directed to the synthesis of 4-nitro-3′,5′-bis(trifluoromethyl)-[1,1′-biphenyl]-3-carbaldehyde (KJW012-084) in an aspect of the invention.

To a solution of (4-nitro-3′,5′-bis(trifluoromethyl)-[1,1′-biphenyl]-3-yl)methanol (KJW012-082) (1 eq.) in CH₂Cl₂ was added Dess-Martin periodinane in one portion. After 30 min, the reaction was diluted with CH₂C₂ and then washed twice with saturated aqueous NaHCO₃. The organic layer was dried (Na₂SO₄) and concentrated in vacuo. The crude product was purified by flash column chromatography (silica gel, 0-20% EtOAc-hexane) to afford the title compound. ¹H NMR (400 MHz, Chloroform-d) δ 10.53 (s, 1H), 8.30 (d, J=8.4 Hz, 1H), 8.18 (d, J=2.2 Hz, 1H), 8.07 (d, J=1.6 Hz, 2H), 7.99 (dd, J=8.5, 2.2 Hz, 2H). ¹⁹F NMR (376 MHz, Chloroform-d) δ−62.89.

### Example 251

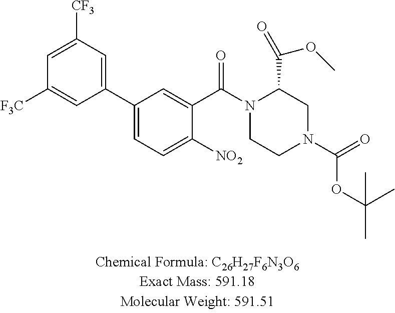 [SMILES: COC(=O)[C@@H]1CN(C(=O)OC(C)(C)C)CCN1C(=O)c1cc(-c2cc(C(F)(F)F)cc(C(F)(F)F)c2)ccc1[N+](=O)[O-]]

This example is directed to the synthesis of 1-(tert-butyl) 3-methyl (S)-4-((4-nitro-3′,5′-bis(trifluoromethyl)-[1,1′-biphenyl]-3-yl)methyl)piperazine-1,3-dicarboxylate (KJW012-085) in an aspect of the invention.

To a stirring dichloroethane solution of 4-nitro-3′,5′-bis(trifluoromethyl)-[1,1′-biphenyl]-3-carbaldehyde (KJW012-084) (1 eq.) and 1-(tert-butyl) 3-methyl (S)-piperazine-1,3-dicarboxylate (1.2 eq.) was added sodium triacetoxyhydroborate (1.8 eq.), and the resulting mixture was stirred for 48 hours. The reaction was quenched with water and concentrated in vacuo. The crude product was purified by flash column chromatography (silica gel, 0-25% EtOAc-hexane) to afford the title compound. ¹H NMR (400 MHz, DMSO-d₆) δ 8.44-8.41 (m, 2H), 8.24-8.09 (m, 3H), 8.07-7.95 (m, 2H), 4.24-4.04 (m, 3H), 3.64 (s, 3H), 3.49-2.56 (bm, 5H), 2.32 (d, J=11.3 Hz, 1H), 1.36 (s, 9H). ¹⁹F NMR (376 MHz, DMSO-d₆) δ−61.16, −61.19. LCMS RT (Method 2)=3.813 min. m/z 592.1 [M+H⁺].

### Example 252

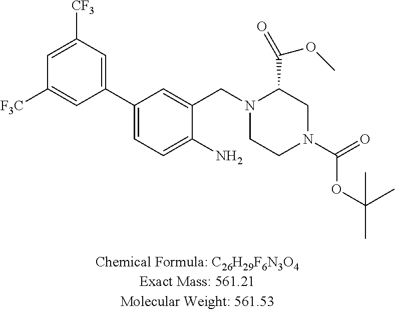 [SMILES: COC(=O)[C@@H]1CN(C(=O)OC(C)(C)C)CCN1Cc1cc(-c2cc(C(F)(F)F)cc(C(F)(F)F)c2)ccc1N]

This example is directed to the synthesis of 1-(tert-butyl) 3-methyl (S)-4-((4-amino-3′,5′-bis(trifluoromethyl)-[1,1′-biphenyl]-3-yl)methyl)piperazine-1,3-dicarboxylate (KJW012-064) in an aspect of the invention.

To a stirring mixture of 1-(tert-butyl) 3-methyl (S)-4-((4-nitro-3′,5′-bis(trifluoromethyl)-[1,1′-biphenyl]-3-yl)methyl)piperazine-1,3-dicarboxylate (KJW012-085) (1 eq.) and iron (10 eq.) in 10% aq. EtOH was added concentrated HCl (0.70 eq.), and the resulting mixture was heated to 65° C. for about 30 min, at which time thin layer chromatography (TLC) (50% EtOAc-hexanes) indicated the reaction was complete. The reaction was neutralized with 1N NaOH, diluted with DCM-MeOH and filtered through CELITE™. The solvent was removed under vacuum and the residue was purified by flash column chromatography (silica gel, 0-60% EtOAc-hexane) to afford the title compound. ¹H NMR (400 MHz, Chloroform-d) δ 7.90 (d, J=1.8 Hz, 2H), 7.73 (dt, J=1.9, 0.9 Hz, 1H), 7.38 (dd, J=8.3, 2.3 Hz, 1H), 7.21 (d, J=2.3 Hz, 1H), 6.74 (d, J=8.3 Hz, 1H), 4.93 (bs, 2H), 3.97 (d, J=12.6 Hz, 1H), 3.76 (s, 3H), 3.54 (ddt, J=13.4, 6.0, 3.3 Hz, 1H), 3.45-3.27 (m, 2H), 3.18 (dd, J=7.3, 3.8 Hz, 1H), 2.99 (ddd, J=11.6, 6.0, 3.3 Hz, 1H), 2.24 (bs, 1H), 1.45 (s, 9H). ¹⁹F NMR (376 MHz, Chloroform-d) δ−62.86. LCMS RT (Method 2)=3.833 min, m/z 561.8 [M+H⁺].

### Example 253

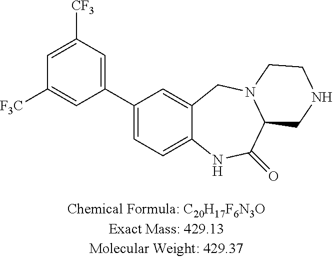 [SMILES: O=C1Nc2ccc(-c3cc(C(F)(F)F)cc(C(F)(F)F)c3)cc2CN2CCNC[C@@H]12]

This example is directed to the synthesis of (S)-8-(3,5-bis(trifluoromethyl)phenyl)-1,3,4,6,11,12a-hexahydrobenzo[e]pyrazino[1,2-a][1,4]diazepin-12 (2H)-one (KJW012-089) in an aspect of the invention.

To a stirring solution of 1-(tert-butyl) 3-methyl (S)-4-((4-amino-3′,5′-bis(trifluoromethyl)-[1,1′-biphenyl]-3-yl)methyl)piperazine-1,3-dicarboxylate (KJW012-064) (1 eq.) in toluene was added trimethylaluminum (3 eq., 2M toluene solution). The resulting mixture was heated to 80° C. for 18 hours, at which time the reaction was cooled in an ice-bath and quenched with saturated aqueous Rochelle's salt. The mixture was extracted with 10% MeOH-DCM, and the organic layer was dried (Na₂SO₄) and concentrated under vacuum. The crude product was purified by flash column chromatography (silica gel, 0-15% MeOH-DCM) to afford the title compound. ¹H NMR (400 MHz, Chloroform-d) δ 7.99-7.98 (m, 2H), 7.88 (s, 1H), 7.62-7.56 (m, 2H), 7.49-7.47 (m, 1H), 7.14 (d, J=8.1 Hz, 1H), 4.30 (d, J=14.3 Hz, 1H), 3.93-3.70 (m, 3H), 3.41 (s, 1H), 3.20 (t, J=11.4 Hz, 1H), 2.92-2.71 (m, 2H), 2.61 (t, J=10.5 Hz, 1H), 1.30-1.23 (m, 1H). ¹⁹F NMR (376 MHz, Chloroform-d) δ−62.85. LCMS RT (Method 2)=3.070 min, m/z 430.1 [M+H⁺].

### Example 254

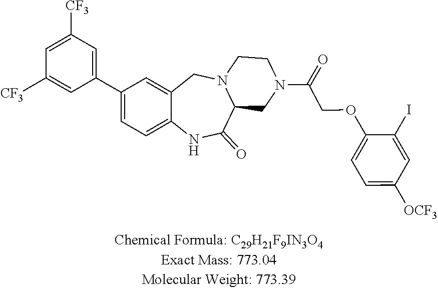 [SMILES: O=C1Nc2ccc(-c3cc(C(F)(F)F)cc(C(F)(F)F)c3)cc2CN2CCN(C(=O)COc3ccc(OC(F)(F)F)cc3I)C[C@@H]12]

This example is directed to the synthesis of (S)-8-(3,5-bis(trifluoromethyl)phenyl)-2-(2-(2-iodo-4-(trifluoromethoxy)phenoxy)acetyl)-1,3,4,6,11,12a-hexahydrobenzo[e]pyrazino[1,2-a][1,4]diazepin-12 (2H)-one (KJW012-090) in an aspect of the invention. See FIG. 12.

The compound was prepared following General Procedure C using (S)-8-(3,5-bis(trifluoromethyl)phenyl)-1,3,4,6,11,12a-hexahydrobenzo[e]pyrazino[1,2-a][1,4]diazepin-12 (2H)-one (KJW012-089) and 2-(2-iodo-4-(trifluoromethoxy)phenoxy)acetic acid (KJW012-049). ¹H NMR (400 MHz, Acetic Acid-d₄) δ 8.02-7.98 (m, 2H), 7.94-7.83 (m, 1H), 7.75-7.69 (m, 2H), 7.65-7.51 (m, 1H), 7.38 (t, J=8.1 Hz, 1H), 7.26 (d, J=8.7 Hz, 1H), 7.04 (d, J=9.0 Hz, 1H), 5.26-5.06 (m, 2H), 5.06-4.89 (m, 1H), 4.49-3.11 (m, 9H). 6 ¹⁹F NMR (376 MHz, Acetic Acid-d₄)] δ−59.34, −59.37, −63.58, −63.60, −63.62, −71.66, −76.82, −79.60. LCMS RT (Method 1)=6.110 min, m/z 773.8 [M+H⁺].

### Example 255

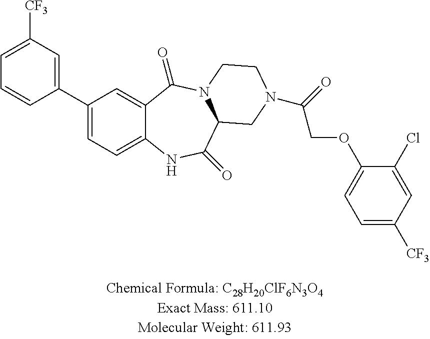 [SMILES: O=C1Nc2ccc(-c3cccc(C(F)(F)F)c3)cc2C(=O)N2CCN(C(=O)COc3ccc(C(F)(F)F)cc3Cl)C[C@@H]12]

This example is directed to the synthesis of (S)-2-(2-(2-chloro-4-(trifluoromethyl)phenoxy)acetyl)-8-(3-(trifluoromethyl)phenyl)-1,3,4,12a-tetrahydrobenzo[e]pyrazino[1,2-a][1,4]diazepine-6,12 (2H, 11H)-dione (KJW011-041) in an aspect of the invention.

The compound was prepared following General Procedure C using (S)-8-(3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW010-088) and 2-(2-chloro-4-(trifluoromethyl)phenoxy)acetic acid (KJW011-036-2). LCMS RT (Method 1)=5.832 min, m/z 612.2 [M+H⁺].

### Example 256

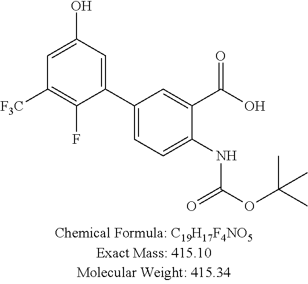 [SMILES: CC(C)(C)OC(=O)Nc1ccc(-c2cc(O)cc(C(F)(F)F)c2F)cc1C(=O)O]

This example is directed to the synthesis of 4-((tert-butoxycarbonyl)amino)-2′-fluoro-5′-hydroxy-3′-(trifluoromethyl)-[1,1′-biphenyl]-3-carboxylic acid (KJW012-029) in an aspect of the invention.

The compound was prepared following General Procedure K using (2-fluoro-5-hydroxy-3-(trifluoromethyl)phenyl)boronic acid. ¹H NMR (400 MHz, DMSO-d₆) δ 13.83 (s, 1H), 10.57 (s, 1H), 10.19 (s, 1H), 8.41 (d, J=8.8 Hz, 1H), 8.11 (dd, J=2.4, 1.4 Hz, 1H), 7.77 (ddd, J=8.8, 2.4, 1.5 Hz, 1H), 7.27-7.08 (m, 1H), 1.50 (s, 9H). ¹⁹F NMR (376 MHz, DMSO-d₆) δ−60.20, −60.24, −134.88-135.00 (M, 1H). LCMS RT (Method 2)=3.433 min, m/z 315.8 [M-BOC+H⁺].

### Example 257

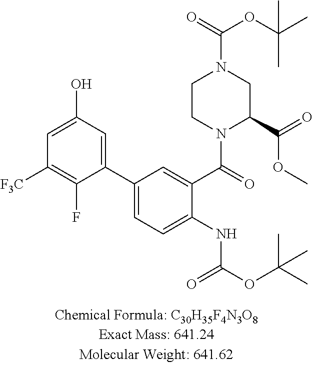 [SMILES: COC(=O)[C@@H]1CN(C(=O)OC(C)(C)C)CCN1C(=O)c1cc(-c2cc(O)cc(C(F)(F)F)c2F)ccc1NC(=O)OC(C)(C)C]

This example is directed to the synthesis of 1-(tert-butyl) 3-methyl (S)-4-(4-((tert-butoxycarbonyl)amino)-2′-fluoro-5′-hydroxy-3′-(trifluoromethyl)-[1,1′-biphenyl]-3-carbonyl)piperazine-1,3-dicarboxylate (KJW012-034) in an aspect of the invention.

The compound was prepared following General Procedure A using 1-(tert-butyl) 3-methyl (S)-piperazine-1,3-dicarboxylate and 4-((tert-butoxycarbonyl)amino)-2′-fluoro-5′-hydroxy-3′-(trifluoromethyl)-[1,1′-biphenyl]-3-carboxylic acid and was used without purification. ¹⁹F NMR (376 MHz, Chloroform-d) δ−61.51-74.34 (m, 4H).

### Example 258

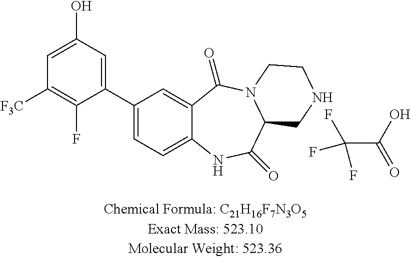

This example is directed to the synthesis of (S)-8-(2-fluoro-5-hydroxy-3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-035) in an aspect of the invention.

The compound was prepared following General Procedure B using 1-(tert-butyl) 3-methyl (S)-4-(4-((tert-butoxycarbonyl)amino)-2′-fluoro-5′-hydroxy-3′-(trifluoromethyl)-[1,1′-biphenyl]-3-carbonyl)piperazine-1,3-dicarboxylate (KJW012-035). LCMS RT (Method 3)=2.754 min, m/z 409.8 [M+H⁺].

### Example 259

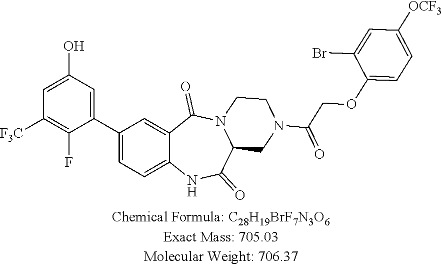 [SMILES: O=C1Nc2ccc(-c3cc(O)cc(C(F)(F)F)c3F)cc2C(=O)N2CCN(C(=O)COc3ccc(OC(F)(F)F)cc3Br)C[C@@H]12]

This example is directed to the synthesis of (S)-2-(2-(2-bromo-4-(trifluoromethoxy)phenoxy)acetyl)-8-(2-fluoro-5-hydroxy-3-(trifluoromethyl)phenyl)-1,3,4,12a-tetrahydrobenzo[e]pyrazino[1,2-a][1,4]diazepine-6,12 (2H, 11H)-dione (KJW012-037) in an aspect of the invention.

The compound was prepared following General Procedure C using (S)-8-(2-fluoro-5-hydroxy-3-(trifluoromethyl)phenyl)-1,3,4,12a-tetrahydrobenzo[e]pyrazino[1,2-a][1,4]diazepine-6,12 (2H, 11H)-dione 2,2,2-trifluoroacetate (KJW012-035) and 2-(2-bromo-4-(trifluoromethoxy)phenoxy)acetic acid (KJW011-027). LCMS RT (Method 1)=5.543 min, m/z 708.0 [M+2H⁺].

### Example 260

This example demonstrates the agonist RXFP2 agonist activity of exemplary compounds of formula (I) in an aspect of the invention.

Compound efficacy and EC₅₀ were determined by measuring cAMP induction in vitro. HEK293T cells stably expressing RXFP2 (HEK-RXFP2) produce cAMP when RXFP2 is activated, which can be measured in an antibody-based immunoassay utilizing a fluorescent readout (cAMP Gs HiRange Kit, PerkinElmer, Waltham, MA). HEK-RXFP2 cells were seeded in 96-well plates in serum-free DMEM and incubated overnight at 37° C., 5% CO₂. Plates were then treated with a compound titration up to 25 μM or INSL3 control, in the presence of isobutylmethylxanthine phosphodiesterase inhibitor (IBMX) at 200 μM to amplify the cAMP signal, for 1 hour at 37° C., 5% CO₂ before being processed as per kit instructions. Table 7 lists the efficacy and EC₅₀ of compounds tested under different experimental conditions. Protocols were modified since more potent compounds had responses too high to measure under initial conditions. In condition (a), 20,000 cells were seeded per well in the 96 well plate, and efficacy was determined by normalizing the maximum compound cAMP response to the 1 nM INSL3 cAMP response (% Efficacy 1 nM INSL3 (a)). In condition (b), 10,000 cells were seeded per well, and compound response was normalized to the 1 nM INSL3 cAMP response (% Efficacy 1 nM INSL3 (b)). In condition (c), 7,500 cells were seeded per well, compound response was normalized to the 100 nM INSL3 cAMP response, and IBMX was omitted (% Efficacy 100 nM INSL3 (c)). A range is given when compounds were tested multiple times. Where compounds were tested under multiple experimental conditions, the EC₅₀ listed for the corresponding condition is marked (a) through (c) to correlate. When EC₅₀ could not be calculated from the dose-response curve, EC₅₀ is listed as not determined (ND).

TABLE 7

| | | | % EFF | |
|---|---|---|---|---|
| | % EFF | % EFF | vs. | |
| | vs. 1 nM | vs. 1 nM | 100 nM | |
| Example | INSL3 ^{(a)} | INSL3 ^{(b)} | INSL3 ^{(c)} | EC₅₀ (μM) |
| 40 | | 47.69 | | ND |
| 41 | | 33.78 | | ND |
| 55 | 20.07-42.35 | | | 0.02442-0.05935 |
| 64 | 56.29-60.35 | | | 0.037-0.0566 |
| 68 | 33.99 | | | 0.00856 |
| 101 | 35.5 | | | 0.02451 |
| 104 | | 67.7 | | 0.0177 |
| 105 | 143.62 | 81.13-152.31 | | ND (a) |
| | | | | 0.0842-0.219 ^{(b)} |
| 106 | | 50.30 | | 0.0315 |
| 107 | 27.96 | | | ND |
| 108 | | 38.19 | | ND |
| 112 | 12.93 | | | ND |
| 115 | 3.69 | | | ND |
| 117 | | 77.84 | | 0.0156 |
| 118 | 43.71 | | | ND |
| 120 | | 70.76 | | 0.0086 |
| 121 | 28.28 | | | ND |
| 123 | | 113.66-166.88 | 38.35-49.8 | 0.018-0.0233^{(b)} |
| | | | | 0.0462-0.07374^{(c)} |
| 127 | | 84.99 | | 0.012 |
| 130 | | 54.37 | | ND |
| 132 | | 68.09 | | ND |
| 136 | | 92.5-107.57 | | 0.067-0.0894 |
| 137 | | 53.62 | | ND |
| 141 | | 95.53-115.85 | | 0.036-0.0545 |
| 142 | | 80.32 | | 0.048 |
| 146 | | 47.43 | | ND |
| 150 | | 103.32 | | 0.0222 |
| 151 | | 0.85 | | ND |
| 153 | | 149.80-150.94 | 45.16-86.79 | 0.0236-0.05018 ^{(b)} |
| | | | | 0.1006-0.3188 ^{(c)} |
| 157 | | 108.89 | | 0.0391 |
| 158 | | | 48.99-81.93 | 0.167-1.571 |
| 162 | | 130.93 | | 0.121 |
| 163 | | 114.25 | | ND |
| 164 | | 126.02 | | ND |
| 166 | | 132.67 | | 0.2361 |
| 167 | | 113.26 | | 0.1423 |
| 168 | | 131.20 | | ND |
| 172 | | 70.66 | | ND |
| 173 | | 114.71 | | 0.06421 |
| 177 | | | 17.49 | ND |
| 178 | | | 52.68 | ND |
| 179 | | | 28.25 | ND |
| 180 | | | 23.13 | ND |
| 185 | | | 63.16 | 0.4519 |
| 187 | | | 87.28-147.65 | 0.1276-0.5684 |
| 191 | | | 66.75 | 0.1887 |
| 192 | | | 77.31 | 0.07778 |
| 196 | | | 109.23 | 0.9838 |
| 197 | | | 104.06 | ND |
| 202 | | | 102.23 | 0.04073 |
| 206 | | | 60.89 | 0.5626 |
| 231 | 0.04 | | | ND |
| 254 | | | 49.35 | 8.607 |
| 255 | | 59.15 | | 0.0097 |
| 259 | | | 67.97 | 0.06208 |

ND: not determined

### Example 261

This example demonstrates the secondary luciferase cAMP screening of positive homogeneous time resolved fluorescence (HTRF) hits.

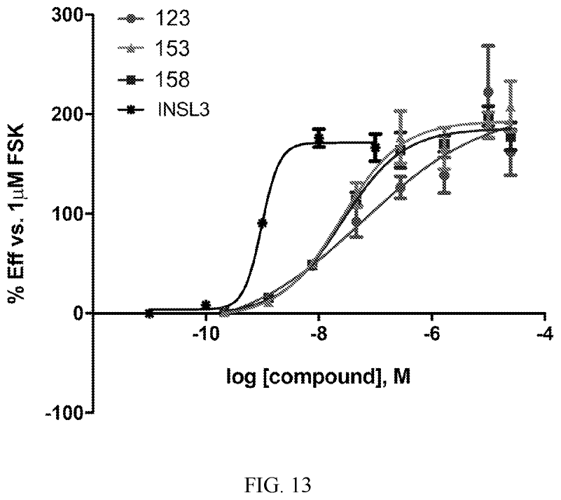

A confirmatory cAMP assay was done to eliminate false positives using a HEK293T-CRE-Luc cell line stably transfected with RXFP2 (HEK-CRE-Luc-RXFP2), as this cell line expresses luciferase under the control of the cAMP response element (CRE). Activation of the RXFP2 receptor increases intracellular cAMP levels, inducing luciferase transcription in the nucleus. Thus, luciferase activity in these cells is proportional to cAMP accumulation. For this assay, cells were seeded in 96-well flat-bottom opaque plates at 20,000 cells/well in 60 μL/well of serum-free DMEM medium and allowed to attach overnight at 37° C., 5% CO₂. The next morning, cells were treated with 1 μL/well of compound (25 μM-0.21 nM), 1 μM forskolin, or DMSO vehicle. Cells were also treated with 4 μL of INSL3 (100-0.01 nM) (Phoenix Pharmaceuticals, Burlingame, CA) or vehicle serum-free DMEM. Cells were incubated with the treatments for 3 hours at 37° C., 5% CO₂, then rested 30 minutes at room temperature for equilibration, after which 65 μL/well of substrate AMPLITE™ Luciferase Reporter Gene Assay Kit (AAT Bioquest, Sunnyvale, CA) was added. Plates were incubated for 40 minutes at room temperature protected from light, and then luciferase output was read on a CLARIOstar plate reader (BMG Labtech, Germany). FIG. 13 shows the results of a Luciferase cAMP assay in HEK-CRE-Luc-RXFP2 cells after treatment with representative RXFP2 agonists of Examples 123, 153, and 158, in comparison to the RXFP2 cognate ligand INSL3 (mean±SEM of 3 technical replicates). Efficacy of compounds and INSL3 was normalized to 1 μM forskolin cAMP response as 100% activity and DMSO vehicle as 0% activity. Further, agonist EC₅₀ and Eₘₐₓ relative to 10 nM INSL3 response values were calculated and are shown in Table 8. These results showed that the compounds of Examples 153 and 158 are potent RXFP2 agonists.

TABLE 8

| | | Eₘₐₓ |
|---|---|---|
| | | (% Eff vs. |
| Example | EC₅₀ (nM) | 10 nM INSL3) |
| 123 | 58.5 | 117.1 |
| 153 | 24.6 | 109.6 |
| 158 | 25.8 | 105.5 |

### Example 262

This example describes an HTRF cAMP counter-screen in HEK-RXFP1 cells in an aspect of the invention.

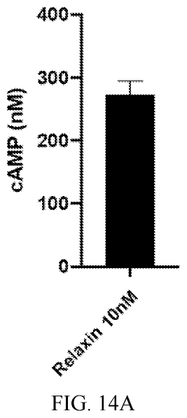

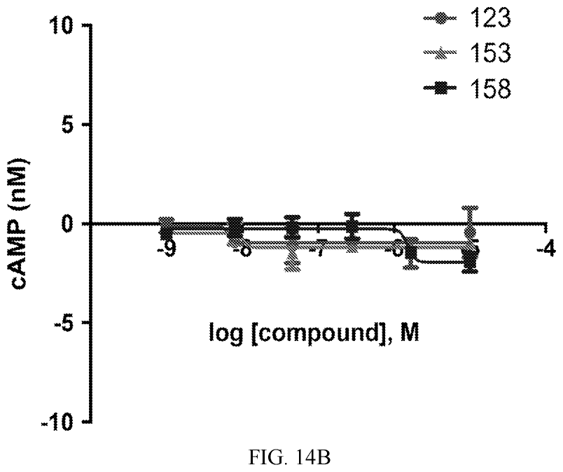

HEK293T cells stably transfected with RXFP1 (HEK-RXFP1) were used to test compound specificity towards the RXFP2 receptor using the HTRF Gs dynamic cAMP assay kit (PerkinElmer, Waltham, MA). This is a competitive immunoassay where a time-resolved fluorescence resonance energy (TR-FRET) signal is produced when exogenous d2-labeled cAMP (acceptor) binds to europium cryptate-conjugated anti-cAMP antibody (donor). No signal is produced when endogenous cAMP binds to the antibody. Thus, the TR-FRET signal is inversely proportional to the concentration of cAMP produced by the cells. The HTRF cAMP assay was carried out in the presence of isobutylmethylxanthine phosphodiesterase inhibitor (IBMX) at 200 μM to amplify the cAMP signal. For this assay, cells were seeded in 96-well flat-bottom opaque plates at 30,000 cells/well in 60 μL/well of serum-free DMEM medium and allowed to attach overnight at 37° C., 5% CO₂. The next morning, cells were treated with 1 μL/well of compound (10 μM-1.2 nM), 1 μM forskolin, or DMSO vehicle. Cells were also treated with 4 μL of 10 nM Relaxin2 (Peprotech, Cranbury, NJ) as a positive control or vehicle (serum-free DMEM+IBMX). Plates were incubated for 1 hour at 37° C., 5% CO₂, after which 16 μL/well of kit cAMP-d₂ and 16 μL/well of anti-cAMP antibody were added as per manufacturer protocol. Cells were incubated for 1 hour at room temperature, and then the signal was read on a CLARIOstar plate reader (BMG Labtech, Germany). FIGS. 14A and 14B show the RXFP1 cAMP response after treatment with representative RXFP2 agonists of Examples 123, 153, and 158 (mean±SEM of 3 technical replicates). These results show that the RXFP2 agonists do not activate the highly homologous RXFP1 receptor. Relaxin2 is the cognate ligand of RXFP1 and is used as a positive control for RXFP1 receptor activation.

### Example 263

This example demonstrates a PRESTO-Tango GPCRome counter-screen in transiently transfected HTLA cells in an aspect of the invention.

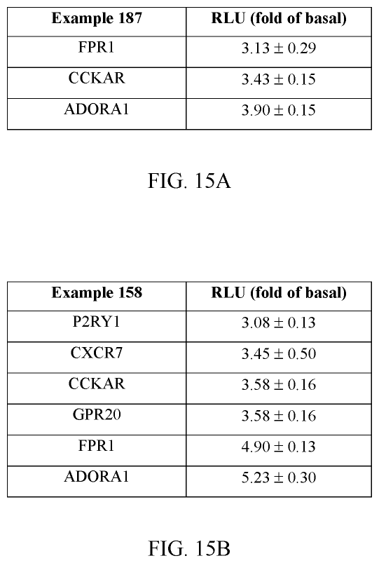

The PRESTO-Tango GPCRome assay, which measures G-protein coupled receptors (GPCR) mediated beta-arrestin recruitment, was used to perform a HTS of related GPCRs to study specificity and selectivity of the RXFP2 agonists. A total of 320 GPCR Tango constructs were tested. Each of the GPCR constructs used in the assay contained a FLAG tag, the GPCR gene of interest, a Vasopressin 2 C-terminal tail, the TEV protease cleavage site, and the tetracycline-controlled transactivator (tTA) transcription factor. The assay was performed using transiently transfected HTLA cells that stably express the beta-arrestin2-TEV protease fusion protein and a luciferase reporter gene under the control of the tTA transcription factor. Ligand activation of the transfected GPCR construct induces beta-arrestin translocation to the GPCR, where the tobacco etch virus (TEV) protease cleaves the tTA transcription factor, causing activation of luciferase transcription in the nucleus. Thus, luciferase activity in these cells is proportional to beta-arrestin translocation activity. For this assay, HTLA cells were seeded in Poly-L-Lys (PLL)-coated 384-well white clear-bottom plates at 10,000 cell/well in 40 μl/well DMEM supplemented with 10% FBS. Cells were incubated overnight at 37° C., 5% CO₂ to allow them to attach. The next morning, cells were fed 10 μL/well of 50% FBS DMEM 1 hour before transfection. Cells were transfected with 20 ng of plasmid diluted in an equal volume of 0.25 M CaCl₂) and HEPES ((4-(2-hydroxyethyl)-1-piperazineethanesulfonic acid) buffered saline (HBS) (50 mM HEPES, 280 mM NaCl, 10 mM KCl, 1.5 mM Na₂HPO₄, pH 7.00) in a total volume of 6 μl/well using a MICROLAB™ STAR (Hamilton, Reno, NV) with a 384-well pipetting head. After transfection, cells were incubated overnight at 37° C., 5% CO₂. The next morning, medium was removed and replaced with 40 μl/well of fresh 1% FBS DMEM 2 hours before addition of treatments. Cells were treated with 10 μL/well of 10 μM compound diluted in 1% FBS DMEM and incubated overnight at 37° C., 5% C₀₂. The following day, medium was removed and 20 μl/well of BrightGlo reagent (Promega, Madison, WI) diluted 1:20 with Tango assay buffer was added. Plates were incubated for 20 minutes at room temperature protected from light and the luminescence signal was read on a MicroBeta counter (PerkinElmer, Waltham, MA). FIG. 15 shows the results of the PRESTO-Tango GPCRome screening with representative RXFP2 agonists of Example 187 (FIG. 15A) and Example 158 (FIG. 15B) (means±SEM of 4 technical replicates). GPCRs with a minimum of 3.0-fold relative light units (RLU) of basal may have potential agonist activity and are shown in the tables in FIGS. 15A and 15B. Overall, there are very few GPCR hits and the actual RLUs are very close to the basal response, which suggests that the compounds are specific for RXFP2.

### Example 264

This example demonstrates a cytotoxicity assay in HEK293T and HCO cells in an aspect of the invention.

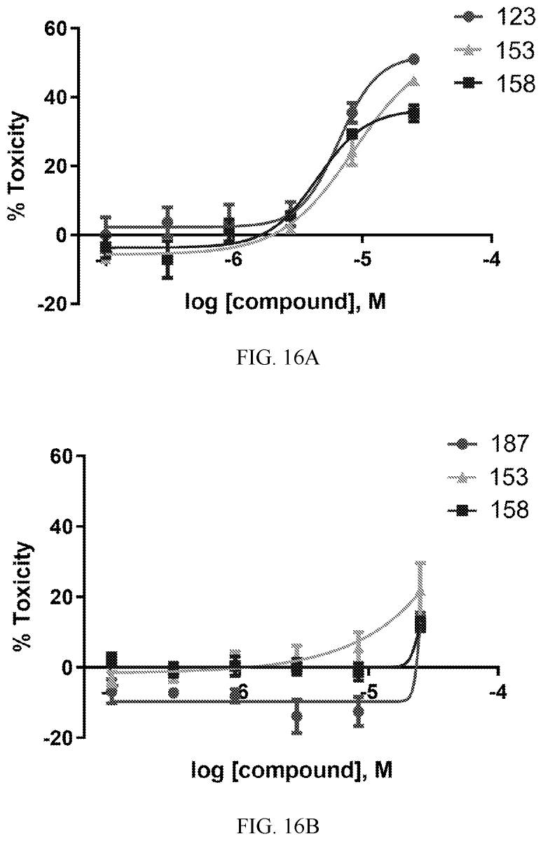

Cytotoxicity induced by the compounds in HEK293T cells and primary human calvarial osteoblasts (HCO) (ScienCell Research Laboratories, San Diego, CA) were tested using the CELLTITER-GLO™ Luminescent Cell Viability Assay (Promega, Madison, WI). This is a luminescence-based assay that determines viable cells by measuring cellular adenosine triphosphate (ATP) production. The luciferase enzyme requires ATP to catalyze the conversion of beetle luciferin to oxyluciferin and light. Thus, luciferase activity is proportional to ATP accumulation in the cells. For this assay, 96-well flat-bottom opaque plates seeded with HEK293T cells at 5,000 cells/well and HCO cells at 3,000 cells/well in 100 μL/well of growth medium (DMEM, 10% FBS, 1× Penicillin/Streptomycin). After incubating overnight at 37° C., 5% CO₂ to allow attachment, cells were treated with 1 μL/well of compound (25-0.1 μM) or DMSO vehicle and incubated an additional 24 hours. Then cells were equilibrated for 30 minutes at room temperature and 100 μL/well of CELLTITER-GLO™ Reagent was added. Plates were placed on an orbital shaker for 2 minutes and incubated at room temperature for 10 minutes to stabilize the luminescent signal, which was read immediately after on a CLARIOstar plate reader (BMG Labtech, Germany). FIG. 16 shows the cytotoxicity results in HEK293T cells (FIG. 16A) and HCO cells (FIG. 16B) normalized to DMSO vehicle as 0% toxicity. The representative RXFP2 agonists of Examples 123, 153, and 158 induced some toxicity in HEK293T cells at 25 μM and 8.3 μM treatment, but not at 2.8 μM or lower (FIG. 16A). In the HCO cells, representative RXFP2 agonists of Examples 187, 153, and 158 induced minimal toxicity at 25 μM and they were not toxic at lower concentrations (FIG. 16B). The HEK293T results represent the means±SEM of 3 technical replicates. The HCO results represent the means±SEM of 3 independent experiments.

### Example 265

This example demonstrates activation of mouse RXFP2 in transiently transfected HEK-CRE-Luc cells in an aspect of the invention.

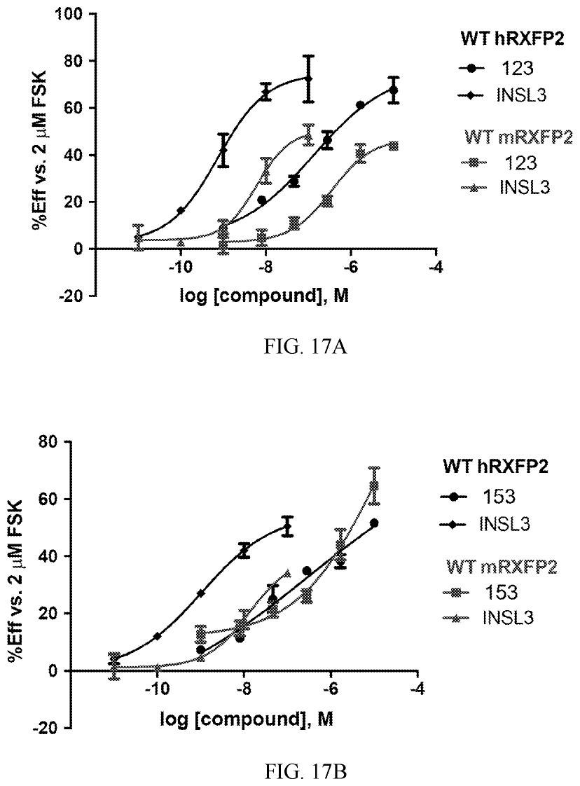

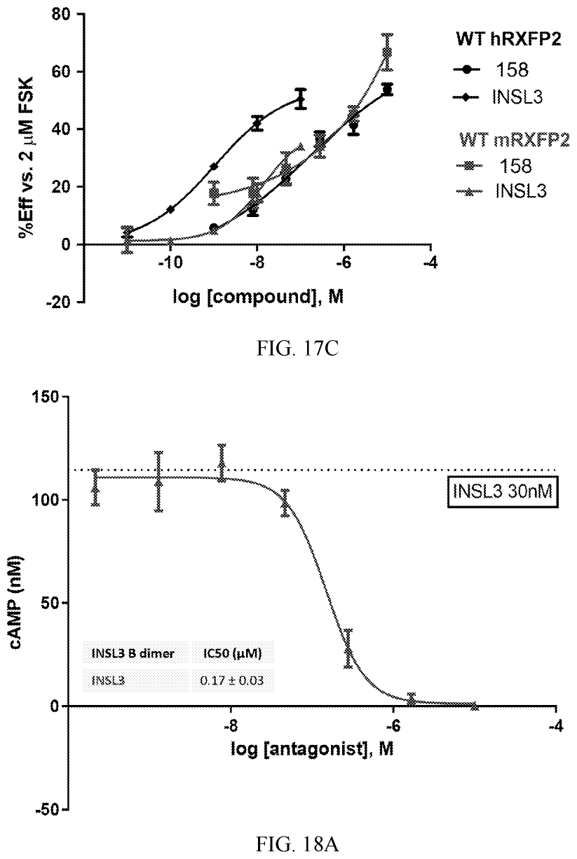

It was of interest if the RXFP2 agonists of formula (I) activate the mouse RXFP2 receptor. HEK-CRE-Luc cells transiently transfected with mouse RXFP2 were used to test agonist activity by measuring induction of cAMP. For this assay, cells were seeded in 6-well flat-bottom clear plates at 0.6 million cells/well in 2 mL/well of growth medium (DMEM, 10% FBS, 1× Pen/Strep) and allowed to attach overnight at 37° C., 5% CO₂. The next morning, cells were transfected with 2 μg of plasmid (human or mouse RXFP2) and 6 μL of Lipofectamine2000 (Invitrogen, Waltham, MA) in a total volume of 200 μl/well OPTI-MEM™ I (Thermo Fisher, Waltham, MA) reduced serum medium and incubated an additional 24 hours. The transfected cells were then harvested and seeded in 96-well flat-bottom opaque plates at 30,000 cells/well in 60 μL/well of serum-free DMEM medium and incubated overnight to attach. The next morning, cells were treated with 1 μL/well of compound (10 μM-1.2 nM), 2 μM forskolin, or DMSO vehicle, and with 4 μL of positive control INSL3 (100-0.01 nM) (Phoenix Pharmaceuticals, Burlingame, CA) or vehicle serum-free DMEM. Cells were incubated with the treatments for 3 hours at 37° C., 5% CO₂, then rested 30 minutes at room temperature for equilibration, after which 65 μL/well of substrate AMPLITE™ Luciferase Reporter Gene Assay Kit (AAT Bioquest, Sunnyvale, CA) were added. Plates were incubated for 40 minutes at room temperature protected from light, and the signal was read on a CLARIOstar plate reader (BMG Labtech, Germany). FIG. 17 shows the results of mouse RXFP2 activation after treatment with INSL3 and representative RXFP2 agonists of Examples 123 (FIG. 17A), 153 (FIG. 17B), and 158 (FIG. 17C), in comparison to human RXFP2 response (means±SEM of 3 technical replicates). Efficacies of the agonist compounds and INSL3 were normalized to 2 μM forskolin cAMP response as 100% activity and DMSO vehicle as 0% activity for each plasmid. These results demonstrated that the RXFP2 agonists of formula (I) can activate the mouse RXFP2 receptor at levels comparable to human RXFP2 activation. This suggests that RXFP2 agonists of formula (I) can be used for pre-clinical testing in mouse models of disease.

### Example 266

This example shows the characterization of agonist-receptor interactions using an INSL3 antagonist in an aspect of the invention.

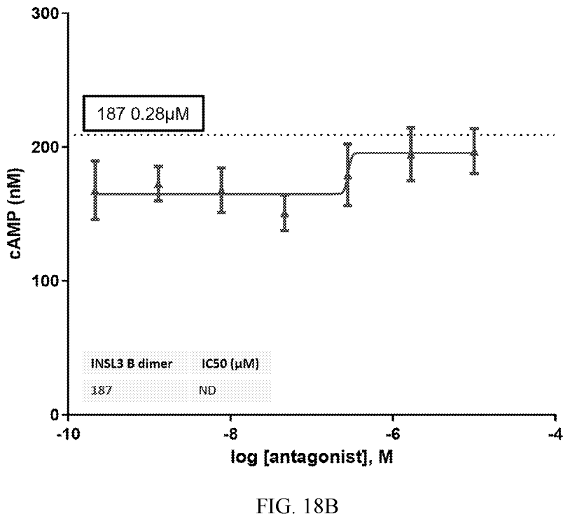

Previous publications have characterized the INSL3/RXFP2 binding model, showing that the B-chain of INSL3 binds with high affinity to the extracellular leucine-rich repeat (LRR) domain of RXFP2. To determine if the RXFP2 agonists interact with the receptor in a similar manner, an INSL3 antagonist (INSL3 B dimer) was used to co-treat HEK-RXFP2 cells with RXFP2 agonists or INSL3 and measure induction of cAMP using the HTRF assay. This INSL3 antagonist consists of a dimer formed by two INSL3 B-chains and has been shown to bind the LRR domain of RXFP2 without inducing a cAMP response. The HTRF cAMP assay was carried out in the presence of 200 μM IBMX to amplify the cAMP signal. For this assay, cells were seeded in 96-well flat-bottom opaque plates at 7,500 cells/well in 60 μL/well of serum-free DMEM medium and allowed to attach overnight at 37° C. and 5% CO₂. The next morning, cells were treated with 2 μL of INSL3 B dimer antagonist (10 μM-0.21 nM) and 2 μL of 30 nM INSL3 or 4 μL of vehicle (serum-free DMEM+IBMX). Cells were also treated with 1 μL/well of 0.28 μM compound, 2 μM forskolin, or DMSO vehicle. Cells were incubated for 1 hour at 37° C., 5% CO₂, after which kit cAMP-d₂ and anti-cAMP antibody were added as previously described. Plates were incubated for 1 hour at room temperature, and then the signal was read on a CLARIOstar plate reader (BMG Labtech, Germany). FIG. 18 shows the effects of the INSL3 B dimer antagonist (FIG. 18A) on the cAMP response induced by INSL3 and the representative RXFP2 agonist of Example 187 (FIG. 18B) (means±SEM of 3 independent experiments). As expected, the INSL3 B dimer antagonist inhibited cAMP response induced by INSL3 in a dose-dependent manner. On the contrary, the antagonist had no effect on the cAMP response induced by the compound of Example 187. These results suggest that the agonist activates RXFP2 in a different manner than INSL3, and the LRR domain of RXFP2 is most likely not involved in agonist-receptor interactions.

### Example 267

This example shows the characterization of agonist-receptor interactions using chimeric receptors in an aspect of the invention.

To further investigate the involvement of the RXFP2 extracellular and transmembrane domains in agonist activation of the receptor, two complementary chimeras were created using the RXFP1 receptor, which is not activated by the agonists of formula (I), to transiently transfect HEK293T cells and measured changes in cAMP induction using the HTRF assay.

### Construction of Chimeric Receptors

Chimeric receptors were generated by IN-FUSION™ (Takara Bio USA, Inc., San Jose, CA) mutagenesis using full-length human RXFP1 and RXFP2 receptor constructs. Polymerase chain reaction (PCR) was performed using PfuTurbo high-fidelity DNA polymerase (Agilent Technologies, Santa Clara, CA) and overlapping primers with at least 15 bp sequence homology within the fusion site. PCR products were digested with Dpnl restricton enzyme at 37° C. overnight, run on a 0.8% agarose gel and purified using the GENEJET™ Gel extraction kit (Thermo Scientific, Waltham, MA). The purified DNA fragments were then ligated into the BamHI and XhoI pre-digested pcDNA3.1™/Zeo(+) AmpR mammalian expression vector using the In-Fusion HD enzyme premix (Clontech, Mountain View, CA) at 50° C. for 15 minutes, and the resulting product was used to transform Stellar competent cells (Takara Bio USA, Inc., San Jose, CA). The clones were fully sequenced to confirm the correct fusion of both receptor fragments. Chimera RXFP2-1 contained the extracellular domain of RXFP2 and the transmembrane domain of RXFP1. Accordingly, chimera RXFP1-2 contained the extracellular domain of RXFP1 and the transmembrane domain of RXFP2.

Induction of cAMP Signaling in Transiently Transfected HEK293T Cells

The chimeric receptors were used to transiently transfect HEK293T cells and measure differences in cAMP induction by INSL3 and the compound of Example 187. HEK293T cells were seeded in 6-well flat-bottom clear plates at 0.5 million cells/well in 2 mL/well of growth medium (DMEM, 10% FBS, 1× Pen/Strep) and allowed to attach overnight at 37° C. and 5% CO₂. The next morning, cells were transfected with 2 μg of plasmid (chimeric receptors, WT RXFP2, or WT RXFP1) and 6 μl of Lipofectamine2000 (Invitrogen, Waltham, MA) in a total volume of 200 μl/well OPTI-MEM™ I (Thermo Fisher, Waltham, MA) reduced serum medium and allowed to incubate an additional 24 hours. The next day, transfected cells were seeded in 96-well flat-bottom opaque plates at 30,000 cells/well in 60 μL/well of serum-free DMEM medium and allowed to attach overnight at 37° C. at 5% CO₂. The next morning, cells were treated with 1 μL/well of compound (25 μM-0.25 nM), 2 μM forskolin, or DMSO vehicle. Cells were also treated with 4 μL of positive control INSL3 (100-0.01 nM), Relaxin2 (100-10 nM), or vehicle (serum-free DMEM+IBMX). Plates were incubated for 1 hour at 37° C. and 5% CO₂, after which kit cAMP-d₂ and anti-cAMP antibody were added as previously described. Cells were incubated for 1 hour at room temperature, and then the signal was read on a CLARIOstar plate reader (BMG Labtech, Germany). Table 9 shows the changes in cAMP response of the chimeras in comparison to the wild type (WT) receptors after treatment with representative RXFP2 agonist of Example 187 and positive control INSL3 (means±SEM of 3 independent experiments). The compound of Example 187 and INSL3 efficacies for each receptor were normalized to 2 μM forskolin cAMP response as 100% activity and their Eₘₐₓ and EC₅₀ values are reported in Table 9.

TABLE 9

| | | EC₅₀ | | |
|---|---|---|---|---|
| Compound | Receptor | (μM or nM) | | Eₘₐₓ |
| Example 187 | WT RXFP2 | 0.012 ± 0.008 | μM | 86.31 ± 3.38 |
| | RXFP2-1 | ND | | ND |
| | WT RXFP1 | ND | | ND |
| | RXFP1-2 | 0.016 ± 0.002 | μM | 96.47 ± 0.43 |
| INSL3 (control) | WT RXFP2 | 0.50 ± 0.35 | nM | 78.38 ± 11.24 |
| | RXFP2-1 | 0.74 ± 0.61 | nM | 92.30 ± 21.82 |

| | | |
|---|---|---|
| WT RXFP1 | ND | ND |
| RXFP1-2 | ND | ND |

The results in Table 9 showed that the chimera RXFP2-1 had no response to the compound of Example 187 but responded to INSL3 at levels comparable to WT RXFP2. On the contrary, the RXFP1-2 chimera did not respond to INSL3 but responded to the compound of Example 187 at levels comparable to WT RXFP2, and to Relaxin at levels comparable to WT RXFP1. These results suggest that the extracellular domain of RXFP2 is not involved in the compound of Example 187's activation of the receptor, and points to the transmembrane domain as the primary region for agonist interaction.

### Expression of FLAG Tagged Chimeric Receptors on the Cell Surface

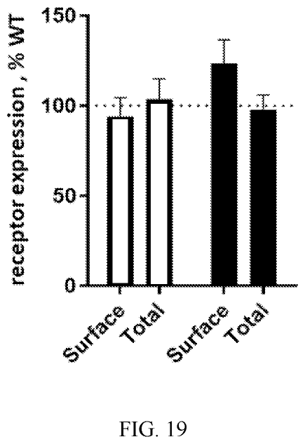

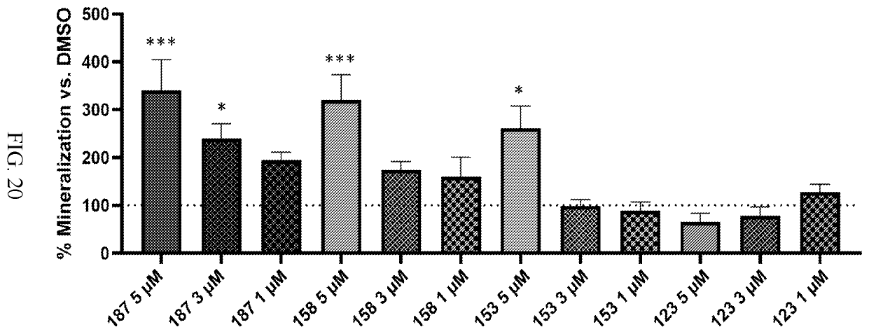

All receptor constructs contain a FLAG tag that allows measurement of receptor expression on the cell surface via flow cytometry. For this assay, HEK293T cells were seeded in 6-well flat-bottom clear plates at 0.6 million cells/well in 2 mL/well of growth medium (DMEM, 10% FBS, 1× Pen/Strep) and allowed to attach overnight at 37° C. and 5% CO₂. The next morning, cells were transfected with 2 μg of plasmid (empty vector pcDNA3.1, chimeric receptors, WT RXFP2 or WT RXFP1) and 6 μL of Lipofectamine2000 (Invitrogen) in a total volume of 200 μl/well OPTI-MEM™ I (Thermo Fisher, Waltham, MA) reduced serum medium and incubated at 37° C. and 5% CO₂. Twenty-four hours after transfection, cells were harvested in 1.5 mL/well of phosphate-buffered saline (PBS)+5 mM ethylenediaminetetraacetic acid (EDTA). 750 μL of the cell suspension was used for surface staining and 750 μL permeabilized for total staining. Cells were centrifuged at 2,000×rpm for 3 minutes, resuspended in 400 μl PBS and fixed for 10 minutes in 3.7% formaldehyde. Cells were centrifuged, washed twice with 0.5 mL of stain buffer for surface detection (2% fetal bovine serum (FBS), trsi-buffered saline (TBS), and 1 mM CaCl₂)) or with permeabilization buffer for total staining (stain buffer+0.2% polysorbate 20 (TWEEN™ 20)) and incubated with 0.5 μg anti-FLAG M1 Ab (Sigma-Aldrich, St. Louis, MO) for 1 hour at 4° C. in 100 μL of the respective buffer. Cells were washed again with 1 mL of stain or permeabilization buffer and incubated with 1 μg ALEXA FLUOR™ 488 goat anti-mouse IgG (Invitrogen, Waltham, MA) for 20 minutes at 4° C. protected from light in 100 μL of the respective buffer. Cells were washed a final time with 1 mL of the respective buffer and resuspended in 300 μl stain buffer for analysis on an ACCURI™ C₆ flow cytometer (BD Biosciences, Franklin Lakes, NJ). Cells transfected with the empty vector were used to establish background staining. FIG. 19 shows the surface and total expression of the chimeric receptors normalized to the expression of the respective WT receptor (means±SEM of 3 independent experiments). The expression of the RXFP2-1 chimera was normalized to the expression of WT RXFP2 and the RXFP1-2 normalized to WT RXFP1. The results demonstrate that the chimeras are expressed on the cell surface at similar levels as the WT receptors.

### Characterization of Agonist Biological Activity in Primary HCO Cells

The biological activity of the RXFP2 agonists was tested in primary HCO cells (ScienCell Research Laboratories, San Diego, CA) by measuring their ability to induce osteoblast mineralization. A fluorescent-based assay (OSTEOIMAGE™ Mineralization Assay, Lonza, Switzerland) was used that measures hydroxyapatite, which is the main mineral component of bone. For this assay, HCO cells were seeded in 0.1% gelatin-coated 96 well black-walled flat-bottom clear plates at 7,000 cells/well in 100 μL/well of growth medium (DMEM, 10% FBS, 1× Pen/Strep) and allowed to attach overnight at 37° C. and 5% CO₂. The next day, growth medium was changed to 100 μL/well of mineralization medium (growth medium+10 mM β-glycerophosphate, 50 μg/ml ascorbic acid, 10 nM dexamethasone), along with the respective treatments. Cells were treated with 1 μL/well of compound (1, 3, or 5 μM) or vehicle (serum-free DMEM, 0.05% DMSO). Medium and treatments were replaced every 2-3 days and cells were incubated at 37° C. and 5% CO₂ for 14 days. Hydroxyapatite deposits were evaluated using the OSTEOIMAGE™ Mineralization Assay (Lonza, Switzerland). At treatment day 14, cells were rested 30 minutes at room temperature for equilibration, washed with 200 μL/well PBS, and fixed in 100 μL/well 4% formaldehyde for 15 minutes. Cells were washed once with 200 μL/well assay wash buffer and 100 μL/well of assay staining reagent was added as per manufacturer's protocol. Cells were incubated at room temperature for 30 minutes, protected from light. After staining, cells were washed 3 times with 200 μL/well wash buffer and wells were filled with a final 200 μL/well wash buffer. Mineralization was quantified using a CLARIOstar plate reader (BMG Labtech, Germany) and representative images of the hydroxyapatite bone-like nodules were captured using a fluorescence microscope (Nikon Eclipse TS100 attached to an Olympus DP70 camera). FIG. 20 shows the mineralization results in HCO cells after treatment with representative RXFP2 agonists of Examples 187, 158, 153, and 123. Mineralization activity induced by compounds was normalized to DMSO vehicle as 100% mineralization. The results show a significant increase in mineralization with the compounds of Examples 187, 158, and 153 at 5 μM. Example 123 is a compound from the same chemotype that had weaker activity in the HTRF cAMP screen, and does not seem to increase mineralization in osteoblasts. The results represent the means±SEM of 3 independent experiments. *p<0.05, ***p<0.001 vs. DMSO, one-way ANOVA.

### Example 268

This example shows the characterization of agonist biological activity and specificity in vivo in an aspect of the invention.

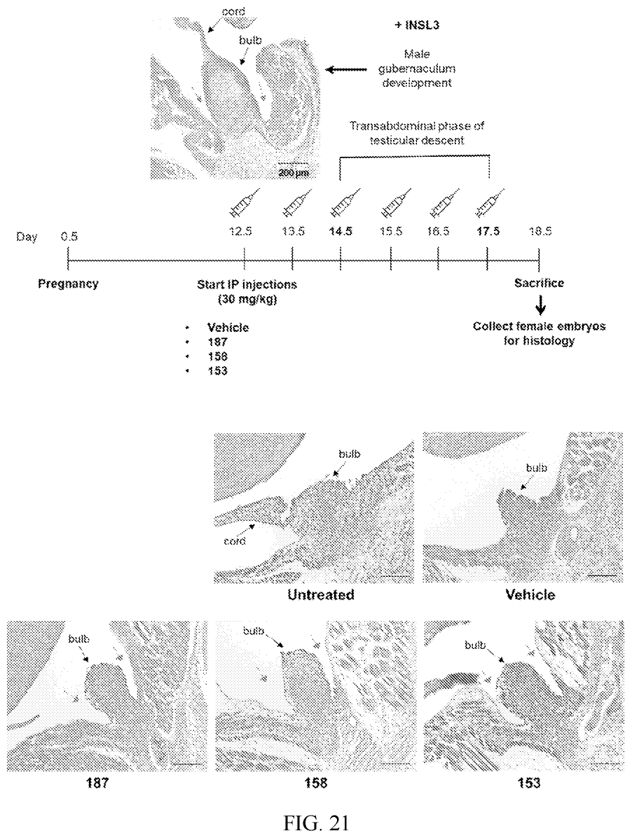

The role of INSL3 and RXFP2 in gubernaculum development for the initial transabdominal descent of the testis during embryogenesis has been widely characterized in Insl3^{-/-} and Rxfp2^{-/-} mouse models of cryptorchidism. At embryonic day 14.5, INSL3 starts driving gubemaculum development for the transabdominal stage of testicular descent in males, which is finalized by embryonic day 17.5. Representative histological sections of the developed male gubemaculum at embryonic day 18.5 were taken. The gubernaculum is composed of the gubernacular bulb and cord, and it has begun invagination into the abdominal wall in preparation for the inguinoscrotal stage of testicular descent, which will finalize after birth. In female mice, there is no production of INSL3 during embryogenesis, therefore the gubernaculum does not develop but remains present as a vestigial structure. The hypothesis for our experiment is that by injecting pregnant females with the RXFP2 agonists during this embryonic development window, gubemaculum development could be induced in female embryos, which has been previously shown in female embryos overexpressing INSL3. FIG. 21 shows a schematic representation of the assay principle. Pregnant C57BL/6J female mice were given 6 consecutive intraperitoneal injections with 30 mg/kg of representative RXFP2 agonists of Examples 187, 158, and 153 or vehicle (60% Phosal-40% PEG300) from embryonic day 12.5 to 17.5. At embryonic day 18.5, the females were sacrificed to extract the embryos for hematoxylin and eosin (H&E) histological analysis of gubernaculum morphology. For this assay, whole embryos were fixed in 10% formaldehyde for 24 hours. Afterwards, embryos were washed twice with PBS for 1 hour, followed by 3 consecutive washes with increasing concentrations of ethanol (30%, 50% and 70%) for 30 minutes. Fixed embryos were prepared for histology using an automatic tissue processor (Leica TP1020, Leica Biosystems, Germany), embedded in paraffin wax, and 5-7 μm sagittal serial sections were cut using an automated rotary microtome (Leica RM2255, Leica Biosystems, Germany). Sections were de-waxed using HISTO-CLEAR™ (National Diagnostics, Atlanta, GA), hydrated through several washes in decreasing concentrations of ethanol (100%, 70% and 50%), and rinsed with water. The sections were stained with Harris hematoxylin, rinsed with water, and treated with Blueing and Clarifier reagents. Sections were rinsed again with water, counterstained with eosin and dehydrated though several washes with 100% ethanol. Finally, sections were cleaned in HISTO-CLEAR™ (National Diagnostics, Atlanta, GA) and mounted with coverslips using PERMOUNT™ mounting medium (Fisher Scientific, Waltham, MA). Representative gubernaculum images were taken using an AXIO Scope.A1 microscope connected to an Axiocam MRc5 camera (Zeiss, Germany). Representative H&E sections of the gubernaculum in embryonic day 18.5 female embryos from pregnant females that were untreated (control), injected with vehicle or with 30 mg/kg of compounds of Examples 158, 153, and 187 were taken. The compound-treated female embryos exhibited a male-like invagination of the gubemaculum, which was absent in the untreated and vehicle controls. This suggests that the RXFP2 agonists are specific and active in vivo. At least 4 embryos were analyzed per treatment group.

### Example 269

This example shows the pharmacokinetic parameters for (S)-8-(3,5-bis(trifluoromethyl)phenyl)-2-(2-(2-iodo-4-(trifluoromethoxy)phenoxy)acetyl)-1,3,4,12a-tetrahydrobenzo[e]pyrazino[1,2-a][1,4]diazepine-6,12 (2H, 11H)-dione (KJW012-055) in an aspect of the invention.

The pharmacokinetic parameters for (S)-8-(3,5-bis(trifluoromethyl)phenyl)-2-(2-(2-iodo-4-(trifluoromethoxy)phenoxy)acetyl)-1,3,4,12a-tetrahydrobenzo[e]pyrazino[1,2-a][1,4]diazepine-6,12 (2H, 11H)-dione (KJW012-055), as prepared in Example 187, were measured in female C57/BL6 mice. The compound was formulated with a carrier comprising 75% polyethylene glycol (PEG) 300 and 25% of 40% aq. 2-hydroxypropyl-beta-cyclodextrin (HPBCD). The results are shown in Table 10.

TABLE 10

| | Route (dosing level) | | |
|---|---|---|---|
| | | | PO_{B} |
| | IV | PO_{A} | (QD × 3) |
| Parameter | (3 mg/kg) | (10 mg/kg) | (10 mg/kg) |
| Total clearance after IV | 15.6 | NA | NA |
| administration-CL_{obs} | | | |
| (mL/min/kg) | | | |
| Half life-t_{1/2} | 6.56 | 3.89 | 5.88 |
| Concentration-C₀ | 3593 | 366 | 389 |
| (mg/L) | | | |
| Maximum serum | 3.254 | NA | NA |
| concentration (μL/L) | | | |
| Area under the curve- | 3191 | 2615 | 3261 |
| AUCₗₐₛₜ (μg-h/mL) | | | |
| Area under the curve- | 3199 | 2647 | 3269 |
| AUC_{inf} (μg-h/mL) | | | |
| Mean residence time- | 3.47 | NA | NA |
| MRTₗₐₛₜ (h) | | | |
| Volume of distribution | 3.38 | NA | NA |
| in steady state- | | | |
| V_{SS}obs (L/kg) | | | |
| Bioavailability-F (%) | NA | 24.8 | 30.7 |

NA: not applicable
PO_{A} is a single administration at 10 mg/kg
PO_{B} is a single administration for 3 consecutive days at 10 mg/kg

### Example 270

This example shows the study of the RXFP2 agonist of Example 187's bone anabolic role in vivo in an aspect of the invention.

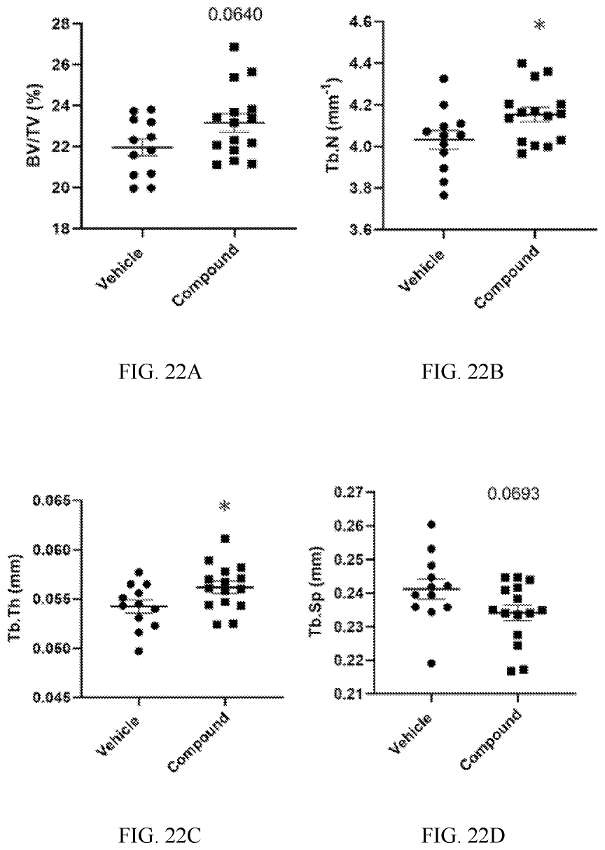

Eight week-old while-type C57BL/6J female mice (Jackson Laboratory) were randomly allocated into 2 treatment groups (vehicle or compound). Mice were treated every other day (Monday, Wednesday, and Friday) with RXFP2 agonist of Example 187 or vehicle formulation (75% PEG 300±25% of 40% aq. HPBCD) for a total of 8 weeks by oral gavage at a concentration of 10 mg/kg using 18G, 1.5-inch, 2 mm ball flexible polytetrafluoroethylene (PTFE) sterile plastic feeding needles. Mice were anesthetized with 2% isoflurane for less than 3 minutes before administrating the treatments to prevent disserts and esophageal lesions. At the end of the treatment, mice were euthanized by isoflurane inhalation overdose, and the lumbar spine was collected, cleaned from muscle tissue, wrapped in a PBS-soaked gauze and frozen at −20° C. until use. All samples were shipped to University of Arkansas for Medical Sciences (UAMS) for analysis. Frozen L3 vertebral bodies were allowed to thaw for at least 2 hours at room temperature before scanning on a Scanco 40 instrument (Scanco Medical, Switzerland) using a slice resolution of 12 μm isotropic voxel size, effective energy of 55 kVp, X-ray tube current of 114 mA, and 200 ms integration time. For the trabecular bone quantification of the L3 vertebrae, the region of interest for analysis comprised the entire vertebral body, including the maximum number of slices possible between both growth plates, applying a grayscale threshold (lower threshold 220, upper threshold 1000) and Gaussian noise filter (sigma 0.8, support 1). The following microCT parameters were calculated and shown in FIG. 22: bone volume per tissue volume (BV/TV, %) (FIG. 22A), trabecular number (Tb.N, mm¹) (FIG. 22B), trabecular thickness (Tb.Th, mm) (FIG. 22C), and trabecular separation (Tb.Sp, mm) (FIG. 22D). The microCT analysis results showed a significant increase in Tb.N and Tb.Th in the compound treated mice compared to vehicle treated mice. A small non-significant improvement in BV/TV and Tb.Sp after compound treatment was also observed. These results suggest that the compound of Example 187 is able to increase bone formation. The results represent the mean±SEM of 12-15 mice per group. *p<0.05 vs vehicle, two tailed unpaired Student's t-test.

### Example 271

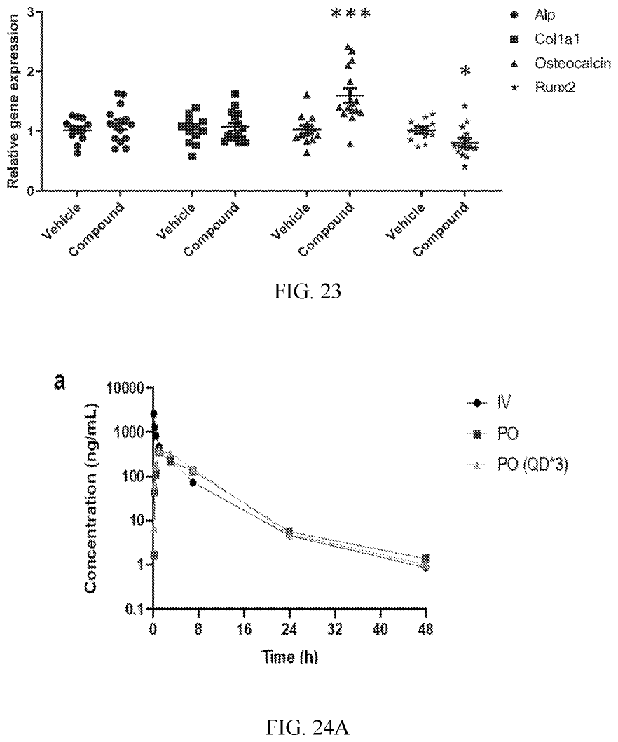

The compound of Example 187 was tested for gene expression levels of osteoblast markers in tibias. FIG. 23 depicts the gene expression levels of osteoblast markers in tibias from WT and INSL3 female mice resulting from treatment with vehicle or compound of Example 187, measured by quantitative RT-PCR. Results represent the mean±SEM of 7 mice per group. *p<0.05, **p<0.01 vs. WT using Student's t-test.

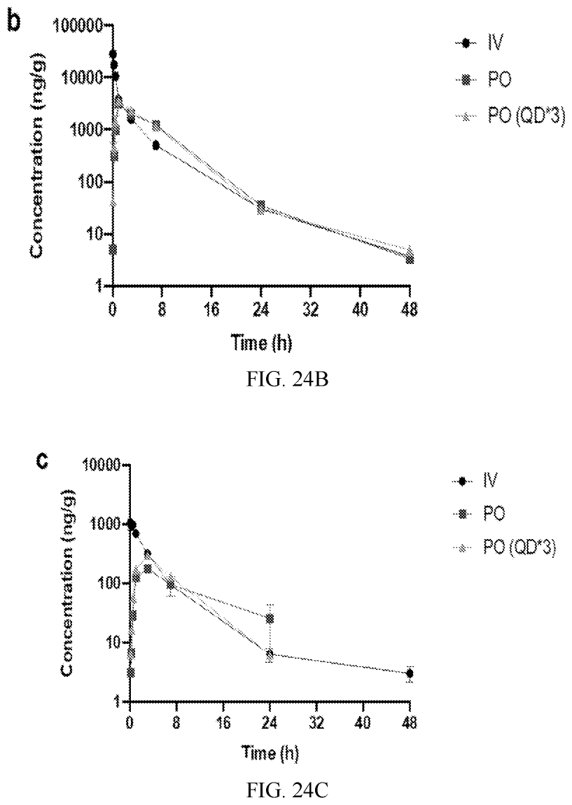

The compound of Example 187 was also tested for its pharmacokinetic properties. FIG. 24 depicts the results of a pharmacokinetic study on the compound of Example 187, after one 3 mg/kg IV administration, one 10 mg/kg PO administration, and three 10 mg/kg PO administrations (QD*3) in female mice. FIG. 24A depicts the plasma profile. FIG. 24B depicts the liver profile and FIG. 24C depicts the bone profiles. The actual concentration (ng/g) is the detected value (ng/mL) multiplied by 4. Drug vehicle is 25% aq. 40% HP-b-CD-75% PEG300. Three mice were used per time point. Results are expressed as the mean±SEM. Table 11a sets forth the plasma profile data depicted in FIG. 24A. Table 11b depicts the liver profile data depicted in FIG. 24B. Table 11c depicts the bone profile data depicted in FIG. 24C.

TABLE 11a
Plasma Profile Data

| | Dosing | | | | | | | AUC_% | | | | |
|---|---|---|---|---|---|---|---|---|---|---|---|---|
| | Level | Cl_obs | | tₘₐₓ | C₀ | AUCₗₐₛₜ | AUC_{Inf} | Extrap | MRT | AUCₗₐₛₜ/D | Vₛₛ_{_{—}}_{obs} | F |
| Route | (mg/kg) | (ml/min/kg} | t_{1/2}(h) | (h) | (ng/mL) | (h*ng/mL) | (h*ng/mL) | (%) | (h) | (h*mg/mL) | (L/kg) | (%) |
| IV | 3 | 15.6 | 6.56 | NA | 3593 | 3191 | 3199 | 0.258 | 3.47 | 1064 | 3.38 | NA |
| PO | 10 | NA | 3.89 | 1.00 | 366 | 2615 | 2647 | 1.20 | NA | 262 | NA | 24.8 |
| PO | 10 | NA | 5.88 | 1.00 | 389 | 3261 | 3269 | 0.265 | NA | 326 | NA | 30.7 |
| (QD*3) | | | | | | | | | | | | |

TABLE 11b
Liver Profile Data

| | Dosing | | | | | | |
|---|---|---|---|---|---|---|---|
| | Level | tₘₐₓ | Cₘₐₓ | AUCₗₐₛₜ | AUC_{Inf} | Cₘₐₓ Ratio | AUCₗₐₛₜ Ratio |
| Route | (mg/kg) | (h) | (ng/g) | (h*ng/g) | (h*ng/g) | (Liver/Plasma) | (Liver/Plasma) |
| IV | 3 | 0.083 | 28040 | 26492 | 26524 | 10.9 | 8.30 |
| PO | 10 | 1.00 | 3214 | 24146 | 24170 | 8.78 | 9.23 |
| PO | 10 | 1.00 | 3570 | 24927 | 24965 | 9.18 | 7.64 |
| (QD*3) | | | | | | | |

TABLE 11c
Bone Profile Data

| | Dosing | | | | | | |
|---|---|---|---|---|---|---|---|
| | Level | tₘₐₓ | Cₘₐₓ | AUCₗₐₛₜ | AUC_{Inf} | Cₘₐₓ Ratio | AUCₗₐₛₜ Ratio |
| Route | (mg/kg) | (h) | (ng/g) | (h*ng/g) | (h*ng/g) | (Bone/Plasma | (Bone/Plasma) |
| IV | 3 | 0.083 | 1072 | 3650 | 3687 | 0.419 | 1.14 |
| PO | 10 | 3.00 | 174 | 1897 | 2190 | 0.475 | 0.725 |
| PO | 10 | 3.000 | 298 | 2539 | 2573 | 0.766 | 0.779 |
| (QD*3) | | | | | | | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-29. (canceled)

30. A compound of formula (I) 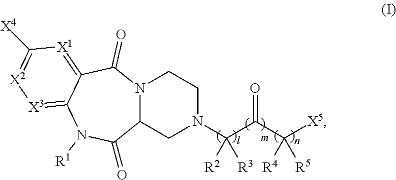
wherein
X¹, X², and X³ are the same or different and each is CH or N;
X⁴ is an optionally substituted aryl or heteroaryl;
X⁵ is selected from the group consisting of 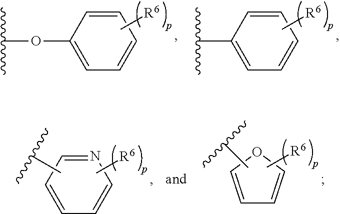
R¹ is hydrogen or alkyl;
R², R³, R⁴, and R⁵ are the same or different and each is hydrogen, alkyl, or halo;
each instance of R⁶ is the same or different and is selected from the group consisting of alkyl, cycloalkyl, heterocycloalkyl, halo, haloalkyl, hydroxy, cyano, nitro, amino, alkylamino, dialkylamino, alkoxy, haloalkoxy, alkylthio, alkylsulfonyl, haloalkylsulfonyl, alkylsulfonamido, carboxylato, —C(═NH)OH, aryl, heteroaryl, and 2-oxoazetidinyl; or
two instances of R⁶ along with the cyclic moiety to which they are bound form phenyl, cycloalkyl, or —O—C(R⁷R⁸)—O—;
R⁷ and R⁸ are the same or different and each is hydrogen, alkyl, or halo;
l is 0 or an integer of 1 to 6;
m is 0 or 1;
n is 0 or an integer of 1 to 6; and
p is 0 or an integer of 1 to 4;
or a pharmaceutically acceptable salt and/or an enantiomer thereof.

31. The compound of claim 30 or a pharmaceutically acceptable salt and/or enantiomer thereof, wherein X¹, X², and X³ are each CH.

32. The compound of claim 30 or a pharmaceutically acceptable salt and/or enantiomer thereof, wherein one of X¹, X², and X³ is N and the remaining two are each CH.

33. The compound of claim 30 or a pharmaceutically acceptable salt and/or enantiomer thereof, wherein R¹ is hydrogen.

34. The compound of claim 30 or a pharmaceutically acceptable salt and/or enantiomer thereof, wherein R², R³, R⁴, and R⁵ are each hydrogen.

35. The compound of claim 30 or a pharmaceutically acceptable salt and/or enantiomer thereof, wherein X⁴ is selected from the group consisting of 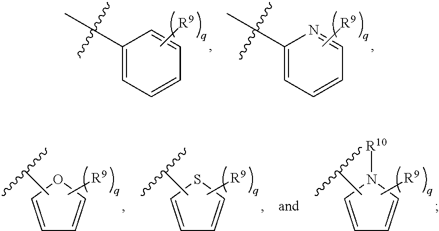
wherein
each instance of R⁹ is the same or different and is selected from the group consisting of alkyl, cycloalkyl, halo, haloalkyl, hydroxy, cyano, nitro, amino, alkylamino, dialkylamino, alkoxy, haloalkoxy, alkylthio, alkylsulfonyl, carboxylato, and —C(═NH)OH; or
two instances of R⁹ along with the cyclic moiety to which they are bound form phenyl, cycloalkyl, or —O—C(R⁷R⁸)—O—;
R¹⁰ is hydrogen or alkyl; and
q is 0 or an integer of 1 to 4.

36. The compound of claim 35 or a pharmaceutically acceptable salt and/or
enantiomer thereof, wherein X⁴ is 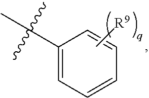
each instance of R⁹ is the same or different and is selected from the group consisting of alkyl, cycloalkyl, halo, haloalkyl, hydroxy, cyano, nitro, haloalkoxy, alkylthio, alkylsulfonyl, and —C(═NH)OH; or
two instances of R⁹ along with the cyclic moiety to which they are bound form —O—C(R⁷R⁸)—O—; and
q is 0 or an integer of 1 to 3.

37. The compound of claim 36 or a pharmaceutically acceptable salt and/or enantiomer thereof, wherein each instance of R⁹ is the same or different and is selected from the group consisting of alkyl, cyclopropyl, fluoro, chloro, trifluoromethyl, and hydroxy; and q is 1 or 2.

38. The compound of claim 35 or a pharmaceutically acceptable salt and/or enantiomer thereof, wherein
X⁴ is 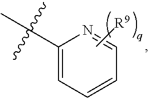
each instance of R⁹ is the same or different and is selected from the group consisting of alkyl, cycloalkyl, halo, haloalkyl, nitro, amino, alkylamino, dialkylamino, and haloalkoxy; and
q is 0 or an integer of 1 to 2.

39. The compound of claim 35 or a pharmaceutically acceptable salt and/or
enantiomer thereof, wherein X⁴ is 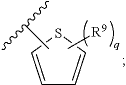
R⁹ is selected from the group consisting of alkyl, halo, and haloalkyl; and
q is 0 or 1.

40. The compound of claim 30, or a pharmaceutically acceptable salt and/or enantiomer thereof, wherein 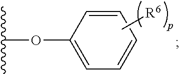
X⁵ is
each instance of R⁶ is the same or different and is selected from the group consisting of alkyl, pyrrolidinyl, halo, haloalkyl, cyano, alkoxy, haloalkoxy, alkylthio, alkylsulfonyl, haloalkylsulfonyl, alkylsulfonamido, and 2-oxoazetidinyl; or
two instances of R⁶ along with the cyclic moiety to which they are bound form phenyl or —O—C(R⁷R⁸)—O—;
R⁷ and R⁸ are the same or different and each is hydrogen or halo;
l is 0; m is 1; n is an integer of 1 to 6; and
p is 0 or an integer of 1 to 4.

41. The compound of claim 40 or a pharmaceutically acceptable salt and/or enantiomer thereof, wherein
(i) p is 1, and R⁶ is fluoro, trifluoromethyl, cyano, trifluoromethoxy, difluoromethoxy, methylthio, methylsulfonyl, trifluoromethylsulfonyl, methylsulfon(methyl)amido, and 2-oxoazetidinyl; or
(ii) p is 2, and each instance of R⁶ is the same or different and is selected from the group consisting of methyl, fluoro, chloro, bromo, iodo, trifluoromethyl, cyano, methoxy, trifluoromethoxy, and difluoromethoxy; or
two instances of R⁶ along with the cyclic moiety to which they are bound form phenyl or —O—CF₂—O—; or
(iii) p is 3, and each instance of R⁶ is the same or different and is selected from the group consisting of methyl, fluoro, trifluoromethyl, cyano, and trifluoromethoxy; or
(iv) p is 4, and each instance of R⁶ is the same or different and is selected from the group consisting of methyl and cyano.

42. The compound of claim 30 or a pharmaceutically acceptable salt and/or enantiomer thereof, wherein
X⁵ is 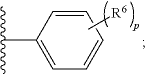
each instance of R⁶ is the same or different and is selected from the group consisting of alkyl, cycloalkyl, heterocycloalkyl, halo, haloalkyl, hydroxy, cyano, nitro, amino, alkylamino, dialkylamino, alkoxy, haloalkoxy, alkylthio, alkylsulfonyl, haloalkylsulfonyl, alkylsulfonamido, carboxylato, —C(═NH)OH, aryl, heteroaryl, and 2-oxoazetidinyl; or
two instances of R⁶ along with the cyclic moiety to which they are bound form phenyl or —O—C(R⁷R⁸)—O—;
R⁷ and R⁸ are the same or different and each is hydrogen, alkyl, or halo; and
either
(i) l is 0; m is 0; and n is an integer of 1 to 6;
(ii) l is 0; m is 1; and n is 0; or
(iii) l is an integer of 1 to 6; m is 1; and n is 0;
and
p is 0 or an integer of 1 to 3.

43. The compound of claim 42 or a pharmaceutically acceptable salt and/or enantiomer thereof, wherein
each instance of R⁶ is the same or different and is selected from the group consisting of hydroxy and alkoxy; or
two instances of R⁶ along with the cyclic moiety to which they are bound form —O—CH₂—O—.

44. The compound of claim 30 or a pharmaceutically acceptable salt and/or enantiomer thereof, wherein
X⁵ is 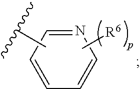
each instance of R⁶ is the same or different and is selected from the group consisting of alkyl, cycloalkyl, heterocycloalkyl, halo, haloalkyl, hydroxy, cyano, nitro, amino, alkylamino, dialkylamino, alkoxy, haloalkoxy, alkylthio, alkylsulfonyl, haloalkylsulfonyl, alkylsulfonamido, carboxylato, —C(═NH)OH, aryl, heteroaryl, and 2-oxoazetidinyl; or
two instances of R⁶ along with the cyclic moiety to which they are bound form phenyl;
l is 0; m is 0; and n is an integer of 1 to 6; and
p is 0 or an integer of 1 to 3.

45. The compound of claim 44 or a pharmaceutically acceptable salt and/or enantiomer thereof, wherein
each instance of R⁶ is the same or different and is selected from the group consisting of halo and haloalkyl; or
two instances of R⁶ along with the cyclic moiety to which they are bound form phenyl; and
p is 0 or an integer of 1 or 2.

46. The compound of claim 30 or a pharmaceutically acceptable salt and/or enantiomer thereof, wherein
X⁵ is 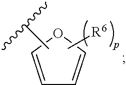
each instance of R⁶ is the same or different and is selected from the group consisting of alkyl, cycloalkyl, heterocycloalkyl, halo, haloalkyl, hydroxy, cyano, nitro, amino, alkylamino, dialkylamino, alkoxy, haloalkoxy, alkylthio, alkylsulfonyl, haloalkylsulfonyl, alkylsulfonamido, carboxylato, —C(═NH)OH, aryl, heteroaryl, and 2-oxoazetidinyl; or
two instances of R⁶ along with the cyclic moiety to which they are bound form phenyl;
l is 0; m is 1; and n is 0; and
p is 0 or an integer of 1 to 3.

47. The compound of claim 46 or a pharmaceutically acceptable salt and/or enantiomer thereof, wherein
each instance of R⁶ is haloalkyl, or
two instances of R⁶ along with the cyclic moiety to which they are bound form phenyl; and
p is 1 or 2.

48. The compound of claim 30 or a pharmaceutically acceptable salt and/or enantiomer thereof, wherein the compound of formula (I) is an S-enantiomer.

49. The compound of claim 30 or a pharmaceutically acceptable salt and/or enantiomer thereof, wherein the compound of formula (I) is an R-enantiomer.

50. The compound of claim 30 selected from the compounds listed in Tables 1, 2, 3, 4, 5, and 6 of the specification, a racemic mixture thereof, or an enantiomer thereof.

51. The compound of claim 30 or a pharmaceutically acceptable salt and/or enantiomer thereof, that is

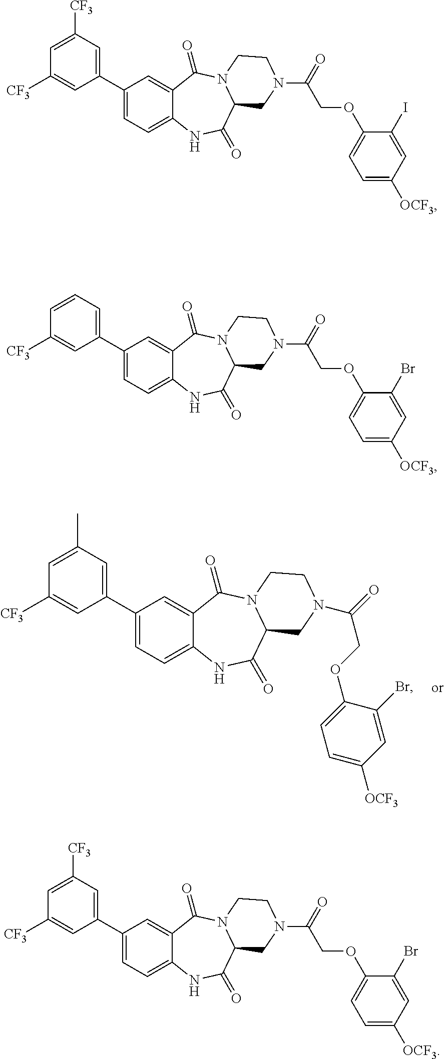 [SMILES: O=C1Nc2ccc(-c3cc(C(F)(F)F)cc(C(F)(F)F)c3)cc2C(=O)N2CCN(C(=O)COc3ccc(OC(F)(F)F)cc3I)C[C@@H]12 ; O=C1Nc2ccc(-c3cccc(C(F)(F)F)c3)cc2C(=O)N2CCN(C(=O)COc3ccc(OC(F)(F)F)cc3Br)C[C@@H]12 ; Cc1cc(-c2ccc3c(c2)C(=O)N2CCN(C(=O)COc4ccc(OC(F)(F)F)cc4Br)C[C@H]2C(=O)N3)cc(C(F)(F)F)c1 ; O=C1Nc2ccc(-c3cc(C(F)(F)F)cc(C(F)(F)F)c3)cc2C(=O)N2CCN(C(=O)COc3ccc(OC(F)(F)F)cc3Br)C[C@@H]12]

52. A pharmaceutical composition comprising the compound of claim 30 or a pharmaceutically acceptable salt and/or enantiomer thereof and at least one carrier.

53. A method of treating a disorder mediated by relaxin family peptide receptor 2 (RXFP2) in a subject, the method comprising administering an effective amount of a compound of claim 30 or a pharmaceutically acceptable salt and/or enantiomer thereof.

54. The method according to claim 53, wherein the disorder mediated by RXFP2 is a bone disorder, hypogonadism, cryptorchidism, polycystic ovary syndrome, cancer, infertility, or an ocular wound.

55. The method according to claim 54, wherein the bone disorder is osteoporosis, osteopenia, or osteogenesis imperfect, and/or the cancer is testicular cancer, prostate cancer, or thyroid cancer.

56. A method of activating a functional activity of relaxin family peptide receptor 2 (RXFP2) in a subject comprising administering to the subject an effective amount of a compound of claim 30 or a pharmaceutically acceptable salt and/or enantiomer thereof.

57. The method of claim 56, wherein the activating results in growing bone or muscle in a subject.
